(19) United States
(12) Patent Application Publication
(10) Pub. No.: US 2025/0319184 A1
(43) Pub. Date: Oct. 16, 2025

(54) CHIMERIC, TRANSMEMBRANE PROTEINS WITH BIDIRECTIONAL SIGNALLING ACTIVITY

(71) Applicant: Gadeta B.V., Utrecht (NL)

(72) Inventors: Laurens Sand, Utrecht (NL); Haakan Norell, Oegstgeest (NL)

(21) Appl. No.: 18/259,031

(22) PCT Filed: Dec. 23, 2021
(86) PCT No.: PCT/EP2021/087591

§ 371 Date: Jun. 22, 2023

(30) Foreign Application Priority Data
Dec. 23, 2020 (EP) 20217164.1
Dec. 23, 2020 (EP) 20217167.4

(51) Int. Cl. A61K 40/31; A61K 40/11; A61K 40/42; C07K 14/705; C07K 14/725

(52) U.S. Cl. CPC A61K 40/31; A61K 40/11; A61K 40/421; A61K 40/4215; A61K 40/4232; C07K 14/7051; C07K 14/70532; C07K 14/70575; C07K 14/70578; A61K 2239/22; C07K 2319/03

(57) ABSTRACT

Disclosed herein are polynucleotides and vectors encoding improved immunotherapeutics, polypeptides encoded by the polynucleotides and/or vectors, cells expressing the polypeptides, and pharmaceutical compositions comprising the polynucleotides, vectors, polypeptides, and/or cells.

### BACKGROUND

Engineered cells hold great potential both for research and therapeutic applications. However, despite increased efforts to generate new and more advanced engineered cells, a number of challenges remain that limit the efficiency of engineered cell production and the efficacy of therapeutic use.

### SUMMARY

In an aspect, there is provided a polynucleotide encoding each of the monomers of a heterodimeric receptor, wherein said polynucleotide comprises at least one nucleic acid encoding a polypeptide other than said monomers inserted between the nucleic acids encoding each of said monomers, and wherein said nucleic acids are operably linked to the same promoter sequence.

In an embodiment, the promoter sequence is selected from the group of EF1α, MSCV, EF1 alpha-HTLV-1 hybrid promoter, Moloney murine leukemia virus (MoMuLV or MMLV), Gibbon Ape Leukemia virus (GALV), murine mammary tumor virus (MuMTV or MMTV), Rous sarcoma virus (RSV), MHC class II, clotting Factor IX, insulin promoter, PDX1 promoter, CD11, CD4, CD2, gp47 promoter, PGK, Beta-globin, UbC, and MND.

In an embodiment, the polynucleotide comprises a nucleotide sequence inserted between each of the nucleic acids which facilitates their co-expression.

In an embodiment, the nucleotide sequence which facilitates the co-expression of the nucleic acids encodes a 2A self-cleaving peptide or is an IRES sequence.

In an embodiment, the 2A self-cleaving peptide is selected from a T2A, a P2A, an E2A, or an F2A peptide.

In an embodiment, the polynucleotide is tricistronic or tetracistronic.

In an embodiment, the heterodimeric receptor is an exogenous antigen-recognition receptor.

In an embodiment, the exogenous antigen-recognition receptor is selected from a B-cell receptor heavy and light chain heterodimer, a Toll-like receptor 1 and 2 heterodimer, a phagocytic receptor Mac-1, a CD94 NKG2C or NKG2E receptor, a T-cell receptor, an αβT-cell receptor, a γδT-cell receptor, and functional fragments thereof

In an embodiment, the exogenous antigen-recognition receptor is an αβT-cell receptor, a γδT-cell receptor, or a functional fragment thereof.

In an embodiment, the polynucleotide comprises A, B, C, or D, wherein:
(A) is a nucleic acid represented by (i)-(ii)-(iii), wherein:
(i) is a nucleic acid encoding an α chain of an αβT-cell receptor or a functional fragment thereof,
(ii) is at least one nucleic acid encoding a polypeptide other than an α or β chain of an αβT-cell receptor or a functional fragment thereof, and;
(iii) is a nucleic acid encoding a β chain of an αβT-cell receptor or a functional fragment thereof, wherein (ii) is inserted between (i) and (iii)
(B) is a nucleic acid represented by (iv)-(v)-(vi), wherein:
(iv) is a nucleic acid encoding a β chain of an αβT-cell receptor or a functional fragment thereof,
(v) is at least one nucleic acid encoding a polypeptide other than an α or β chain of an αβT-cell receptor or a functional fragment thereof, and;
(vi) is a nucleic acid encoding an α chain of an αβT-cell receptor or a functional fragment thereof, wherein (v) is inserted between (iv) and (vi)
(C) is a nucleic acid represented by (vii)-(viii)-(ix), wherein:
(vii) is a nucleic acid encoding a γ chain of a γδT-cell receptor or a functional fragment thereof,
(viii) is at least one nucleic acid encoding a polypeptide other than a γ or δ chain of a γδT-cell receptor or a functional fragment thereof, and;
(ix) is a nucleic acid encoding a δ chain of a γδT-cell receptor or a functional fragment thereof, wherein (viii) is inserted between (vi) and (ix)
(D) is a nucleic acid represented by (x)-(xi)-(xii), wherein:
(x) is a nucleic acid encoding a δ chain of a γδT-cell receptor or a functional fragment thereof,
(xi) is at least one nucleic acid encoding a polypeptide other than a γ or δ chain of a γδT-cell receptor or a functional fragment thereof, and;
(xii) is a nucleic acid encoding a γ chain of a γδT-cell receptor or a functional fragment thereof,
wherein (xi) is inserted between (x) and (xii)

In an embodiment, A, B, C, and/or D are such that:
(i) and (vi) are nucleic acids comprising a nucleotide sequence encoding a polypeptide having at least 60%, 70%, 80%, 90%, 95%, or 100% identity or similarity with an amino acid sequence selected from SEQ ID NOs: 199, 210, 214, 216, 218, and 220, preferably selected from SEQ ID NOs: 210, 216, and 220, and/or;
(iii) and (iv) are nucleic acids comprising a nucleotide sequence encoding a polypeptide having at least 60%, 70%, 80%, 90%, 95%, or 100% identity or similarity with an amino acid sequence selected from SEQ ID NOs: 198, 211, 215, 217, 219, and 221, preferably selected from SEQ ID NOs: 211, 217, and 221, and/or;
(vii) and (xii) are nucleic acids comprising a nucleotide sequence encoding a polypeptide having at least 60%, 70%, 80%, 90%, 95%, or 100% identity or similarity with an amino acid sequence selected from SEQ ID NOs: 85, 86, 87, 89, 91, 93, 94, 95, 96, 101, 104, 106, 108, 110, 112, 113, 115, 117, 119, 121, 123, 125, 127, 129, 130, and 132, preferably selected from SEQ ID NOs: 85, 86, 87, 94, 95, 96, 101, 113, 115, 117, 119, 127, and 130, and/or;
(ix) and (x) are nucleic acids comprising a nucleotide sequence encoding a polypeptide having at least 60%, 70%, 80%, 90%, 95%, or 100% identity or similarity with an amino acid sequence selected from SEQ ID NOs: 82, 83, 84, 88, 90, 92, 97, 98, 99, 100, 102, 103, 105, 107, 109, 111, 114, 116, 118, 120, 122, 124, 126, 128, 131, and 133, preferably selected from SEQ ID NOs: 82, 83, 84, 97, 98, 99, 100, 102, 114,116, 118, 126, and 131.

In an embodiment, the polynucleotide comprises a nucleic acid inserted between the nucleic acids encoding each of the receptor monomers which encodes a chimeric bidirectional signaling transmembrane protein able to transduce at least two intracellular signals, said protein comprising:
an extracellular ligand domain, able to interact with the extracellular domain of its interaction partner
a transmembrane domain, and
a heterologous intracellular signaling domain transducing a first signal after binding of the extracellular ligand domain to its interaction partner,
   wherein the second intracellular signal is transduced via the intracellular domain of the interaction partner.

In an embodiment, the chimeric bidirectional signaling transmembrane protein is not a protein comprising or consisting of the extracellular ligand domain and the transmembrane domain of the ICOSL and the heterologous intracellular signaling domain of 41BB.

In an embodiment, the at least two intracellular signals are inducible.

In an embodiment, the at least two intracellular signals are generated in one single cell.

In an embodiment, the interaction partner comprises:
an extracellular domain able to interact with the extracellular ligand domain of the chimeric protein,
a transmembrane domain, and
an intracellular domain transducing a second signal after binding of the extracellular domain of the interaction partner to the extracellular ligand domain of the chimeric protein.

In an embodiment, the at least two, optionally inducible, intracellular signals contribute to an improvement of a biological parameter and/or function of a cell expressing the chimeric protein and/or an improvement of a biological parameter and/or function induced by such a cell. The biological parameter and/or function may be selected from proliferation, cellular survival, cytotoxicity, antitumor activity, persistence and/or tumor cell killing. In an embodiment, the cell is an immune cell, preferably a T or NK cell.

In an embodiment, the chimeric protein is such that:
a. The extracellular ligand domain is from or derived from a type I transmembrane protein and the heterologous intracellular signaling domain is from or derived from a type II transmembrane protein or
b. The extracellular ligand domain is from or derived from a type II transmembrane protein and the heterologous intracellular signaling domain is from or derived from a type I transmembrane protein.

In an embodiment,
the extracellular ligand domain comprises an amino acid sequence from a tumor necrosis factor superfamily member, a cytokine, a C-type lectin, an immunoglobulin superfamily member, or an antibody or antigen-binding fragment thereof; and
the heterologous intracellular signaling domain comprises an amino acid sequence from a tumor necrosis factor receptor superfamily member, a cytokine receptor, or a C-type lectin receptor.

In an embodiment,
the extracellular ligand domain comprises an amino acid sequence from 41BBL, OX40L, CD86, or RANK, and
the heterologous intracellular signaling domain comprises an amino acid sequence from OX40, 41BB, NKp80, or IL18RAP.

In an embodiment,
the extracellular ligand domain comprises an amino acid sequence from 41BBL, OX40L, CD86, RANK, or CD70, and
the heterologous intracellular signaling domain comprises an amino acid sequence from OX40, 41BB, NKp80, IL18RAP, or IL2RB.

In an embodiment,
(a) the extracellular ligand domain comprises an amino acid sequence from 41BBL and the heterologous intracellular signaling domain comprises an amino acid sequence from OX40, preferably wherein the extracellular ligand domain is from or is derived from a type II transmembrane protein 41BBL and the heterologous intracellular signaling domain is from or is derived from a type I transmembrane protein OX40,
(b) the extracellular ligand domain comprises an amino acid sequence from CD86 and the heterologous intracellular signaling domain comprises an amino acid sequence from OX40, preferably wherein the extracellular ligand domain is from or is derived from a type I transmembrane protein CD86 and the heterologous intracellular signaling domain is from or is derived from a type I transmembrane protein OX40,
(c) the extracellular ligand domain comprises an amino acid sequence from 41BBL and the heterologous intracellular signaling domain comprises an amino acid sequence from NKp80, preferably wherein the extracellular ligand domain is from or is derived from a type II transmembrane protein 41BBL and the heterologous intracellular signaling domain is from or is derived from a type II transmembrane protein NpK80,
(d) the extracellular ligand domain comprises an amino acid sequence from RANK and the heterologous intracellular signaling domain comprises an amino acid sequence from IL18RAP, preferably wherein the extracellular ligand domain is from or is derived from a type I transmembrane protein RANK and the heterologous intracellular signaling domain is from or is derived from a type I transmembrane protein IL18RAP,
(e) the extracellular ligand domain comprises an amino acid sequence from RANK and the heterologous intracellular signaling domain comprises an amino acid sequence from OX40, preferably wherein the extracellular ligand domain is from or is derived from a type I transmembrane protein RANK and the heterologous intracellular signaling domain is from or is derived from a type I transmembrane protein OX40,
(f) the extracellular ligand domain comprises an amino acid sequence from RANK and the heterologous intracellular signaling domain comprises an amino acid sequence from 41BB, preferably wherein the extracellular ligand domain is from or is derived from a type I transmembrane protein RANK and the heterologous intracellular signaling domain is from or is derived from a type I transmembrane protein 41BB,
(g) the extracellular ligand domain comprises an amino acid sequence from OX40L and the heterologous intracellular signaling domain comprises an amino acid sequence from 41BB, preferably wherein the extracellular ligand domain is from or is derived from a type II transmembrane protein OX40L and the heterologous intracellular signaling domain is from or is derived from a type I transmembrane protein 41BB, or
(h) the extracellular ligand domain comprises an amino acid sequence from CD86 and the heterologous intracellular signaling domain comprises an amino acid sequence from IL18RAP, preferably wherein the extracellular ligand domain is from or is derived from a type I transmembrane protein CD86 and the heterologous intracellular signaling domain is from or is derived from a type I transmembrane protein IL18RAP.

In an embodiment,
(a) the extracellular ligand domain comprises an amino acid sequence from 41BBL and the heterologous intracellular signaling domain comprises an amino acid sequence from OX40, preferably wherein the extracellular ligand domain is from or is derived from a type 11 transmembrane protein 41BBL and the heterologous intracellular signaling domain is from or is derived from a type I transmembrane protein OX40,
(b) the extracellular ligand domain comprises an amino acid sequence from CD86 and the heterologous intracellular signaling domain comprises an amino acid sequence from OX40, preferably wherein the extracellular ligand domain is from or is derived from a type I transmembrane protein CD86 and the heterologous intracellular signaling domain is from or is derived from a type I transmembrane protein OX40,
(c) the extracellular ligand domain comprises an amino acid sequence from 41BBL and the heterologous intracellular signaling domain comprises an amino acid sequence from NKp80, preferably wherein the extracellular ligand domain is from or is derived from a type 11 transmembrane protein 41BBL and the heterologous intracellular signaling domain is from or is derived from a type 11 transmembrane protein NpK80,
(d) the extracellular ligand domain comprises an amino acid sequence from RANK and the heterologous intracellular signaling domain comprises an amino acid sequence from IL18RAP, preferably wherein the extracellular ligand domain is from or is derived from a type I transmembrane protein RANK and the heterologous intracellular signaling domain is from or is derived from a type I transmembrane protein IL18RAP,
(e) the extracellular ligand domain comprises an amino acid sequence from RANK and the heterologous intracellular signaling domain comprises an amino acid sequence from OX40, preferably wherein the extracellular ligand domain is from or is derived from a type I transmembrane protein RANK and the heterologous intracellular signaling domain is from or is derived from a type I transmembrane protein OX40,
(f) the extracellular ligand domain comprises an amino acid sequence from RANK and the heterologous intracellular signaling domain comprises an amino acid sequence from 41BB, preferably wherein the extracellular ligand domain is from or is derived from a type I transmembrane protein RANK and the heterologous intracellular signaling domain is from or is derived from a type I transmembrane protein 41BB,
(g) the extracellular ligand domain comprises an amino acid sequence from OX40L and the heterologous intracellular signaling domain comprises an amino acid sequence from 41BB, preferably wherein the extracellular ligand domain is from or is derived from a type 11 transmembrane protein OX40L and the heterologous intracellular signaling domain is from or is derived from a type I transmembrane protein 41BB,
(h) the extracellular ligand domain comprises an amino acid sequence from CD86 and the heterologous intracellular signaling domain comprises an amino acid sequence from IL18RAP, preferably wherein the extracellular ligand domain is from or is derived from a type I transmembrane protein CD86 and the heterologous intracellular signaling domain is from or is derived from a type I transmembrane protein IL18RAP,
(i) the extracellular ligand domain comprises an amino acid sequence from CD70 and the heterologous intracellular signaling domain comprises an amino acid sequence from OX40, preferably wherein the extracellular ligand domain is from or is derived from a type 11 transmembrane protein CD70 and the heterologous intracellular signaling domain is from or is derived from a type I transmembrane protein OX40, or
(j) the extracellular ligand domain comprises an amino acid sequence from 41BBL and the heterologous intracellular signaling domain comprises an amino acid sequence from OX40 and an amino acid sequence from IL2RB, preferably wherein the extracellular ligand domain is from or is derived from a type II transmembrane protein 41BBL and the heterologous intracellular signaling domain is from or is derived from a type I transmembrane protein OX40 and from a type I transmembrane protein IL2RB.

In an embodiment, the chimeric bidirectional signaling transmembrane protein identified under a) is represented by an amino acid sequence having at least 80% identity or similarity with SEQ ID NO: 45, 46, 57, 58, 59, 60, 61, 62, 63, 64, or 65.

In an embodiment, the chimeric bidirectional signaling transmembrane protein identified under a) is represented by an amino acid sequence having at least 80% identity or similarity with SEQ ID NO: 45, 46, 57, 58, 59, 60, 61, 62, 63, 64, 65, 178, or 179.

In an embodiment, the chimeric bidirectional signaling transmembrane protein identified under b) is represented by an amino acid sequence having at least 80% identity or similarity with SEQ ID NO:52, 53, or 73.

In an embodiment, the chimeric bidirectional signaling transmembrane protein identified under c) is represented by an amino acid sequence having at least 80% identity or similarity with SEQ ID NO:47 or 48.

In an embodiment, the chimeric bidirectional signaling transmembrane protein identified under d) is represented by an amino acid sequence having at least 80% identity or similarity with SEQ ID NO:78.

In an embodiment, the chimeric bidirectional signaling transmembrane protein identified under e) is represented by an amino acid sequence having at least 80% identity or similarity with SEQ ID NO: 76.

In an embodiment, the chimeric bidirectional signaling transmembrane protein identified under f) is represented by an amino acid sequence having at least 80% identity or similarity with SEQ ID NO: 77.

In an embodiment, the chimeric bidirectional signaling transmembrane protein identified under g) is represented by an amino acid sequence having at least 80% identity or similarity with SEQ ID NO: 49, 50, or 51.

In an embodiment, the chimeric bidirectional signaling transmembrane protein identified under h) is represented by an amino acid sequence having at least 80% identity or similarity with SEQ ID NO: 71 or 72.

In an embodiment, the chimeric bidirectional signaling transmembrane protein identified under i) is represented by an amino acid sequence having at least 80% identity or similarity with SEQ ID NO: 182 or 183.

In an embodiment, the chimeric bidirectional signaling transmembrane protein identified under j) is represented by an amino acid sequence having at least 80% identity or similarity with SEQ ID NO: 179.

In an embodiment, the chimeric bidirectional signaling transmembrane protein does not contain an ITAM or an intracellular domain from a TCR signaling complex.

In another aspect, there is provided a polynucleotide encoding the chimeric bidirectional signaling transmembrane protein as defined herein.

In another aspect, there is provided a vector comprising a polynucleotide as defined herein. In an embodiment, the vector is a viral vector. In an embodiment, the viral vector is a lentiviral vector.

In another aspect, there is provided a polypeptide encoded by a polynucleotide or by a vector as defined herein.

In an embodiment there is provided, a cell comprising a polynucleotide as defined herein, or a vector as defined earlier herein, preferably wherein said cell expresses a chimeric protein as defined herein, more preferably wherein said cell also expresses the interaction partner.

In an embodiment, there is provided a population of cells, wherein the population of cells comprises at least one cell as defined earlier herein.

In an embodiment, the cells or the population of cells are immune cells, preferably T cells or NK cells. In an embodiment, the population of cells further comprises at least one cell that expresses an exogenous antigen-recognition receptor.

In an embodiment, the population of cells that expresses an exogenous antigen-recognition receptor also expresses the chimeric bidirectional signaling transmembrane protein as defined earlier herein.

In an embodiment, the exogenous antigen-recognition receptor is a chimeric antigen receptor, a T cell receptor, an alpha-beta T cell receptor, or a gamma-delta T cell receptor.

In an embodiment, the population of cells is a population of T cells, preferably alpha-beta T cells that express a gamma-delta T cell receptor.

In an embodiment, the population of cells as defined herein is such that, wherein upon exposure of the cells that express the chimeric bidirectional signaling transmembrane protein as defined herein to cells that express or present an antigen that binds to the exogenous antigen-recognition receptor, proliferation, cellular survival, cytotoxicity, antitumor activity, persistence and/or tumor cell killing of the population of said cells is increased by at least 10% compared to a corresponding population of cells that do not express the chimeric protein.

In an aspect, a chimeric bidirectional signaling transmembrane protein, a polynucleotide, a vector, a cell, or a population of cells as defined earlier herein are for use for treating a disease or a condition wherein the at least two, optionally inducible, intracellular signals contribute to an improvement of a biological parameter and/or function of a cell expressing the chimeric protein and/or an improvement of a biological parameter and/or function induced by such a cell, said biological parameter contributing to the treatment of the disease or condition.

In an aspect, a chimeric bidirectional signaling transmembrane protein, a polynucleotide, a vector, a cell, or a population of cells as defined earlier herein, are for use wherein:
the biological parameter selected from proliferation, cellular survival, cytotoxicity, antitumor activity, persistence and/or tumor cell killing,
the cell is an immune cell and/or
the disease is cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent application contains at least one drawing executed in color. Copies of this patent or patent application with color drawings will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides one illustrative example of MIDIS function. A MIDIS protein is expressed by an engineered cell. Binding of the extracellular ligand domain to an interaction partner induces multi-directional signaling comprising at least one “inside out” signal mediated by an intracellular signaling domain of the interaction partner (signal 1) and at least one “outside-in” signal mediated by the heterologous intracellular signaling domain of the MIDIS protein (signal 2). The first signaling pathway and the second signaling pathway can jointly induce a target biological outcome.

FIG. 2 shows a schematic of the constructs used to introduce gamma-delta TCR and MIDIS proteins of the disclosure. P2A and T2A represent self-cleaving peptides. The diagrams to the right illustrate the domains present extracellularly (top) and intracellularly (bottom), with the horizontal lines representing the membrane/transmembrane domain.

FIG. 3 shows the cytotoxic effects of TEGs co-expressing MIDIS proteins of the disclosure or control proteins. TEGs were co-incubated with HT-29 cells ectopically expressing luciferase-tdTomato at effector to target (E:T) ratio of 1:1. Serial stimulation of TEGs was continued for 3 stimulations (TEGs from donor 1, upper panels) or 5 stimulations (TEGs from donor 2, lower panels). * P<0.05 41BBL-OX40 vs indicated “competitor”.

FIG. 4 shows proliferation of TEGs co-expressing MIDIS proteins of the disclosure or control proteins. TEGs were co-incubated with HT-29 cells at an effector to target (E:T) ratio of 1:1. The effector cells were stained with cell trace violet (CTV), and dilution of the dye was used as a marker for proliferation. * P<0.05 41BBL-OX40 vs indicated “competitor”.

FIG. 5 shows the number of cells expressing the transduced gamma delta TCR after 3 rounds of co-culture stimulation with HT-29 cells (donor 1) or five rounds of co-culture stimulation (donor 2). TEGs co-expressing MIDIS proteins of the disclosure or control proteins were co-incubated with HT-29 cells at effector to target (E:T) ratio of 1:1, and the number of TEGs determined by flow cytometry. * P<0.05 41BBL-OX40 vs indicated “competitor”.

FIG. 6 shows the proportion of TEGs that co-express the exhaustion markers LAG-3 and TIM-3 after 3 stimulations with HT-29 cells (donor 1) or 5 stimulations (donor 2). TEGs co-expressing MIDIS proteins of the disclosure or control proteins were co-incubated with HT-29 cells at effector to target (E:T) ratio of 1:1, and the number of TEGs that co-express the exhaustion markers determined by flow cytometry. * P<0.05 41BBL-OX40 vs indicated “competitor”.

FIG. 7 shows the cytotoxic effects of TEGs co-expressing MIDIS proteins of the disclosure or control proteins TEGs from a third representative donor co-incubated with HT-29, RPMI-8226, and MZ1851 RC target cells. Serial stimulation of TEGs was continued for 3 stimulations (HT-29), 4 stimulations (RPMI-8226) or 5 stimulations (MZ1851 RC). The left panels show cytotoxicity data from the first stimulation, while the right panels show cytotoxicity data from the final stimulation, with PAM treatment.

FIG. 8A shows cytotoxic effects of TEGs co-expressing 41BBL-OX40 MIDIS protein. The TEGs were co-incubated with target HT-29 tumor cells ectopically expressing luciferase-tdTomato at E:T ratio 1:1. TEGs were transferred to plates with fresh target cells after 3 days, and residual target cell viability was measured by luciferase assay.

FIG. 8B shows IFNγ production by TEGs co-expressing 41BBL-OX40 MIDIS protein. The TEGs were co-incubated with target HT-29 tumor cells at E:T ratio 1:1. TEGs were transferred to plates with fresh target cells after 3 days, and IFNγ production was measured by ELISA. * P<0.05 CSD (41BBL-OX40).

FIG. 8C shows cytotoxic effects of TEGs co-expressing 41BBL or 41BBL-OX40 MIDIS protein with γ4δ5TCR or γ4δ5TCR alone. The TEGs were co-incubated with target HT-29 tumor cells ectopically expressing luciferase-tdTomato at E:T ratio 1:1. TEGs were transferred to plates with fresh target cells after 7 days. * P<0.05 (41BBL-OX40 vs “competitor”). Residual target cell viability was measured by luciferase assay.

FIG. 9A shows average radiance over time after administering 1.0×10\hat{ }6 of TEGs to mice harboring HT-29 tumors. 0.5×10\hat{ }6 HT-29 luciferase-tdTomato cells were injected into the flank of NSG mice on day −14 (n=6 per group). On day 0, TEGs expressing a γδ TCR of the disclosure, with or without the 41BBL-OX40 MIDIS protein were systemically administrated. Bioluminescence (BLI) and tumor volume were measured weekly.

FIG. 9B shows tumor volume over time after administering 1.0×10\hat{ }6 of TEGs to mice harboring HT-29 tumors. 0.5×10\hat{ }6 HT-29 luciferase-tdTomato cells were injected into the flank of NSG mice on day −14 (n=6 per group). On day 0, TEGs expressing a γδ TCR of the disclosure, with or without the 41BBL-OX40 MIDIS protein were systemically administrated. Bioluminescence (BLI) and tumor volume were measured weekly.

FIG. 10 shows survival over time after administering 1.0×10\hat{ }6 of TEGs to mice harboring HT-29 tumors. 0.5×10\hat{ }6 HT-29 luciferase-tdTomato cells were injected into the flank of NSG mice on day −14 (n=6 per group). On day 0, TEGs expressing a γδ TCR of the disclosure, with or without the 41BBL-OX40 MIDIS protein were systemically administrated.

FIG. 11A shows a schematic of the constructs used to introduce γδ TCR and MIDIS proteins of the disclosure. P2A and T2A represent self-cleaving peptides. The diagrams to the right illustrate the domains present extracellularly (top) and intracellularly (bottom).

FIG. 11B shows cytotoxic effects of TEGs co-expressing OX40L-41BB MIDIS proteins. The TEGs were co-incubated with MZ1851 RC target cells ectopically expressing luciferase-tdTomato at effector to target (E:T) ratio of 1:1, with transfer to fresh target cells and addition of pamidronate (10 μm) seven days later (new stimulation), and measurement of residual target cell viability by luciferase assay seven days after the second stimulation.

FIG. 12A shows a schematic of the constructs used to introduce γδ TCR and MIDIS proteins of the disclosure. P2A and T2A represent self-cleaving peptides. The diagrams to the right illustrate the domains present extracellularly (top) and intracellularly (bottom).

FIG. 12B shows cytotoxic effects of TEGs co-expressing CD86-OX40 MIDIS proteins. The TEGs were co-incubated with HT-29 target cells ectopically expressing luciferase-tdTomato at effector to target (E:T) ratio of 1:1, with transfer to fresh target cells and addition of pamidronate (10 μm) seven days later (new stimulation), and measurement of residual target cell viability by luciferase assay seven days after the second stimulation * P<0.05 (CD86P276-OX40).

FIG. 13 shows surface expression of gamma-delta TCR and 41BBL 12 days after transduction with MIDIS vectors of the disclosure.

FIG. 14 shows surface expression of gamma-delta TCR and 41BBL 12 days after transduction with MIDIS vectors of the disclosure.

FIG. 15 provides schematics of non-limiting examples of constructs used to introduce MIDIS proteins of the disclosure and additional exogenous antigen-recognition receptors into cells. P2A and T2A represent self-cleaving peptides. The diagrams to the right illustrate the domains present extracellularly (top) and intracellularly (bottom).

FIG. 16 illustrates physiology driven by the MIDIS (41BBL-OX40). The first two panels on the left (γ-eGFP-δ) depict flow cytometry plots without the MIDIS before and after target stimulation. The middle panels illustrate flow cytometry plots of engineered cells comprising constructs expressing 4-1BB ligands with the cytoplasmic portion of the 4-1BB ligands truncated (γ-41BBLmincyto-δ). The right panels illustrate the flow cytometry plots of the engineered cells comprising the MIDIS (e.g. 41BBL with intact cytoplasmic portion to interact with OX-40). As shown by the right panels, the engineered cells comprising the MIDIS had exhibited enhanced effector function and biologic signal.

FIG. 17 provides one illustrative example of MIDIS function when an additional activation is needed to induce signaling. A MIDIS protein is expressed by an engineered immune cell. Binding of the extracellular ligand domain to an interaction partner, induces at least one “inside out” signal mediated by an intracellular signaling domain of the interaction partner (signal 1) and activation of the TCR together with binding of the extracellular ligand domain to an interaction partner induces at least one “outside-in” signal mediated by the heterologous intracellular signaling domain of the MIDIS protein (signal 2).

FIG. 18 shows cytotoxic effects of TEGs co-expressing 41BBL13W-OX40rev MIDIS protein. The TEGs were co-incubated with target HT-29 tumor cells ectopically expressing luciferase-tdTomato at E:T ratio 1:1. TEGs were transferred to plates with fresh target cells after 7 days, and residual target cell viability was measured by luciferase assay and depicted in relative luminescence units. * P<0.05 41BBL-OX40 vs indicated “competitor”. ** P<0.01 41BBL-OX40 vs indicated “competitor”.

FIG. 19A shows a schematic of the constructs used to introduce γδ TCR and CD86 MIDIS proteins of the disclosure with constructs comprising a sequence encoding a CD8-Q8 tag as control protein. P2A and T2A represent self-cleaving peptides. The diagrams to the right illustrate the domains present extracellularly (top) and intracellularly (bottom).

FIG. 19B shows a schematic of the constructs used to introduce γδ TCR and RANK MIDIS proteins of the disclosure with constructs comprising a sequence encoding eGFP as control protein and constructs encoding γδ TCR alone as further controls. P2A and T2A represent self-cleaving peptides. The diagrams to the right illustrate the domains present extracellularly (top) and intracellularly (bottom).

FIG. 20A provides schematics of non-limiting examples of constructs used to introduce expression of MIDIS proteins of the disclosure and additional exogenous antigen-recognition receptors into cells. T2A represents self-cleaving peptides. In addition, it shows expression of exogenous antigen-recognition receptors and MIDIS or control proteins by alpha-beta T cells measured by flow cytometry (bottom panels). Alpha-beta T cells were transduced with indicated constructs and stained with anti-Fab antibody after 12 days production to assess surface expression and therewith inclusion of both vectors containing 41BBL (y-axis) or CD19.BB.Z (x-axis), depicted in the panels below. % positive expression is shown in the quadrants.

FIG. 20B shows cytotoxic effects of CAR-T cells co-expressing anti CD19BBz CAR (19BBz) with or without 41BBL-OX40 MIDIS or control proteins. The CAR-T cells were co-incubated with CD19 (NALM6) positive target cells ectopically expressing luciferase-tdTomato at effector to target (E:T) ratio of 1:1, with serial transfer to fresh target cells every three days, and measurement of residual target cell viability by luciferase assay after first (left) or fifth (right) stimulation and depicted in relative luminescence units (RLU). * P<0.05 41BBL-OX40 vs indicated “competitor”. ** P<0.01 41BBL-OX40 vs indicated “competitor”.

FIG. 21A provides schematics of non-limiting examples of the constructs used to introduce expression of γδ TCR and MIDIS proteins of the disclosure. P2A and T2A represent self-cleaving peptides. The diagrams to the right illustrate the domains present extracellularly (top) and intracellularly (bottom).

FIG. 21B shows surface expression of Jurkat T cells co-expressing MIDIS proteins. Jurkat T cells with or without MIDIS or control proteins were stained for CD70 (x-axis) and γδ TCR (y-axis). Expression was determined by flow cytometry. Jurkat T cells were transduced with fixed MOI of vectors included in the disclosure. A week after transduction surface expression of CD70 and γδ TCR expression were assessed as outlined in Example 1; percentage shows positive CD70 expression.

FIG. 22A provides schematics of non-limiting examples of constructs used to introduce expression of γδ TCR and MIDIS into cells, including MIDIS with multiple signaling domains, and a control without MIDIS. P2A and T2A represent self-cleaving peptides. The diagrams to the right illustrate the domains present extracellularly (top) and intracellularly (bottom).

FIG. 22B shows surface expression of TEGs co-expressing MIDIS proteins. TEGs with or without MIDIS or control proteins were stained for 41BBL and γδ TCR. Expression was determined by flow cytometry. After 12 day production as outlined in Example 1, alpha-beta T cells were stained for 41BBL (x-axis) and γδ TCR (y-axis) to assess surface expression. % positive expression is shown in the quadrants of depicted panels (panel titles correspond to the constructs of FIG. 22A).

FIG. 23 shows effects on expansion of γδ T cells co-expressing MIDIS proteins. γδ T cells with or without MIDIS or control proteins were expanded for 22 Days with TransAct (left) or plate bound anti γδ TCR with anti CD28 antibodies (right). Expansion was measured by counting cells at Day 0 and Harvest day.

FIG. 24A shows a schematic of tricistronic constructs used in Example 14 to introduce expression of a gamma-delta TCR and additional proteins of the disclosure with varying positions for the gamma-chain-encoding and delta-chain-encoding nucleic acids. P2A and T2A represent self-cleaving peptides.

FIG. 24B shows surface expression of gamma-delta TCR and endogenous alpha-beta TCR of αβT-cells transduced with multicistronic vectors of the disclosure, measured by flow cytometry.

FIG. 25A shows surface expression of a defined gamma-delta TCR (SEQ ID NO: 90 and 91) by percentage TEGs of all viable T cells corrected per donor. * P<0.05 (γ-eGFP-δ vs “competitor”).

FIG. 25B shows donor corrected median fluorescence intensity (MFI) of a defined gamma delta TCR (SEQ ID NO: 90 and 91) surface at 8-day production after transduction with multicistronic vectors of the disclosure. * P<0.05 (γ-eGFP-δ vs “competitor”).

FIG. 25C shows donor corrected % γδ TCR+αβTCR− of all viable T cells. Alpha beta T cells were transduced with a defined gamma delta TCR (SEQ ID NO: 90 and 91) and stained for γδ TCR and αβ TCR at 8-day production after transduction with multicistronic vectors of the disclosure. * P<0.05 (γ-eGFP-δ vs “competitor”).

FIG. 26A shows surface expression of a defined gamma-delta TCR (SEQ ID NO: 111 and 112) as percentage TEGs of all viable T cells corrected per donor. * P<0.05 (γ-eGFP-δ vs “competitor”).

FIG. 26B shows donor corrected median fluorescence intensity (MFI) of a defined gamma delta TCR (SEQ ID NO: 111 and 112) surface at 8-day production after transduction with multicistronic vectors of the disclosure. * P<0.05 (γ-eGFP-δ vs “competitor”).

FIG. 27A shows a schematic of tricistronic constructs used in Example 14 to introduce expression of a gamma delta TCR and additional proteins 41BBL-OX40 MIDIS (Panel (i)) or CD8-Q8 (Panel (ii)) of the disclosure, with varying positions for the gamma-chain-encoding and delta-chain-encoding nucleic acids. P2A and T2A represent self-cleaving peptides.

FIG. 27B shows donor corrected median fluorescence intensity (MFI) of a defined gamma delta TCR (SEQ ID NO: 90 and 91) surface at 8-day production after transduction with vectors of the disclosure comprising a 41BBL-OX40 encoding nucleic acid. * P<0.05 (γ-eGFP-δ vs “competitor”).

FIG. 27C shows donor corrected % γδ TCR⁺αβ TCR⁻ of all viable T cells. Alpha beta T cells were transduced with a defined gamma delta TCR (SEQ ID NO: 90 and 91) and stained for γδ TCR and αβ TCR at 8-day production after transduction with vectors of the disclosure comprising a 41BBL-OX40 encoding nucleic acid. * P<0.05 (γ-eGFP-δ vs “competitor”).

FIG. 27D shows donor corrected median fluorescence intensity (MFI) of a defined gamma delta TCR (SEQ ID NO: 90 and 91) surface at 8-day production after transduction with vectors of the disclosure comprising a Q8 encoding nucleic acid. * P<0.05 (γ-eGFP-δ vs “competitor”).

FIG. 27E shows donor corrected % γδ TCR⁺αβ TCR⁻ of all viable T cells. Alpha beta T cells were transduced with a defined gamma delta TCR (SEQ ID NO: 90 and 91) and stained for γδ TCR and αβ TCR at 8 day production after transduction with vectors of the disclosure comprising a CD8-Q8 encoding nucleic acid. * P<0.05 (γ-eGFP-δ vs “competitor”).

FIG. 28A shows a schematic of tetracistronic constructs used in Example 14 to introduce expression of gamma-delta TCR and additional proteins 41BBL-OX40 MIDIS and eGFP of the disclosure with varying positions for the gamma-chain-encoding and delta-chain-encoding nucleic acids. P2A and T2A represent self-cleaving peptides.

FIG. 28B shows surface expression of a defined gamma-delta TCR (SEQ ID NO: 90 and 91) from a tetracistronic construct by percentage TEGs of all viable T cells corrected per donor. * P<0.05 (γ-eGFP-δ vs “competitor”).

FIG. 28C shows donor corrected % γδ TCR⁺αβ TCR⁻ of all viable T cells. TEGs were transduced with a defined gamma delta TCR (SEQ ID NO: 90 and 91) and stained for γδ TCR and ap TCR at 8 day production after transduction with tetracistronic vectors of the disclosure comprising a 41BBL-OX40 encoding nucleic acid. * P<0.05 (γ-41BBL-OX40-eGFP-δ vs “competitor”).

FIG. 29A shows a schematic of tricistronic constructs used in Example 15 to introduce expression of a defined gamma-delta TCR and additional proteins. P2A and T2A represent self-cleaving peptides.

FIG. 29B shows cytotoxic effects of TEGs expressing a defined gamma delta TCR (SEQ ID NO: 90 and 91) with varying positions for gamma- and delta-chain encoding nucleic acids in the constructs, compared untransduced T cells (UNTR). The TEGs were co-incubated with target HT-29 tumor cells ectopically expressing luciferase-tdTomato at E:T ratio 1:1 (donor 2) and pamidronate (10 μm) for 3 days. * P<0.05 (γ-eGFP-δ vs “competitor”). Residual target cell viability was measured by luciferase assay.

FIG. 29C shows IFNγ production by TEGs expressing a defined gamma delta TCR (SEQ ID NO: 90 and 91) with varying positions for gamma- and delta-chain encoding nucleic acids in the constructs. The TEGs were co-incubated with target HT-29 tumor cells at E:T ratio 1:1 (donor 2) and pamidronate (10 μm) for 3 days. IFNγ production was measured by ELISA. * P<0.05 (γ-eGFP-δ vs “competitor”).

FIG. 30A shows cytotoxic effects of TEGs expressing a defined gamma delta TCR (SEQ ID NO: 90 and 91) with varying positions for gamma- and delta-chain encoding nucleic acids in the constructs, compared to untransduced T cells (UNTR). The TEGs were co-incubated with target HT-29 tumor cells ectopically expressing luciferase-tdTomato at E:T ratio 1:1 (donor 3) for 3 days and addition of pamidronate (10 μm). * P<0.05 (δ-eGFP-γ vs “competitor”). Residual target cell viability was measured by luciferase assay.

FIG. 30B shows IFNγ production by TEGs expressing a defined gamma delta TCR (SEQ ID NO: 90 and 91) with varying positions for gamma- and delta-chain encoding nucleic acids in the constructs. The TEGs were co-incubated with target HT-29 tumor cells at E:T ratio 1:1 (donor 3) and pamidronate (10 μm) for 3 days. IFNγ production was measured by ELISA. * P<0.05 (δ-eGFP-γ vs “competitor”).

FIG. 30C shows cytotoxic effects of TEGs expressing a defined gamma delta TCR (SEQ ID NO: 111 and 112) with varying positions for gamma- and delta-chain encoding nucleic acids in the constructs, compared to untransduced T cells (UNTR). The TEGs were co-incubated with target RKO tumor cells ectopically expressing luciferase-tdTomato at E:T ratio 0.11:1 (donor 1) for 3 days. * P<0.05 (γ-eGFP-δ vs “competitor”). Residual target cell viability was measured by luciferase assay.

FIG. 31A shows cytotoxic effects of TEGs co-expressing a defined gamma delta TCR (SEQ ID NO: 90 and 91) and 41BBL-OX40 MIDIS with varying positions for gamma- and delta-chain encoding nucleic acids in the constructs compared to untransduced T cells (UNTR). The TEGs were co-incubated with target HT-29 tumor cells ectopically expressing luciferase-tdTomato at E:T ratio 0.3:1 (donor 1) and pamidronate (10 μm) for 3 days. * P<0.05 (γ-41BBLOX40-δ vs “competitor”). Residual target cell viability was measured by luciferase assay.

FIG. 31B shows cytotoxic effects of TEGs co-expressing a defined gamma delta TCR (SEQ ID NO: 90 and 91) and CD8-Q8 with varying positions for gamma- and delta-chain encoding nucleic acids in the constructs, compared untransduced T cells (UNTR). The TEGs were co-incubated with target HT-29 tumor cells ectopically expressing luciferase-tdTomato at E:T ratio 0.3:1 (donor 2) and pamidronate (10 μm) for 3 days. * P<0.05 (γ-41BBLOX40-δ vs “competitor”). Residual target cell viability was measured by luciferase assay.

FIG. 32A shows a schematic of tricistronic constructs used in Example 16 to introduce expression of a defined gamma delta TCR and additional proteins 41BBL-OX40 MIDIS or eGFP. P2A and T2A represent self-cleaving peptides.

FIG. 32B shows cytotoxic effects of TEGs co-expressing a defined gamma delta TCR (SEQ ID NO: 90 and 91) and 41BBL-OX40 with varying positions for gamma- and delta-chain encoding nucleic acids in the constructs, compared untransduced T cells (UNTR). The TEGs were co-incubated with target HT-29 tumor cells ectopically expressing luciferase-tdTomato at E:T ratio 0.3:1 (donor 2) and pamidronate (10 μm) for 3 days. ** P<0.01 (γ-41BBLOX40-δ vs “6-41BBLOX40-γ”) * P<0.05 (6-41BBLOX40-γ vs “UNTR”). Residual target cell viability was measured by luciferase assay.

FIG. 32C shows cytotoxic effects of TEGs co-expressing a defined gamma delta TCR (SEQ ID NO: 111 and 112) and eGFP with varying positions for gamma- and delta-chain encoding nucleic acids in the constructs, compared untransduced T cells (UNTR). The TEGs were co-incubated with target RKO tumor cells ectopically expressing luciferase-tdTomato at E:T ratio 3:1 (donor 3) for 3 days. * P<0.05 (γ-41BBLOX40-δ vs “δ-41BBLOX40-γ”) or (δ-41BBLOX40-γ vs “UNTR”). Residual target cell viability was measured by luciferase assay.

FIG. 33A shows a schematic of tricistronic constructs used in Example 17 to introduce expression of a defined alpha-beta TCR and additional protein eGFP. P2A and T2A represent self-cleaving peptides.

FIG. 33B shows ratio of functional αβTCR expressed at the cell surface compared to intracellular αβTCR expressed by the cell. Jurkat 76 T cells were transduced with defined αβTCR and eGFP. Three days after transduction cells were stained for surface expressed αβTCR by anti-CD3ε followed by fixation and staining of αβTCR. * P<0.05 (β-eGFP-α vs “competitor”)

FIG. 33C shows a schematic of tricistronic constructs used in Example 17 to introduce expression of a defined alpha-beta TCR and additional protein eGFP with varying order for the beta- and alpha-encoding nucleic acids. P2A and T2A represent self-cleaving peptides.

FIG. 33D shows ratio of functional αβTCR expressed at the cell surface compared to intracellular αβTCR expressed by the cell. Jurkat 76 T cells were transduced with defined αβTCR and eGFP. Three days after transduction cells were stained for surface expressed αβTCR by anti-CD3ε followed by fixation and staining of αβTCR. ** P<0.01 (β-eGFP-α vs “competitor”).

### DETAILED DESCRIPTION

Engineered cells hold great potential both for research and therapeutic applications. For example, certain engineered immune cells have provided landmark advances in the treatment of some types of cancer for which no effective treatments were previously available. However, despite increased efforts to generate new and more advanced engineered cells, a number of challenges remain that limit success in the field. Examples of these challenges include difficulties in generating sufficient numbers of the desired engineered cells, limited proliferative ability or lifespan of the engineered cells, limited fitness of the engineered cells, limited induction of effector function upon antigen recognition, and exhaustion.

Disclosed herein are multi-directional signal transducer (MIDIS) proteins that can enhance multiple aspects of engineered cell manufacturing and clinical applications. MIDIS proteins are engineered fusion proteins that contain an extracellular ligand domain that binds to an interaction partner, a transmembrane domain, and a heterologous intracellular signaling domain (from or derived from a different protein than the extracellular ligand domain). When the extracellular ligand binds to its interaction partner, multi-directional signaling is induced that comprises at least one “outside-in” signal mediated by the heterologous intracellular signaling domain of the MIDIS protein, and at least one “inside-out” signal mediated by an intracellular signaling domain of the interaction partner. Throughout the application, the expression “MIDIS protein” may be replaced by the expression “chimeric bidirectional signaling transmembrane protein” as later described herein.

The ability of MIDIS proteins to induce combinations of signaling pathways in both directions is shown to induce a range of target biological outcomes and functions, for example, enhanced cellular proliferation, enhanced cellular survival, and greater magnitude and persistence of immune effector functions, such as cytotoxicity and production of inflammatory mediators. The wording “target biological outcome” or “biological outcome” may be replaced by “biological parameter”.

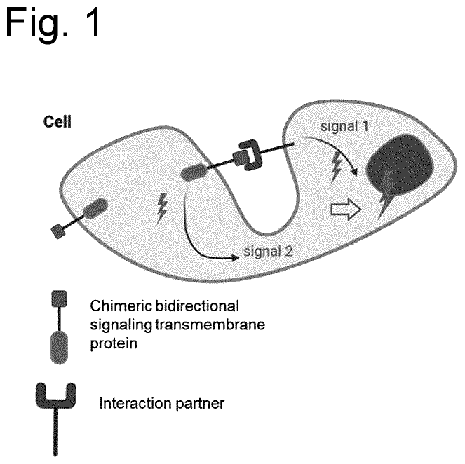

FIG. 1 provides one illustrative example of MIDIS function. A MIDIS protein is expressed by an engineered cell (cell 1). Binding of the extracellular ligand domain to an interaction partner induces multi-directional signaling comprising at least one “inside out” signal mediated by an intracellular signaling domain of the interaction partner (signal 1) and at least one “outside-in” signal mediated by the heterologous intracellular signaling domain of the MIDIS protein (signal 2). The first signaling pathway and the second signaling pathway can jointly induce a target biological outcome. In some embodiments, the multi-directional signaling is or comprises bi-directional signaling, e.g., one signaling pathway mediated by the heterologous intracellular signaling domain of the MIDIS protein and one signaling pathway mediated by the intracellular signaling domain of the interaction partner. In some embodiments, the multi-directional signaling comprises multi-dimensional signaling, e.g., more than one signaling pathway mediated by the heterologous intracellular signaling domain of the MIDIS protein and/or more than one signaling pathway mediated by the intracellular signaling domain of the interaction partner, as disclosed herein.

The multi-directional signaling can modulate biological parameters and/or functions in/of the cell expressing the chimeric bidirectional signaling transmembrane protein to achieve the target biological outcome as disclosed herein. In other words, the “at least two intracellular signals” can contribute to an improvement of a biological parameter of a cell expressing the chimeric bidirectional signaling transmembrane protein and/or an improvement of a biological parameter induced by such cell. Depending on the combination of signaling pathways and cell types, various biological functions and/or parameters can be modulated, including but not limited to cellular proliferation, cellular survival, magnitude of immune effector function, duration of immune effector function, a cytotoxic response (e.g., against a cancer cell), an anti-cancer response, cellular differentiation, cellular dedifferentiation, and cellular transdifferentiation. One or multiple biological functions and/or parameters of the cell may be modulated/improved. Multiple biological functions and/or parameters may be modulated, for example, any combination of induced or reduced biological functions and/or parameters that contributes to a target biological outcome. In this context, a target biological outcome may be the treatment, cure of a disease or condition as later explained herein. For example, multiple biological functions can be induced in a cell and/or a biological function can be induced and another one can be reduced.

Certain MIDIS proteins (or chimeric bidirectional signaling transmembrane protein) disclosed herein combine an amino acid sequence from a type I transmembrane protein with an amino acid sequence from a type II transmembrane protein. In some embodiments, such MIDIS proteins exhibit surprising and unexpected effects, as type I and type II transmembrane proteins cannot be readily combined into a functional protein. For example, many attempts to fuse an amino acid sequence from a type I transmembrane protein to an amino acid sequence from type II transmembrane protein fail to yield a functional protein, for example, due to an altered N-terminal or C-terminal location of one of the amino acid sequences, inability of the resulting protein to adopt a functional conformation, tertiary structure, transmembrane orientation, or a combination thereof.

In some examples provided herein, the extracellular ligand domain comprises an amino acid sequence that is from or derived from a type I transmembrane protein, and the heterologous intracellular signaling domain comprises an amino acid sequence that is from or derived from a type II transmembrane protein. In some examples provided herein, the extracellular ligand domain comprises an amino acid sequence that is from or derived from a type II transmembrane protein, and the heterologous intracellular signaling domain comprises an amino acid sequence that is from or derived from a type I transmembrane protein (for example, an extracellular ligand domain from 41BBL, and an intracellular signaling domain from OX40).

In some embodiments, part or all of an extracellular ligand domain and/or a heterologous intracellular signaling domain of a MIDIS (or chimeric bidirectional signaling transmembrane protein) comprises an amino acid sequence that is inverted compared to a wild type amino acid sequence (i.e. expressed as a retro-protein). In some embodiments, such MIDIS proteins (or chimeric bidirectional signaling transmembrane protein) exhibit surprising and unexpected effects, as in many cases retro-proteins do not retain the functionality of the parent protein, e.g., due to a failure to adopt a functional conformation and/or tertiary structure.

In some embodiments, a MIDIS protein (or chimeric bidirectional signaling transmembrane protein) combines an amino acid sequence from a type I transmembrane protein with an amino acid sequence from a type II transmembrane protein, and contains at least one amino acid sequence that is inverted compared to a wild type amino acid sequence. Functionality of such a MIDIS protein can be surprising and unexpected based on a lack of expectation of success combining sequences from type I and type II transmembrane proteins into a functioning fusion protein, and a lack of expectation of success in obtaining a functional retro-protein domain.

### I. Definitions

An “extracellular ligand domain” of a MIDIS protein is capable of binding to an interaction partner, and induces signaling mediated by an intracellular domain of the interaction partner upon binding. Throughout the application, the expression “MIDIS protein” may be replaced by the expression “chimeric bidirectional signaling transmembrane protein”. An extracellular ligand domain can comprise an amino acid sequence that is from or derived from a protein disclosed herein, for example, a wild type protein, a variant derived from a wild type protein with one or more amino acid insertions, deletions, and/or substitutions relative to the wild type protein sequence, or another protein disclosed herein. An extracellular ligand domain can be from or derived from, for example, a protein that is expressed on a cell surface, a tumor necrosis factor superfamily member, an immune co-receptor ligand, an immunoglobulin superfamily member, a cytokine, a naturally-occurring or a synthetic peptide ligand of the interaction partner, a metal-dependent hydrolase family member that binds to the interaction partner, or an antigen-binding protein disclosed herein, such as an antigen-binding fragment of an antibody, a single chain variable fragment (scFv), a DARPin, or other antigen-binding proteins disclosed herein. Non-limiting examples of extracellular ligand domains include amino acid sequences from or derived from 41BBL, OX40L, CD86, RANK, and CD70. An extracellular ligand domain may be or may be derived from a type I or a type II transmembrane protein.

In an embodiment, an extracellular ligand domain is a tumor necrosis factor superfamily member or a molecule derived thereof and is derived from a type II transmembrane protein and is therefore a type II molecule.

In an embodiment, an extracellular ligand domain is an immunoglobulin superfamily member or is derived thereof and is derived from a type I transmembrane protein and is therefore a type I molecule.

A “heterologous intracellular signaling domain” of a MIDIS protein refers to an intracellular signaling domain present in a MIDIS protein that is from or derived from a different protein than the extracellular ligand domain. A signaling pathway mediated by the heterologous intracellular signaling domain is induced upon binding of the extracellular ligand domain to an interaction partner. The heterologous intracellular signaling domain can be from or derived from, for example, a protein that is a type I or type II transmembrane protein. The presence of a heterologous intracellular signaling domain in a MIDIS protein does not necessarily preclude the presence of an intracellular signaling domain from the same protein as the extracellular ligand domain, but indicates that at least one intracellular signaling domain from a different protein is present in the MIDIS. For example, in some cases, a heterologous intracellular signaling domain can be appended to a full length wild type transmembrane protein, where the full length wild type transmembrane protein includes the extracellular ligand domain and a (non-heterologous) intracellular signaling domain. In other cases, the MIDIS does not contain an intracellular signaling domain from the same protein as the extracellular ligand domain. A heterologous intracellular signaling domain can comprise an amino acid sequence that is from a protein disclosed herein, for example, a wild type protein, a variant derived from a wild type protein with one or more amino acid insertions, deletions, and/or substitutions relative to the wild type protein sequence, or another protein disclosed herein. A heterologous intracellular signaling domain can be from or derived from, for example, a transmembrane protein, a tumor necrosis factor receptor superfamily member, a receptor tyrosine kinase, a cytokine receptor, a C-type lectin receptor, a cytoplasmic protein that participates in signaling pathway, or any other suitable protein disclosed herein. Non-limiting examples of heterologous intracellular signaling domains include amino acid sequences from or derived from 41BB, OX40, NKp80, or IL18RAP.

An “interaction partner” of a MIDIS protein is present on the surface of a cell and is capable of binding to the extracellular ligand domain of the MIDIS. Binding of the interaction partner to the extracellular ligand domain of the MIDIS induces signaling mediated by an intracellular domain of the interaction partner. Therefore, in an embodiment, the interaction partner comprises:
an extracellular domain able to interact with the extracellular ligand domain of the chimeric bidirectional signaling transmembrane protein,
a transmembrane domain, and
an intracellular domain transducing a second signal after binding of the extracellular domain of the interaction partner to the extracellular ligand domain of the chimeric bidirectional signaling transmembrane protein.

The terms “nucleic acid”, “nucleic acid molecule”, and “polynucleotide” are used interchangeably herein.

A polynucleotide described herein may comprise one or more nucleic acids encoding a polypeptide operably linked to (i.e., in a functional relationship with) a regulatory sequence, for example a promoter. Such a polynucleotide may alternatively be referred to herein as “nucleic acid construct” or “construct”.

As used herein, a regulatory sequence refers to any genetic element that is known to the skilled person to drive or otherwise regulate expression of nucleic acids in a cell. Such sequences include without limitation promoters, transcription terminators, enhancers, repressors, silencers, kozak sequences, polyA sequences, and the like. A regulatory sequence can, for example, be inducible, non-inducible, constitutive, cell-cycle regulated, metabolically regulated, and the like. A regulatory sequence may be a promoter. Non-limiting examples of suitable promoters include EF1α, MSCV, EF1 alpha-HTLV-1 hybrid promoter, Moloney murine leukemia virus (MoMuLV or MMLV), Gibbon Ape Leukemia virus (GALV), murine mammary tumor virus (MuMTV or MMTV), Rous sarcoma virus (RSV), MHC class II, clotting Factor IX, insulin promoter, PDX1 promoter, CD11, CD4, CD2, gp47 promoter, PGK, Beta-globin, UbC, MND, and derivatives (i.e. variants) thereof. Examples of these promoters are further described in Poletti and Mavilio (2021), Viruses 13:8; 1526, Kuroda et al. (2008), J Gene Med 10(11):1163-1175, Milone et al. (2009), Mol Ther 17:8; 1453-1464, and Klein et al. (2008), J Biomed Biotechnol 683505, all of which are incorporated herein by reference in their entireties.

A polynucleotide described herein may be multicistronic. “Multicistronic” (alternatively referred to herein as “polycistronic”) can refer to the transcription of the polynucleotide resulting in an mRNA from which at least two distinct polypeptides are translated. This, for example, may be achieved by a polynucleotide comprising at least two nucleic acids encoding distinct polypeptides, preferably operably linked to the same promoter. In some embodiments, at least two, at least three, at least four, at least five, or at least six, preferably at least three or at least four, polypeptides are expressed by a polynucleotide described herein. A polynucleotide described herein may be tricistronic (i.e., three distinct polypeptides may be expressed). A polynucleotide described herein may be tetracistronic (i.e., four distinct polypeptides may be expressed). A multicistronic polynucleotide may comprise additional nucleotide sequences facilitating the co-expression of the encoded polypeptides, which are described later herein. A polynucleotide may be comprised in a vector as described later herein.

A “wild type” protein amino acid sequence can refer to a sequence that is naturally occurring and encoded by a germline genome. A species can have one wild type sequence, or two or more wild type sequences (for example, with one canonical wild type sequence and one or more non-canonical wild type sequences). A wild type protein amino acid sequence can be a mature form of a protein that has been processed to remove N-terminal and/or C-terminal residues, for example, to remove a signal peptide.

An amino acid sequence that is “derived from” a wild type sequence or other amino acid sequence disclosed herein can refer to an amino acid sequence that differs by one or more amino acids compared to the reference amino acid sequence, for example, containing one or more amino acid insertions, deletions, or substitutions as disclosed herein.

Within the context of the application a protein is represented by an amino acid sequence and correspondingly a nucleic acid molecule or a polynucleotide is represented by a nucleic acid sequence. Identity and similarity between sequences: Throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each amino acid sequence described herein by virtue of its identity or similarity percentage with a given amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of ths sequence), maximizing the number of matches and minimizes the number of gaps. Default settings can be used and preferred program is Needle for pairwise alignment (in an embodiment, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an embodiment, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used)

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged | Ser (S), Thr (T), Asn (N), and |
| Residues | Gln (Q) |
| Aliphatic Uncharged | Gly (G), Ala (A), Val (V), Leu (L), |
| Residues | and Ile (I) |
| Non-polar Uncharged | Cys (C), Met (M), and Pro (P) |
| Residues | |
| Aromatic Residues | Phe (F), Tyr (Y), and Trp (W) |

Alternative conservative amino acid residue substitution classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, |
| | M, R, T, V, W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, |
| formation | R, S, P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to lie or Val; Lys to Arg; Gln or Glu; Met to Leu or lie; Phe to Met, Leu or Tyr; Ser to Thr; Thr to Ser; Trp to Tyr; Tyr to Trp or Phe; and, Val to lie or Leu.

An “exogenous antigen-recognition receptor” is a receptor capable of recognizing an antigen, which receptor is artificially introduced into an engineered cell. Non-limiting examples of exogenous antigen-recognition receptors include chimeric antigen receptors (CARs) and TCRs (where the TCR is artificially introduced into the cell, for example, a cell that does not otherwise express a TCR, or expresses a different TCR). An exogenous antigen-recognition receptor can be, for example, a transgenic TCR, an alpha beta TCR, or a gamma delta TCR.

As used herein, the term “chimeric antigen receptor” or “CAR” refers to an artificial exogenous antigen recognition receptor that can induce signaling in an engineered cell that expresses the CAR upon binding of the CAR to an antigen, for example, an antigen associated with a cancer or infectious disease. A CAR generally induces signaling in the engineered cell that expresses the CAR but not in a cell that expresses or presents the antigen bound by the CAR. A CAR comprises at least one extracellular targeting domain, at least one transmembrane domain, and at least one intracellular signaling domain. In some cases, a CAR comprises a hinge domain. A CAR extracellular targeting domain can be, comprise, or be derived from, for example, a monoclonal antibody, a recombinant antibody, a human antibody, a humanized antibody, or a functional derivative, variant or fragment thereof, including, but not limited to, a heavy chain variable domain (VH), a light chain variable domain (VL), a Fab, a Fab′, a F(ab′)2, an Fv, a single-chain Fv (scFv), a minibody, a diabody, a single-domain antibody such as a VHH, and any combination thereof. A CAR extracellular targeting domain can be, comprise, or be derived from, for example, a DARPin, a non-antibody domain (e.g., from or derived from a receptor or a receptor ligand, for example, APRIL). The intracellular signaling domain of a CAR can induce or reduce activity of an engineered cell comprising the CAR. An intracellular signaling domain of a CAR can be or can comprise a truncated portion of a signaling domain of another molecule. In some cases, intracellular domain of the CAR can be involved in regulating primary activation of a TCR complex in either a stimulatory manner or an inhibitory manner. In some embodiments, the intracellular signaling domain of the CAR is involved in inducing T cell activation and/or a cytotoxic response against cells that express the antigen that is bound by the CAR. In some cases CARs are also referred to as artificial T cell receptors, chimeric immunoreceptors, or chimeric T cell receptors.

As used herein, the term “heterodimeric receptor” includes any receptor which is a macromolecular complex formed by two protein monomers which are different to each other. The term may further be understood to include functional heterodimeric fragments or parts of receptors. As non-limiting examples, the term includes a signal transduction moiety of a B-cell receptor (which is an Ig-α/Ig-β heterodimer (CD79)), B-cell receptor heavy and light chain, a Toll-like receptor 1 and 2 heterodimer, an integrin like αvβ5, a phagocytic receptor Mac-1, an MHC, a CD94 NKG2C or NKG2E receptor, a T-cell receptor (TCR), an alpha beta (αβ) TCR, a gamma delta (γδ) TCR, and any other receptor or functional fragment or part thereof that may occur as a heterodimer.

An “antigen” is a molecule or molecular structure that an antigen receptor or an antigen-binding protein can recognize (for example, bind to). An antigen can be or can comprise, for example, a peptide, a polypeptide, a carbohydrate, a chemical, a moiety, a non-peptide antigen, a phosphoantigen, a tumor-associated antigen, a neoantigen, a tumor microenvironment antigen, a microbial antigen, a viral antigen, a bacterial antigen, an autoantigen, a glycan-based antigen, a peptide-based antigen, a lipid-based antigen, or any combination thereof. In some embodiments, an antigen is capable of inducing an immune response. In some examples, an antigen binds to an antigen receptor or antigen-binding protein, or induces an immune response, when present in a complex e.g., presented by MHC. In some cases, an antigen adopts a certain conformation in order to bind to an antigen receptor or antigen-binding protein, and/or to induce an immune response, e.g., adopts a conformation in response to the presence or absence of one or more metabolites. Antigen can refer to a whole target molecule, a whole complex, a or a fragment of a target molecule or complex that binds to an antigen receptor or an antigen-binding protein. Antigen receptors that recognize antigens include exogenous antigen-recognition receptors disclosed herein and other antigen-recognition receptors, such as endogenous T cell receptors.

A “TEG” is a T cell engineered to express a defined γδ TCR as disclosed herein. In a non-limiting example, a TEG can be an alpha-beta T cell that is engineered to express a defined γδ TCR. Within the context of the application, the expression “engineered cell” refers to a cell that has been modified using recombinant DNA technology. In an embodiment, an “engineered cell” has been transformed, modified or transduced to comprise a heterologous nucleic acid molecule. In an embodiment, said cell expresses a protein encoded by said nucleic acid molecule.

### II. Extracellular Part of the Midis Protein

### A. Extracellular Ligand Domain

MIDIS proteins (i.e. chimeric bidirectional signaling transmembrane protein) of the disclosure comprise at least one extracellular ligand domain. An extracellular ligand domain of a MIDIS protein is capable of binding to an interaction partner, and inducing signaling mediated by the interaction partner.

Accordingly, the invention provides a chimeric bidirectional signaling transmembrane protein (MIDIS) able to transduce at least two intracellular signals, said protein comprising:
an extracellular ligand domain, able to interact with the extracellular domain of its interaction partner
a transmembrane domain, and
a heterologous intracellular signaling domain transducing a first signal after binding of the extracellular ligand domain to its interaction partner,
   wherein the second intracellular signal is transduced via the intracellular domain of the interaction partner.

In an embodiment, the at least two intracellular signals are inducible.

In an embodiment, the MIDIS proteins (i.e. chimeric bidirectional signaling transmembrane proteins) are able to transduce at least two intracellular signals, said proteins comprising:
an extracellular ligand domain, able to interact with the extracellular domain of its interaction partner
a transmembrane domain, and
a heterologous intracellular signaling domain transducing a first signal after binding of the extracellular ligand domain to its interaction partner,
   wherein the second intracellular signal is transduced via the intracellular domain of the interaction partner and wherein the chimeric protein is not a protein comprising or consisting of the extracellular ligand domain and the transmembrane domain of the ICOSL and the heterologous intracellular signaling domain of 41BB.

A chimeric protein comprising or consisting of the extracellular ligand domain and the transmembrane domain of the ICOSL and the heterologous intracellular signaling domain of 41BB as disclaimed above may be represented by SEQ ID NO:137 or by an amino acid sequence having at least 97%, or at least 98%, or at least 98.5% or at least 99% or at least 99.5% or at least 100% identity with SEQ ID NO:137 over its whole length.

In an embodiment, the chimeric bidirectional signaling transmembrane protein does not comprise the extracellular ligand domain and the transmembrane domain of the ICOSL. Such protein may be represented by SEQ ID NO: 138 or by an amino acid sequence having at least 97%, or at least 98%, or at least 98.5% or at least 99% or at least 99.5% or at least 100% identity with SEQ ID NO:138 over its whole length.

In an embodiment, the chimeric bidirectional signaling transmembrane protein does not comprise the extracellular ligand domain of the ICOSL. Such protein may be represented by SEQ ID NO: 139 or by an amino acid sequence having at least 97%, or at least 98%, or at least 98.5% or at least 99% or at least 99.5% or at least 100% identity with SEQ ID NO:139 over its whole length.

ICOS is highly expressed on peripheral Treg and is involved in the development and suppressive function of these cells (Akbari O, Nat Med. 2002 September; 8(9):1024-32. doi: 10.1038/nm745. Epub 2002 Jul. 29. PMID: 12145647, Busse M, J Immunol. (2012) 189:1975-82. doi: 10.4049/jimmunol.1103581 and Tuettenberg A, J Immunol. 2009 Mar. 15; 182(6):3349-56. doi: 10.4049/jimmunol.0802733. PMID: 19265111). ICOS activation sensitizes T cells for anti-inflammatory IL10 signaling (Tuettenberg A, J Immunol. 2009 Mar. 15; 182(6):3349-56. doi: 10.4049/jimmunol.0802733. PMID: 19265111). In view of the biological properties of ICOS and its ligand ICOSL, it is preferred that ICOS signaling is not induced by the chimeric protein disclosed herein. For this reason, we define three possible types of disclaimers above by excluding the presence of SEQ ID NO:137, 138 or 139 (or sequences having at least 97%, or at least 98%, or at least 98.5% or at least 99% or at least 99.5% or at least 100% identity with SEQ ID NO: 137, 138 or 139 over their whole length) as (part of the) chimeric bidirectional signaling transmembrane protein of the disclosure. In an embodiment, the interaction partner is not ICOS.

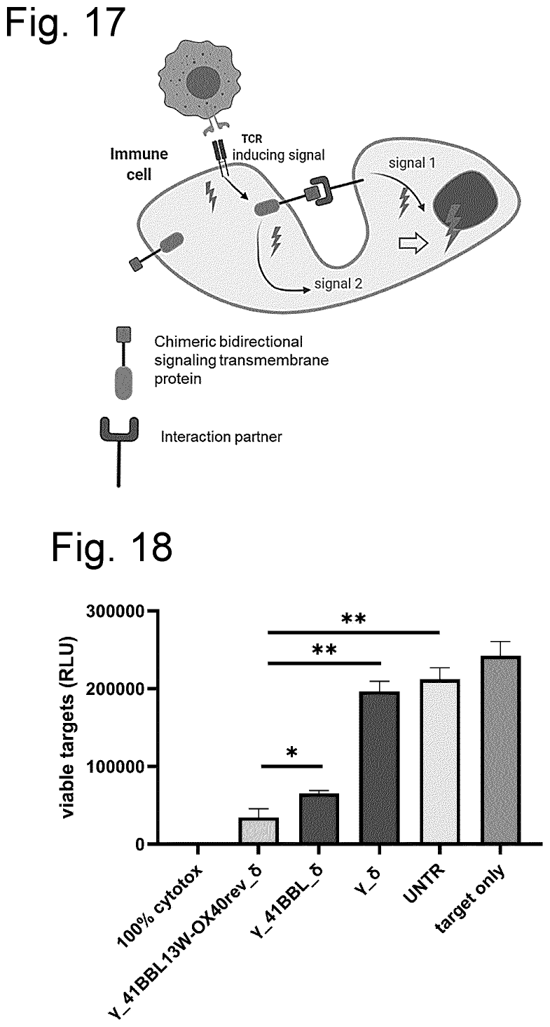

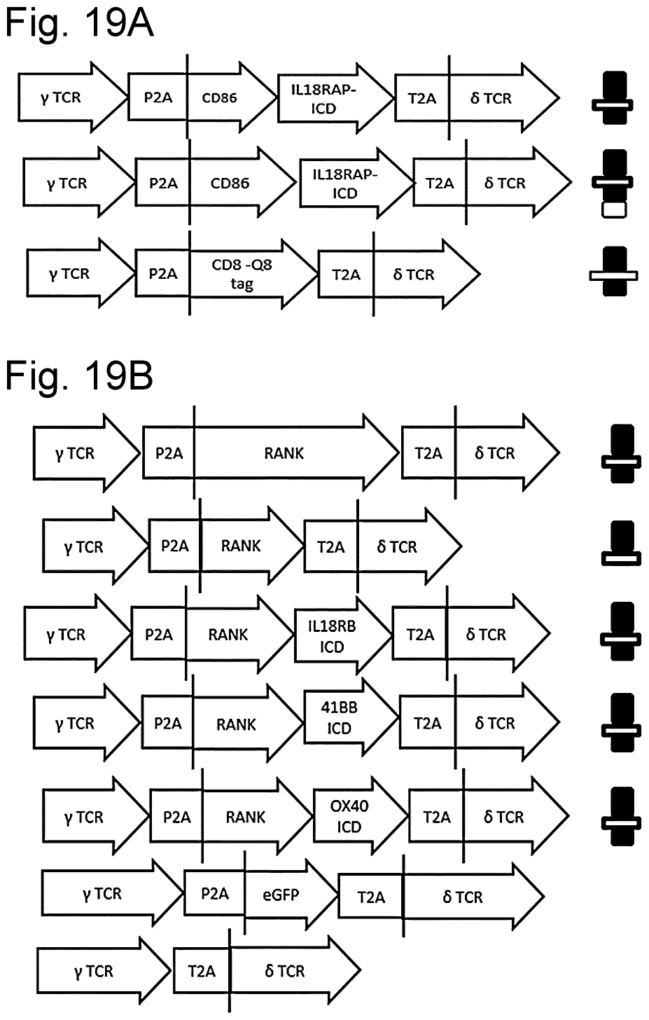

An extracellular ligand domain can be selected based on its ability to induce signaling mediated by a desired interaction partner. In some cases, an extracellular ligand domain can be selected based on its ability to elicit signaling mediated by the heterologous intracellular signaling domain of the MIDIS protein upon binding to the interaction partner. The “at least two intracellular signals” are optionally inducible. It means that the chimeric bidirectional signaling transmembrane protein may be considered as having two configurations: one wherein no signal is induced and one wherein “at least two intracellular signals” are induced upon interaction of the extracellular ligand domain of the chimeric protein with the extracellular ligand domain of its interaction partner. These “at least two intracellular signals” may occur simultaneously or sequentially. The inducibility of these “at least two intracellular signals” is attractive as the chimeric protein is controllable by the interaction partner and vice versa. This inducibility may be assessed using techniques known to the skilled person and depending on the identity of the heterologous intracellular signaling domain of the chimeric protein and of the intracellular domain of the interaction partner. In addition, one of these “at least two intracellular signals” may depend on the activation of a cell leading to the expression of the interaction partner. In addition, one of these “at least two intracellular signals” may depend on the activation or signaling of an additional receptor, for example but not limited to the TCR. A non-limiting example of activation or signaling of an additional receptor is the IL2 pathway with expression of CD25 (IL2Ra) upon TCR activation and IL2 release upon TCR signaling. A non-limiting illustrative example of an embodiment wherein the “at least two intracellular signals” are inducible is shown in FIG. 17.

An extracellular ligand domain can comprise an amino acid sequence that is from or derived from a protein that is expressed on a cell surface. In some embodiments the protein expressed on a cell surface has agonist activity on a cognate receptor.

The extracellular ligand domain can comprise an amino acid sequence that is from or derived from a type I transmembrane protein. In some embodiments, the extracellular ligand domain comprises an amino acid sequence that is from or derived from a type II transmembrane protein.

The extracellular ligand domain can comprise an amino acid sequence that is from or derived from a tumor necrosis factor superfamily member. In some cases, the extracellular ligand domain comprises an amino acid sequence that is from or derived from an immune co-receptor ligand, for example, an immune co-stimulatory ligand. In some embodiments, the extracellular ligand domain comprises an amino acid sequence that is from or derived from an immunoglobulin superfamily member. The extracellular ligand domain can comprise an amino acid sequence that is from or derived from 41BBL, OX40L, CD86, or RANK. The extracellular ligand domain can comprise an amino acid sequence that is from or derived from 41BBL, OX40L, CD86, RANK, or CD70. In some embodiments, the extracellular ligand domain comprises an amino acid sequence that is from or derived from 41BBL. In an embodiment, the extracellular ligand domain is from or derived from 41BBL which is a type II transmembrane protein. In some embodiments, the extracellular ligand domain comprises an amino acid sequence that is from or derived from OX40L. In some embodiments, the extracellular ligand domain comprises an amino acid sequence that is from or derived from CD86. In some embodiments, the extracellular ligand domain comprises an amino acid sequence that is from or derived from RANK. In some embodiments, the extracellular ligand domain comprises an amino acid sequence that is from or derived from CD70.

The extracellular ligand domain can comprise an amino acid sequence that is from or derived from a receptor, for example, an ion channel, GPCR, or receptor tyrosine kinase. In some embodiments, the extracellular ligand domain comprises an amino acid sequence that is from or derived from a tumor necrosis factor receptor superfamily member. In some embodiments, the extracellular ligand domain comprises an amino acid sequence that is from or derived from an immune co-receptor.

The extracellular ligand domain can comprise an amino acid sequence that is from or derived from a cytokine. The extracellular ligand domain can comprise an amino acid sequence that is from or derived from a C-type lectin. The extracellular ligand domain can comprise an amino acid sequence that is from or derived from a soluble protein, for example, a secreted or cytoplasmic protein.

An extracellular ligand domain can comprise a peptide ligand of an interaction partner, for example, a naturally-occurring or a synthetic peptide ligand.

An extracellular ligand domain can comprise an amino acid sequence that is from or derived from an antigen-binding protein. Non-limiting examples of antigen-binding proteins include antibodies, variable regions (e.g., variable chain heavy region (VH) and/or variable chain light region (VL)), short chain variable fragments (scFv), single domain antibodies, Fab, Fab′, F(ab′)2, dimers and trimers of Fab conjugates, Fv, minibodies, diabodies, triabodies, tetrabodies, affibodies, ankyrin proteins, ankyrin repeats, DARPins, monobodies, nanobodies, avimers, adnectins, anticalins, Fynomers, Kunitz domains, knottins, or β-hairpin mimetics. In some embodiments, an extracellular ligand domain comprises one or more single-chain variable fragments (scFvs). A scFv (single-chain variable fragment) is a fusion protein that can comprise VH and VL domains connected by a peptide linker. Manipulation of the orientation of the VH and VL domains and the linker length can be used to create different forms of molecules that can be monomeric, dimeric (diabody), trimeric (triabody), or tetrameric (tetrabody). Minibodies are scFv-CH3 fusion proteins that assemble into bivalent dimers. In some embodiments, an extracellular ligand domain comprises one or more DARPins. In some embodiments, an extracellular ligand domain comprises one or more complementarity determining regions (CDRs) from an antibody or T cell receptor, for example, one, three or six CDRs. Antigen-binding fragments derived from monoclonal antibodies can be, for example, chimeric, humanized or fully human.

An extracellular ligand domain can be selected based on its binding affinity for a desired interaction partner. In some embodiments, an extracellular ligand domain binds to an interaction partner with a KD of, for example, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 900 pM, less than about 800 pM, less than about 700 pM, less than about 600 pM, less than about 500 pM, less than about 400 pM, less than about 300 pM, less than about 200 pM, less than about 100 pM, less than about 50 pM, less than about 10 pM, less than about 1 pM, less than about 500 fM, or less than about 100 fM.

An extracellular ligand domain can comprise an amino acid sequence that is from or derived from a wild type protein amino acid sequence. A wild type protein amino acid sequence can refer to a sequence that is naturally occurring and encoded by a germline genome. A species can have one wild type sequence, or two or more wild type sequences (for example, with one canonical wild type sequence and one or more non-canonical wild type sequences). A wild type protein amino acid sequence can be a mature form of a protein that has been processed to remove N-terminal and/or C-terminal residues, for example, to remove a signal peptide.

An extracellular ligand domain can comprise an amino acid sequence that is modified compared to a wild type protein amino acid sequence or any other amino acid sequence disclosed herein, for example, to achieve a desirable level of expression, surface expression, stability, resistance to aggregation, resistance to degradation, affinity for an interaction partner, or level of signaling mediated by an interaction partner. An extracellular ligand domain can comprise an amino acid sequence that is modified compared to a wild type protein amino acid sequence or an amino acid sequence disclosed herein, for example, to promote folding of the MIDIS into a biologically active conformation. In some embodiments, part or all of an extracellular ligand domain comprises an amino acid sequence that is inverted compared to a wild type amino acid sequence (i.e. expressed as a retro-protein).

An extracellular ligand domain can comprise, consist essentially of, or consist of an amino acid sequence with at least a minimal level of sequence identity compared to a wild type protein amino acid sequence or any other amino acid sequence disclosed herein. In an embodiment, such extracellular ligand domain having at least a minimal level of sequence identity compared to a given amino acid sequence is functional and therefore encompassed by the invention as long as this extracellular ligand domain is able to bind or interact with the extracellular domain of its interaction partner. The level of binding or interaction should be detectable using an assay known to the skilled person. Examples of suitable assays are western blotting or FACS, ELISA or SPR assays. Depending on the extracellular ligand domain used, the skilled person will know which assay is the most appropriate. For example for OX40, NFKB signaling will be assessed, for 41BBL the binding of 41BB will be assessed. In an embodiment, the activity of the extracellular ligand domain is assessed when said extracellular ligand domain is still comprised within the full length transmembrane molecule it originates from. For example, an extracellular ligand domain can comprise, consist essentially of, or consist of an amino acid sequence with at least 80%, at least 85%, at least 90%, at least 91%, at least 92%, at least 93%, at least 94%, at least 95%, at least 96%, at least 97%, at least 98%, at least 98.5%, at least 99%, or at least 99.5% sequence identity to a wild type protein amino acid sequence or any other amino acid sequence disclosed herein, for example, any one of SEQ ID NOs: 01-06, or 174. In cases where part or all of an extracellular ligand domain comprises an amino acid sequence that is inverted compared to a wild type amino acid sequence (i.e. expressed as a retro-protein), the wild type protein amino acid sequence can be inverted prior to calculating sequence identity.

In some embodiments, an extracellular ligand domain can comprise, consist essentially of, or consist of an amino acid sequence that is a wild type protein amino acid sequence or any other amino acid sequence disclosed herein, for example, any one of SEQ ID NOs: 01-06. Another example is any one of SEQ ID NOs: 01-06, or 174.

Table 1 provides non-limiting examples of amino acid sequences that an extracellular domain or extracellular ligand domain of the disclosure can comprise, consist of, consist essentially of, or be derived from. EC: extracellular.

TABLE 1

| SEQ ID | | | |
|---|---|---|---|
| NO: | Name | Description | Sequence |
| 01 | 41BBL-EC | WT 41BBL | ACPWAVSGARASPGSAASPRLREGPELSPDDPAGLLDLR |
| | | EC domain | QGMFAQLVAQNVLLIDGPLSWYSDPGLAGVSLTGGLSYKE |
| | | | DTKELVVAKAGVYYVFFQLELRRVVAGEGSGSVSLALHLQ |
| | | | PLRSAAGAAALALTVDLPPASSEARNSAFGFQGRLLHLSAG |
| | | | QRLGVHLHTEARARHAWQLTQGATVLGLFRVTPEIPAGLP |
| | | | SPRSE |
| 02 | OX40L | WT OX40L | QVSHRYPRIQSIKVQFTEYKKEKGFILTSQKEDEIMKVQNNS |
| | | EC domain | VIINCDGFYLISLKGYFSQEVNISLHYQKDEEPLFQLKKVRS |
| | | | VNSLMVASLTYKDKVYLNVTTDNTSLDDFHVNGGELILIHQ |
| | | | NPGEFCVL |
| 03 | CD86 | WT CD86 EC | MDPQCTMGLSNILFVMAFLLSGAAPLKIQAYFNETADLPCQ |
| | | domain | FANSQNQSLSELVVFWQDQENLVLNEVYLGKEKFDSVHSK |
| | | | YMGRTSFDSDSWTLRLHNLQIKDKGLYQCIIHHKKPTGMIRI |
| | | | HQMNSELSVLANFSQPEIVPISNITENVYINLTCSSIHGYPEP |
| | | | KKMSVLLRTKNSTIEYDGVMQKSQDNVTELYDVSISLSVSF |
| | | | PDVTSNMTIFCILETDKTRLLSSPFSIELEDPQPPPDHIP |
| 04 | RANK | WT RANK EC | MAPRARRRRPLFALLLLCALLARLQVALQIAPPCTSEKHYE |
| | | domain | HLGRCCNKCEPGKYMSSKCTTTSDSVCLPCGPDEYLDSW |
| | | | NEEDKCLLHKVCDTGKALVAVVAGNSTTPRRCACTAGYH |
| | | | WSQDCECCRRNTECAPGLGAQHPLQLNKDTVCKPCLAGY |
| | | | FSDAFSSTDKCRPWTNCTFLGKRVEHHGTEKSDAVCSSSL |
| | | | PARKPPNEPHVYLP |
| 05 | Reverse | 41BBL core | MLLLVTSLLLCELPHPAFLLIPDQGMFAQLVAQNVLLIDGPL |
| | 41BBL | protein in a | SWYSDPGLAGVSLTGGLSYKEDTKELVVAKAGVYYVFFQL |
| | | type I | ELRRVVAGEGSGSVSLALHLQPLRSAAGAAALALTVDLPPA |
| | | orientated | SSEARNSAFGFQGRLLHLSAGQRLGVHLHTEARARHAWQ |
| | | way with IgK | LTQGATVLGLFRVTPEIPAGLPSPRSERLDLLGAPDDPSLE |
| | | leader | PGERLRPSAASGPSARA |
| | | sequence and | |
| | | inverted | |
| | | extracellular | |
| | | N-terminal | |
| | | part | |
| 06 | 41BBL- | WT 41BBL | ACPWAVSGARASPGSAASPRLREGPELSPDDPAGLLDLR |
| | CD86 IgV | EC domain | QGMFAQLVAQNVLLIDGPLSWYSDPGLAGVSLTGGLSYKE |
| | domain | with CD86 | DTKELVVAKAGVYYVFFQLELRRVVAGEGSGSVSLALHLQ |
| | | IgV domain | PLRSAAGAAALALTVDLPPASSEARNSAFGFQGRLLHLSAG |
| | | linked to it | QRLGVHLHTEARARHAWQLTQGATVLGLFRVTPEIPAGLP |
| | | with GGGS | SPRSESLNGGGGSGGGGSGGGGGGGGSGGGGSTSAP |
| | | linker | LKIQAYFNETADLPCQFANSQNQSLSELVVFWQDQENLVL |
| | | | NEVYLGKEKFDSVHSKYMGRTSFDSDSWTLRLHNLQIKDK |
| | | | GLYQCIIHHKKPTGMIRIHQMNSELSVLAN |
| 174 | CD70 | WT CD70 EC | QRFAQAQQQLPLESLGWDVAELQLNHTGPQQDPRLYWQ |
| | | domain | GGPALGRSFLHGPELDKGQLRIHRDGIYMVHIQVTLAICSST |
| | | | TASRHHPTTLAVGICSPASRSISLLRLSFHQGCTIASQRLTP |
| | | | LARGDTLCTNLTGTLLPSRNTDETFFGVQWVRP |

An extracellular ligand domain can comprise an amino acid sequence with one or more amino acid insertions, deletions, or substitutions compared to a wild type protein amino acid sequence or any other amino acid sequence disclosed herein.

For example, an extracellular ligand domain can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to a wild type protein amino acid sequence or any other amino acid sequence disclosed herein, for example, any one of SEQ ID NOs: 01-06. Another example is any one of SEQ ID NOs: 01-06, or 174.

In some embodiments, an extracellular ligand domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to a wild type protein amino acid sequence or any other amino acid sequence disclosed herein, for example, any one of SEQ ID NOs: 01-06. Another example is any one of SEQ ID NOs: 01-06, or 174.

In some embodiments, an extracellular ligand domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid insertions relative to a wild type protein amino acid sequence or any other amino acid sequence disclosed herein, for example, any one of SEQ ID NOs: 01-06. Another example is any one of SEQ ID NOs: 01-06, or 174.

The one or more insertions can be at the N-terminus, C-terminus, within the amino acid sequence, or a combination thereof. The one or more insertions can be contiguous, non-contiguous, or a combination thereof.

In some embodiments, an extracellular ligand domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid deletions relative to a wild type protein amino acid sequence or any other amino acid sequence disclosed herein, for example, any one of SEQ ID NOs: 01-06. Another example is any one of SEQ ID NOs: 01-06, or 174.

In some embodiments, an extracellular ligand domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to a wild type protein amino acid sequence or any other amino acid sequence disclosed herein, for example, any one of SEQ ID NOs: 01-06. Another example is any one of SEQ ID NOs: 01-06, or 174.

In some embodiments, an extracellular ligand domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid deletions relative to a wild type protein amino acid sequence or any other amino acid sequence disclosed herein, for example, any one of SEQ ID NOs: 01-06. Another example is any one of SEQ ID NOs: 01-06, or 174.

The one or more deletions can be at the N-terminus, C-terminus, within the amino acid sequence, or a combination thereof. The one or more deletions can be contiguous, non-contiguous, or a combination thereof.

In some embodiments, an extracellular ligand domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to a wild type protein amino acid sequence or any other amino acid sequence disclosed herein, for example, any one of SEQ ID NOs: 01-06. Another example is any one of SEQ ID NOs: 01-06, or 174.

In some embodiments, an extracellular ligand domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to a wild type protein amino acid sequence or any other amino acid sequence disclosed herein, for example, any one of SEQ ID NOs: 01-06. Another example is any one of SEQ ID NOs: 01-06, or 174.

In some embodiments, an extracellular ligand domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to a wild type protein amino acid sequence or any other amino acid sequence disclosed herein, for example, any one of SEQ ID NOs: 01-06. Another example is any one of SEQ ID NOs: 01-06, or 174.

The one or more substitutions can be at the N-terminus, C-terminus, within the amino acid sequence, or a combination thereof. The one or more substitutions can be contiguous, non-contiguous, or a combination thereof. The one or more substitutions can be conservative, non-conservative, or a combination thereof.

A conservative amino acid substitution can be a substitution of one amino acid for another amino acid of similar biochemical properties (e.g., charge, size, and/or hydrophobicity). A non-conservative amino acid substitution can be a substitution of one amino acid for another amino acid with different biochemical properties (e.g., charge, size, and/or hydrophobicity). A conservative amino acid change can be, for example, a substitution that has minimal effect on the secondary or tertiary structure of a polypeptide.

A MIDIS protein of the disclosure can have any suitable number of extracellular ligand domains. In some embodiments a MIDIS Protein has one extracellular ligand domain. In some embodiments, a MIDIS Protein has two extracellular ligand domains. In some embodiments, a MIDIS protein has 1, 2, 3, 4, 5, 6, 7, 8, 9, or 10 extracellular ligand domain(s). In some embodiments, a MIDIS protein has at least 1, at least 2, at least 3, at least 4, at least 5, at least 6, at least 7, at least 8, at least 9, or at least 10 extracellular ligand domain(s). In some embodiments, a MIDIS protein has at most 1, at most 2, at most 3, at most 4, at most 5, at most 6, at most 7, at most 8, at most 9, or at most 10 extracellular ligand domain(s).

### B. Interaction Partner of the Extracellular Ligand Domain

An interaction partner of an extracellular ligand domain is present on the surface of a cell and upon binding of the extracellular ligand domain to the interaction partner, signaling via an intracellular domain of the interaction partner is induced. Induction of the signaling pathway can contribute to a range of target biological outcomes and biological functions disclosed herein, for example, enhanced cellular proliferation, survival, and greater magnitude and duration of immune effector functions.

An interaction partner may be a co-immune receptor.

In an embodiment, the cell comprises, preferably co-expresses the chimeric bidirectional signaling transmembrane protein and the interaction partner, each as a transmembrane protein. In an embodiment, there is no cell comprising or expressing the interaction partner and that will not comprise or will not express the signal bidirectional signaling transmembrane protein. The interaction partner may be endogenously expressed on a cell and said cell may be transduced or transform with the chimeric bidirectional signaling transmembrane protein. Alternatively, both the interaction partner and the chimeric bidirectional signaling transmembrane protein may be transduced into the same cell.

This embodiment wherein one single cell is able to transduce the at least two intracellular signals originating from the chimeric bidirectional signaling transmembrane protein is attractive as said cell relies on an endogenous type of signaling and is self-activating or self-sufficient in a cell surface expression regulated fashion and may not need any other signal to improve a biological parameter and/or a function and/or to improve a biological parameter and/or function induced by such a cell.

In an embodiment, the interaction partner of the chimeric bidirectional signaling transmembrane protein comprises:
an extracellular domain able to interact with the extracellular ligand domain of the chimeric protein,
a transmembrane domain, and
an intracellular domain transducing a second signal after binding of the extracellular domain of the interaction partner to the extracellular ligand domain of the chimeric protein.

In some embodiments, binding of the extracellular ligand domain to the interaction partner modulates a second signaling pathway, for example, induces, or increases or decreases activity of the second signaling pathway. In some embodiments, the interaction partner is present in a signaling complex and upon binding of the extracellular ligand domain of the MIDIS to the interaction partner, signaling mediated by the interaction partner is modulated, e.g., signaling mediated by the signaling complex is increased or decreased. In some embodiments, upon binding of the extracellular ligand domain to the interaction partner, activity of a first signaling pathway is reduced and a different signaling pathway is induced. An interaction partner can be selected based on its ability to modulate (e.g., induce) a signaling pathway that is associated with a desired biological outcome or biological function.

In some embodiments, the MIDIS protein binds to the interaction partner as a monomer. In some embodiments, the MIDIS protein forms a dimer when bound to the interaction partner. In some embodiments, the MIDIS protein forms a trimer when bound to the interaction partner. In some embodiments, the MIDIS protein binds to the interaction partner as a tetramer, a pentamer, a hexamer, or a multimer. When bound as a multimer (e.g., a dimer, trimer, tetramer, pentamer, hexamer, or higher order multimer), the MIDIS protein can form a homo-multimer (e.g., homodimer, homotrimer, homotetramer, homopentamer, homohexamer, or higher order homomultimer). In some cases, the MIDIS protein binds to the interaction partner as a hetero-multimer (e.g., a heterodimer, heterotrimer, heterotetramer, heteropentamer, heterohexamer, or higher order heteromultimer).

In some embodiments, the interaction partner that binds to the extracellular ligand domain is expressed by an immune cell. In some embodiments, the interaction partner is expressed by a leukocyte, such as a lymphocyte, e.g., a T cell. In some embodiments, the interaction partner is expressed by a cancer cell. In some embodiments, the interaction partner is expressed by a mammalian cell. In some embodiments, the interaction partner is expressed by a human cell. In some embodiments, the interaction partner is expressed by an alpha-beta T cell, a gamma-delta T cell, CD4+ T cell, CD8+ T cell, a T effector cell, a lymphocyte, a B cell, an NK cell, an NKT cell, a myeloid cell, a monocyte, a macrophage, a neutrophil, a basophil, a dendritic cell, an eosinophil, a granulocyte, a helper T cell, a memory T cell, a Langerhans cell, a lymphoid cell, an innate lymphoid cell (ILC), a mast cell, a megakaryocyte, a plasma cell, a thymocyte, a fibroblast, a keratinocyte, a mesenchymal stem cell, an endothelial cell, a stromal cell, or any mixture or combination of cells thereof. In some embodiments, the interaction partner is expressed by a primary cell. In some embodiments, the interaction partner is expressed by a cell that is not a primary cell.

In an embodiment, the interaction partner of the chimeric bidirectional signaling transmembrane protein of the disclosure is not more important for Treg development and function compared to general T cell development and function. In an embodiment, the expression of the interaction partner is inducible. In an embodiment, this expression is induced upon TCR activation.

In some embodiments, the interaction partner is expressed by a cell that is the same cell type as the cell that expresses the MIDIS protein. In some embodiments, the MIDIS protein and the interaction partner are both expressed by the same cell.

An interaction partner can be a receptor, for example, for example a tumor necrosis factor receptor superfamily member. The interaction partner can be, for example, 41BB, OX40, RANKL, or IL18RAP (IL18RB). Another example is 41BB, OX40, RANKL, IL18RAP, or CD27. In some embodiments, the interaction partner is 41BB. In some embodiments, the interaction partner is OX40. In some embodiments, the interaction partner is RANKL. In some embodiments, the interaction partner is IL18RAP. In some embodiments, the interaction partner is CD27. In an embodiment the interaction partner is not ICOS.

In some embodiments, an interaction partner is an immunoglobulin superfamily member, or an immune co-receptor, for example an activating immune co-receptor, such as CD86. In some embodiments, an interaction partner is a cytokine receptor. In some embodiments, an interaction partner is a C-type lectin receptor. In some embodiments, the interaction partner is an ion channel, GPCR, serine peptidase, integrin, tetraspanin, or receptor tyrosine kinase. In some embodiments, an interaction partner is a tumor necrosis factor superfamily member that comprises an intracellular domain that can mediate signaling. In some embodiments, the interaction partner is 41BBL or OX40L.

In an embodiment, the at least two, optionally inducible, intracellular signals transduced by the chimeric bidirectional signaling transmembrane protein contribute to an improvement of a biological parameter and/or function of a cell expressing the chimeric protein and/or an improvement of a biological parameter and/or function induced by such a cell.

In some embodiments, upon binding of the extracellular ligand domain to the interaction partner, at least one, at least two, at least three, at least four, at least five, or at least six signaling pathways are induced that are mediated by the intracellular domain of the interaction partner. In some embodiments, upon binding of the extracellular ligand domain to the interaction partner, one, two, three, four, five, or six signaling pathways are induced that are mediated by the intracellular domain of the interaction partner. In some embodiments, upon binding of the extracellular ligand domain to the interaction partner, one signaling pathway is induced that is mediated by the intracellular domain of the interaction partner.

### C. Additional Extracellular Domains

The extracellular part of the MIDIS protein can comprise one or more additional extracellular domains as well as the one or more extracellular ligand domains.

In some embodiments, a MIDIS protein comprises one or more additional extracellular domains from the same protein as the extracellular ligand domain, e.g., stretches of amino acids that do not participate in binding to an interaction partner, or do not induce signaling mediated by an interaction partner that binds to the extracellular ligand domain. In some embodiments, an additional extracellular domain does not participate in binding to the interaction partner, but increases or decreases a level of signaling mediated by the interaction partner.

In some embodiments, a MIDIS protein comprises an additional extracellular domain that is from or derived from the same protein as the transmembrane domain, e.g., the same protein or a different protein than the heterologous intracellular signaling domain. In some embodiments, such an additional extracellular domain does not induce signaling mediated by an interaction partner.

In some embodiments, a MIDIS protein comprises an additional extracellular domain that is from or derived from the same protein as the heterologous intracellular signaling domain. In some embodiments, such an additional extracellular domain does not induce signaling mediated by an interaction partner. In some cases, an additional extracellular domain can be selected based on its ability to elicit signaling in mediated by the heterologous intracellular signaling domain of the MIDIS protein upon binding of the extracellular ligand domain to the interaction partner.

An additional extracellular domain can be or can comprise a cleavage site, for example, an ADAM family cleavage site or a metalloprotease family cleavage site. An additional extracellular domain can be or can comprise a multimerization domain (e.g., a domain that facilitates formation of a homo- or hetero-dimer, trimer, tetramer, pentamer, hexamer, or higher order multimer, such as a tenascin-C oligomerization domain, a thrombospondin oligomerization domain, or a GCN4 oligomerization domain). An additional extracellular domain can be or can comprise a cellular localization motif, e.g., a lipid raft localization motif or a nuclear localization motif. An additional extracellular domain can be or can comprise a target peptide, e.g., a signal peptide. An additional extracellular domain can comprise a linker.

An additional extracellular domain can comprise an amino acid sequence that is from or derived from a wild type protein amino acid sequence. An additional extracellular domain can comprise an amino acid sequence that is from or derived from any protein or type of protein disclosed elsewhere herein. An additional extracellular domain can comprise an amino acid sequence that is modified compared to a wild type protein amino acid sequence or any other amino acid sequence disclosed herein, for example, to achieve a desirable level of expression, surface expression, stability, resistance to aggregation, resistance to shedding, or resistance to degradation. An additional extracellular domain can comprise an amino acid sequence that is modified compared to a wild type protein amino acid sequence or an amino acid sequence disclosed herein, for example, to promote folding of the MIDIS into a biologically active conformation. In some embodiments, part or all of an additional extracellular domain comprises an amino acid sequence that is inverted compared to a wild type amino acid sequence (i.e. expressed as a retro-protein).

An additional extracellular domain can comprise an amino acid sequence with one or more amino acid insertions, deletions, or substitutions compared to a wild type protein amino acid sequence or any other amino acid sequence as disclosed elsewhere herein. An additional extracellular domain can comprise at least a minimal level of sequence identity compared to a wild type protein amino acid sequence or any other amino acid sequence as disclosed elsewhere herein.

### III. Intracellular Domains

### D. Heterologous Intracellular Signaling Domain

MIDIS proteins of the disclosure comprise at least one heterologous intracellular signaling domain. “Heterologous” refers to the fact that the intracellular signaling domain is from or is derived from a different protein than the extracellular ligand domain. A signaling pathway mediated by the heterologous intracellular signaling domain is induced upon binding of the extracellular ligand domain to an interaction partner. The induction of the signaling pathway can contribute to a range of target biological outcomes and biological functions disclosed herein, for example, enhanced cellular proliferation, survival, and greater magnitude and duration of immune effector functions.

A heterologous intracellular signaling domain can be selected based on its ability to induce a signaling pathway that is associated with a desired biological outcome or biological function. A heterologous intracellular signaling domain can comprise an amino acid sequence that is from or derived from a transmembrane protein, for example, a protein that is expressed on a cell surface. The heterologous intracellular signaling domain can comprise an amino acid sequence that is from or derived from a type I transmembrane protein. In some embodiments, the heterologous intracellular signaling domain comprises an amino acid sequence that is from or derived from a type II transmembrane protein.

The heterologous intracellular signaling domain can comprise an amino acid sequence that is from or derived from a tumor necrosis factor receptor superfamily member. The heterologous intracellular signaling domain can comprise an amino acid sequence that is from or derived from an immunoglobulin superfamily member. The heterologous intracellular signaling domain can comprise an amino acid sequence that is from or derived from a cytokine receptor. The heterologous intracellular signaling domain can comprise an amino acid sequence that is from or derived from a C-lectin family member. The heterologous intracellular signaling domain can comprise an amino acid sequence that is from or derived from 41BB, OX40, NKp80, or IL18RAP. The heterologous intracellular signaling domain can comprise an amino acid sequence that is from or derived from 41BB, OX40, NKp80, IL18RAP, or IL2RB. In some embodiments, the heterologous intracellular signaling domain comprises an amino acid sequence that is from or derived from 41BB. In some embodiments, the heterologous intracellular signaling domain comprises an amino acid sequence that is from or derived from OX40. In some embodiment, the heterologous intracellular signaling domain comprises an amino acid sequence that is from or derived from OX40 and is from or derived from a type I transmembrane OX40 protein. In some embodiments, the heterologous intracellular signaling domain comprises an amino acid sequence that is from or derived from NKp80. In some embodiments, the heterologous intracellular signaling domain comprises an amino acid sequence that is from or derived from IL18RAP. In some embodiments, the heterologous intracellular signaling domain comprises an amino acid sequence that is from or derived from IL2RB.

The heterologous intracellular signaling domain can comprise an amino acid sequence that is from or derived from a receptor, for example, an ion channel, GPCR, serine protease, an immunoglobulin superfamily member, complement receptor, TIR domain containing receptor, or receptor tyrosine kinase. The heterologous intracellular signaling domain can comprise an amino acid sequence that is from or derived from a cytokine receptor. The heterologous intracellular signaling domain can comprise an amino acid sequence that is from or derived from a C-type lectin receptor. The heterologous intracellular signaling domain can comprise an amino acid sequence that is from or derived a cytoplasmic protein that participates in a signaling pathway. The heterologous intracellular signaling domain can comprise an amino acid sequence that is from or derived a nuclear protein that participates in a signaling pathway.

In some embodiments, the heterologous intracellular signaling domain comprises an amino acid sequence that is from or derived from an intracellular domain of a tumor necrosis factor superfamily member. In some embodiments, the heterologous intracellular signaling domain comprises an amino acid sequence that is from or derived from an intracellular domain of an immune co-receptor. In some cases, the heterologous intracellular signaling domain comprises an amino acid sequence that is from or derived from an intracellular domain of an immune co-receptor ligand that contains a signaling domain, for example, an intracellular signaling domain of an immune co-stimulatory ligand. In many cases it is not necessary to use the entire chain, for example, a truncated portion of the signaling domain can be used in the heterologous intracellular signaling domain.

The heterologous intracellular signaling domain can be structurally distinct from intracellular domains found in chimeric antigen receptors and similar chimeric proteins. For example, the heterologous intracellular signaling domain can lack one or more components associated with TCR complex signaling. In some embodiments, the heterologous intracellular signaling domain does not contain an ITAM. In some embodiments, the heterologous intracellular signaling domain contains a hemITAM but does not contain an ITAM. In some embodiments, the heterologous intracellular signaling domain is not phosphorylated upon binding of the MIDIS protein to the interaction partner. In some embodiments, the heterologous intracellular signaling domain does not contain an intracellular domain from a CD3 chain, for example does not contain an intracellular domain of a CD3 zeta chain. In some embodiments, the heterologous intracellular signaling domain does not contain an intracellular domain from a TCR signaling complex. In some embodiments, the heterologous intracellular signaling domain is phosphorylated upon binding of the MIDIS protein to the interaction partner.

A heterologous intracellular signaling domain can comprise an amino acid sequence that is from or derived from a wild type protein amino acid sequence. A heterologous intracellular signaling domain can comprise an amino acid sequence that is modified compared to a wild type protein amino acid sequence or any other amino acid sequence disclosed herein, for example, to achieve a desirable level of expression, surface expression, stability, resistance to aggregation, resistance to degradation, signaling strength, or affinity for a protein that participates in downstream signaling, e.g., an adapter protein. A heterologous intracellular signaling domain can comprise an amino acid sequence that is modified compared to a wild type protein amino acid sequence or an amino acid sequence disclosed herein, for example, to promote folding of the MIDIS into a biologically active conformation. In some embodiments, part or all of a heterologous intracellular signaling domain comprises an amino acid sequence that is inverted compared to a wild type amino acid sequence (i.e. expressed as a retro-protein).

A heterologous intracellular signaling domain can comprise, consist essentially of, or consist of an amino acid sequence with at least a minimal level of sequence identity compared to a wild type protein amino acid sequence or any other amino acid sequence disclosed herein. For example, a heterologous intracellular signaling domain can comprise, consist essentially of, or consist of an amino acid sequence with at least 80%, at least 85%, at least 90%, at least 91%, at least 92%, at least 93%, at least 94%, at least 95%, at least 96%, at least 97%, at least 98%, at least 98.5%, at least 99%, or at least 99.5% sequence identity to a wild type protein amino acid sequence or any other amino acid sequence disclosed herein, for example, any one of SEQ ID NOs: 07-19. Another example is any one of SEQ ID NOs: 07-19, or 175. In an embodiment, such heterologous intracellular signaling domain having at least a minimal level of sequence identity compared to a given amino acid sequence is functional and therefore encompassed by the invention as long as this intracellular signaling domain is able to transduce a first signal after binding of the extracellular ligand domain to its interaction partner. The first signal should be detectable using an assay known to the skilled person. Examples of suitable assays are western blotting or FACS, luminescence assays. Depending on the identity of the heterologous intracellular signaling domain used, the skilled person will know which assay is appropriate to use. A NfκB reporter assay may be used to assess the activity of said heterologous intracellular domain. In an embodiment, the activity of the heterologous intracellular signaling domain is assessed when said intracellular signaling domain is still comprised within the full-length transmembrane molecule it originates from.

In cases where part or all of a heterologous intracellular signaling domain comprises an amino acid sequence that is inverted compared to a wild type amino acid sequence (i.e. expressed as a retro-protein), the wild type protein amino acid sequence can be inverted prior to calculating sequence identity. In some embodiments, a heterologous intracellular signaling domain can comprise, consist essentially of, or consist of an amino acid sequence that is a wild type protein amino acid sequence or any other amino acid sequence disclosed herein, for example, any one of SEQ ID NOs: 07-19. Another example is any one of SEQ ID NOs: 07-19, or 175.

Table 2 provides non-limiting examples of amino acid sequences that intracellular domains and heterologous intracellular signaling domain of the disclosure can comprise, consist of, consist essentially of, or be derived from.

TABLE 2

| SEQ ID | | | |
|---|---|---|---|
| NO: | Name | Description | Sequence |
| 07 | OX40-ICD | From WT | RDQRLPPDAHKPPGGGSFRTPIQEEQADAHSTLAKI |
| | | OX40 | |
| 08 | OX40-ICD- | Inverted | IKALTSHADAQEEQIPTRFSGGGPPKHADPPLRQDR |
| | REV | OX40-ICD | |
| 09 | OX40-ICD- | From WT OX40, | VAAILGLGLVLGLLGPLAILLALYLLRRDQRLPPDAHKPPGGG |
| | TM | includes | SFRTPIQEEQADAHSTLAKI |
| | | OX40 TM | |
| | | domain | |
| 10 | 41 BB-ICD | From WT | KRGRKKLLYIFKQPFMRPVQTTQEEDGCSCRFPEEEEGGCE |
| | | 41BB | L |
| 11 | 41BB-ICD- | Inverted | MLGKRGRKKLLYIFKQPFMRPVQTTQEEDGCSCRFPEEEEG |
| | REV | 41 BB-ICD | GCEL |
| 12 | NKp80-ICD | NKp80-ICD | MQDEDGYMTLNVQSKKRSSAQTSQLTFKDYSVTLHW |
| 13 | IL18RAP- | From WT | AASALLYRHWIEIVLLYRTYQSKDQTLGDKKDFDAFVSYAKW |
| | ICD | IL18RAP | SSFPSEATSSLSEEHLALSLFPDVLENKYGYSLCLLERDVAPG |
| | | | GVYAEDIVSIIKRSRRGIFILSPNYVNGPSIFELQAAVNLALDDQ |
| | | | TLKLILIKFCYFQEPESLPHLVKKALRVLPTVTWRGLKSVPPNS |
| | | | RFWAKMRYHMPVKNSQGFTWNQLRITSRIFQWKGLSRTETT |
| | | | GRSSQPKEW |
| 14 | 41BB-ICD- | From WT | IISFFLALTSTALLFLLFFLTLRFSVVKRGRKKLLYIFKQPFMRP |
| | TM | 41BB, | VQTTQEEDGCSCRFPEEEEGGCEL |
| | | includes | |
| | | 41BB TM | |
| | | domain | |
| 15 | OX40-ICD- | From WT | IKALTSHADAQEEQIPTRFSGGGPPKHADPPLRQDR RLLYLA |
| | ALYLLR | OX40 | |
| 16 | OX40-ICD- | Inverted | ALYLLRRDQRLPPDAHKPPGGGSFRTPIQEEQADAHSTLAKI |
| | ALYLLR- | OX40-ICD- | |
| | REV | AYLLR | |
| 17 | TRAF | TRAF | PIQEEQ |
| | domain | domain | |
| | OX40 | OX40 | |
| 18 | YMTLN motif | YMTLN motif | YMTLN |
| 19 | TRAF | TRAF | QTTQEEDGCSCRFPEEEE |
| | domain 41BB | domain 41BB | |
| 175 | IL2RB ICD | IL2RB | VLHTPDQGQLEQLSLYADTNLPLLQTVKDRELVELPSIEPALG |
| | | truncated | GPSFSSSPFPSSLWKQVDGGHESSLQSFFKSPDPTNCKLVK |
| | | signaling | KLWPGTNRCN |
| | | domain | |
| 176 | CD70 ICD | From WT | MLGPEEGSGCSVRRRPYG |
| | | CD70 ICD | |

A heterologous intracellular signaling domain can comprise an amino acid sequence with one or more amino acid insertions, deletions, or substitutions compared to a wild type protein amino acid sequence or any other amino acid sequence disclosed herein.

For example, a heterologous intracellular signaling domain can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to a wild type protein amino acid sequence or any other amino acid sequence disclosed herein, for example, any one of SEQ ID NOs: 07-19. Another example is any one of SEQ ID NOs: 07-19, or 175.

In some embodiments, a heterologous intracellular signaling domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to a wild type protein amino acid sequence or any other amino acid sequence disclosed herein, for example, any one of SEQ ID NOs: 07-19. Another example is any one of SEQ ID NOs: 07-19, or 175.

In some embodiments, a heterologous intracellular signaling domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid insertions relative to a wild type protein amino acid sequence or any other amino acid sequence disclosed herein, for example, any one of SEQ ID NOs: 07-19. Another example is any one of SEQ ID NOs: 07-19, or 175.

The one or more insertions can be at the N-terminus, C-terminus, within the amino acid sequence, or a combination thereof. The one or more insertions can be contiguous, non-contiguous, or a combination thereof.

In some embodiments, a heterologous intracellular signaling domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid deletions relative to a wild type protein amino acid sequence or any other amino acid sequence disclosed herein, for example, any one of SEQ ID NOs: 07-19. Another example is any one of SEQ ID NOs: 07-19, or 175.

In some embodiments, a heterologous intracellular signaling domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to a wild type protein amino acid sequence or any other amino acid sequence disclosed herein, for example, any one of SEQ ID NOs: 07-19. Another example is any one of SEQ ID NOs: 07-19, or 175.

In some embodiments, a heterologous intracellular signaling domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid deletions relative to a wild type protein amino acid sequence or any other amino acid sequence disclosed herein, for example, any one of SEQ ID NOs: 07-19. Another example is any one of SEQ ID NOs: 07-19, or 175.

The one or more deletions can be at the N-terminus, C-terminus, within the amino acid sequence, or a combination thereof. The one or more deletions can be contiguous, non-contiguous, or a combination thereof.

In some embodiments, a heterologous intracellular signaling domain comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to a wild type protein amino acid sequence or any other amino acid sequence disclosed herein, for example, any one of SEQ ID NOs: 07-19. Another example is any one of SEQ ID NOs: 07-19, or 175.

In some embodiments, a heterologous intracellular signaling domain comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to a wild type protein amino acid sequence or any other amino acid sequence disclosed herein, for example, any one of SEQ ID NOs: 07-19. Another example is any one of SEQ ID NOs: 07-19, or 175.

In some embodiments, a heterologous intracellular signaling domain comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to a wild type protein amino acid sequence or any other amino acid sequence disclosed herein, for example, any one of SEQ ID NOs: 07-19. Another example is any one of SEQ ID NOs: 07-19, or 175.

The one or more substitutions can be at the N-terminus, C-terminus, within the amino acid sequence, or a combination thereof. The one or more substitutions can be contiguous, non-contiguous, or a combination thereof. The one or more substitutions can be conservative, non-conservative, or a combination thereof.

In some embodiments, the heterologous intracellular signaling domain signals as a monomer. In some embodiments, the heterologous intracellular signaling domain signals as a dimer. In some embodiments, the heterologous intracellular signaling domain signals as a trimer. In some embodiments, the heterologous intracellular signaling domain signals as a tetramer, a pentamer, a hexamer, or a multimer. When signaling as a multimer (e.g., a dimer, trimer, tetramer, pentamer, hexamer, or higher order multimer), the heterologous intracellular signaling domain can signal as a homo-multimer (e.g., homodimer, homotrimer, homotetramer, homopentamer, homohexamer, or higher order homomultimer). In some cases, the heterologous intracellular signaling domain signals as a hetero-multimer (e.g., a heterodimer, heterotrimer, heterotetramer, heteropentamer, heterohexamer, or higher order heteromultimer). In some embodiments, the heterologous intracellular signaling domain signals in a different conformation or as a different multimer than a full length wild type protein from which the heterologous intracellular signaling domain is from or derived from.

A MIDIS protein of the disclosure can have any suitable number of heterologous intracellular signaling domains. In some embodiments a MIDIS Protein has one heterologous intracellular signaling domain. In some embodiments, a MIDIS Protein has two heterologous intracellular signaling domains. In some embodiments, a MIDIS protein has 1, 2, 3, 4, 5, 6, 7, 8, 9, or 10 heterologous intracellular signaling domain(s). In some embodiments, a MIDIS protein has at least 1, at least 2, at least 3, at least 4, at least 5, at least 6, at least 7, at least 8, at least 9, or at least 10 heterologous intracellular signaling domain(s). In some embodiments, a MIDIS protein has at most 1, at most 2, at most 3, at most 4, at most 5, at most 6, at most 7, at most 8, at most 9, or at most 10 heterologous intracellular signaling domain(s).

In some embodiments, a MIDIS protein comprises two heterologous intracellular signaling domains that are from or derived from 41BB, OX40, NKp80, IL18RAP, or IL2RB. In some embodiments, a MIDIS protein comprises a heterologous intracellular signaling domain that is from or derived from OX40, and a heterologous domain that is from or derived from 41BB, NKp80, IL18RAP, or IL2RB. In some embodiments, a MIDIS protein comprises a heterologous intracellular signaling domain that is from or derived from OX40, and a heterologous intracellular signalling domain that is from or derived from IL2RB.

In some embodiments, upon binding of the extracellular ligand domain to the interaction partner, at least one, at least two, at least three, at least four, at least five, or at least six signaling pathways are induced that are mediated by the heterologous intracellular signaling domain. In some embodiments, upon binding of the extracellular ligand domain to the interaction partner, one, two, three, four, five, or six signaling pathways are induced that are mediated by the heterologous intracellular signaling domain. In some embodiments, upon binding of the extracellular ligand domain to the interaction partner, one signaling pathway is induced that is mediated by the heterologous intracellular signaling domain.

### E. Additional Intracellular Domains

A MIDIS protein can comprise one or more additional intracellular domains as well as the one or more heterologous intracellular signaling domains.

In some embodiments, a MIDIS protein comprises one or more additional intracellular domains from or derived from the same protein as the heterologous intracellular signaling domain, e.g., stretches of amino acids that do not participate in signaling. In some embodiments, an additional intracellular domain does not directly participate in signaling (e.g., does not bind a signaling pathway component or undergo a chemical or structural change as part of a signaling pathway), but increases or decreases a level of signaling mediated by the heterologous intracellular signaling domain.

In some embodiments, a MIDIS protein comprises an additional intracellular domain that is from or derived from the same protein as the transmembrane domain, which can be e.g., the same protein or a different protein than the extracellular ligand domain. Such an intracellular domain can comprise a signaling domain or can lack a signaling domain.

In some embodiments, a MIDIS protein comprises an intracellular domain that is from or derived from the same protein as the extracellular ligand domain. Such an intracellular domain can lack a signaling domain or can comprise a different signaling domain to the heterologous intracellular signaling domain that is present in the MIDIS. In some embodiments, one or more amino acids are added to achieve sequence similarity and/or structural similarity to the protein that is the source of the extracellular ligand domain. For example, in some embodiments, the amino acids MLG can be added to the intracellular N-terminus of a MIDIS protein that contains a 41BBL extracellular ligand domain.

An additional intracellular domain can be or can comprise a cleavage site, for example, an ADAM family cleavage site or a metalloprotease family cleavage site. An additional intracellular domain can be or can comprise a multimerization domain (e.g., a domain that facilitates formation of a homo- or hetero-dimer, trimer, tetramer, pentamer, hexamer, or higher order multimer, such as a tenascin-C oligomerization domain, a thrombospondin oligomerization domain, or a GCN4 oligomerization domain). An additional intracellular domain can be or can comprise a target peptide, e.g. a signal peptide. An additional intracellular domain can be or can comprise a cellular localization motif, e.g., a lipid raft localization motif or a nuclear localization motif. An additional intracellular domain can comprise a linker.

An additional intracellular domain can comprise an amino acid sequence that is from or derived from a wild type protein amino acid sequence. An additional intracellular domain can comprise an amino acid sequence that is from or derived from any protein or type of protein disclosed elsewhere herein. An additional intracellular domain can comprise an amino acid sequence that is modified compared to a wild type protein amino acid sequence or any other amino acid sequence disclosed herein, for example, to achieve a desirable level of expression, surface expression, stability, resistance to aggregation, resistance to degradation, signaling strength, or affinity for a protein that participates in downstream signaling, e.g., an adapter protein. An additional intracellular domain can comprise an amino acid sequence that is modified compared to a wild type protein amino acid sequence or an amino acid sequence disclosed herein, for example, to promote folding of the MIDIS into a biologically active conformation. In some embodiments, part or all of an additional intracellular domain comprises an amino acid sequence that is inverted compared to a wild type amino acid sequence (i.e. expressed as a retro-protein).

An additional intracellular domain can comprise an amino acid sequence with one or more amino acid insertions, deletions, or substitutions compared to a wild type protein amino acid sequence or any other amino acid sequence as disclosed elsewhere herein. An additional intracellular domain can comprise at least a minimal level of sequence identity compared to a wild type protein amino acid sequence or any other amino acid sequence as disclosed elsewhere herein.

In some embodiments, the entire intracellular part of a MIDIS protein of the disclosure (containing the one or more heterologous intracellular signaling domain(s) and any additional intracellular domains) can be structurally distinct from intracellular domains found in chimeric antigen receptors and similar chimeric proteins. For example, the entire intracellular part of a MIDIS protein can lack one or more components associated with TCR complex signaling. In some embodiments, the entire intracellular part of a MIDIS protein does not contain an ITAM (e.g., contains a hemITAM but not an ITAM, or does not contain a hemITAM or an ITAM). In some embodiments, the entire intracellular part of a MIDIS protein is not phosphorylated upon binding of the MIDIS protein to the interaction partner. In some embodiments, an intracellular part of a MIDIS protein is phosphorylated upon binding of the MIDIS protein to the interaction partner. In some embodiments, the entire intracellular part of a MIDIS protein does not contain an intracellular domain from a CD3 chain, for example does not contain an intracellular domain of a CD3 zeta chain, or does not contain an intracellular domain from any CD3 chain. In some embodiments, the entire intracellular part of a MIDIS protein does not contain an intracellular domain from a TCR signaling complex.

### IV. Transmembrane Domain

MIDIS proteins of the disclosure comprise a transmembrane domain that connects the extracellular ligand domain to the heterologous intracellular signaling domain.

In some embodiments, part or all of the transmembrane domain is from the same protein as the extracellular ligand domain. In cases where part or all of the transmembrane domain is from the same protein as the extracellular ligand domain, the transmembrane domain and the extracellular ligand domain can be part of a contiguous amino acid sequence (e.g., that matches or corresponds to a wild type sequence), or can be separated by one or more amino acid insertions, deletions, and/or substitutions. In an embodiment, the transmembrane domain or part thereof is from or derived from the same protein as the extracellular ligand domain.

In some embodiments, part or all of the transmembrane domain is from the same protein as the heterologous intracellular signaling domain. In cases where part or all of the transmembrane domain is from the same protein as the heterologous intracellular signaling domain, the transmembrane domain and the heterologous intracellular signaling domain can be part of a contiguous amino acid sequence (e.g., that matches or corresponds to a wild type sequence), or can be separated by one or more amino acid insertions, deletions, and/or substitutions.

In some embodiments, part or all of the transmembrane domain is from or derived from a different protein than the extracellular ligand domain and the heterologous intracellular signaling domain. As a non-limiting example, a MIDIS protein of the disclosure may comprise an extracellular ligand domain that comprises an amino acid sequence that is from or derived from CD70, a transmembrane domain that comprises an amino acid sequence that is from or derived from 41BBL, and a heterologous intracellular signaling domain that comprises an amino acid sequence that is from or derived from OX40.

A transmembrane domain can comprise an amino acid sequence that is from or derived from a transmembrane protein, for example, a protein that is expressed on a cell surface. The transmembrane domain can comprise an amino acid sequence that is from or derived from a type I transmembrane protein. In some embodiments, the transmembrane domain comprises an amino acid sequence that is from or derived from a type II transmembrane protein.

The transmembrane domain can comprise an amino acid sequence that is from or derived from a tumor necrosis factor receptor superfamily member. The transmembrane domain can comprise an amino acid sequence that is from or derived from 41BB, OX40, NKp80, RANK, or IL18RAP. The transmembrane domain can comprise an amino acid sequence that is from or derived from 41BB, OX40, NKp80, RANK, IL18RAP, or CD70. In some embodiments, the transmembrane domain comprises an amino acid sequence that is from or derived from 41BB. In some embodiments, the transmembrane domain comprises an amino acid sequence that is from or derived from OX40. In some embodiments, the transmembrane domain comprises an amino acid sequence that is from or derived from NKp80. In some embodiments, the transmembrane domain comprises an amino acid sequence that is from or derived from RANK. In some embodiments, the transmembrane domain comprises an amino acid sequence that is from or derived from IL18RAP. In some embodiments, the transmembrane domain comprises an amino acid sequence that is from or derived from CD70.

In some embodiments, the transmembrane domain comprises an amino acid sequence that is from or derived from a tumor necrosis factor superfamily member or an immunoglobulin superfamily. The transmembrane domain can comprise an amino acid sequence that is from or derived from 41BBL, OX40L, CD86, or RANK. The transmembrane domain can comprise an amino acid sequence that is from or derived from 41BBL, OX40L, CD86, RANK, or CD70. In some embodiments, the transmembrane domain comprises an amino acid sequence that is from or derived from 41BBL. In some embodiments, the transmembrane domain comprises an amino acid sequence that is from or derived from OX40L. In some embodiments, the transmembrane domain comprises an amino acid sequence that is from or derived from CD86. In some embodiments, the transmembrane domain comprises an amino acid sequence that is from or derived from RANK. In some embodiments, the transmembrane domain comprises an amino acid sequence that is from or derived from CD70.

The transmembrane domain can comprise an amino acid sequence that is from or derived from a receptor, for example, an ion channel, GPCR, selectin family member, cytokine receptor, adhesion molecule, or receptor tyrosine kinase. The transmembrane domain can comprise an amino acid sequence that is from or derived from a cytokine receptor. The transmembrane domain can comprise an amino acid sequence that is from or derived from a C-type lectin or C type lectin receptor. In some embodiments, the transmembrane domain comprises an amino acid sequence that is from or derived from an immune co-receptor. In some cases, the transmembrane domain comprises an amino acid sequence that is from or derived from an immune co-receptor ligand, for example, an immune co-stimulatory ligand.

In an aspect, a transmembrane domain is from an alpha chain of a T cell receptor (TCR), beta chain of a TCR, CD8, CD4, CD28, CD45, PD-1 and/or CD152.

A transmembrane domain can comprise an amino acid sequence that is from or derived from a wild type protein amino acid sequence. A transmembrane domain can comprise an amino acid sequence that is modified compared to a wild type protein amino acid sequence or any other amino acid sequence disclosed herein, for example, to achieve a desirable level of expression, surface expression, stability, resistance to aggregation, resistance to degradation, signaling strength, localization, or multimerization of the MIDIS protein. A transmembrane domain can comprise an amino acid sequence that is modified compared to a wild type protein amino acid sequence or an amino acid sequence disclosed herein, for example, to promote folding of the MIDIS into a biologically active conformation. In some embodiments, part or all of a transmembrane domain comprises an amino acid sequence that is inverted compared to a wild type amino acid sequence (i.e. expressed as a retro-protein). A transmembrane domain can comprise an artificial hydrophobic sequence. In some embodiments, a transmembrane domain can comprise a cellular localization motif, e.g., a lipid raft localization motif or a nuclear localization motif.

In one non-limiting example, a MIDIS of the disclosure can contain an extracellular ligand domain from RANK, and a transmembrane domain from IL18RAP. In some embodiments, inclusion of the transmembrane domain from IL18RAP induces formation of the MIDIS into a dimeric state, unlike wild type RANK, which can function as a trimer. In the same way, transmembrane domains of the disclosure can induce formation of the MIDIS into a monomeric or multimeric state that is different than the state adopted by the full length wild type version of the protein the extracellular ligand domain is from or derived from, and/or that is different than the full length wild type version of the protein the heterologous intracellular domain is from or derived from.

A transmembrane domain can comprise, consist essentially of, or consist of an amino acid sequence with at least a minimal level of sequence identity compared to a wild type protein amino acid sequence or any other amino acid sequence disclosed herein. For example, a transmembrane domain can comprise, consist essentially of, or consist of an amino acid sequence with at least 80%, at least 85%, at least 90%, at least 91%, at least 92%, at least 93%, at least 94%, at least 95%, at least 96%, at least 97%, at least 98%, at least 98.5%, at least 99%, or at least 99.5% sequence identity to a wild type protein amino acid sequence or any other amino acid sequence disclosed herein, for example, any one of SEQ ID NOs: 20-27. Another example is any one of SEQ ID NOs: 20-27, or 177. In an embodiment, such transmembrane domain having at least a minimal level of sequence identity compared to a given amino acid sequence is functional and therefore encompassed by the invention as long as this transmembrane domain is able to induce a multimerization of the chimeric bidirectional signaling transmembrane protein comprising it upon binding of the extracellular domain of its interaction partner. The level of binding or interaction should be detectable using an assay known to the skilled person. Examples of suitable assays are western blotting or FACS, single photon microscopy assays.

In cases where part or all of a transmembrane domain comprises an amino acid sequence that is inverted compared to a wild type amino acid sequence (i.e. expressed as a retro-protein), the wild type protein amino acid sequence can be inverted prior to calculating sequence identity. In some embodiments, a transmembrane domain can comprise, consist essentially of, or consist of an amino acid sequence that is a wild type protein amino acid sequence or any other amino acid sequence disclosed herein, for example, any one of SEQ ID NOs: 20-27. Another example is any one of SEQ ID NOs: 20-27, or 177

Table 3 provides non-limiting examples of amino acid sequences that a transmembrane domain of the disclosure can comprise, consist of, consist essentially of, or be derived from.

TABLE 3

| SEQ | | | |
|---|---|---|---|
| ID | | | |
| NO: | Name | Description | Sequence |
| 20 | OX40-TM | From WT | VAAILGLGLVLGLLGPLAILL |
| | | OX40 | |
| 21 | OX40L-TM | From WT | LLLVASVIQGLGLLLCFTYICLHFSAL |
| | | OX40L | |
| 22 | 41BB-TM | From WT | IISFFLALTSTALLFLLFFLTLRFSVV |
| | | 41BB | |
| 23 | 41BBL-TM | From WT | WALVAGLLLLLLLAAACAVFL |
| | | 41BBL | |
| 24 | NKp80-TM | From WT | ILLGISGTVNGILTLTLISLI |
| | | NKp80 | |
| 25 | CD86-TM | From WT | WITAVLPTVIICVMVFCLILW |
| | | CD86 | |
| 26 | IL18RAP-TM | From WT | GVVLLYILLGTIGTLVAVL |
| | | IL18RAP | |
| 27 | RANK-TM | From WT | GLIILLLFASVALVAAIIFGV |
| | | RANK | |
| 177 | CD70-TM | TM CD70 | VLRAALVPLVAGLVICLVVCI |

A transmembrane domain can comprise an amino acid sequence with one or more amino acid insertions, deletions, or substitutions compared to a wild type protein amino acid sequence or any other amino acid sequence disclosed herein.

For example, a transmembrane domain can comprise an amino acid sequence with at least 1, at least 2, at least 3, at least 4, at least 5, at least 6, at least 7, at least 8, at least 9, or at least 10 amino acid insertions relative to a wild type protein amino acid sequence or any other amino acid sequence disclosed herein, for example, any one of SEQ ID NOs: 20-27. Another example is any one of SEQ ID NOs: 20-27, or 177. In some embodiments, a transmembrane domain comprises an amino acid sequence with at most 1, at most 2, at most 3, at most 4, at most 5, at most 6, at most 7, at most 8, at most 9, or at most 10 amino acid insertions relative to a wild type protein amino acid sequence or any other amino acid sequence disclosed herein, for example, any one of SEQ ID NOs: 20-27. Another example is any one of SEQ ID NOs: 20-27, or 177. In some embodiments, a transmembrane domain comprises an amino acid sequence with 1, 2, 3, 4, 5, 6, 7, 8, 9, or 10 amino acid insertions relative to a wild type protein amino acid sequence or any other amino acid sequence disclosed herein, for example, any one of SEQ ID NOs: 20-27. Another example is any one of SEQ ID NOs: 20-27, or 177. The one or more insertions can be at the N-terminus, C-terminus, within the amino acid sequence, or a combination thereof. The one or more insertions can be contiguous, non-contiguous, or a combination thereof.

In some embodiments, a transmembrane domain comprises an amino acid sequence with at least 1, at least 2, at least 3, at least 4, at least 5, at least 6, at least 7, at least 8, at least 9, or at least 10 amino acid deletions relative to a wild type protein amino acid sequence or any other amino acid sequence disclosed herein, for example, any one of SEQ ID NOs: 20-27. Another example is any one of SEQ ID NOs: 20-27, or 177. In some embodiments, a transmembrane domain comprises an amino acid sequence with at most 1, at most 2, at most 3, at most 4, at most 5, at most 6, at most 7, at most 8, at most 9, or at most 10 amino acid deletions relative to a wild type protein amino acid sequence or any other amino acid sequence disclosed herein, for example, any one of SEQ ID NOs: 20-27. Another example is any one of SEQ ID NOs: 20-27, or 177. In some embodiments, a transmembrane domain comprises an amino acid sequence with 1, 2, 3, 4, 5, 6, 7, 8, 9, or 10 amino acid deletions relative to a wild type protein amino acid sequence or any other amino acid sequence disclosed herein, for example, any one of SEQ ID NOs: 20-27. Another example is any one of SEQ ID NOs: 20-27, or 177. The one or more deletions can be at the N-terminus, C-terminus, within the amino acid sequence, or a combination thereof. The one or more deletions can be contiguous, non-contiguous, or a combination thereof.

In some embodiments, a transmembrane domain comprises an amino acid sequence with at least 1, at least 2, at least 3, at least 4, at least 5, at least 6, at least 7, at least 8, at least 9, or at least 10, amino acid substitutions relative to a wild type protein amino acid sequence or any other amino acid sequence disclosed herein, for example, any one of SEQ ID NOs: 20-27. Another example is any one of SEQ ID NOs: 20-27, or 177. In some embodiments, a transmembrane domain comprises an amino acid sequence with at most 1, at most 2, at most 3, at most 4, at most 5, at most 6, at most 7, at most 8, at most 9, or at most 10 amino acid substitutions relative to a wild type protein amino acid sequence or any other amino acid sequence disclosed herein, for example, any one of SEQ ID NOs: 20-27. Another example is any one of SEQ ID NOs: 20-27, or 177. In some embodiments, a transmembrane domain comprises an amino acid sequence with 1, 2, 3, 4, 5, 6, 7, 8, 9, or 10 amino acid sequence or any other amino acid sequence disclosed herein, for example, any one of SEQ ID NOs: 20-27. Another example is any one of SEQ ID NOs: 20-27, or 177. The one or more substitutions can be at the N-terminus, C-terminus, within the amino acid sequence, or a combination thereof. The one or more substitutions can be contiguous, non-contiguous, or a combination thereof. The one or more substitutions can be conservative, non-conservative, or a combination thereof.

### V. Linkers

MIDIS proteins of the disclosure can comprise one or more linkers that connect amino acid sequences of the disclosure, for example, amino acid sequences from or derived from different proteins. A linker can connect, for example, an extracellular ligand domain to a transmembrane domain, a heterologous intracellular signaling domain to a transmembrane domain, one extracellular ligand domain to a second extracellular ligand domain or an additional extracellular domain, one heterologous intracellular signaling domain to another heterologous intracellular signaling domain or an additional intracellular domain, or any domain disclosed herein to another amino acid sequence.

A linker or can allow for separation and flexibility of the domains it separates, for example, a transmembrane domain and an extracellular ligand domain. The length of a linker can be adjusted to alter the ability of a domain to bind to, for example, an interaction partner (for the extracellular ligand domain), or a factor that participates in a signaling pathway (e.g., for the heterologous intracellular signaling domain).

A linker sequence can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length. In some embodiments, a linker is at least 1, at least 3, at least 5, at least 7, at least 9, at least 11, or at least 15 amino acids in length. In some embodiments, a linker is at most 5, at most 7, at most 9, at most 11, at most 15, at most 20, at most 25, or at most 50 amino acids in length.

A flexible linker can have a sequence containing stretches of glycine and serine residues. The small size of the glycine and serine residues provides flexibility, and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid linker can have, for example, an alpha helix-structure. An alpha-helical rigid linker can act as a spacer between protein domains.

A linker can comprise any of the sequences in Table 4, or repeats thereof (e.g., 2, 3, 4, 5, 6, 7, 8, 9, or 10 repeats of any of SEQ ID NOs: 28-44).

TABLE 4

| SEQ | | |
|---|---|---|
| ID | | |
| NO: | Description | Sequence |
| 28 | Flexible linker | TSGS |
| 29 | Flexible linker | GGGGS |
| 30 | Flexible linker | GGGS |
| 31 | Flexible linker | GG |
| 32 | Flexible linker | KESGSVSSEQLAQFRSLD |
| 33 | Flexible linker | EGKSSGSGSESKST |
| 34 | Flexible linker | GSAGSAAGSGEF |
| 35 | Rigid linker | EAAAK |
| 36 | Rigid linker | EAAAR |
| 37 | Rigid linker | PAPAP |
| 38 | Rigid linker | AEAAAKEAAAKA |
| 39 | Rigid linker | ILTHDSSIRYLQEIYNSNNQKIVNLKEKVAQLEAQCQEPCKDTVQIHDITG |
| 40 | Flexible linker | GGS |
| 41 | Flexible linker | SLNGGGGSGGGGSGGGGSGGGGSGGGGSTS |
| 42 | Flexible linker | SGGSGGGGSGGGSGGGGSLQ |
| 43 | Flexible linker | SGGGSGGGGSGGGGSGGGGSGGGSLQ |
| 44 | Flexible linker | GGGGSGGGGSGGGGS |

In some embodiments, a MIDIS protein comprises a linker with at least 1, at least 2, at least 3, at least 4, or at least 5 amino acid insertions, deletions, or substitutions relative to any of SEQ ID NOs: 28-44. The insertions, deletions, or substitutions can be at the N-terminus, the C-terminus, within the sequence, or a combination thereof. The insertions, deletions, or substitutions can be contiguous or non-contiguous. In some cases, the substitutions are conservative. In some cases, the substitutions are non-conservative.

In some embodiments, a MIDIS protein of the disclosure does not contain any linkers, for example, the MIDIS protein is a direct fusion of amino acid sequences from other proteins with no intervening amino acid sequence.

### VI. Exemplary Midis Proteins

A chimeric bidirectional signaling transmembrane protein (MIDIS) of the disclosure is able to transduce at least two intracellular signals, said protein comprising:
an extracellular ligand domain, able to interact with the extracellular domain of its interaction partner
a transmembrane domain, and
a heterologous intracellular signaling domain transducing a first signal after binding of the extracellular ligand domain to its interaction partner,
   wherein the second intracellular signal is transduced via the intracellular domain of the interaction partner.

In an embodiment, the at least two intracellular signals are inducible.

In an embodiment, the chimeric bidirectional signaling transmembrane protein able to transduce at least two intracellular signals, comprises:
an extracellular ligand domain, able to interact with the extracellular domain of its interaction partner
a transmembrane domain, and
a heterologous intracellular signaling domain transducing a first signal after binding of the extracellular ligand domain to its interaction partner,
   wherein the second intracellular signal is transduced via the intracellular domain of the interaction partner and wherein the chimeric protein is not a protein comprising or consisting of the extracellular ligand domain and the transmembrane domain of the ICOSL and the heterologous intracellular signaling domain of 41BB.

A chimeric protein comprising or consisting of the extracellular ligand domain and the transmembrane domain of the ICOSL and the heterologous intracellular signaling domain of 41BB as disclaimed above may be represented by SEQ ID NO:137 or by an amino acid sequence having at least 97%, or at least 98%, or at least 98.5% or at least 99% or at least 99.5% or at least 100% identity with SEQ ID NO:137 over its whole length.

In an embodiment, the chimeric bidirectional signaling transmembrane protein does not comprise the extracellular ligand domain and the transmembrane domain of the ICOSL. Such protein may be represented by SEQ ID NO: 138 or by an amino acid sequence having at least 97%, or at least 98%, or at least 98.5% or at least 99% or at least 99.5% or at least 100% identity with SEQ ID NO:138 over its whole length.

In an embodiment, the chimeric bidirectional signaling transmembrane protein does not comprise the extracellular ligand domain of the ICOSL. Such protein may be represented by SEQ ID NO: 139 or by an amino acid sequence having at least 97%, or at least 98%, or at least 98.5% or at least 99% or at least 99.5% or at least 100% identity with SEQ ID NO:139 over its whole length.

In an embodiment, the chimeric bidirectional signaling transmembrane protein able to transduce at least two, optionally inducible, intracellular signals, comprises:
an extracellular ligand domain, able to interact with the extracellular domain of its interaction partner wherein the extracellular ligand domain is represented by a sequence having at least 80% identity with one of SEQ ID NO: 1-6 as identified in table 1,
a transmembrane domain represented by a sequence having at least 80% identity with one of SEQ ID NO: 20-27 as identified in table 3, and
a heterologous intracellular signaling domain transducing a first signal after binding of the extracellular ligand domain to its interaction partner, wherein the heterologous intracellular signaling domain is represented by a sequence having at least 80% identity with one of SEQ ID NO: 7-19 as identified in table 2,
   wherein the second intracellular signal is transduced via the intracellular domain of the interaction partner. In this embodiment, the sequence identity may be at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the chimeric bidirectional signaling transmembrane protein able to transduce at least two, optionally inducible, intracellular signals, comprises:
an extracellular ligand domain, able to interact with the extracellular domain of its interaction partner wherein the extracellular ligand domain is represented by a sequence having at least 80% identity with one of SEQ ID NO: 1-6, or 174 as identified in table 1,
a transmembrane domain represented by a sequence having at least 80% identity with one of SEQ ID NO: 20-27, or 177 as identified in table 3, and
a heterologous intracellular signaling domain transducing a first signal after binding of the extracellular ligand domain to its interaction partner, wherein the heterologous intracellular signaling domain is represented by a sequence having at least 80% identity with one of SEQ ID NO: 7-19, or 175 as identified in table 2,
   wherein the second intracellular signal is transduced via the intracellular domain of the interaction partner. In this embodiment, the sequence identity may be at least 80%, 81%, 82%, 83%, 84%, 85%, 86%, 87%, 88%, 89%, 90%, 91%, 92%, 93%, 94%, 95%, 96%, 97%, 98%, 99% or 100%.

In one embodiment, the transmembrane domain and the extracellular ligand domain are from the same proteins. Non-limiting examples are CD86-OX40, 41BBL-OX40, OX40L-41BB.

In an embodiment, the chimeric bidirectional signaling transmembrane protein able to transduce at least two, optionally inducible, intracellular signals in one single cell comprises an extracellular ligand domain which is from or derived from a type I transmembrane protein and a heterologous intracellular signaling domain which is from or derived from a type I transmembrane protein. In an embodiment, the chimeric bidirectional signaling transmembrane protein able to transduce at least two, optionally inducible, intracellular signals in one single cell comprises an extracellular ligand domain which is from or derived from a type II transmembrane protein and a heterologous intracellular signaling domain which is from or derived from a type II transmembrane protein.

In an embodiment, the chimeric bidirectional signaling transmembrane protein able to transduce at least two, optionally inducible, intracellular signals in one single cell comprises:
a. an extracellular ligand domain which is from or derived from a type I transmembrane protein and a heterologous intracellular signaling domain which is from or derived from a type II transmembrane protein, or
b. an extracellular ligand domain which is from or derived from a type II transmembrane protein and a heterologous intracellular signaling domain which is from or derived from a type I transmembrane protein.

Such chimeric proteins comprising part of a type I and part of a type II transmembrane protein exhibit surprising and unexpected effects, as type I and type II transmembrane proteins cannot be readily combined into a functional protein. For example, many attempts to fuse an amino acid sequence from a type I transmembrane protein to an amino acid sequence from type II transmembrane protein fail to yield a functional protein, for example, due to an altered N-terminal or C-terminal location of one of the amino acid sequences, inability of the resulting protein to adopt a functional conformation, tertiary structure, transmembrane orientation, or a combination thereof. Surprisingly some of these chimeric proteins have been successfully generated in the experimental part and have been found active.

In an embodiment, the chimeric bidirectional signaling transmembrane protein comprises:
an extracellular ligand domain comprising an amino acid sequence from a tumor necrosis factor superfamily member, a cytokine, a C-type lectin, an immunoglobulin superfamily member, or an antibody or antigen-binding fragment thereof; and
a heterologous intracellular signaling domain comprising an amino acid sequence from a tumor necrosis factor receptor superfamily member, a cytokine receptor, or a C-type lectin receptor.

In an embodiment, the chimeric bidirectional signaling transmembrane protein comprises:
an extracellular ligand domain comprising an amino acid sequence from 41BBL, OX40L, CD86, or RANK, and
a heterologous intracellular signaling domain comprising an amino acid sequence from OX40, 41BB, NKp80, or IL18RAP.

In an embodiment, the chimeric bidirectional signaling transmembrane protein comprises:
an extracellular ligand domain comprising an amino acid sequence from 41BBL, OX40L, CD86, RANK, or CD70, and
a heterologous intracellular signaling domain comprising an amino acid sequence from OX40, 41BB, NKp80, IL18RAP, or IL2RB.

In an embodiment, the chimeric bidirectional signaling transmembrane protein comprises:
(a) the extracellular ligand domain comprises an amino acid sequence from 41BBL and the heterologous intracellular signaling domain comprises an amino acid sequence from OX40, preferably wherein the extracellular ligand domain is from or is derived from a type II transmembrane protein 41BBL and the heterologous intracellular signaling domain is from or is derived from a type I transmembrane protein OX40,
(b) the extracellular ligand domain comprises an amino acid sequence from CD86 and the heterologous intracellular signaling domain comprises an amino acid sequence from OX40, preferably wherein the extracellular ligand domain is from or is derived from a type I transmembrane protein CD86 and the heterologous intracellular signaling domain is from or is derived from a type I transmembrane protein OX40,
(c) the extracellular ligand domain comprises an amino acid sequence from 41BBL and the heterologous intracellular signaling domain comprises an amino acid sequence from NKp80, preferably wherein the extracellular ligand domain is from or is derived from a type II transmembrane protein 41BBL and the heterologous intracellular signaling domain is from or is derived from a type II transmembrane protein NpK80,
(d) the extracellular ligand domain comprises an amino acid sequence from RANK and the heterologous intracellular signaling domain comprises an amino acid sequence from IL18RAP, preferably wherein the extracellular ligand domain is from or is derived from a type I transmembrane protein RANK and the heterologous intracellular signaling domain is from or is derived from a type I transmembrane protein IL18RAP,
(e) the extracellular ligand domain comprises an amino acid sequence from RANK and the heterologous intracellular signaling domain comprises an amino acid sequence from OX40, preferably wherein the extracellular ligand domain is from or is derived from a type I transmembrane protein RANK and the heterologous intracellular signaling domain is from or is derived from a type I transmembrane protein OX40,
(f) the extracellular ligand domain comprises an amino acid sequence from RANK and the heterologous intracellular signaling domain comprises an amino acid sequence from 41BB, preferably wherein the extracellular ligand domain is from or is derived from a type I transmembrane protein RANK and the heterologous intracellular signaling domain is from or is derived from a type I transmembrane protein 41BB,
(g) the extracellular ligand domain comprises an amino acid sequence from OX40L and the heterologous intracellular signaling domain comprises an amino acid sequence from 41BB, preferably wherein the extracellular ligand domain is from or is derived from a type 11 transmembrane protein OX40L and the heterologous intracellular signaling domain is from or is derived from a type I transmembrane protein 41BB, or
(h) the extracellular ligand domain comprises an amino acid sequence from CD86 and the heterologous intracellular signaling domain comprises an amino acid sequence from IL18RAP, preferably wherein the extracellular ligand domain is from or is derived from a type I transmembrane protein CD86 and the heterologous intracellular signaling domain is from or is derived from a type I transmembrane protein IL18RAP.

In an embodiment, the chimeric bidirectional signaling transmembrane protein comprises:
(a) the extracellular ligand domain comprises an amino acid sequence from 41BBL and the heterologous intracellular signaling domain comprises an amino acid sequence from OX40, preferably wherein the extracellular ligand domain is from or is derived from a type 11 transmembrane protein 41BBL and the heterologous intracellular signaling domain is from or is derived from a type I transmembrane protein OX40,
(b) the extracellular ligand domain comprises an amino acid sequence from CD86 and the heterologous intracellular signaling domain comprises an amino acid sequence from OX40, preferably wherein the extracellular ligand domain is from or is derived from a type I transmembrane protein CD86 and the heterologous intracellular signaling domain is from or is derived from a type I transmembrane protein OX40,
(c) the extracellular ligand domain comprises an amino acid sequence from 41BBL and the heterologous intracellular signaling domain comprises an amino acid sequence from NKp80, preferably wherein the extracellular ligand domain is from or is derived from a type 11 transmembrane protein 41BBL and the heterologous intracellular signaling domain is from or is derived from a type 11 transmembrane protein NpK80,
(d) the extracellular ligand domain comprises an amino acid sequence from RANK and the heterologous intracellular signaling domain comprises an amino acid sequence from IL18RAP, preferably wherein the extracellular ligand domain is from or is derived from a type I transmembrane protein RANK and the heterologous intracellular signaling domain is from or is derived from a type I transmembrane protein IL18RAP,
(e) the extracellular ligand domain comprises an amino acid sequence from RANK and the heterologous intracellular signaling domain comprises an amino acid sequence from OX40, preferably wherein the extracellular ligand domain is from or is derived from a type I transmembrane protein RANK and the heterologous intracellular signaling domain is from or is derived from a type I transmembrane protein OX40,
(f) the extracellular ligand domain comprises an amino acid sequence from RANK and the heterologous intracellular signaling domain comprises an amino acid sequence from 41BB, preferably wherein the extracellular ligand domain is from or is derived from a type I transmembrane protein RANK and the heterologous intracellular signaling domain is from or is derived from a type I transmembrane protein 41BB,
(g) the extracellular ligand domain comprises an amino acid sequence from OX40L and the heterologous intracellular signaling domain comprises an amino acid sequence from 41BB, preferably wherein the extracellular ligand domain is from or is derived from a type 11 transmembrane protein OX40L and the heterologous intracellular signaling domain is from or is derived from a type I transmembrane protein 41BB,
(h) the extracellular ligand domain comprises an amino acid sequence from CD86 and the heterologous intracellular signaling domain comprises an amino acid sequence from IL18RAP, preferably wherein the extracellular ligand domain is from or is derived from a type I transmembrane protein CD86 and the heterologous intracellular signaling domain is from or is derived from a type I transmembrane protein IL18RAP,
(i) the extracellular ligand domain comprises an amino acid sequence from CD70 and the heterologous intracellular signaling domain comprises an amino acid sequence from OX40, preferably wherein the extracellular ligand domain is from or is derived from a type 11 transmembrane protein CD70 and the heterologous intracellular signaling domain is from or is derived from a type I transmembrane protein OX40, or
(j) the extracellular ligand domain comprises an amino acid sequence from 41BBL and the heterologous intracellular signaling domain comprises an amino acid sequence from OX40 and an amino acid sequence from IL2RB, preferably wherein the extracellular ligand domain is from or is derived from a type II transmembrane protein 41BBL and the heterologous intracellular signaling domain is from or is derived from a type I transmembrane protein OX40 and from a type I transmembrane protein IL2RB.

Each of these chimeric proteins has been generated in the experimental part and their functionality has been confirmed (see e.g., Examples 3-5, 10).

In an embodiment, the chimeric bidirectional signaling transmembrane protein identified under a) is represented by an amino acid sequence having at least 80% identity or similarity with SEQ ID NO: 45, 46, 57, 58, 59, 60, 61, 62, 63, 64, or 65 as identified in table 5.

In an embodiment, the chimeric bidirectional signaling transmembrane protein identified under a) is represented by an amino acid sequence having at least 80% identity or similarity with SEQ ID NO: 45, 46, 57, 58, 59, 60, 61, 62, 63, 64, 65, 178, or 179 as identified in table 5.

In an embodiment, the chimeric bidirectional signaling transmembrane protein identified under b) is represented by an amino acid sequence having at least 80% identity or similarity with SEQ ID NO:52, 53, or 73 as identified in table 5.

In an embodiment, the chimeric bidirectional signaling transmembrane protein identified under c) is represented by an amino acid sequence having at least 80% identity or similarity with SEQ ID NO:47 or 48 as identified in table 5.

In an embodiment, the chimeric bidirectional signaling transmembrane protein identified under d) is represented by an amino acid sequence having at least 80% identity or similarity with SEQ ID NO:78 as identified in table 5.

In an embodiment, the chimeric bidirectional signaling transmembrane protein identified under e) is represented by an amino acid sequence having at least 80% identity or similarity with SEQ ID NO: 76 as identified in table 5.

In an embodiment, the chimeric bidirectional signaling transmembrane protein identified under f) is represented by an amino acid sequence having at least 80% identity or similarity with SEQ ID NO: 77 as identified in table 5.

In an embodiment, the chimeric bidirectional signaling transmembrane protein identified under g) is represented by an amino acid sequence having at least 80% identity or similarity with SEQ ID NO: 49, 50, or 51 as identified in table 5.

In an embodiment, the chimeric bidirectional signaling transmembrane protein identified under h) is represented by an amino acid sequence having at least 80% identity or similarity with SEQ ID NO: 71 or 72 as identified in table 5.

In an embodiment, the chimeric bidirectional signaling transmembrane protein identified under i) is represented by an amino acid sequence having at least 80% identity or similarity with SEQ ID NO: 182 or 183 as identified in table 5.

In an embodiment, the chimeric bidirectional signaling transmembrane protein identified under j) is represented by an amino acid sequence having at least 80% identity or similarity with SEQ ID NO: 179 as identified in table 5.

In this embodiment, the sequence identity or similarity may be at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the chimeric bidirectional signaling transmembrane protein does not contain an ITAM or an intracellular domain from a TCR signaling complex. In this context in an embodiment, an ITAM motif is “YxxL/I-x6-8-YxxL/I” wherein x stands for any amino acid. X6-8 means any stretch of 6, 7 or 8 amino acids, Y is Tyrosine, L is Leucine, I is Isoleucine (PFAM source https://pfam.xfam.org/family/ITAM or https://www.sciencedirect.com/science/article/abs/pii/S0962892406001498 article).

Non-limiting examples of MIDIS protein sequences, and sequences that can be included in MIDIS proteins, are provided in Table 5.

TABLE 5

| SEQ | | | |
|---|---|---|---|
| ID | | | |
| NO: | Name | Description | Sequence |
| 45 | 41BBL-OX40 | WT 41BBL | MLGRDQRLPPDAHKPPGGGSFRTPIQEEQADAHSTLAKITSGS |
| | | with 41BBL | YASDASLDPEAPWPPAPRARACRVLPWALVAGLLLLLLLAAAC |
| | | TM and OX40 | AVFLACPWAVSGARASPGSAASPRLREGPELSPDDPAGLLDL |
| | | intracellular | RQGMFAQLVAQNVLLIDGPLSWYSDPGLAGVSLTGGLSYKED |
| | | signaling | TKELVVAKAGVYYVFFQLELRRVVAGEGSGSVSLALHLQPLRS |
| | | domain | AAGAAALALTVDLPPASSEARNSAFGFQGRLLHLSAGQRLGVH |
| | | | LHTEARARHAWQLTQGATVLGLFRVTPEIPAGLPSPRSE |
| 46 | 41BBL13W- | 41BBL ligand | MLGIKALTSHADAQEEQIPTRFSGGGPPKHADPPLRQDRTSGS |
| | OX40rev | binding | WPPAPRARACRVLPWALVAGLLLLLLLAAACAVFLACPWAVSG |
| | | domain, TM | ARASPGSAASPRL |
| | | domain, and | REGPELSPDDPAGLLDLRQGMFAQLVAQNVLLIDGPLSWYSD |
| | | partial | PGLAGVSLTGGLSYKEDTKELVVAKAGVYYVFFQLELRRVVAG |
| | | intracellular | EGSGSVSLALHLQPLRSAAGAAALALTVDLPPASSEARNSAFG |
| | | domain with | FQGRLLHLSAGQRLGVHLHTEARARHAWQLTQGATVLGLFRV |
| | | deletion of the | TPEIPAGLPSPRSE |
| | | first 12 amino | |
| | | acids | |
| | | containing a | |
| | | putative | |
| | | casein kinase | |
| | | I motif; and an | |
| | | OX40 | |
| | | intracellular | |
| | | signaling | |
| | | domain that is | |
| | | inverted | |
| 47 | 41BBL- | 41BBL WT | MQDEDGYMTLNVQSKKRSSAQTSQLTFKDYSVTLHWYASDAS |
| | NKp80 | with 41BBL | LDPEAPWPPAPRARACRVLPWALVAGLLLLLLLAAACAVFLAC |
| | | TM without | PWAVSGARASPGSAASPRLREGPELSPDDPAGLLDLRQGMFA |
| | | first 2 amino | QLVAQNVLLIDGPLSWYSDPGLAGVSLTGGLSYKEDTKELVVA |
| | | acids with | KAGVYYVFFQLELRRVVAGEGSGSVSLALHLQPLRSAAGAAAL |
| | | NKp80 | ALTVDLPPASSEARNSAFGFQGRLLHLSAGQRLGVHLHTEARA |
| | | intracellular | RHAWQLTQGATVLGLFRVTPEIPAGLPSPRSE |
| | | signaling | |
| | | domain | |
| 48 | 41BBLmincyto- | 41BBL ligand | MQDEDGYMTLNVQSKKRSSAQTSQLTFKDYSVTLHWYKWAL |
| | NKp80 | domain and | VAGLLLLLLLAAACAVFLACPWAVSGARASPGSAASPRLREGP |
| | | TM linked to | ELSPDDPAGLLDLRQGMFAQLVAQNVLLIDGPLSWYSDPGLAG |
| | | an NKp80 | VSLTGGLSYKEDTKELVVAKAGVYYVFFQLELRRVVAGEGSGS |
| | | intracellular | VSLALHLQPLRSAAGAAALALTVDLPPASSEARNSAFGFQGRL |
| | | signaling | LHLSAGQRLGVHLHTEARARHAWQLTQGATVLGLFRVTPEIPA |
| | | domain, | GLPSPRSE |
| | | lacking the | |
| | | intracellular | |
| | | sequence of | |
| | | 41BBL | |
| 49 | OX40L-41BB | WT OX40L | MLGKRGRKKLLYIFKQPFMRPVQTTQEEDGCSCRFPEEEEGG |
| | | including TM | CELTSGSERVQPLEENVGNAARPRFERNKLLLVASVIQGLGLLL |
| | | and IC | CFTYICLHFSALQVSHRYPRIQSIKVQFTEYKKEKGFILTSQKED |
| | | domain and | EIMKVQNNSVIINCDGFYLISLKGYFSQEVNISLHYQKDEEPLFQ |
| | | 41BB | LKKVRSVNSLMVASLTYKDKVYLNVTTDNTSLDDFHVNGGELIL |
| | | intracellular | IHQNPGEFCVL |
| | | signaling | |
| | | domain | |
| | | flipped in | |
| | | structural | |
| | | orientation | |
| 50 | OX40L- | OX40L WT | MLGKRGRKKLLYIFKQPFMRPVQTTQEEDGCSCRFPEEEEGG |
| | 41BB | including TM | CELGGSAERVQPLEENVGNAARPRFERNKLLLVASVIQGLGLL |
| | | and IC | LCFTYICLHFSALQVSHRYPRIQSIKVQFTEYKKEKGFILTSQKE |
| | | domain with | DEIMKVQNNSVIINCDGFYLISLKGYFSQEVNISLHYQKDEEPLF |
| | | 41BB | QLKKVRSVNSLMVASLTYKDKVYLNVTTDNTSLDDFHVNGGEL |
| | | intracellular | ILIHQNPGEFCVL |
| | | signaling | |
| | | domain | |
| | | separated by | |
| | | GGSA linker | |
| 51 | OX40L- | OX40L ligand | MLGLECGGEEEEPFRCSCGDEEQTTQVPRMFPQKFIYLLKKR |
| | 41BBrev | binding and | GRKLGGSAERVQPLEENVGNAARPRFERNKLLLVASVIQGLGL |
| | | TM domain | LLCFTYICLHFSALQVSHRYPRIQSIKVQFTEYKKEKGFILTSQK |
| | | with 41BB | EDEIMKVQNNSVIINCDGFYLISLKGYFSQEVNISLHYQKDEEPL |
| | | intracellular | FQLKKVRSVNSLMVASLTYKDKVYLNVTTDNTSLDDFHVNGGE |
| | | signaling | LILIHQNPGEFCVL |
| | | domain that is | |
| | | inverted | |
| 52 | CD86-OX40 | CD86 WT | MDPQCTMGLSNILFVMAFLLSGAAPLKIQAYFNETADLPCQFA |
| | | including TM | NSQNQSLSELVVFWQDQENLVLNEVYLGKEKFDSVHSKYMGR |
| | | and IC | TSFDSDSWTLRLHNLQIKDKGLYQCIIHHKKPTGMIRIHQMNSE |
| | | domain linked | LSVLANFSQPEIVPISNITENVYINLTCSSIHGYPEPKKMSVLLRT |
| | | to OX40 | KNSTIEYDGVMQKSQDNVTELYDVSISLSVSFPDVTSNMTIFCIL |
| | | intracellular | ETDKTRLLSSPFSIELEDPQPPPDHIPWITAVLPTVIICVMVFCLIL |
| | | signaling | WKWKKKKRPRNSYKCGTNTMEREESEQTKKREKIHIPERSDE |
| | | domain | AQRVFKSSKTSSCDKSDTCFGSGRDQRLPPDAHKPPGGGSF |
| | | | RTPIQEEQADAHSTLAKI |
| 53 | CD86delP276- | CD86 ligand | MDPQCTMGLSNILFVMAFLLSGAAPLKIQAYFNETADLPCQFA |
| | OX40 | binding | NSQNQSLSELVVFWQDQENLVLNEVYLGKEKFDSVHSKYMGR |
| | | domain and | TSFDSDSWTLRLHNLQIKDKGLYQCIIHHKKPTGMIRIHQMNSE |
| | | TM domain, | LSVLANFSQPEIVPISNITENVYINLTCSSIHGYPEPKKMSVLLRT |
| | | with a | KNSTIEYDGVMQKSQDNVTELYDVSISLSVSFPDVTSNMTIFCIL |
| | | deletion of | ETDKTRLLSSPFSIELEDPQPPPDHIPWITAVLPTVIICVMVFCLIL |
| | | amino acids | WKWKKKKRPGRDQRLPPDAHKPPGGGSFRTPIQEEQADAHS |
| | | 277-329 of | TLAKI |
| | | CD86, and an | |
| | | OX40 | |
| | | intracellular | |
| | | signaling | |
| | | domain | |
| 54 | 41BBL | WT 41BBL | YASDASLDPEAPWPPAPRARACRVLPWALVAGLLLLLLLAAAC |
| | | | AVFLACPWAVSGARASPGSAASPRLREGPELSPDDPAGLLDL |
| | | | RQGMFAQLVAQNVLLIDGPLSWYSDPGLAGVSLTGGLSYKED |
| | | | TKELVVAKAGVYYVFFQLELRRVVAGEGSGSVSLALHLQPLRS |
| | | | AAGAAALALTVDLPPASSEARNSAFGFQGRLLHLSAGQRLGVH |
| | | | LHTEARARHAWQLTQGATVLGLFRVTPEIPAGLPSPRSE |
| 55 | 41BBL- | WT 41BBL of | MSKSTGSWALVAGLLLLLLLAAACAVFLACPWAVSGARASPGS |
| | mincyto | which | AASPRLREGPELSPDDPAGLLDLRQGMFAQLVAQNVLLIDGPL |
| | | intracellular | SWYSDPGLAGVSLTGGLSYKEDTKELVVAKAGVYYVFFQLELR |
| | | domain is | RVVAGEGSGSVSLALHLQPLRSAAGAAALALTVDLPPASSEAR |
| | | swapped for | NSAFGFQGRLLHLSAGQRLGVHLHTEARARHAWQLTQGATVL |
| | | small linker | GLFRVTPEIPAGLPSPRSE |
| | | MSKSTGS | |
| 56 | 41BBL13W | WT 41BBL | MEWALVAGLLLLLLLAAACAVFLACPWAVSGARASPGSAASPR |
| | | with deletion | LREGPELSPDDPAGLLDLRQGMFAQLVAQNVLLIDGPLSWYSD |
| | | of the first 12 | PGLAGVSLTGGLSYKEDTKELVVAKAGVYYVFFQLELRRVVAG |
| | | amino acids | EGSGSVSLALHLQPLRSAAGAAALALTVDLPPASSEARNSAFG |
| | | containing a | FQGRLLHLSAGQRLGVHLHTEARARHAWQLTQGATVLGLFRV |
| | | putative | TPEIPAGLPSPRSE |
| | | casein kinase | |
| | | I motif | |
| 57 | 41BBL- | 41BBL WT | MLGIKALTSHADAQEEQIPTRESGGGPPKHADPPLRQDRTSGS |
| | OX40rev | including TM | YASDASLDPEAPWPPAPRARACRVLPWALVAGLLLLLLLAAAC |
| | | and IC | AVFLACPWAVSGARASPGSAASPRLREGPELSPDDPAGLLDL |
| | | domain | RQGMFAQLVAQNVLLIDGPLSWYSDPGLAGVSLTGGLSYKED |
| | | without first 2 | TKELVVAKAGVYYVFFQLELRRVVAGEGSGSVSLALHLQPLRS |
| | | amino acids | AAGAAALALTVDLPPASSEARNSAFGFQGRLLHLSAGQRLGVH |
| | | with inverted | LHTEARARHAWQLTQGATVLGLFRVTPEIPAGLPSPRSE |
| | | OX40 | |
| | | intracellular | |
| | | signaling | |
| | | domain | |
| 58 | 41BBL-rev | 41BBL ligand | MLLLVTSLLLCELPHPAFLLIPDQGMFAQLVAQNVLLIDGPLSW |
| | extra- | binding | YSDPGLAGVSLTGGLSYKEDTKELVVAKAGVYYVFFQLELRRV |
| | OX40tm-cyto | domain core | VAGEGSGSVSLALHLQPLRSAAGAAALALTVDLPPASSEARNS |
| | | in a type I | AFGFQGRLLHLSAGQRLGVHLHTEARARHAWQLTQGATVLGL |
| | | orientation | FRVTPEIPAGLPSPRSERLDLLGAPDDPSLEPGERLRPSAASG |
| | | with lgK | PSARAVAAILGLGLVLGLLGPLAILLALYLLRRDQRLPPDAHKPP |
| | | leader | GGGSFRTPIQEEQADAHSTLAKI |
| | | sequence and | |
| | | inverted | |
| | | extracellular | |
| | | N-terminal | |
| | | part with a | |
| | | OX40 TM and | |
| | | intracellular | |
| | | signaling | |
| | | domain | |
| 59 | 41BBLmincyto- | WT 41BBL | MLGRDQRLPPDAHKPPGGGSFRTPIQEEQADAHSTLAKITSGS |
| | OX40 | EC and TM | WALVAGLLLLLLLAAACAVFLACPWAVSGARASPGSAASPRLR |
| | | linked to IC | EGPELSPDDPAGLLDLRQGMFAQLVAQNVLLIDGPLSWYSDP |
| | | signaling | GLAGVSLTGGLSYKEDTKELVVAKAGVYYVFFQLELRRVVAGE |
| | | domain of | GSGSVSLALHLQPLRSAAGAAALALTVDLPPASSEARNSAFGF |
| | | OX40 | QGRLLHLSAGQRLGVHLHTEARARHAWQLTQGATVLGLFRVT |
| | | separated by | PEIPAGLPSPRSE |
| | | a TSGS linker | |
| 60 | 41BBL13W- | WT 41BBL | MEIKALTSHADAQEEQIPTRFSGGGPPKHADPPLRQDRRLLWP |
| | OX40rev | ligand | PAPRARACRVLPWALVAGLLLLLLLAAACAVFLACPWAVSGAR |
| | | binding, TM | ASPGSAASPRLREGPELSPDDPAGLLDLRQGMFAQLVAQNVL |
| | | and IC | LIDGPLSWYSDPGLAGVSLTGGLSYKEDTKELVVAKAGVYYVF |
| | | domain | FQLELRRVVAGEGSGSVSLALHLQPLRSAAGAAALALTVDLPP |
| | | without first | ASSEARNSAFGFQGRLLHLSAGQRLGVHLHTEARARHAWQLT |
| | | 12 amino | QGATVLGLFRVTPEIPAGLPSPRSE |
| | | acids of the | |
| | | IC domain | |
| | | linked to the | |
| | | inverted | |
| | | OX40 IC | |
| | | signaling | |
| | | domain | |
| 61 | 41BBL- | WT 41BBL | MLGIKALTSHADAQEEQIPTRFSGGGPPKHADPPLRQDRTSGS |
| | mincyto- | EC and TM | WALVAGLLLLLLLAAACAVFLACPWAVSGARASPGSAASPRLR |
| | OX40rev | linked to IC | EGPELSPDDPAGLLDLRQGMFAQLVAQNVLLIDGPLSWYSDP |
| | | signaling | GLAGVSLTGGLSYKEDTKELVVAKAGVYYVFFQLELRRVVAGE |
| | | domain of | GSGSVSLALHLQPLRSAAGAAALALTVDLPPASSEARNSAFGF |
| | | inverted | QGRLLHLSAGQRLGVHLHTEARARHAWQLTQGATVLGLFRVT |
| | | OX40 | PEIPAGLPSPRSE |
| | | separated by | |
| | | a TSGS linker | |
| 62 | 41BBL- | 41BBL WT | MLGIKALTSHADAQEEQIPTRFSGGGPPKHADPPLRQDRRLLY |
| | OX40ENrev | EC, TM and | LATSGSYASDASLDPEAPWPPAPRARACRVLPWALVAGLLLLL |
| | | IC without first | LLAAACAVFLACPWAVSGARASPGSAASPRLREGPELSPDDP |
| | | 2 amino acids | AGLLDLRQGMFAQLVAQNVLLIDGPLSWYSDPGLAGVSLTGGL |
| | | linked to | SYKEDTKELVVAKAGVYYVFFQLELRRVVAGEGSGSVSLALHL |
| | | inverted | QPLRSAAGAAALALTVDLPPASSEARNSAFGFQGRLLHLSAGQ |
| | | OX40 | RLGVHLHTEARARHAWQLTQGATVLGLFRVTPEIPAGLPSPRS |
| | | enlarged | E |
| | | (including | |
| | | amino acids | |
| | | ALYLLR) IC | |
| | | signaling | |
| | | domain | |
| 63 | 41BBL- | 41BBL WT | MLGALYLLRRDQRLPPDAHKPPGGGSFRTPIQEEQADAHSTLA |
| | OX40EN | EC, TM and | KITSGSYASDASLDPEAPWPPAPRARACRVLPWALVAGLLLLL |
| | | IC without first | LLAAACAVFLACPWAVSGARASPGSAASPRLREGPELSPDDP |
| | | 2 amino acids | AGLLDLRQGMFAQLVAQNVLLIDGPLSWYSDPGLAGVSLTGGL |
| | | linked to | SYKEDTKELVVAKAGVYYVFFQLELRRVVAGEGSGSVSLALHL |
| | | OX40 | QPLRSAAGAAALALTVDLPPASSEARNSAFGFQGRLLHLSAGQ |
| | | enlarged | RLGVHLHTEARARHAWQLTQGATVLGLFRVTPEIPAGLPSPRS |
| | | (including | E |
| | | amino acids | |
| | | ALYLLR) | |
| | | intracellular | |
| | | signaling | |
| | | domain | |
| 64 | 41BBLmincyto- | WT 41BBL | MLGRDQRLPPDAHKPPGGGSFRTPIQEEQADAHSTLAKISLNG |
| | 13alink- | EC and TM | GGGSGGGGSGGGGSGGGGSGGPWALVAGLLLLLLLAAACAV |
| | OX40 | linked with a | FLACPWAVSGARASPGSAASPRLREGPELSPDDPAGLLDLRQ |
| | | 13 amino acid | GMFAQLVAQNVLLIDGPLSWYSDPGLAGVSLTGGLSYKEDTKE |
| | | linker to the | LVVAKAGVYYVFFQLELRRVVAGEGSGSVSLALHLQPLRSAAG |
| | | OX40 IC | AAALALTVDLPPASSEARNSAFGFQGRLLHLSAGQRLGVHLHT |
| | | signaling | EARARHAWQLTQGATVLGLFRVTPEIPAGLPSPRSE |
| | | domain | |
| 65 | OX40Lmincyto- | OX40L WT | MLGLECGGEEEEPFRCSCGDEEQTTQVPRMFPQKFIYLLKKR |
| | 41BBrev | EC and TM | GRKLLLVASVIQGLGLLLCFTYICLHFSALQVSHRYPRIQSIKVQ |
| | | linked to the | FTEYKKEKGFILTSQKEDEIMKVQNNSVIINCDGFYLISLKGYFS |
| | | inverted IC | QEVNISLHYQKDEEPLFQLKKVRSVNSLMVASLTYKDKVYLNV |
| | | signaling | TTDNTSLDDFHVNGGELILIHQNPGEFCVL |
| | | domain by a | |
| | | short linker | |
| 66 | OX40L WT | OX40L WT | MERVQPLEENVGNAARPRFERNKLLLVASVIQGLGLLLCFTYIC |
| | | | LHFSALQVSHRYPRIQSIKVQFTEYKKEKGFILTSQKEDEIMKVQ |
| | | | NNSVIINCDGFYLISLKGYFSQEVNISLHYQKDEEPLFQLKKVRS |
| | | | VNSLMVASLTYKDKVYLNVTTDNTSLDDFHVNGGELILIHQNPG |
| | | | EFCVL |
| 67 | OX40Lmincyto- | OX40L WT, | MLGKRGRKKLLYIFKQPFMRPVQTTQEEDGCSCRFPEEEEGG |
| | 41BB | EC and | CELGGSLLLVASVIQGLGLLLCFTYICLHFSALQVSHRYPRIQSI |
| | | TMlinked to | KVQFTEYKKEKGFILTSQKEDEIMKVQNNSVIINCDGFYLISLKG |
| | | 41BB IC | YFSQEVNISLHYQKDEEPLFQLKKVRSVNSLMVASLTYKDKVYL |
| | | signaling | NVTTDNTSLDDFHVNGGELILIHQNPGEFCVL |
| | | domain by a | |
| | | GGS linker | |
| 68 | CD86 WT | CD86 WT | MDPQCTMGLSNILFVMAFLLSGAAPLKIQAYFNETADLPCQFA |
| | | | NSQNQSLSELVVFWQDQENLVLNEVYLGKEKFDSVHSKYMGR |
| | | | TSFDSDSWTLRLHNLQIKDKGLYQCIIHHKKPTGMIRIHQMNSE |
| | | | LSVLANFSQPEIVPISNITENVYINLTCSSIHGYPEPKKMSVLLRT |
| | | | KNSTIEYDGVMQKSQDNVTELYDVSISLSVSFPDVTSNMTIFCIL |
| | | | ETDKTRLLSSPFSIELEDPQPPPDHIPWITAVLPTVIICVMVFCLIL |
| | | | WKWKKKKRPRNSYKCGTNTMEREESEQTKKREKIHIPERSDE |
| | | | AQRVFKSSKTSSCDKSDTCF |
| 69 | CD86- | CD86 with a | MDPQCTMGLSNILFVMAFLLSGAAPLKIQAYFNETADLPCQFA |
| | delP276 | deletion of the | NSQNQSLSELVVFWQDQENLVLNEVYLGKEKFDSVHSKYMGR |
| | | IC part up to | TSFDSDSWTLRLHNLQIKDKGLYQCIIHHKKPTGMIRIHQMNSE |
| | | the point | LSVLANFSQPEIVPISNITENVYINLTCSSIHGYPEPKKMSVLLRT |
| | | which is | KNSTIEYDGVMQKSQDNVTELYDVSISLSVSFPDVTSNMTIFCIL |
| | | shown to be | ETDKTRLLSSPFSIELEDPQPPPDHIPWITAVLPTVIICVMVFCLIL |
| | | involved in | WKWKKKKRP |
| | | correct | |
| | | cellular | |
| | | localization of | |
| | | CD86 | |
| | | (deletion of | |
| | | amino acid | |
| | | 277-329 of | |
| | | CD86) | |
| 70 | CD86mincyto | CD86 WT | MDPQCTMGLSNILFVMAFLLSGAAPLKIQAYFNETADLPCQFA |
| | | without IC | NSQNQSLSELVVFWQDQENLVLNEVYLGKEKFDSVHSKYMGR |
| | | domain | TSFDSDSWTLRLHNLQIKDKGLYQCIIHHKKPTGMIRIHQMNSE |
| | | | LSVLANFSQPEIVPISNITENVYINLTCSSIHGYPEPKKMSVLLRT |
| | | | KNSTIEYDGVMQKSQDNVTELYDVSISLSVSFPDVTSNMTIFCIL |
| | | | ETDKTRLLSSPFSIELEDPQPPPDHIPWITAVLPTVIICVMVFCLIL |
| | | | W |
| 71 | CD86mincyto- | CD86 WT EC | MDPQCTMGLSNILFVMAFLLSGAAPLKIQAYFNETADLPCQFA |
| | IL18RAP | and TM linked | NSQNQSLSELVVFWQDQENLVLNEVYLGKEKFDSVHSKYMGR |
| | | to IC domain | TSFDSDSWTLRLHNLQIKDKGLYQCIIHHKKPTGMIRIHQMNSE |
| | | of IL18RAP | LSVLANFSQPEIVPISNITENVYINLTCSSIHGYPEPKKMSVLLRT |
| | | | KNSTIEYDGVMQKSQDNVTELYDVSISLSVSFPDVTSNMTIFCIL |
| | | | ETDKTRLLSSPFSIELEDPQPPPDHIPWITAVLPTVIICVMVFCLIL |
| | | | WSALLYRHWIEIVLLYRTYQSKDQTLGDKKDFDAFVSYAKWSS |
| | | | FPSEATSSLSEEHLALSLFPDVLENKYGYSLCLLERDVAPGGVY |
| | | | AEDIVSIIKRSRRGIFILSPNYVNGPSIFELQAAVNLALDDQTLKLI |
| | | | LIKFCYFQEPESLPHLVKKALRVLPTVTWRGLKSVPPNSRFWA |
| | | | KMRYHMPVKNSQGFTWNQLRITSRIFQWKGLSRTETTGRSSQ |
| | | | PKEW |
| 72 | CD86- | CD86 WT EC, | MDPQCTMGLSNILFVMAFLLSGAAPLKIQAYFNETADLPCQFA |
| | IL18RAP | TM and IC | NSQNQSLSELVVFWQDQENLVLNEVYLGKEKFDSVHSKYMGR |
| | | domain linked | TSFDSDSWTLRLHNLQIKDKGLYQCIIHHKKPTGMIRIHQMNSE |
| | | to IL18RAP | LSVLANFSQPEIVPISNITENVYINLTCSSIHGYPEPKKMSVLLRT |
| | | IC domain | KNSTIEYDGVMQKSQDNVTELYDVSISLSVSFPDVTSNMTIFCIL |
| | | | ETDKTRLLSSPFSIELEDPQPPPDHIPWITAVLPTVIICVMVFCLIL |
| | | | WKWKKKKRPRNSYKCGTNTMEREESEQTKKREKIHIPERSDE |
| | | | AQRVFKSSKTSSCDKSDTCFSALLYRHWIEIVLLYRTYQSKDQT |
| | | | LGDKKDFDAFVSYAKWSSFPSEATSSLSEEHLALSLFPDVLEN |
| | | | KYGYSLCLLERDVAPGGVYAEDIVSIIKRSRRGIFILSPNYVNGP |
| | | | SIFELQAAVNLALDDQTLKLILIKFCYFQEPESLPHLVKKALRVLP |
| | | | TVTWRGLKSVPPNSRFWAKMRYHMPVKNSQGFTWNQLRITS |
| | | | RIFQWKGLSRTETTGRSSQPKEW |
| 73 | CD86IgV- | WT 41BBL | MLGRDQRLPPDAHKPPGGGSFRTPIQEEQADAHSTLAKITSGS |
| | 41BBL-OX40 | EC, TM and | YASDASLDPEAPWPPAPRARACRVLPWALVAGLLLLLLLAAAC |
| | | IC without first | AVFLACPWAVSGARASPGSAASPRLREGPELSPDDPAGLLDL |
| | | two amino | RQGMFAQLVAQNVLLIDGPLSWYSDPGLAGVSLTGGLSYKED |
| | | acids. | TKELVVAKAGVYYVFFQLELRRVVAGEGSGSVSLALHLQPLRS |
| | | An CD86 IgV | AAGAAALALTVDLPPASSEARNSAFGFQGRLLHLSAGQRLGVH |
| | | domain was | LHTEARARHAWQLTQGATVLGLFRVTPEIPAGLPSPRSESLNG |
| | | linked with a | GGGSGGGGSGGGGSGGGGSGGGGSTSAPLKIQAYFNETADL |
| | | 30 amino acid | PCQFANSQNQSLSELVVFWQDQENLVLNEVYLGKEKFDSVHS |
| | | linker to WT | KYMGRTSFDSDSWTLRLHNLQIKDKGLYQCIIHHKKPTGMIRIH |
| | | 41BBL EC | QMNSELSVLAN |
| | | domain. The | |
| | | OX40 IC | |
| | | signaling | |
| | | domain was | |
| | | linked to the | |
| | | IC domain of | |
| | | 41BBLby | |
| | | TSGS linker | |
| 74 | RANK WT | RANK WT | MAPRARRRRPLFALLLLCALLARLQVALQIAPPCTSEKHYEHLG |
| | | | RCCNKCEPGKYMSSKCTTTSDSVCLPCGPDEYLDSWNEEDK |
| | | | CLLHKVCDTGKALVAVVAGNSTTPRRCACTAGYHWSQDCECC |
| | | | RRNTECAPGLGAQHPLQLNKDTVCKPCLAGYFSDAFSSTDKC |
| | | | RPWTNCTFLGKRVEHHGTEKSDAVCSGSRKPPNEPHVYLPGL |
| | | | IILLLFASVALVAAIIFGVCYRKKGKALTANLWHWINEACGRLSG |
| | | | DKESSGDSCVSTHTANFGQQGACEGVLLLTLEEKTFPEDMCY |
| | | | PDQGGVCQGTCVGGGPYAQGEDARMLSLVSKTEIEEDSFRQ |
| | | | MPTEDEYMDRPSQPTDQLLFLTEPGSKSTPPFSEPLEVGEND |
| | | | SLSQCFTGTQSTVGSESCNCTEPLCRTDWTPMSSENYLQKEV |
| | | | DSGHCPHWAASPSPNWADVCTGCRNPPGEDCEPLVGSPKR |
| | | | GPLPQCAYGMGLPPEEEASRTEARDQPEDGADGRLPSSARA |
| | | | GAGSGSSPGGQSPASGNVTGNSNSTFISSGQVMNFKGDIIVVY |
| | | | VSQTSQEGAAAAAEPMGRPVQEETLARRDSFAGNGPRFPDP |
| | | | CGGPEGLREPEKASRPVQEQGGAKA |
| 75 | RANKmincyto | RANK WT | MAPRARRRRPLFALLLLCALLARLQVALQIAPPCTSEKHYEHLG |
| | | without the IC | RCCNKCEPGKYMSSKCTTTSDSVCLPCGPDEYLDSWNEEDK |
| | | domain | CLLHKVCDTGKALVAVVAGNSTTPRRCACTAGYHWSQDCECC |
| | | | RRNTECAPGLGAQHPLQLNKDTVCKPCLAGYFSDAFSSTDKC |
| | | | RPWTNCTFLGKRVEHHGTEKSDAVCSGSRKPPNEPHVYLPGL |
| | | | IILLLFASVALVAAIIFGV |
| 76 | RANK-OX40 | EC domain of | MAPRARRRRPLFALLLLCALLARLQVALQIAPPCTSEKHYEHLG |
| | | RANK linked | RCCNKCEPGKYMSSKCTTTSDSVCLPCGPDEYLDSWNEEDK |
| | | to TM and IC | CLLHKVCDTGKALVAVVAGNSTTPRRCACTAGYHWSQDCECC |
| | | of OX40 | RRNTECAPGLGAQHPLQLNKDTVCKPCLAGYFSDAFSSTDKC |
| | | | RPWTNCTFLGKRVEHHGTEKSDAVCSGSRKPPNEPHVYLPVA |
| | | | AILGLGLVLGLLGPLAILLALYLLRRDQRLPPDAHKPPGGGSFRT |
| | | | PIQEEQADAHSTLAKI |
| 77 | RANK-41BB | EC domain of | MAPRARRRRPLFALLLLCALLARLQVALQIAPPCTSEKHYEHLG |
| | | RANK linked | RCCNKCEPGKYMSSKCTTTSDSVCLPCGPDEYLDSWNEEDK |
| | | to TM and IC | CLLHKVCDTGKALVAVVAGNSTTPRRCACTAGYHWSQDCECC |
| | | domain of | RRNTECAPGLGAQHPLQLNKDTVCKPCLAGYFSDAFSSTDKC |
| | | 41BB | RPWTNCTFLGKRVEHHGTEKSDAVCSGSRKPPNEPHVYLPIIS |
| | | | FFLALTSTALLFLLFFLTLRFSVVKRGRKKLLYIFKQPFMRPVQT |
| | | | TQEEDGCSCRFPEEEEGGCEL |
| 78 | RANK- | EC domain of | MAPRARRRRPLFALLLLCALLARLQVALQIAPPCTSEKHYEHLG |
| | IL18RAP | RANK linked | RCCNKCEPGKYMSSKCTTTSDSVCLPCGPDEYLDSWNEEDK |
| | | to the TM and | CLLHKVCDTGKALVAVVAGNSTTPRRCACTAGYHWSQDCECC |
| | | IC domain of | RRNTECAPGLGAQHPLQLNKDTVCKPCLAGYFSDAFSSTDKC |
| | | IL 18RAP | RPWTNCTFLGKRVEHHGTEKSDAVCSGSRKPPNEPHVYLPGV |
| | | | VLLYILLGTIGTLVAVLAASALLYRHWIEIVLLYRTYQSKDQTLGD |
| | | | KKDFDAFVSYAKWSSFPSEATSSLSEEHLALSLFPDVLENKYG |
| | | | YSLCLLERDVAPGGVYAEDIVSIIKRSRRGIFILSPNYVNGPSIFE |
| | | | LQAAVNLALDDQTLKLILIKFCYFQEPESLPHLVKKALRVLPTVT |
| | | | WRGLKSVPPNSRFWAKMRYHMPVKNSQGFTWNQLRITSRIFQ |
| | | | WKGLSRTETTGRSSQPKEW |
| 79 | OX40WT | WT OX40L | MERNKLLLVASVIQGLGLLLCFTYICLHFSALQVSHRYPRIQSIK |
| | mincyto | without IC | VQFTEYKKEKGFILTSQKEDEIMKVQNNSVIINCDGFYLISLKGY |
| | | domain | FSQEVNISLHYQKDEEPLFQLKKVRSVNSLMVASLTYKDKVYL |
| | | (deletion of | NVTTDNTSLDDFHVNGGELILIHQNPGEFCVL |
| | | first 18 amino | |
| | | acids) | |
| 80 | CD8-Q8 (Q8) | CD34 epitope | MGLVRRGARAGPRIPRGWTALCLLSLLPSGFMAELPTQGTFS |
| | | linked CD8 | NVSTNVSPAKPTTTPAPRPPTPAPTIASQPLSLRPEACRPAAG |
| | | stalk, TM and | GAVHTRGLDFACDIYIWAPLAGTCGVLLLSLVITLYCNHRNRRR |
| | | IC domain; | VCKCPRPVV |
| | | tricistronic | |
| | | control | |
| 81 | eGFP | Enhanced | MVSKGEELFTGVVPILVELDGDVNGHKFSVSGEGEGDATYGKL |
| | | GFP, | TLKFICTTGKLPVPWPTLVTTLTYGVQCFSRYPDHMKQHDFFK |
| | | tricistronic | SAMPEGYVQERTIFFKDDGNYKTRAEVKFEGDTLVNRIELKGID |
| | | control protein | FKEDGNILGHKLEYNYNSHNVYIMADKQKNGIKVNFKIRHNIED |
| | | without a | GSVQLADHYQQNTPIGDGPVLLPDNHYLSTQSALSKDPNEKR |
| | | cellular | DHMVLLEFVTAAGITLGMDELYK |
| | | function | |
| 178 | 41BBL-13W- | WT 41BBL | MLGRDQRLPPDAHKPPGGGSFRTPIQEEQADAHSTLAKITSGS |
| | OX40 | ligand | WPPAPRARACRVLPWALVAGLLLLLLLAAACAVFLACPWAVSG |
| | | binding, TM | ARASPGSAASPRLREGPELSPDDPAGLLDLRQGMFAQLVAQN |
| | | and IC | VLLIDGPLSWYSDPGLAGVSLTGGLSYKEDTKELVVAKAGVYY |
| | | domain | VFFQLELRRVVAGEGSGSVSLALHLQPLRSAAGAAALALTVDL |
| | | without first | PPASSEARNSAFGFQGRLLHLSAGQRLGVHLHTEARARHAWQ |
| | | 12 amino | LTQGATVLGLFRVTPEIPAGLPSPRSE |
| | | acids of the | |
| | | IC domain | |
| | | linked to the | |
| | | OX40 IC | |
| | | signaling | |
| | | domain | |
| 179 | 41BBL- | Extracellular | MLGVLHTPDQGQLEQLSLYADTNLPLLQTVKDRELVELPSIEPA |
| | OX40-IL2RB | 41BBL ligand | LGGPSFSSSPFPSSLWKQVDGGHESSLQSFFKSPDPTNCKLV |
| | | domain, a | KKLWPGTNRCNGSGRDQRLPPDAHKPPGGGSFRTPIQEEQA |
| | | transmembra | DAHSTLAKITSGSYASDASLDPEAPWPPAPRARACRVLPWALV |
| | | ne domain | AGLLLLLLLAAACAVFLACPWAVSGARASPGSAASPRLREGPE |
| | | from 41BBL, | LSPDDPAGLLDLRQGMFAQLVAQNVLLIDGPLSWYSDPGLAGV |
| | | and an OX40 | SLTGGLSYKEDTKELVVAKAGVYYVFFQLELRRVVAGEGSGSV |
| | | and IL2RB | SLALHLQPLRSAAGAAALALTVDLPPASSEARNSAFGFQGRLL |
| | | heterologous | HLSAGQRLGVHLHTEARARHAWQLTQGATVLGLFRVTPEIPAG |
| | | intracellular | LPSPRSE |
| | | signaling | |
| | | domain | |
| 180 | CD70 | WT CD70 | MLGPEEGSGCSVRRRPYGCVLRAALVPLVAGLVICLVVCIQRF |
| | | | AQAQQQLPLESLGWDVAELQLNHTGPQQDPRLYWQGGPALG |
| | | | RSFLHGPELDKGQLRIHRDGIYMVHIQVTLAICSSTTASRHHPT |
| | | | TLAVGICSPASRSISLLRLSFHQGCTIASQRLTPLARGDTLCTNL |
| | | | TGTLLPSRNTDETFFGVQWVRP |
| 181 | CD70mincyto | CD70 EC and | MLGCVLRAALVPLVAGLVICLVVCIQRFAQAQQQLPLESLGWD |
| | | TM | VAELQLNHTGPQQDPRLYWQGGPALGRSFLHGPELDKGQLRI |
| | | | HRDGIYMVHIQVTLAICSSTTASRHHPTTLAVGICSPASRSISLL |
| | | | RLSFHQGCTIASQRLTPLARGDTLCTNLTGTLLPSRNTDETFFG |
| | | | VQWVRP |
| 182 | CD70mincyto- | CD70 EC and | MLGRDQRLPPDAHKPPGGGSFRTPIQEEQADAHSTLAKITSGS |
| | OX40 | TM linked to | GCVLRAALVPLVAGLVICLVVCIQRFAQAQQQLPLESLGWDVA |
| | | IC signaling | ELQLNHTGPQQDPRLYWQGGPALGRSFLHGPELDKGQLRIHR |
| | | domain of | DGIYMVHIQVTLAICSSTTASRHHPTTLAVGICSPASRSISLLRLS |
| | | OX40 | FHQGCTIASQRLTPLARGDTLCTNLTGTLLPSRNTDETFFGVQ |
| | | separated by | WVRP |
| | | a TSGS linker | |
| 183 | CD70- | CD70 EC | MLGRDQRLPPDAHKPPGGGSFRTPIQEEQADAHSTLAKITSGS |
| | 41BBL-OX40 | linked to | YASDASLDPEAPWPPAPRARACRVLPWALVAGLLLLLLLAAAC |
| | | 41BBL TM | AVFLQRFAQAQQQLPLESLGWDVAELQLNHTGPQQDPRLYW |
| | | and IC with IC | QGGPALGRSFLHGPELDKGQLRIHRDGIYMVHIQVTLAICSSTT |
| | | signaling | ASRHHPTTLAVGICSPASRSISLLRLSFHQGCTIASQRLTPLAR |
| | | domain of | GDTLCTNLTGTLLPSRNTDETFFGVQWVRP |
| | | OX40 | |
| | | separated by | |
| | | a TSGS linker | |

A MIDIS protein can comprise, consist essentially of, or consist of an amino acid sequence with at least a minimal level of sequence identity compared to an amino acid sequence disclosed herein. For example, a MIDIS protein can comprise, consist essentially of, or consist of an amino acid sequence with at least 80%, at least 85%, at least 90%, at least 91%, at least 92%, at least 93%, at least 94%, at least 95%, at least 96%, at least 97%, at least 98%, at least 98.5%, at least 99%, or at least 99.5% sequence identity to an amino acid sequence disclosed herein, for example, any one of SEQ ID NOs: 45-53, 57-65,67,71-73,76-78. Another example is any one of SEQ ID NOs: 45-53, 57-65,67,71-73,76-78, 178-179, or 182-183. In an embodiment, such chimeric bidirectional signaling transmembrane protein having at least a minimal level of sequence identity compared to a given amino acid sequence is functional and therefore encompassed by the invention as long as this chimeric protein is able to transduce at least two, optionally inducible, intracellular signals and/or is able to induce an improvement of a biological parameter and/or function in a cell expressing it and/or is able to induce an improvement of a biological parameter and/or function induced by such a cell. The transduction of these at least two, optionally inducible, intracellular signals should be detectable using an assay known to the skilled person. Examples of suitable assays are western blotting, luminescence reporter or FACS assays. The improvement of a biological parameter and/or function should also be detectable using an assay known to the skilled person. Depending on the parameter and/or function, the skilled person would know which assay may be used.

In some embodiments, a MIDIS protein can comprise, consist essentially of, or consist of an amino acid sequence that is a wild type protein amino acid sequence or any other amino acid sequence disclosed herein, for example, any one of SEQ ID NOs: 45-53, 57-65,67,71-73,76-78. Another example is any one of SEQ ID NOs: 45-53, 57-65,67,71-73,76-78, 178-179, or 182-183.

A MIDIS protein can comprise an amino acid sequence with one or more amino acid insertions, deletions, or substitutions compared to an amino acid sequence disclosed herein.

For example, a MIDIS protein can comprise an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid insertions relative to an amino acid sequence disclosed herein, for example, any one of SEQ ID NOs: 45-53, 57-65,67,71-73,76-78. Another example is any one of SEQ ID NOs: 45-53, 57-65,67,71-73,76-78, 178-179, or 182-183.

In some embodiments, a MIDIS protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to an amino acid sequence disclosed herein, for example, any one of SEQ ID NOs: 45-53, 57-65,67,71-73,76-78. Another example is any one of SEQ ID NOs: 45-53, 57-65,67,71-73,76-78, 178-179, or 182-183.

In some embodiments, a MIDIS protein comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid insertions relative to an amino acid sequence disclosed herein, for example, any one of SEQ ID NOs: 45-53, 57-65,67,71-73,76-78. Another example is any one of SEQ ID NOs: 45-53, 57-65,67,71-73,76-78, 178-179, or 182-183.

The one or more insertions can be at the N-terminus, C-terminus, within the amino acid sequence, or a combination thereof. The one or more insertions can be contiguous, non-contiguous, or a combination thereof.

In some embodiments, a MIDIS protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid deletions relative to an amino acid sequence disclosed herein, for example, any one of SEQ ID NOs: 45-53, 57-65,67,71-73,76-78. Another example is any one of SEQ ID NOs: 45-53, 57-65,67,71-73,76-78, 178-179, or 182-183.

In some embodiments, a MIDIS protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to an amino acid sequence disclosed herein, for example, any one of SEQ ID NOs: 45-53, 57-65,67,71-73,76-78. Another example is any one of SEQ ID NOs: 45-53, 57-65,67,71-73,76-78, 178-179, or 182-183.

In some embodiments, a MIDIS protein comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid deletions relative to an amino acid sequence disclosed herein, for example, any one of SEQ ID NOs: 45-53, 57-65,67,71-73,76-78. Another example is any one of SEQ ID NOs: 45-53, 57-65,67,71-73,76-78, 178-179, or 182-183.

The one or more deletions can be at the N-terminus, C-terminus, within the amino acid sequence, or a combination thereof. The one or more deletions can be contiguous, non-contiguous, or a combination thereof.

In some embodiments, a MIDIS protein comprises an amino acid sequence with at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, or at least 50 amino acid substitutions relative to an amino acid sequence disclosed herein, for example, any one of SEQ ID NOs: 45-53, 57-65,67,71-73,76-78. Another example is any one of SEQ ID NOs: 45-53, 57-65,67, 71-73,76-78, 178-179, or 182-183.

In some embodiments, a MIDIS protein comprises an amino acid sequence with at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to an amino acid sequence disclosed herein, for example, any one of SEQ ID NOs: 45-53, 57-65,67,71-73,76-78. Another example is any one of SEQ ID NOs: 45-53, 57-65,67, 71-73,76-78, 178-179, or 182-183.

In some embodiments, a MIDIS protein comprises an amino acid sequence with 1, 2, 3, 4, 5, 6, 7, 8, 9, 10, 11, 12, 13, 14, 15, 16, 17, 18, 19, 20, 25, 30, 35, 40, 45, or 50 amino acid substitutions relative to an amino acid sequence disclosed herein, for example, any one of SEQ ID NOs: 45-53, 57-65,67,71-73,76-78. Another example is any one of SEQ ID NOs: 45-53, 57-65,67, 71-73,76-78, 178-179, or 182-183.

The one or more substitutions can be at the N-terminus, C-terminus, within the amino acid sequence, or a combination thereof. The one or more substitutions can be contiguous, non-contiguous, or a combination thereof. The one or more substitutions can be conservative, non-conservative, or a combination thereof.

### VII. Engineered Cells

The disclosure further provides cells comprising the polynucleotides and/or vectors described herein, and preferably expressing the polypeptides encoded by the polynucleotides and/or vectors. Accordingly, disclosed herein, in some aspects, are engineered cells or populations thereof that express one or more MIDIS protein(s) (i.e. chimeric bidirectional signaling transmembrane protein). Expression of one or more MIDIS proteins in an engineered cell or population thereof can be used as a strategy to overcome limitations that hamper the production and use of engineered cells, for example, difficulties in generating sufficient numbers of the desired engineered cells, limited cytotoxic effect, limited immune stimulatory effect, limited proliferative ability or lifespan of the engineered cells, limited induction of effector function upon engineered cell recognition of antigen, and engineered cell exhaustion. Within the context of the application, the expression “engineered cell” refers to a cell that has been modified using recombinant DNA technology. In an embodiment, an “engineered cell” has been transformed, modified or transduced to comprise a heterologous nucleic acid molecule. In an embodiment, said cell expresses a protein encoded by said nucleic acid molecule.

Disclosed herein, in some aspects, are cells or populations thereof that comprise and preferably express one or more chimeric bidirectional signaling transmembrane protein(s). Expression of one or more of these chimeric proteins in a cell or population thereof can be used as a strategy to overcome the same limitations as identified in previous paragraph.

In the application, the wording “engineered cell” may be replaced by “modified cell” or “transformed cell” or “transduced cell”.

In the application, the wording “an engineered cell comprising a heterologous nucleic acid molecule”, or “an engineered cell expressing a chimeric bidirectional signaling transmembrane protein” may be replaced by the wording “a cell comprising a heterologous nucleic acid molecule” or “a cell expressing a chimeric bidirectional signaling transmembrane protein”. The same applies to population comprising such a cell.

In an embodiment, there is provided a cell comprising a chimeric bidirectional signaling transmembrane protein as defined earlier herein. In an embodiment, this cell comprises a polynucleotide encoding said chimeric protein. In an embodiment, this cell comprises a vector comprising said polynucleotide. In an embodiment, this cell expresses said chimeric protein. In an embodiment, this cell also comprises, preferably express the interaction partner as defined herein. In an embodiment, a population of cells is provided comprising such a cell.

When the extracellular ligand domain binds to its interaction partner, multi-directional signaling is induced that comprises at least one “outside-in” signal mediated by the heterologous intracellular signaling domain of the MIDIS protein, and at least one “inside-out” signal mediated by an intracellular signaling domain of the interaction partner. In an embodiment, the multidirectional signaling is bidirectional signaling. The “inside-out” and “outside-in” signaling pathways can jointly induce a target biological outcome. In some embodiments, the “inside-out” and “outside-in” signaling pathways can jointly reduce a target biological outcome. In some embodiments, the “inside-out” and “outside-in” signaling pathways can jointly favor a target biological outcome In contrast, many constructs introduced into engineered cells only elicit one-way signaling, and/or only one-way signaling contributes to a target biological outcome.

Through the application, the wording “target biological outcome” may be replaced by “biological parameter and/or biological function”.

Therefore in an embodiment, the chimeric bidirectional signaling transmembrane protein is able to transduce at least two, optionally inducible, intracellular signals that contribute to an improvement of a biological parameter and/or function of a cell expressing the chimeric protein and/or an improvement of a biological parameter and/or function induced by such a cell.

A target biological outcome (i.e. a biological parameter and/or biological function) can be or can comprise, for example, cellular proliferation, cellular survival, magnitude of immune effector function, duration of immune effector function, cytotoxic effects on a cell (e.g., a cancer cell), production of inflammatory mediators, an anti-cancer immune response, cellular differentiation, cellular dedifferentiation.

In an embodiment, the biological parameter and/or function is selected from proliferation, cellular survival, cytotoxicity, antitumor activity, persistence and/or tumor cell killing,

A target biological outcome or biological parameter and/or function can include a cytotoxic response, e.g., against cancer cell. A cytotoxic response may be determined directly (e.g., by measuring cell lysis or survival of target cells). Alternatively or in addition, a cytotoxic response may be determined by measuring the production of molecules associated with such a response, for example a production of a cytokine such as interferon gamma (IFNγ). Suitable measurement assays, for example luminescence assays to determine cytotoxicity and ELISA to determine IFNγ production are known to the skilled person and further non-limiting examples are provided in the experimental section.

In some embodiments, an exogenous antigen-recognition receptor can contribute to a target biological outcome. For example, in some embodiments, a cytotoxic response is not induced against cells that express the interaction partner of the MIDIS protein, but rather is induced against cells that express or present an antigen recognized by an exogenous antigen-recognition receptor. For example, the interaction partner can be expressed by engineered immune cells and can support fitness and effector function of the engineered immune cells that express the exogenous antigen-recognition receptor. In some embodiments, the MIDIS protein can enhance an immune response induced by an exogenous antigen-recognition receptor, but does not alter specificity of the immune response (e.g., does not induce an immune response against cells that express the interaction partner).

Therefore in one embodiment, a population of cells is provided wherein at least one cell expresses the chimeric bidirectional signaling transmembrane protein and preferably the interaction partner and wherein the population of cells further comprises at least one cell that expresses an exogenous antigen-recognition receptor.

Therefore in one embodiment, the cell comprising, preferably expressing the chimeric bidirectional signaling transmembrane protein and its interaction partner also comprises preferably expresses an exogenous antigen-recognition receptor.

Alternatively, in another embodiment, there is a cell comprising, preferably expressing the chimeric bidirectional signaling transmembrane protein, there is a distinct cell expressing its interaction partner. The cell comprising preferably expressing an exogenous antigen-recognition receptor may be the same as the one expressing the chimeric bidirectional signaling transmembrane protein or the same as the expressing the interaction partner or a distinct one.

Alternatively, in another embodiment, a cell population is provided with at least one cell comprising, preferably expressing the chimeric bidirectional signaling transmembrane protein and its interaction partner and at least one distinct cell comprising preferably expressing an exogenous antigen-recognition receptor.

In some embodiments, an exogenous antigen-recognition receptor does not contribute to a target biological outcome.

Multi-directional signaling induced by a MIDIS protein can modulate a biological function, for example, a target biological function of an engineered cell that expresses the MIDIS protein, a cell that expresses the interaction partner, or a combination thereof. In an embodiment, the at least two, optionally inducible, intracellular signals transduced by the chimeric bidirectional signaling transmembrane protein is able to modulate (increase or decrease) a biological function or parameter of a cell expressing said chimeric protein and the interaction partner. In this context, the biological parameter and/or function is selected from proliferation, cellular survival, cytotoxicity, antitumor activity, persistence and/or tumor cell killing

A target biological function of the engineered cell can be or can comprise, for example, survival, proliferation, immune effector function, a cytotoxic response (e.g., against a cancer cell), an anti-cancer response, cellular differentiation, cellular dedifferentiation, or cellular transdifferentiation. The target biological function of the engineered cell can be induced. The target biological function of the engineered cell can be reduced. In some embodiments, a target biological function of an engineered cell is elicited by or directed against cells that express or present an antigen recognized by an antigen-recognition receptor. For example, in some embodiments, where a target biological function comprises a cytotoxic response against cancer cells, the engineered cells can kill cancer cells based on recognition of an antigen by an antigen-recognition receptor (e.g., an exogenous antigen-recognition receptor), but not based on expression of the interaction partner.

In some embodiments, an exogenous antigen recognition receptor and the MIDIS protein each contribute to the same biological function of an engineered cell. In some embodiments, an exogenous antigen recognition receptor and the MIDIS protein do not contribute to the same biological function of the engineered cell. In some embodiments, an exogenous antigen recognition receptor and the MIDIS protein each contribute to different biological functions of the engineered cell.

In some embodiments, upon exposure to a cell that expresses the interaction partner, the target biological function of the engineered cell is modulated for at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold longer than a corresponding cell that does not express the MIDIS protein.

In some embodiments, upon exposure to a cell that expresses the interaction partner, the target biological function of the engineered cell is increased by at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold compared to a corresponding cell that does not express the MIDIS protein.

In an embodiment, upon exposure to a cell that expresses the chimeric, bidirectional signaling transmembrane protein and its interaction partner, the proliferation, cellular survival, cytotoxicity, antitumor activity, persistence and/or tumor cell killing of said cell is increased by at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold compared to a corresponding cell that does not express the chimeric protein.

In an embodiment, a population of cells that expresses the chimeric bidirectional signaling transmembrane protein to cells that express or present an antigen that binds to the exogenous antigen-recognition receptor, proliferation, cellular survival, cytotoxicity, antitumor activity, persistence and/or tumor cell killing of the population of said cells is increased by at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold compared to a corresponding population of cells that do not express the chimeric protein.

The assessment of proliferation, cellular survival, cytotoxicity, antitumor activity, persistence and/or tumor cell killing can be carried out using assays known to the skilled person. Examples of such assays are disclosed in the experimental part.

In some embodiments, upon exposure to a cell that expresses the interaction partner, the target biological function of the engineered cell is decreased by at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold compared to a corresponding cell that does not express the MIDIS protein.

An engineered cell can be a mammalian cell. An engineered cell can be a human cell. An engineered cell can be an immune cell. In some embodiments, an engineered cell of the disclosure is an immune cell, a T cell, an alpha-beta T cell, a gamma-delta T cell, a Jurkat cell, a CD4+ T cell, CD8+ T cell, a T effector cell, a lymphocyte, a B cell, an NK cell, an NKT cell, a myeloid cell, a monocyte, a macrophage, or a neutrophil. In some embodiments, an engineered cell of the disclosure is a T cell, preferably an αβT cell or a γδT cell, more preferably an αβT cell. In some cases, an engineered cell is a primary cell. In some cases, an engineered cell is not a primary cell.

In some embodiments, an engineered cell of the disclosure is a basophil, a dendritic cell, an eosinophil, a granulocyte, a helper T cell, a Langerhans cell, a lymphoid cell, an innate lymphoid cell (ILC), a macrophage, a mast cell, a megakaryocyte, a memory T cell, a monocyte, a myeloid cell, a plasma cell, a thymocyte, or any mixture or combination of cells thereof. Any of the aforementioned cells can be engineered, for example to express an exogenous antigen-recognition receptor or to comprise a polynucleic acid provided herein.

In some embodiments, an engineered cell comprises a deletion or disruption of one or more genes in the genome, for example, a deletion or disruption of a TRAC gene, a TCRB gene, an immune checkpoint gene, or a combination thereof.

F. Cell that Expresses the Interaction Partner

Disclosed herein are compositions and methods that comprise a cell that expresses an interaction partner capable of binding to an extracellular ligand domain of a MIDIS protein. A biological function of the cell that expresses the interaction partner can be or can comprise, for example, cellular survival, proliferation, immune effector function, a cytotoxicity (also called cytotoxic response (e.g., against a cancer cell)), an anti-cancer response (also called antitumor activity), tumor cell killing, persistence, cellular differentiation, cellular dedifferentiation, or cellular transdifferentiation.

In an embodiment, the biological parameter and/or function which is improved by the at least two, optionally inducible, intracellular signals is selected from proliferation, cellular survival, cytotoxicity, antitumor activity, persistence and/or tumor cell killing.

The biological function of the cell that expresses the interaction partner can be induced. The biological function of the cell that expresses the interaction partner can be reduced.

In some embodiments, the biological function of the cell that expresses the interaction partner does not include death of the cell that expresses the interaction partner (e.g., via apoptosis, necroptosis, or any other cell death pathway). In other embodiments, the biological function of the cell that expresses the interaction partner includes death of the cell that expresses the interaction partner (e.g., via apoptosis, necroptosis, or any other cell death pathway).

In some embodiments, the cell that expresses the interaction partner also expresses an antigen-recognition receptor disclosed herein (e.g., an exogenous antigen recognition receptor). In some embodiments, a biological function of the cell that expresses the interaction partner is elicited by or directed against cells that express or present an antigen recognized by the antigen-recognition receptor. For example, in some embodiments, where a biological function of the cell that expresses the interaction partner comprises a cytotoxic response against cancer cells, the cell that expresses the interaction partner can kill cancer cells based on recognition of an antigen by an antigen-recognition receptor (e.g., an exogenous antigen-recognition receptor). A cytotoxic response can be a cytotoxic response against cells (e.g., cancer cells) that do not express the interaction partner, or express it only at low levels.

In some embodiments, an exogenous antigen recognition receptor and the MIDIS protein each contribute to the same biological function of the cell that expresses the interaction partner. In some embodiments, an exogenous antigen recognition receptor and the MIDIS protein do not contribute to the same biological function of the cell that expresses the interaction partner. In some embodiments, an exogenous antigen recognition receptor and the MIDIS protein each contribute to different biological functions of the cell that expresses the interaction partner.

In some embodiments, upon exposure to an engineered cell that expresses a MIDIS protein, the target biological function of the cell that expresses the interaction partner is modulated for at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold longer than upon exposure to a corresponding engineered cell that does not express the MIDIS protein.

In some embodiments, upon exposure to an engineered cell that expresses a MIDIS protein, the target biological function of the cell that expresses the interaction partner is increased by at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold compared to upon exposure to a corresponding engineered cell that does not express the MIDIS protein.

In some embodiments, upon exposure to an engineered cell that expresses a MIDIS protein, the target biological function of the cell that expresses the interaction partner is decreased by at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold compared to upon exposure to a corresponding engineered cell that does not express the MIDIS protein.

A cell that expresses the interaction partner can be a mammalian cell. A cell that expresses the interaction partner can be a human cell. A cell that expresses the interaction partner can be an immune cell. In some embodiments, a cell that expresses the interaction partner of the disclosure is an immune cell, a T cell, an alpha-beta T cell, a gamma-delta T cell, CD4+ T cell, CD8+ T cell, a T effector cell, a lymphocyte, a B cell, an NK cell, an NKT cell, a myeloid cell, a monocyte, a macrophage, or a neutrophil. In some embodiments, cell that expresses the interaction partner of the disclosure is a T cell. In some embodiments, cell that expresses the interaction partner is a fibroblast, a keratinocyte, a mesenchymal stem cell, an endothelial cell, or a stromal cell. In some embodiments, the cell that expresses the interaction partner is a cancer cell.

In some embodiments, the engineered cell and the cell that expresses the interaction partner are the same cell type. In some embodiments, the engineered cell and the cell that expresses the interaction partner are different cell types. In some embodiments, the engineered cell and the cell that expresses the interaction partner are the same cell, i.e., a cell that co-expresses the MIDIS protein and the interaction partner. In some embodiments, the engineered cell and the cell that expresses the interaction partner are not the same cell.

### G. Exogenous Antigen-Recognition Receptor

An engineered cell of the disclosure can express an exogenous antigen-recognition receptor, for example, co-express an exogenous antigen-recognition receptor and a MIDIS protein of the disclosure.

In an embodiment, a cell comprises and preferably expresses a chimeric bidirectional signaling transmembrane protein, its interaction partner and an exogenous antigen-recognition receptor. A population of cells comprising such cell is also encompassed herein.

An exogenous antigen-recognition receptor is a receptor capable of recognizing an antigen, which receptor is artificially introduced into an engineered cell. Non-limiting examples of exogenous antigen-recognition receptors include chimeric antigen receptors (CARs) and TCRs (where the TCR is artificially introduced into the cell, for example, a cell that does not otherwise express a TCR, or expresses a different TCR).

In the context of the disclosure, “gamma”, “γ”, and “g” are used interchangeably to refer to a γ chain of a γδ TCR. “Delta”, “b”, and “d” are used interchangeably to refer to a δ chain of a γδ TCR. “Alpha”, “α”, and “a” are used interchangeably to refer to an α chain of an αβ TCR. “Beta”, “β”, and “b” are used interchangeably to refer to a β chain of an αβ TCR.

An exogenous antigen recognition receptor can be a transgenic TCR. An exogenous antigen recognition receptor can be an alpha beta TCR (for example, an alpha beta TCR introduced into a cell that does not otherwise express an alpha-beta TCR, or expresses a different alpha-beta TCR). An exogenous antigen recognition receptor can be a gamma-delta TCR (for example, a gamma-delta TCR introduced into a cell that does not otherwise express a gamma-delta TCR, such as an alpha-beta T cell, or a cell that expresses a different a gamma-delta TCR).

An αβ TCR, also referred to as an alpha-beta TCR, can be composed of two protein chains, T-cell receptor α and T-cell receptor β. αβ TCRs recognize a composite ligand of a peptide antigen bound to an MHC molecule. MHC molecules are highly polymorphic glycoproteins encoded by genes in the major histocompatibility complex (MHC). Two classes of MHC molecules (class I and class II) are bound in their nonpolymorphic (constant) domains by CD8 and CD4 molecules that distinguish two different functional classes of αβ T cells. CD8 binds MHC class I molecules; CD4 binds MHC class II molecules. In an aspect, a TCR can be or can comprise at least one of: an alpha chain of a TCR or a beta chain of a TCR. A second type of TCR, composed of a γ and a δ chain, is structurally similar to the αβ TCR but binds different ligands, including nonpeptide ligands. In an aspect, a TCR can be or can comprise at least one of: a gamma chain of a TCR or a delta chain of a TCR.

In an embodiment, a cell comprises and preferably expresses a chimeric bidirectional signaling transmembrane protein, its interaction partner and an exogenous antigen-recognition receptor which is a chimeric antigen receptor, a TCR, an alpha-beta TCR or a gamma-delta TCR. In an embodiment, the cell is an alpha-beta T cells that comprises and preferably expresses a chimeric bidirectional signaling transmembrane protein, its interaction partner and a gamma-delta TCR. A population of cells comprising such cell is also encompassed herein.

In some embodiments, an exogenous γδTCR (also referred to as a gamma-delta TCR) can be introduced into a cell, such as a T cell. In some embodiments, an exogenous γδTCR is introduced in an alpha-beta T cell or a gamma-delta T cell, preferably an alpha-beta T cell. In an aspect, an engineered cell expressing a γδTCR, or a method comprising introducing a γδTCR into an immune cell, such as an αβT cell, can overcome clonal heterogeneity of tumor cells in patients with advanced cancer, an improvement over αβTCR-based approaches. In an aspect, this improvement may be due to the distinct HLA-independent activation cues of the γδTCR, such as activation that involves changes in lipid metabolism. In an aspect, a γδTCR therapeutic (e.g., also expressing a MIDIS protein of the disclosure) may be administered to a subject comprising a cancer with a low mutational load. In some embodiments, a gamma-delta TCR of the disclosure binds a target, such as CD277 on a cancer cell. Binding of a γδTCR therapeutic can comprise recognition of spatial and/or conformational changes in CD277 expressed on a target, e.g., a conformation change in response to one or more metabolites. In some embodiments, activation of a γδTCR comprises binding to a complex that comprises one or more proteins from a BTNA1, 2, or 3 family. In some embodiments, activation of a γδTCR comprises binding to a complex that comprises CD277. In some embodiments, activation of a γδTCR comprises binding to a complex that comprises CD277 and BTN2A1. In some embodiments, the γδTCR that that recognizes a spatial and/or conformational change in CD277, binds to a complex that comprises one or more proteins from a BTNA1, 2, or 3 family, binds to a complex that comprises CD277, binds to a complex that comprises CD277 and BTNA2, or a combination thereof, is a γδTCR that comprises a γ9 chain or a variable region thereof, and a δ2 chain or a variable domain thereof.

Where the exogenous antigen-recognition receptor is a γδTCR, the γδTCR can comprise (a) a γ-chain selected from the group consisting of γ2, γ3, γ4, γ5, γ8, γ9, and γ11; (b) a δ-chain selected from the group consisting of δ1, δ2, δ3, and δ5; or (c) any combination of (a) and (b). In some embodiments, the γ-chain is the γ9 chain and the δ-chain is the δ2 chain. In some embodiments, the γ-chain is the γ4 chain and the δ-chain is the δ5 chain.

In some embodiments, the γ-chain is the γ2 chain and the δ-chain is the δ1 chain. In some embodiments, the γ-chain is the γ3 chain and the δ-chain is the δ1 chain. In some embodiments, the γ-chain is the γ4 chain and the δ-chain is the δ1 chain. In some embodiments, the γ-chain is the γ5 chain and the δ-chain is the δ1 chain. In some embodiments, the γ-chain is the γ8 chain and the δ-chain is the δ1 chain. In some embodiments, the γ-chain is the γ9 chain and the δ-chain is the δ1 chain. In some embodiments, the γ-chain is the γ11 chain and the δ-chain is the δ1 chain.

In some embodiments, the γ-chain is the γ2 chain and the δ-chain is the δ2 chain. In some embodiments, the γ-chain is the γ3 chain and the δ-chain is the δ2 chain. In some embodiments, the γ-chain is the γ4 chain and the δ-chain is the δ2 chain. In some embodiments, the γ-chain is the γ5 chain and the δ-chain is the δ2 chain. In some embodiments, the γ-chain is the γ8 chain and the δ-chain is the δ2 chain. In some embodiments, the γ-chain is the γ9 chain and the δ-chain is the δ2 chain. In some embodiments, the γ-chain is the γ11 chain and the δ-chain is the δ2 chain.

In some embodiments, the γ-chain is the γ2 chain and the δ-chain is the δ3 chain. In some embodiments, the γ-chain is the γ3 chain and the δ-chain is the δ3 chain. In some embodiments, the γ-chain is the γ4 chain and the δ-chain is the δ3 chain. In some embodiments, the γ-chain is the γ5 chain and the δ-chain is the δ3 chain. In some embodiments, the γ-chain is the γ8 chain and the δ-chain is the δ3 chain. In some embodiments, the γ-chain is the γ9 chain and the δ-chain is the δ3 chain. In some embodiments, the γ-chain is the γ11 chain and the δ-chain is the δ3 chain.

In some embodiments, the γ-chain is the γ2 chain and the δ-chain is the δ5 chain. In some embodiments, the γ-chain is the γ3 chain and the δ-chain is the δ5 chain. In some embodiments, the γ-chain is the γ4 chain and the δ-chain is the δ5 chain. In some embodiments, the γ-chain is the γ5 chain and the δ-chain is the δ5 chain. In some embodiments, the γ-chain is the γ8 chain and the δ-chain is the δ5 chain. In some embodiments, the γ-chain is the γ9 chain and the δ-chain is the δ5 chain. In some embodiments, the γ-chain is the γ11 chain and the δ-chain is the δ5 chain.

In some embodiments, an exogenous antigen-recognition receptor comprises a variable domain from a γ-chain and/or a variable domain from a δ-chain. Variable domains can be indicated by a V preceding the γ-chain and δ-chain designations, e.g., Vγ2, Vγ3, Vγ4, Vγ5, Vγ8, Vγ9, Vγ11, Vδ1, Vδ2, Vδ3, and Vδ5.

In some embodiments, where the exogenous antigen-recognition receptor is a γδTCR, the TCR can comprise (a) a variable domain of a γ-chain selected from the group consisting of Vγ2, Vγ3, Vγ4, Vγ5, Vγ8, Vγ9, and Vγ11; (b) a variable domain of a δ-chain selected from the group consisting of Vδ1, Vδ2, Vδ3, and Vδ5; or (c) any combination of (a) and (b), e.g., as indicated herein for the γ and 6 chains. In some embodiments, the γ-chain variable domain is the Vγ9 and the δ-chain variable domain is the Vδ2. In some embodiments, the γ-chain variable domain is the Vγ4 and the δ-chain variable domain is the Vδ5.

In some embodiments, an exogenous antigen-recognition receptor comprises a constant domain from a γ-chain and/or a constant domain from a δ-chain. Constant domains can be indicated by a C preceding the γ-chain and δ-chain designations, e.g., Cγ1, Cγ2 and Cδ.

In some embodiments, where the exogenous antigen-recognition receptor is a γδTCR, the TCR can comprise (a) a constant domain of a γ-chain selected from the group consisting of Cγ1 and Cγ2; (b) a constant domain of a δ-chain Cδ; or (c) any combination of (a) and (b), e.g., as indicated herein for the γ and δ chains. In some embodiments, the γ-chain constant domain is the Cγ1 and the δ-chain constant domain is the Cδ. In some embodiments, the γ-chain constant domain is the Cγ2 and the δ-chain constant domain is the Cδ.

An exogenous antigen-recognition receptor can comprise a Vγ9Vδ2 TCR or functional fragment thereof. The Vγ9Vδ2 TCR can comprise at least one of a γ-TCR amino acid sequence or a δ-TCR amino acid sequence capable of recognizing a CD277 protein on a cell surface of a cell (e.g. tumor cell). In some embodiments, the receptor comprises a variant or a fragment of at least one of a γ-TCR amino acid sequence or a δ-TCR amino acid sequence capable of recognizing a CD277 protein on a cell surface of a target cell. The present disclosure contemplates exogenous antigen-recognition receptors comprising any portion or fragment or variation of a γδTCR capable of recognizing a cell (e.g. tumor cell) via a CD277 cell surface molecule.

In some embodiments, the exogenous antigen-recognition receptor comprises a variant or a fragment of at least one of a γ-TCR amino acid sequence and/or a δ-TCR amino acid sequence capable of recognizing an EPCR protein on a cell surface of a target cell. The present disclosure contemplates exogenous antigen-recognition receptors comprising any portion or fragment or variation of a γδTCR capable of recognizing a cell (e.g. tumor cell) via an EPCR cell surface molecule. Variable domain and CDR3 regions for such a γδTCR are identified in table 6: SEQ ID NO:101-102.

In some embodiments, the exogenous antigen-recognition receptor comprises a variant or a fragment of at least one of a γ-TCR amino acid sequence and/or a δ-TCR amino acid sequence capable of recognizing annexin A2 on a cell surface of a target cell. The present disclosure contemplates exogenous antigen-recognition receptors comprising any portion or fragment or variation of a γδTCR capable of recognizing a cell (e.g. tumor cell) via an annexin A2 surface molecule. Variable domain and CDR3 regions for such a γδTCR are identified in table 6: SEQ ID NO:130 and 131.

In some embodiments, the exogenous antigen-recognition receptor comprises a variant or a fragment of at least one of a γ-TCR amino acid sequence and/or a δ-TCR amino acid sequence capable of recognizing aberrant HLA protein expression on a cell surface of a target cell. The present disclosure contemplates exogenous antigen-recognition receptors comprising any portion or fragment or variation of a γδTCR capable of recognizing a cell (e.g. tumor cell) via an aberrant HLA protein expression on the cell surface. In some embodiments, the exogenous antigen-recognition receptor comprises a variant or a fragment of at least one of a γ-TCR amino acid sequence and/or a δ-TCR amino acid sequence capable of recognizing cancers in an MHC-unrestricted manner. Variable domain and CDR3 regions for such a γδTCR are identified in table 6: SEQ ID NO: 82 and 85.

In some embodiments, the exogenous antigen-recognition receptor comprises at least a portion of a Cγ or Cδ region and at least a portion of a Vγ or a Vδ region of a γδTCR. In some embodiments, the exogenous antigen-recognition receptor comprises at least a portion of a Cγ or Cδ region and at least a CDR3 domain of either a Vγ or a Vδ domain of a γδTCR. In some embodiments, the exogenous antigen-recognition receptor comprises all CDR regions of the Vγ9Vδ2 TCR, and all of the CDR regions can be involved in binding to a cell surface molecule (e.g. CD277 molecule) on the surface of a cell In some embodiments, the exogenous antigen-recognition receptor comprises all CDR regions of the Vγ4Vδ5 TCR, and all of the CDR regions can be involved in binding to a cell surface molecule (e.g. EPCR molecule) on the surface of a cell. In some embodiments, the exogenous antigen-recognition receptor comprises all CDR regions of the Vγ5Vδ1 TCR, and all of the CDR regions can be involved in binding to a cell surface molecule (e.g. HLA molecule) on the surface of a cell. In some embodiments, the exogenous antigen-recognition receptor comprises all CDR regions of the Vγ8Vδ3 TCR, and all of the CDR regions can be involved in binding to a cell surface molecule (e.g. annexin A2) on the surface of a cell.

Gamma-delta TCRs useful in compositions and methods of the disclosure, and sequences thereof, have been disclosed for example, in patent applications WO2013147606A1, WO2017212074A1, and WO2018211115A1, each of which is incorporated herein by reference in its entirety. These sequences have been identified in table 6.

Non-limiting examples of sequences that an exogenous antigen recognition receptor of the disclosure can comprise, consist essentially of, or consist of are provided in Table 6. In some cases, a γδ TCR comprises a sequence that codes a γ-chain (G), δ-chain (D), a variable domain (TRG, TRD), a CDR (e.g., CDR3) sequence therefrom, a constant domain (TRDC, TRGC1, TRGC2), or a combination thereof selected from Table 6. An example of a suitable TRDC is represented by SEQ ID NO:134, an example of a suitable TRGC1 is represented by SEQ ID NO: 135 and an example of a suitable TRGC2 is represented by SEQ ID NO: 136. Example of a sequence is published (Grunder C., et al, Blood 2012; 120 (26): 5153-5162. doi: https://doi.org/10.1182/blood-2012-05-432427). In some cases, an exogenous antigen-recognition receptor comprises a sequence (e.g., a CDR3 region sequence) with at least 60%, at least 65%, at least 70%, at least 75%, at least 80%, at least 85%, at least 90%, at least 95%, at least 97%, at least 98%, at least 99%, or up to about 100% sequence identity to a sequence in Table 6.

TABLE 6

| SEQ | | |
|---|---|---|
| ID NO: | Name | Sequence |
| 82 | CDR3 VD1 | CALGDSYGGGPLYTDKLIF |
| | Fe11 | |
| 83 | CDR3 VD2 cl3 | CACDLLGYTDKLIF |
| 84 | CDR3 VD2 cl5 | CACDALKRTDTDKLIFG |
| 85 | CDR3 VG5 | CATWDRPEIYYKKLF |
| | Fe11 | |
| 86 | CDR3 VG9 cl3 | CALWEEELGKKIKVF |
| 87 | CDR3 VG9 cl5 | CALWEIQELGKKIKVF |
| 88 | TRD cl3 | MERISSLIHLSLFWAGVMSAIELVPEHQTVPVSIGVPATLRCSMKG |
| | | EAIGNYYINWYRKTQGNTMTFIYREKDIYGPGFKDNFQGDIDIAKN |
| | | LAVLKILAPSERDEGSYYCACDLLGYTDKLIFGKGTRVTVEPR |
| 89 | TRG cl3 | MVSLLHASTLAVLGALCVYGAGHLEQPQISSTKTLSKTARLECVV |
| | | SGITISATSVYWYRERPGEVIQFLVSISYDGTVRKESGIPSGKFEV |
| | | DRIPETSTSTLTIHNVEKQDIATYYCALWEEELGKKIKVFGPGTKLII |
| | | T |
| 90 | TRD cl5 | MERISSLIHLSLFWAGVMSAIELVPEHQTVPVSIGVPATLRCSMKG |
| | | EAIGNYYINWYRKTQGNTMTFIYREKDIYGPGFKDNFQGDIDIAKN |
| | | LAVLKILAPSERDEGSYYCACDALKRTDTDKLIFGKGTRVTVEPR |
| 91 | TRG cl5 | MVSLLHASTLAVLGALCVYGAGHLEQPQISSTKTLSKTARLECVV |
| | | SGITISATSVYWYRERPGEVIQFLVSISYDGTVRKESGIPSGKFEV |
| | | DRIPETSTSTLTIHNVEKQDIATYYCALWEIQELGKKIKVFGPGTKLI |
| | | IT |
| 92 | TRD Fe11 | MVFSSLLCVFVAFSYSGSSVAQKVTQAQSSVSMPVRKAVTLNCL |
| | | YETSWWSYYIFWYKQLPSKEMIFLIRQGSDEQNAKSGRYSVNFK |
| | | KAAKSVALTISALQLEDSAKYFCALGDSYGGGPLYTDKLIFGKGTR |
| | | VTVEPR |
| 93 | TRG Fe11 | MGWALLVLLAFLSPASQKSSNLEGGTKSVTRPTRSSAEITCDLTVI |
| | | NAFYIHWYLHQEGKAPQRLLYYDVSNSKDVLESGLSPGKYYTHT |
| | | PRRWSWILILRNLIENDSGVYYCATWDRPEIYYKKLFGSGTTLVVT |
| 94 | CDR3 VG4 | CATWDGPPYYKKLF |
| | E113 | |
| 95 | CDR3 VG2 F4 | CATWDGQKKLF |
| 96 | CDR3 VG8 Zi11 | CATWDNYKKLF |
| 97 | CDR3 VD5 D37 | CAASSPIRGYTGSDKLIF |
| 98 | CDR3 VD5 | CAASSPIRGYTGSDKLIF |
| | E113 | |
| 99 | CDR3 VD1 F4 | CALGELRYWGIVDKLIF |
| 100 | CDR3 VD1 Zi11 | CALGELRGQISFLYLLGDTTDKLIF |
| 101 | CDR3 VG4 E57 | CATWDGFYYKKLF |
| 102 | CDR3 VD5 E57 | CAASSPIRGYTGSDKLIF |
| 103 | TRD E113 | MAMLLGASVLILWLQPDWVNSQQKNDDQQVKQNSPSLSVQEGR |
| | | ISILNCDYTNSMFDYFLWYKKYPAEGPTFLISISSIKDKNEDGRFTV |
| | | FLNKSAKHLSLHIVPSQPGDSAVYFCAASSPIRGYTGSDKLIFGKG |
| | | TRVTVEPR |
| 104 | TRG E113 | MEWALAVLLAFLSPASQKSSNLEGRTKSVIRQTGSSAEITCDLAE |
| | | GSTGYIHWYLHQEGKAPQRLLYYDSYTSSVVLESGISPGKYDTY |
| | | GSTRKNLRMILRNLIENDSGVYYCATWDGPPYYKKLFGSGTTLVV |
| | | T |
| 105 | TRD F4 | MVFSSLLCVFVAFSYSGSSVAQKVTQAQSSVSMPVRKAVTLNCL |
| | | YETSWWSYYIFWYKQLPSKEMIFLIRQGSDEQNAKSGRYSVNFK |
| | | KAAKSVALTISALQLEDSAKYFCALGELRYWGIVDKLIFGKGTRVT |
| | | VEPR |
| 106 | TRG F4 | MEWALAVLLAFLSPASQKSSNLEGRTKSVIRQTGSSAEITCDLAE |
| | | GSNGYIHWYLHQEGKAPQRLQYYDSYNSKVVLESGVSPGKYYT |
| | | YASTRNNLRLILRNLIENDSGVYYCATWDGQKKLFGSGTTLVVT |
| 107 | TRD Zi11 | MVFSSLLCVFVAFSYSGSSVAQKVTQAQSSVSMPVRKAVTLNCL |
| | | YETSWWSYYIFWYKQLPSKEMIFLIRQGSDEQNAKSGRYSVNFK |
| | | KAAKSVALTISALQLEDSAKYFCALGELRGQISFLYLLGDTTDKLIF |
| | | GKGTRVTVEPR |
| 108 | TRG Zi11 | MVLALALLLAFLPPASQKSSNLEGRTKSVTRPTGSSAVITCDLPVE |
| | | NAVYTHWYLHQEGKAPQRLLYYDSYNSRVVLESGISREKYHTYA |
| | | STGKSLKFILENLIERDSGVYYCATWDNYKKLFGSGTTLVVT |
| 109 | TRD D37 | MAMLLGASVLILWLQPDWVNSQQKNDDQQVKQNSPSLSVQEGR |
| | | ISILNCDYTNSMFDYFLWYKKYPAEGPTFLISISSIKDKNEDGRFTV |
| | | FLNKSAKHLSLHIVPSQPGDSAVYFCAASSPIRGYTGSDKLIFGKG |
| | | TRVTVEPR |
| 110 | TRG D37 | MVLALALLLAFLPPASQKSSNLEGRTKSVTRPTGSSAVITCDLPVE |
| | | NAVYTHWYLHQEGKAPQRLLYYDSYNSRVVLESGISREKYHTYA |
| | | STGKSLKFILENLIERDSGVYYCATWDNYMKLFGSGTTLVVT |
| 111 | TRD E57 | MAMLLGASVLILWLQPDWVNSQQKNDDQQVKQNSPSLSVQEGR |
| | | ISILNCDYTNSMFDYFLWYKKYPAEGPTFLISISSIKDKNEDGRFTV |
| | | FLNKSAKHLSLHIVPSQPGDSAVYFCAASSPIRGYTGSDKLIFGKG |
| | | TRVTVEPR |
| 112 | TRG E57 | MEWALAVLLAFLSPASQKSSNLEGRTKSVIRQTGSSAEITCDLAE |
| | | GSTGYIHWYLHQEGKAPQRLLYYDSYTSSVVLESGISPGKYDTY |
| | | GSTRKNLRMILRNLIENDSGVYYCATWDGFYYKKLFGSGTTLVVT |
| 113 | CDR3 VG8 D37 | CATWDNYMKLF |
| 114 | CDR3 VD3 F2 | CASSYTLKLGDTPGRVRDWKLIF |
| 115 | CDR3 VG4 F2 | CATWDGPPYYKKLF |
| 116 | CDR3 VD1 | CALGDYLGDKYPSYDLLGDTTDKLIF |
| | Ze11 | |
| 117 | CDR3 VG8 | CATWDNYKKLF |
| | Ze11 | |
| 118 | CDR3 VD5 B23 | CAASSPIRGYTGSDKLIF |
| 119 | CDR3 VG8 B23 | CATWDNYKKLF |
| 120 | TRD F2 | MILTVGFSFLFFYRGTLCDKVTQSSPDQTVASGSEVVLLCTYDTV |
| | | YSNPDLFWYRIRPDYSFQFVFYGDNSRSEGADFTQGRFSVKHILT |
| | | QKAFHLVISPVRTEDSATYYCASSYTLKLGDTPGRVRDWKLIFGK |
| | | GTRVTVEPR |
| 121 | TRG F2 | MEWALAVLLAFLSPASQKSSNLEGRTKSVIRQTGSSAEITCDLAE |
| | | GSTGYIHWYLHQEGKAPQRLLYYDSYTSSVVLESGISPGKYDTY |
| | | GSTRKNLRMILRNLIENDSGVYYCATWDGPPYYKKLFGSGTTLVV |
| | | T |
| 122 | TRD Ze11 | MVFSSLLCVFVAFSYSGSSVAQKVTQAQSSVSMPVRKAVTLNCL |
| | | YETSWWSYYIFWYKQLPSKEMIFLIRQGSDEQNAKSGRYSVNFK |
| | | KAAKSVALTISALQLEDSAKYFCALGDYLGDKYPSYDLLGDTTDKL |
| | | IFGKGTRVTVEPR |
| 123 | TRG Ze11 | MVLALALLLAFLPPASQKSSNLEGRTKSVTRPTGSSAVITCDLPVE |
| | | NAVYTHWYLHQEGKAPQRLLYYDSYNSRVVLESGISREKYHTYA |
| | | STGKSLKFILENLIERDSGVYYCATWDNYKKLFGSGTTLVVT |
| 124 | TRD B23 | MAMLLGASVLILWLQPDWVNSQQKNDDQQVKQNSPSLSVQEGR |
| | | ISILNCDYTNSMFDYFLWYKKYPAEGPTFLISISSIKDKNEDGRFTV |
| | | FLNKSAKHLSLHIVPSQPGDSAVYFCAASSPIRGYTGSDKLIFGKG |
| | | TRVTVEPR |
| 125 | TRG B23 | MVLALALLLAFLPPASQKSSNLEGRTKSVTRPTGSSAVITCDLPVE |
| | | NAVYTHWYLHQEGKAPQRLLYYDSYNSRVVLESGISREKYHTYA |
| | | STGKSLKFILENLIERDSGVYYCATWDNYKKLFGSGTTLVVT |
| 126 | CDR3 VD1 B9 | CALGNGNHIGYWRYTDKLIF |
| 127 | CDR3 VG5 B9 | CATWDRLYYKKLF |
| 128 | TRD B9 | MVFSSLLCVFVAFSYSGSSVAQKVTQAQSSVSMPVRKAVTLNCL |
| | | YETSWWSYYIFWYKQLPSKEMIFLIRQGSDEQNAKSGRYSVNFK |
| | | KAAKSVALTISALQLEDSAKYFCALGNGNHIGYWRYTDKLIFGKGT |
| | | RVTVEPR |
| 129 | TRG B9 | MVWALLVLLAFLSPASQKSSNLEGGTKSVTRPTRSSAEITCDLTVI |
| | | NAFYIHWYLHQEGKAPQRLLYYDVSNSKDVLESGLSPGKYYTHT |
| | | PRRWSWILILRNLIENDSGVYYCATWDRLYYKKLFGSGTTLVVT |
| 130 | CDR3 VG8 An2 | CATWDSSKLF |
| 131 | CDR3 VD3 An2 | CAFTGLGDTSHADKLIF |
| 132 | TRG An2 | MLLALALLLAFLPPASQKSSNLEGRTKSVTRPTGSSAVITCDLPVE |
| | | NAVYTHWYLHQEGKAPQRLLYYDSYNSRVVLESGISREKYHTYA |
| | | STGKSLKFILENLIERDSGVYYCATWDSSKLFGSGTTLVVT |
| 133 | TRD An2 | MILTVGFSFLFFYRGTLCDKVTQSSPDQTVASGSEVVLLCTYDTV |
| | | YSNPDLFWYRIRPDYSFQFVFYGDNSRSEGADFTQGRFSVKHILT |
| | | QKAFHLVISPVRTEDSATYYCAFTGLGDTSHADKLIFGKGTRVTV |
| | | EPR |

In some embodiments, an exogenous antigen-recognition receptor is a gamma-delta (γδ) T-cell receptor comprising a delta chain represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 90 or SEQ ID NO: 111. In some embodiments, an exogenous antigen-recognition receptor is a gamma-delta (γδ) T-cell receptor comprising a gamma chain represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 91 or SEQ ID NO: 112.

In some embodiments, an exogenous antigen-recognition receptor is a gamma-delta (γδ) T-cell receptor comprising a delta chain CDR3 region represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 84 or SEQ ID NO: 102. In some embodiments, an exogenous antigen-recognition receptor is a gamma-delta (γδ) T-cell receptor comprising a gamma chain CDR3 region represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 87 or SEQ ID NO: 101.

In some embodiments, an exogenous antigen-recognition receptor is a gamma-delta (γδ) T-cell receptor comprising a delta chain represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 90 and a gamma chain represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 91. In some embodiments, an exogenous antigen-recognition receptor is a gamma-delta (γδ) T-cell receptor comprising a delta chain CDR3 region represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 84 and a gamma chain CDR3 region represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 87.

In some embodiments, an exogenous antigen-recognition receptor is a gamma-delta (γδ) T-cell receptor comprising a delta chain represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 111 and a gamma chain represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 112. In some embodiments, an exogenous antigen-recognition receptor is a gamma-delta (γδ) T-cell receptor comprising a delta chain CDR3 region represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 102 and a gamma chain CDR3 region represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity with SEQ ID NO: 101.

In some embodiments, an exogenous antigen-recognition receptor is an alpha-beta (αβ) T-cell receptor comprising a beta chain represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity to a sequence selected from SEQ ID NOs: 198, 215, and 219. In some embodiments, an exogenous antigen-recognition receptor is an alpha-beta (αβ) T-cell receptor comprising an alpha chain represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity to an amino acid sequence selected from SEQ ID NOs: 199, 214, and 218.

In some embodiments, an exogenous antigen-recognition receptor is an alpha-beta (αβ) T-cell receptor comprising a beta chain CDR3 region represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity to a sequence selected from SEQ ID NOs: 211, 217 and 221. In some embodiments, an exogenous antigen-recognition receptor is an alpha-beta (αβ) T-cell receptor comprising an alpha chain CDR3 region represented by an amino acid sequence having at least 60%, at least 65%, at least 70%, at least 75%, at least 80%, at least 85%, at least 90%, at least 95%, at least 97%, at least 98%, at least 99%, or up to 100% sequence identity or similarity to a sequence selected from SEQ ID NOs: 210, 216, and 220.

An exogenous antigen-recognition receptor can be any chimeric antigen receptor (CAR) known at the time of filing. CARs, also known as artificial T cell receptors, chimeric immunoreceptors, or chimeric T cell receptors, can comprise an extracellular targeting domain, a transmembrane domain, and an intracellular signaling domain. CARs generally induce signaling in the engineered cell that expresses the CAR but not a cell that is recognized by the CAR. A CAR can comprise at least a first targeting domain. Non-limiting examples of CAR targeting domain include, but are not limited to, a monoclonal antibody, a polyclonal antibody, a recombinant antibody, a human antibody, a humanized antibody, or a functional derivative, variant or fragment thereof, including, but not limited to, a Fab, a Fab′, a F(ab′)2, an Fv, a single-chain Fv (scFv), minibody, a diabody, and a single-domain antibody such as a heavy chain variable domain (VH), a light chain variable domain (VL), a DARPin, a monobody, a nanobody, an affibody, a non-antibody domain, and any combination thereof. A non-antibody CAR targeting domain can be from or derived from a receptor or a receptor ligand, for example, APRIL can be used to target BCMA. A CAR may generally comprise a targeting domain, hinge domain (H) or spacer, transmembrane domain (TM) providing anchorage to plasma membrane, and signaling domains responsible of T-cell activation.

In an aspect, a CAR, further comprises a hinge. A hinge can be located at any region of a CAR. In an aspect, a hinge is located between a targeting region and a transmembrane region. In another aspect, a subject CAR comprises a hinge or a spacer. The hinge or the spacer can refer to a segment between the targeting moiety and the transmembrane domain. In some embodiments, a hinge can be used to provide flexibility to a targeting moiety, e.g., scFv. In some embodiments, a hinge can be used to detect the expression of a CAR on the surface of a cell, for example when antibodies to detect the scFv are not functional or available. In some cases, the hinge is derived from an immunoglobulin molecule and may require optimization depending on the location of the first epitope or second epitope on the target. In some cases, a hinge may not belong to an immunoglobulin molecule but instead to another molecule such the native hinge of a CD8 alpha molecule. A CD8 alpha hinge can contain cysteine and proline residues which many play a role in the interaction of a CD8 co-receptor and MHC molecule.

A targeting moiety of a CAR can be linked to an intracellular signaling domain via a transmembrane domain. A transmembrane domain can be a membrane spanning segment. A transmembrane domain of a subject CAR can anchor the CAR to the plasma membrane of a cell, for example an engineered cell. In some embodiments, the membrane spanning segment comprises a polypeptide. The membrane spanning polypeptide linking the targeting moiety and the intracellular signaling domain of the CAR can have any suitable polypeptide sequence. In some cases, the membrane spanning polypeptide comprises a polypeptide sequence of a membrane spanning portion of an endogenous or wild-type membrane spanning protein. In some embodiments, the membrane spanning polypeptide comprises a polypeptide sequence having at least 1 (e.g., at least 2, 3, 4, 5, 6, 7, 8, 9, 10 or greater) amino acid substitutions, deletions, and/or insertions compared to a membrane spanning portion of an endogenous or wild-type membrane spanning protein. In some embodiments, the membrane spanning polypeptide comprises a non-natural polypeptide sequence, such as the sequence of a polypeptide linker. The polypeptide linker may be flexible or rigid. The polypeptide linker can be structured or unstructured. In some embodiments, the membrane spanning polypeptide transmits a signal from an extracellular targeting moiety to an intracellular region of the CAR. In an aspect, a subject CAR can comprise a transmembrane region that connects the targeting moiety to the intracellular region. A transmembrane region can be from or derived from an exogenous cellular transmembrane region. Various transmembrane regions are known in the art and can be from immune cell receptors. In an aspect, a transmembrane domain is from an alpha chain of a T cell receptor (TCR), beta chain of a TCR, CD8, CD4, CD28, CD45, ICOS, PD-1 and/or CD152. A native transmembrane portion of CD28 can be used in a CAR. In other cases, a native transmembrane portion of CD8 alpha can also be used in a subject CAR. In an aspect, the transmembrane domain is from an alpha chain of a TCR. In an aspect, the transmembrane domain is from CD8 and is CD8α.

The intracellular signaling domain of a CAR can comprise a signaling domain, or any derivative, variant, or fragment thereof, involved in cell signaling. The intracellular signaling domain of a CAR can induce activity of an engineered cell comprising the CAR. While usually the signaling domain of another molecule can be employed in a CAR, in many cases it is not necessary to use the entire chain. In some cases, a truncated portion of the signaling domain is used in a CAR of an engineered cell provided herein.

In some embodiments, the CAR intracellular signaling domain comprises multiple signaling domains involved in cell signaling, or any derivatives, variants, or fragments thereof. An intracellular signaling domain used in a CAR can be involved in regulating primary activation of the TCR complex in either a stimulatory way or an inhibitory way. The CAR intracellular signaling domain may be that of a TCR complex. The CAR intracellular signaling domain can comprise a signaling domain of an Fcγ receptor (FcγR), an Fcε receptor (FcεR), an Fcα receptor (FcαR), neonatal Fc receptor (FcRn), CD3, CD3 ζ, CD3 γ, CD3 δ, CD3 ε, CD4, CD5, CD8, CD21, CD22, CD26, CD28, CD32, CD40L (CD154), CD45, CD46, 41BB, OX40, GITR, CD66d, CD79a, CD79b, CD80, CD86, CD278 (also known as ICOS), CD247 ζ, CD247 η, DAP10, DAP12, FYN, LAT, Lck, MAPK, MHC complex, NFAT, NF-κB, PLC-γ, iC3b, C3dg, C3d, and Zap70. In some embodiments, the CAR signaling domain includes an immunoreceptor tyrosine-based activation motif or ITAM. A CAR signaling domain comprising an ITAM can comprise two repeats of the amino acid sequence YxxL/I separated by 6-8 amino acids, wherein each x is independently any amino acid, producing the conserved motif YxxL/Ix(6-8)YxxL/I. A CAR signaling domain comprising an ITAM can be modified, for example, by phosphorylation when the targeting moiety is bound to an epitope. A phosphorylated ITAM can function as a docking site for other proteins, for example proteins involved in various signaling pathways. In some embodiments, the primary CAR signaling domain comprises a modified ITAM domain, e.g., a mutated, truncated, and/or optimized ITAM domain, which has altered (e.g., increased or decreased) activity compared to the native ITAM domain.

In some embodiments, the intracellular signaling domain of a CAR comprises an FcγR signaling domain (e.g., ITAM). The FcγR signaling domain can be selected from FcγRI (CD64), FcγRIIA (CD32), FcγRIIB (CD32), FcγRIIIA (CD16a), and FcγRIIIB (CD16b). In some embodiments, the CAR intracellular signaling domain comprises an FcεR signaling domain (e.g., ITAM). The FcεR signaling domain can be selected from FcεRI and FcεRII (CD23). In some embodiments, the CAR intracellular signaling domain comprises an FcαR signaling domain (e.g., ITAM). The FcαR signaling domain can be selected from FcαRI (CD89) and Fcα/μR. In some embodiments, the CAR intracellular signaling domain comprises a CD3 (signaling domain. In some embodiments, the primary CAR signaling domain comprises an ITAM of CD3 ζ.

In some embodiments, an intracellular signaling domain of a subject CAR comprises an immunoreceptor tyrosine-based inhibition motif or ITIM. A signaling domain comprising an ITIM can comprise a conserved sequence of amino acids (S/I/V/LxYxxI/V/L) that is found in the cytoplasmic tails of some inhibitory receptors of the immune system. A primary CAR signaling domain comprising an ITIM can be modified, for example phosphorylated, by enzymes such as a Src kinase family member (e.g., Lck). Following phosphorylation, other proteins, including enzymes, can be recruited to the ITIM. These other proteins include, but are not limited to, enzymes such as the phosphotyrosine phosphatases SHP-1 and SHP-2, the inositol-phosphatase called SHIP, and proteins having one or more SH2 domains (e.g., ZAP70). A CAR intracellular signaling domain can comprise a signaling domain (e.g., ITIM) of BTLA, CD5, CD31, CD66a, CD72, CMRF35H, DCIR, EPO-R, FcγRIIB (CD32), Fc receptor-like protein 2 (FCRL2), Fc receptor-like protein 3 (FCRL3), Fc receptor-like protein 4 (FCRL4), Fc receptor-like protein 5 (FCRL5), Fc receptor-like protein 6 (FCRL6), protein G6b (G6B), interleukin 4 receptor (IL4R), immunoglobulin superfamily receptor translocation-associated 1 (IRTA1), immunoglobulin superfamily receptor translocation-associated 2 (IRTA2), killer cell immunoglobulin-like receptor 2DL1 (KIR2DL1), killer cell immunoglobulin-like receptor 2DL2 (KIR2DL2), killer cell immunoglobulin-like receptor 2DL3 (KIR2DL3), killer cell immunoglobulin-like receptor 2DL4 (KIR2DL4), killer cell immunoglobulin-like receptor 2DL5 (KIR2DL5), killer cell immunoglobulin-like receptor 3DL1 (KIR3DL1), killer cell immunoglobulin-like receptor 3DL2 (KIR3DL2), leukocyte immunoglobulin-like receptor subfamily B member 1 (LIR1), leukocyte immunoglobulin-like receptor subfamily B member 2 (LIR2), leukocyte immunoglobulin-like receptor subfamily B member 3 (LIR3), leukocyte immunoglobulin-like receptor subfamily B member 5 (LIR5), leukocyte immunoglobulin-like receptor subfamily B member 8 (LIR8), leukocyte-associated immunoglobulin-like receptor 1 (LAIR-1), mast cell function-associated antigen (MAFA), NKG2A, natural cytotoxicity triggering receptor 2 (NKp44), NTB-A, programmed cell death protein 1 (PD-1), PILR, SIGLECL1, sialic acid binding Ig like lectin 2 (SIGLEC2 or CD22), sialic acid binding Ig like lectin 3 (SIGLEC3 or CD33), sialic acid binding Ig like lectin 5 (SIGLEC5 or CD170), sialic acid binding Ig like lectin 6 (SIGLEC6), sialic acid binding Ig like lectin 7 (SIGLEC7), sialic acid binding Ig like lectin 10 (SIGLEC0), sialic acid binding Ig like lectin 11 (SIGLEC11), sialic acid binding Ig like lectin 4 (SIGLEC4), sialic acid binding Ig like lectin 8 (SIGLEC8), sialic acid binding Ig like lectin 9 (SIGLEC9), platelet and endothelial cell adhesion molecule 1 (PECAM-1), signal regulatory protein (SIRP 2), and signaling threshold regulating transmembrane adaptor 1 (SIT). In some embodiments, the CAR intracellular signaling domain comprises a modified ITIM domain, e.g., a mutated, truncated, and/or optimized ITIM domain, which has altered (e.g., increased or decreased) activity compared to the native ITIM domain.

In some embodiments, the CAR intracellular signaling domain comprises at least 2 ITAM domains (e.g., at least 3, 4, 5, 6, 7, 8, 9, or 10 ITAM domains). In some embodiments, the CAR intracellular signaling domain comprises at least 2 ITIM domains (e.g., at least 3, 4, 5, 6, 7, 8, 9, or 10 ITIM domains) (e.g., at least 2 primary signaling domains). In some embodiments, the CAR intracellular signaling domain comprises both ITAM and ITIM domains. In an aspect, an intracellular signaling domain of subject CAR is from an Fcγ receptor (FcγR), an Fcε receptor (FcεR), an Fcα receptor (FcαR), neonatal Fc receptor (FcRn), CD3, CD3ζ, CD3γ, CD3δ, CD3ε, CD4, CD5, CD8, CD21, CD22, CD28, CD32, CD40L (CD154), CD45, CD66d, CD79a, CD79b, CD80, CD86, CD278 (also known as ICOS), CD247 ζ, CD247 η, DAP10, DAP12, FYN, LAT, Lck, MAPK, MHC complex, NFAT, NF-κB, PLC-γ, iC3b, C3dg, C3d, and Zap70. In another aspect, the intracellular signaling domain of a subject CAR is from CD3, CD3ζ, CD3γ, CD3δ, and/or CD3ε. In another aspect, the intracellular signaling domain of a subject CAR is from CD3ζ.

In some cases, a CAR intracellular signaling domain that comprises a co-stimulatory domain. In some embodiments, a CAR co-stimulatory domain, for example from a cellular co-stimulatory molecule, can provide co-stimulatory signals for engineered cell signaling, such as signaling from ITAM and/or ITIM domains, e.g., for the activation and/or deactivation of engineered cell activity. In some embodiments, a CAR costimulatory domain is operable to regulate a proliferative and/or survival signal in the engineered cell. In some embodiments, a CAR co-stimulatory signaling domain comprises a signaling domain of a MHC class I protein, MHC class II protein, TNF receptor protein, immunoglobulin-like protein, cytokine receptor, integrin, signaling lymphocytic activation molecule (SLAM protein), activating NK cell receptor, BTLA, or a Toll ligand receptor. In some embodiments, the CAR costimulatory domain comprises a signaling domain of a molecule selected from the group consisting of: 2B4/CD244/SLAMF4, 4-1BB/TNFSF9/CD137, CD137L, B7-1/CD80, B7-2/CD86, B7-H1/PD-L1, B7-H2, B7-H3, B7-H4, B7-H6, B7-H7, BAFF R/TNFRSF13C, BAFF/BLyS/TNFSF13B, BLAME/SLAMF8, BTLA/CD272, CD100 (SEMA4D), CD103, CD11a, CD11b, CD11c, CD11d, CD150, CD160 (BY55), CD18, CD19, CD2, CD200, CD229/SLAMF3, CD27 Ligand/TNFSF7, CD27/TNFRSF7, CD28, CD29, CD2F-10/SLAMF9, CD30 Ligand/TNFSF8, CD30/TNFRSF8, CD300a/LMIR1, CD4, CD40 Ligand/TNFSF5, CD40/TNFRSF5, CD46, CD48/SLAMF2, CD49a, CD49D, CD49f, CD5, CD53, CD58/LFA-3, CD69, CD7, CD8 α, CD8 β, CD82/Kai-1, CD84/SLAMF5, CD90/Thy1, CD96, CDS, CEACAM1, CRACC/SLAMF7, CRTAM, CTLA-4, DAP12, Dectin-1/CLEC7A, DNAM1 (CD226), DPPIV/CD26, DR3/TNFRSF25, EphB6, GADS, Gi24/VISTA/B7-H5, GITR Ligand/TNFSF18, GITR/TNFRSF18, HLA Class I, HLA-DR, HVEM/TNFRSF14, IA4, ICAM-1, ICOS/CD278, Ikaros, IL2R β, IL2R γ, IL7R α, Integrin α4/CD49d, Integrin α4β1, Integrin α4β7/LPAM-1, IPO-3, ITGA4, ITGA6, ITGAD, ITGAE, ITGAL, ITGAM, ITGAX, ITGB1, ITGB2, ITGB7, KIRDS2, LAG-3, LAT, LIGHT/TNFSF14, LTBR, Ly108, Ly9 (CD229), lymphocyte function associated antigen-1 (LFA-1), Lymphotoxin-α/TNF-β, NKG2C, NKG2D, NKp30, NKp44, NKp46, NKp80 (KLRF1), NTB-A/SLAMF6, OX40 Ligand/TNFSF4, OX40/TNFRSF4, PAG/Cbp, PD-1, PDCD6, PD-L2/B7-DC, PSGL1, RELT/TNFRSF19L, SELPLG (CD162), SLAM (SLAMF1), SLAM/CD150, SLAMF4 (CD244), SLAMF6 (NTB-A), SLAMF7, SLP-76, TACI/TNFRSF13B, TCL1A, TCL1B, TIM-1/KIM-1/HAVCR, TIM-4, TL1A/TNFSF15, TNF RII/TNFRSF1B, TNF-α, TRANCE/RANKL, TSLP, TSLP R, VLA1, and VLA-6. In some embodiments, the CAR costimulatory domain comprises a signaling domain of a molecule selected from the group consisting of: CD3 and CD28.

In some embodiments, the CAR intracellular signaling domain comprises multiple costimulatory domains, for example at least two, e.g., at least 3, 4, or 5 costimulatory domains. In an aspect, a CAR comprises at least 2 or 3 co-stimulatory domains. In an aspect, a CAR comprises at least 2 costimulatory domains, and wherein the at least 2 costimulatory domains are CD28 and CD137. In an aspect, the CAR comprises at least 3 costimulatory domains, wherein the at least 3 costimulatory domains are CD28, CD137, and OX40. Co-stimulatory signaling regions may provide a signal synergistic with the primary effector activation signal and can complete the requirements for activation of a T cell. In some embodiments, the addition of co-stimulatory domains to the CAR can enhance the efficacy and persistence of the engineered cells provided herein.

The CAR can be a CAR that binds to an antigen that is associated with a cancer, for example, an antigen that is over-expressed in a cancer, or a neoantigen. In some cases, the CAR targets CD19. In some embodiments, the CAR targets BCMA.

In some embodiments, a CAR comprises an anti CD19 scFv linked to CD8 stalk and transmembrane domain with 41BB and CD3z intracellular signaling domains (CD19.BB.Z). In some embodiments, a CAR comprises an anti CD19 scFv linked to CD28 stalk and transmembrane domain with CD28 and CD3z intracellular signaling domains (CD19.28.Z). In some embodiments, a CAR comprises an anti EGFR scFv linked to CD8 stalk and transmembrane domain with 41BB and CD3z intracellular signaling domains (EGFR.BB.z). in some embodiments, a CAR comprises NKG2D and CD3z intracellular signaling domain (NKG2D.z). Non-limiting examples of such CARs are described in WO2019/157533, Bloemberg et al. (2020), Mol Ther Methods Clin Dev 16; 238-254, and Zhang et al. (2006), Cancer Res 66:11; 5927-5933, all of which are incorporated herein by reference in their entireties.

In some embodiments, a CAR comprises a sequence having at least 60%, at least 65%, at least 70%, at least 75%, at least 80%, at least 85%, at least 90%, at least 95%, at least 97%, at least 98%, at least 99%, or up to 100% sequence identity or similarity with a sequence selected from SEQ ID NOs: 184, 212, 213 and 222.

In some embodiments, the exogenous antigen-recognition receptor binds to cancer-associated antigen selected from the group consisting of: 707-AP, a biotinylated molecule, a-Actinin-4, abl-bcr alb-b3 (b2a2), abl-bcr alb-b4 (b3a2), adipophilin, AFP, AIM-2, Annexin II, ART-4, BAGE, b-Catenin, bcr-abl, bcr-abl p190 (e1a2), bcr-abl p210 (b2a2), bcr-abl p210 (b3a2), BING-4, CAG-3, CAIX, CAMEL, Caspase-8, CD171, CD277, CD19, CD20, CD22, CD23, CD24, CD30, CD33, CD38, CD44v7/8, CDC27, CDK-4, CEA, CLCA2, Cyp-B, DAM-10, DAM-6, DEK-CAN, EGFRvIII, EGP-2, EGP-40, ELF2, Ep-CAM, EphA2, EphA3, erb-B2, erb-B3, erb-B4, ES-ESO-1a, ETV6/AML, FBP, fetal acetylcholine receptor, FGF-5, FN, G250, GAGE-1, GAGE-2, GAGE-3, GAGE-4, GAGE-5, GAGE-6, GAGE-7B, GAGE-8, GD2, GD3, GnT-V, Gp100, gp75, Her-2, HLA-A*0201-R1701, HMW-MAA, HSP70-2 M, HST-2 (FGF6), HST-2/neu, hTERT, iCE, IL-11Rα, IL-13Rα2, KDR, KIAA0205, K-RAS, L1-cell adhesion molecule, LAGE-1, LDLR/FUT, Lewis Y, MAGE-1, MAGE-10, MAGE-12, MAGE-2, MAGE-3, MAGE-4, MAGE-6, MAGE-A1, MAGE-A2, MAGE-A3, MAGE-A6, MAGE-B1, MAGE-B2, Malic enzyme, Mammaglobin-A, MART-1/Melan-A, MART-2, MC1R, M-CSF, mesothelin, MUC1, MUC16, MUC2, MUM-1, MUM-2, MUM-3, Myosin, NA88-A, Neo-PAP, NKG2D, NPM/ALK, N-RAS, NY-ESO-1, OA1, OGT, oncofetal antigen (h5T4), OS-9, P polypeptide, P15, P53, PRAME, PSA, PSCA, PSMA, PTPRK, RAGE, ROR1, RU1, RU2, SART-1, SART-2, SART-3, SOX10, SSX-2, Survivin, Survivin-2B, SYT/SSX, TAG-72, TEL/AML1, TGFaRII, TGFbRII, TP1, TRAG-3, TRG, TRP-1, TRP-2, TRP-2/INT2, TRP-2-6b, Tyrosinase, VEGF-R2, WT1, α-folate receptor, and κ-light chain.

In some embodiments, the exogenous antigen-recognition receptor binds to a neoantigen or neoepitope. For example, a neoantigen can be an E805G mutation in ERBB2IP. Neoantigen and neoepitopes can be identified by whole-exome sequencing in some cases. A neoantigen and neoepitope target can be expressed by a cancer cell. In some cases, a gene that can comprise a mutation that gives rise to a neoantigen or neoepitope can be ABL1, ACOI 1997, ACVR2A, AFP, AKT1, ALK, ALPPL2, ANAPC1, APC, ARID1A, AR, AR-v7, ASCL2, β2M, BRAF, BTK, C15ORF40, CDH1, CLDN6, CNOT1, CT45A5, CTAGIB, DCT, DKK4, EEF1B2, EEF1DP3, EGFR, EIF2B3, env, EPHB2, ERBB3, ESR1, ESRP1, FAM11 IB, FGFR3, FRG1B, GAGE1, GAGE 10, GATA3, GBP3, HER2, IDH1, JAK1, KIT, KRAS, LMAN1, MABEB 16, MAGEA1, MAGEA10, MAGEA4, MAGEA8, MAGEB 17, MAGEB4, MAGECI, MEK, MLANA, MLL2, MMP13, MSH3, MSH6, MYC, NDUFC2, NRAS, NY-ESO, PAGE2, PAGE5, PDGFRa, PIK3CA, PMEL, pol protein, POLE, PTEN, RAC1, RBM27, RNF43, RPL22, RUNX1, SEC31A, SEC63, SF3B 1, SLC35F5, SLC45A2, SMAP1, SMAP1, SPOP, TFAM, TGFBR2, THAP5, TP53, TTK, TYR, UBR5, VHL, XPOT.

Non-limiting examples of cancer antigens are provided in Table 7.

TABLE 7

| Antigen | Abbreviation |
|---|---|
| 9D7 | 9D7 |
| alpha-fetoprotein | AFP |
| A-kinase achor protein 4 | AKAP-4 |
| Anaplastic lymphoma kinase | ALK |
| Androgen receptor | AR |
| B7-H3 | B7-H3 |
| B melanoma antigen family | BAGE family |
| bcr-abl | bcr-abl |
| BING-4 | BING-4 |
| Brother of the regulator of the imprinted site | BORIS |
| BRAF | BRAF |
| Breast cancer gene 1/2 | BRCA1/2 |
| Carbohydrate antigen 19-9 | CA 19-9 |
| Carbonic anhydrase IX | CA9/CAIX |
| Calcium-activated chloride channel 2 | Ca-CC2 |
| Cancer antigen family | CAGE family |
| CD19 | CD19 |
| CD20 | CD20 |
| CD22 | CD22 |
| CD30 | CD30 |
| CD33/IL3Ra | CD33/IL3Ra |
| CD38 | CD38 |
| CD44v6/7/8 | CD44v6/7/8 |
| CD52 | CD52 |
| Cyclin-dependent kinase 4 | CDK4 |
| Carcinoembryonic antigen | CEA |
| Tyrosine protein kinase Met | c-Met |
| CML66 | CML66 |
| Cyclin-B₁ | Cyclin-B₁ |
| Cytochrome p450 1B1 | CYP1B1 |
| Epidermal growth factor receptor | EGFR |
| Epidermal growth factor receptor variant III | EGFRvIII |
| Epithelial cell adhesion molecule | EpCAM |
| (including EGP2 and EGP40) | (EGP2/EGP40) |
| Ephrin type A receptor 2/3 | EphA2/3 |
| ErbB3/4 | ErbB3/4 |
| ETV6 | ETV6 |
| Fanconi anemia group D2 | FANCD2 |
| Fibroblast activation protein alpha | FAP |
| Fibroblast growth factor receptor 2 | FGFR2 |
| Fibronectin | Fibronectin |
| Fos-related antigen 1 | Fra-1 |
| Folate receptor-a | FRα |
| Fucosyl GM1 | Fucosyl GM1 |
| GAGE family | GAGE family |
| disialoganglioside 2 (GD2) | GD2 |
| GD2/3 | GD2/3 |
| GloboH | GloboH |
| Glycolipid F77 | Glycolipid F77 |
| GM3 | GM3 |
| Gp100/premelanosome protein | Gp100/PMEL |
| Human epidermal growth factor receptor 2 | HER-2 |
| Human high molecular weight melanoma | HMWMAA |
| associated antigen | |
| Human papillomavirus E6/E7 | HPVE6/E7 |
| Isocitrate dehydrogenase 1 | IDH1 |
| Insulin like growth factor 2/Insulin | IGF2/IGF1R |
| like growth factor 1 receptor | |
| IL-11 receptor a | IL11Rα |
| IL-13 receptor α2 | IL13Rα2 |
| IL-3 receptor | IL3R/CD123 |
| Immature laminin receptor | iLRP |
| Neural cell adhesion molecule L1 | L1CAM/CD171 |
| LCK | LCK |
| Legumain | Legumain |
| Lewis Y | Lewis Y |
| MAD-CT-1/2 | MAD-CT-1/2 |
| Melanoma antigen gene family | MAGE family |
| Melanoma antigen recognized by T cells 2 | MART-2 |
| Melanocortin 1 receptor | MC1R |
| MDM2/4 | MDM2/4 |
| Melan-A/Melanoma antigen recognized by T cells | Melan-A/MART-1 |
| Mesothelin | Mesothelin |
| Melanoma-inhibitor of apoptosis | ML-IAP |
| Mucin 1 | MUC1 |
| Multiple targets | Multiple targets |
| MYB | MYB |
| MYCN | MYCN |
| Neural cell adhesion molecule | NCAM/CD56 |
| NKG2D ligands | NKG2D ligands |
| New York esophogeal squamous cell | NY-ESO-1/LAGE-1 |
| carcinoma 1/LAGE-1 | |
| OY-TES1 | OY-TES1 |
| P. polypeptide | P. polypeptide |
| p53 mutant | p53 mutant |
| p53 non-mutant | p53 non-mutant |
| Prostate associated gene 4 | Page4 |
| Poly(a) polymerase | PAP |
| Paired box gene 3/5 | PAX3/5 |
| Platelet derived growth factor receptor beta | PDGFR-β |
| Placenta specific 1 | PLAC1 |
| Polysialic acid | Polysialic acid |
| Proteinase 1/3 | PR1/3 |
| Preferentually expressed antigen in melanoma | PRAME |
| Prostate specific antigen | PSA |
| Prostate stem cell antigen | PSCA |
| Prostate specific membrane antigen | PSMA |
| Ras mutant | Ras mutant |
| Ras non-mutant | Ras non-mutant |
| RGS5 | RGS5 |
| RhoC | RhoC |
| Receptor tyrosine kinase-like orphan | ROR1 |
| receptor 1 | |
| Stomach cancer associated protein | SAP-1 |
| tyrosine phosphatase 1 | |
| Squamous cell carcinoma antigen | SART3 |
| recognized by T cells 3 | |
| Sperm protein 17 | Sp17 |
| Somatostatin receptor 2 | SSTR2 |
| Synovial sarcoma, × breakpoint 2 | SSX2 |
| Staufen1 | Staufen1 |
| STn | STn |
| Survivin | Survivin |
| TAG72 | TAG72 |
| Telomerase | Telomerase |
| Tumor endothelial marker 1/8 | TEM1/8 |
| Transforming growth factor B receptor II | TGF-βRII |
| Tie2 | Tie2 |
| TMEM97/Sigma 2 receptor | TMEM97 |
| Transmembrane protease, serine 2-ERG | TMPRSS2ETS-ERG |
| Tn | Tn |
| Tyrosinase related protein 1/2 | TRP-1/-2 |
| Tyrosinase | Tyrosinase |
| Vascular endothelial grown factor | VEGF |
| Wilm's tumor 1 | WT1 |
| αvβ3 integrin | αvβ3 integrin |
| β-catenin | β-catenin |
| CMV proteins or antigens | |

Epstein-Barr Virus (EBV) protein or antigen

An exogenous antigen recognition receptor can be introduced into an engineered cell via one vector or using different vectors. An exogenous antigen recognition receptor and a MIDIS protein can be expressed as one transcript (e.g., separated by one or more self-cleaving peptide sequence) or as different transcripts. An exogenous antigen recognition receptor and a MIDIS protein can be operably linked and under regulatory control of the same promoter or different promoters. Self-cleaving peptide sequences and promoters are discussed later herein.

In some cases, an exogenous antigen recognition receptor requires an antigen to be presented by MHC for antigen-based activation to occur. In some cases, an exogenous antigen recognition receptor does not require an antigen to be presented by MHC for antigen-based activation to occur.

In an aspect, an engineered cell can comprise a higher ratio of an exogenous antigen-recognition receptor as compared to an endogenous cellular receptor. In certain cases, a higher ratio can be achieved by way of preferential expansion of an engineered cell that expresses a MIDIS protein, for example resulting in a (γδ) single TCR positive phenotype of γδTCR-engineered cells.

In an embodiment, an engineered cell comprises a higher ratio of a γδTCR to αβTCR as compared to an otherwise comparable cell that is not engineered (e.g., does not express a MIDIS protein). In an embodiment, an engineered cell comprises a higher ratio of a γ9δ2TCR to αβTCR as compared to an otherwise comparable cell that is not engineered. In an embodiment, where the engineered cell comprises the MIDIS protein, a predominantly (γδ) single TCR positive phenotype can be achieved in combination with prolonged lifespan of engineered cells.

Any of the engineered cells of the disclosure can comprise a ratio of an exogenous antigen-recognition receptor to endogenous cellular receptor that is at least 1 fold, 2 fold, 3 fold, 4 fold, 5 fold, 6 fold, 7 fold, 8 fold, 9 fold, 10 fold, 11 fold, 12 fold, 13 fold, 14, fold 15 fold, 20 fold, 30 fold, 40 fold, 50 fold, 60 fold, 70 fold, 80 fold, 90 fold, 100 fold, 150 fold, 200 fold, 250 fold, or 300 fold higher than a corresponding non-engineered cell. In an embodiment, an engineered cell comprises an at least about 1 fold, 2 fold, 3 fold, 4 fold, to 5 fold higher ratio of an exogenous antigen-recognition receptor to an endogenous cellular receptor than a corresponding non-engineered cell.

An engineered cell of the disclosure can comprise a ratio of an exogenous antigen recognition receptor to an endogenous cellular receptor that is at least 1:1, 2:1, 3:1, 4:1, 5:1, 6:1, 7:1, 8:1, 9:1, 10:1, 11:1, 12:1, 13:1, 14:1, 15:1, 20:1, 30:1, 40:1, 50:1, 60:1, 70:1, 80:1, 90:1, 100:1, 150:1, 200:1, 250:1, or 300:1.

In some cases, an engineered cell expresses a MIDIS protein and does not express an exogenous antigen-recognition receptor. In some cases, an engineered cell is a tumor-infiltrating lymphocyte that is engineered to express a MIDIS protein but not an exogenous antigen-recognition receptor.

H. Cells that Express or Present an Antigen that Binds to an Exogenous Antigen-Recognition Receptor

Compositions and methods of the disclosure can comprise one or more cells that express or present an antigen that binds to an exogenous antigen-recognition receptor. For example, an exogenous antigen recognition receptor can bind a cancer antigen, and cancer cells can be cells that express or present an antigen that binds to an exogenous antigen-recognition receptor (e.g., target cells).

MIDIS proteins of the disclosure can modulate (e.g., increase or reduce) a response of engineered cells to cells that express or present an antigen that binds to an exogenous antigen-recognition receptor.

A MIDIS protein can modulate proliferation of engineered cells that express an exogenous antigen-recognition receptor upon exposure to cells that express or present the antigen recognized by the exogenous antigen-recognition receptor. A MIDIS protein can increase proliferation of engineered cells that express an exogenous antigen-recognition receptor upon exposure to cells that express or present the antigen recognized by the exogenous antigen-recognition receptor. For example, upon exposure of the population of engineered cells to cells that express or present the antigen that binds to the exogenous antigen-recognition receptor, proliferation of the population of engineered cells can be increased by at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold compared to a corresponding population of engineered cells that do not express the MIDIS protein.

A MIDIS protein can modulate killing of cells that express or present the antigen recognized by the exogenous antigen-recognition receptor. A MIDIS protein can increase killing of cells that express or present the antigen recognized by the exogenous antigen-recognition receptor. For example, upon exposure of the population of engineered cells to cells that express or present the antigen that binds to the exogenous antigen-recognition receptor, killing of the cells that express or present the antigen recognized by the exogenous antigen-recognition receptor can be increased by at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold compared to exposure to a corresponding population of engineered cells that do not express the MIDIS protein.

In some embodiments, upon exposure of the population of engineered cells to cells that express or present an antigen that binds to the exogenous antigen-recognition receptor, an ability of the engineered cells to kill at least 50% of the cells that express or present the antigen persists at least 1 day, at least 2 days, at least 3 days, at least 4 days, at least 5 days, at least 6 days, at least 7 days, at least 8 days, at least 9 days, at least 10 days, at least 14 days, at least 21 days, or at least 28 days longer than upon exposure to a corresponding population of engineered cells that do not express the MIDIS protein.

In some embodiments, upon exposure of the population of engineered cells to cells that express or present an antigen that binds to the exogenous antigen-recognition receptor, an ability of the engineered cells to kill at least 25% of the cells that express or present the antigen persists at least 1 day, at least 2 days, at least 3 days, at least 4 days, at least 5 days, at least 6 days, at least 7 days, at least 8 days, at least 9 days, at least 10 days, at least 14 days, at least 21 days, or at least 28 days longer than upon exposure to a corresponding population of engineered cells that do not express the MIDIS protein.

In some embodiments, upon exposure of the population of engineered cells to cells that express or present an antigen that binds to the exogenous antigen-recognition receptor, an ability of the engineered cells to kill at least 75% of the cells that express or present the antigen persists at least 1 day, at least 2 days, at least 3 days, at least 4 days, at least 5 days, at least 6 days, at least 7 days, at least 8 days, at least 9 days, at least 10 days, at least 14 days, at least 21 days, or at least 28 days longer than upon exposure to a corresponding population of engineered cells that do not express the MIDIS protein.

In some embodiments, upon exposure of the population of engineered cells to cells that express or present an antigen that binds to the exogenous antigen-recognition receptor for at least 5 days, expression of an exhaustion marker by the population of engineered cells is at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold lower compared to upon exposure to a corresponding population of engineered cells that do not express the MIDIS protein.

In some embodiments, upon exposure of the population of engineered cells to cells that express or present an antigen that binds to the exogenous antigen-recognition receptor for at least 10 days, expression of an exhaustion marker by the population of engineered cells is at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold lower compared to upon exposure to a corresponding population of engineered cells that do not express the MIDIS protein.

In some embodiments, upon exposure of the population of engineered cells to cells that express or present an antigen that binds to the exogenous antigen-recognition receptor for at least 15 days, expression of an exhaustion marker by the population of engineered cells is at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold lower compared to upon exposure to a corresponding population of engineered cells that do not express the MIDIS protein.

A MIDIS protein can modulate production of an immune effector molecule in response to cells that express or present the antigen recognized by the exogenous antigen-recognition receptor. A MIDIS protein can increase production of an immune effector molecule in response to cells that express or present the antigen recognized by the exogenous antigen-recognition receptor. In some embodiments, upon exposure of the population of engineered cells to cells that express or present an antigen that binds to the exogenous antigen-recognition receptor, production of an immune effector molecule by the population of engineered cells is at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold higher than a corresponding population of engineered cells that do not express the MIDIS protein.

### I. Methods of Making Engineered Cells

In an embodiment, there is provided a polynucleotide encoding a chimeric bidirectional signaling transmembrane protein as disclosed herein. Such a polynucleotide may further comprise a nucleotide sequence encoding the interaction partner. Such a polynucleotide may also further comprise a nucleotide sequence encoding the exogenous antigen-recognition receptor. In this context, a preferred exogenous antigen-recognition receptor is a gamma-delta TCR. In an embodiment, the polynucleotide comprises the chimeric bidirectional signaling transmembrane protein as disclosed herein and the exogenous antigen recognition receptor. In an embodiment, the polynucleotide comprises the chimeric bidirectional signaling transmembrane protein as disclosed herein and the gamma-delta TCR.

In an embodiment, one single lentiviral vector comprises said polynucleotide comprising the chimeric bidirectional signaling transmembrane protein as disclosed herein and the gamma-delta TCR. This lentiviral vector comprises a tricistronic sequence and 2A self-cleaving peptide sequences connecting the three protein sequences as used in the experimental part (Xu Y., et al (2019), Cancer Immunology, Immunotherapy, 68: 1979-1993 and Pincha M., et al, (2011), Gene Therapy, 18: 750-764). 2A self-cleaving peptide sequences are further discussed later herein.

In an embodiment, the first polynucleotide sequence encodes a gamma chain of the TCR followed by a polynucleotide sequence encoding the chimeric bidirectional signaling transmembrane protein as disclosed herein subsequently followed by a polynucleotide sequence encoding a delta chain of the TCR.

In an embodiment, the first polynucleotide sequence encodes a delta chain of the TCR followed by a polynucleotide sequence encoding the chimeric bidirectional signaling transmembrane protein as disclosed herein subsequently followed by a polynucleotide sequence encoding a gamma chain of the TCR.

Preferred gamma and delta chains of the TCR and preferred chimeric bidirectional signaling transmembrane protein are disclosed herein.

In an embodiment, the polynucleotide encoding a chimeric bidirectional signaling transmembrane protein as disclosed herein is represented by a nucleotide sequence having any one of at least 80%, at least 85%, at least 90%, at least 91%, at least 92%, at least 93%, at least 94%, at least 95%, at least 96%, at least 97%, at least 98%, at least 98.5%, at least 99%, or at least 99.5% or 100% sequence identity to any one of SEQ ID NO: 140-171, 185-186, or 189-190 as identified in Table 20. Table 20 also identifies the amino acid sequence (SEQ ID NO) encoded by each of SEQ ID NO: 140-171, 185-186, or 189-190 and representing some specific exemplified chimeric bidirectional signaling transmembrane proteins.

Alternatively, such a polynucleotide may comprise a nucleotide sequence encoding the chimeric bidirectional signaling transmembrane protein and the interaction partner and does not comprise a nucleotide sequence encoding an exogenous antigen-recognition receptor.

In an embodiment, there is provided a vector comprising a polynucleotide encoding the chimeric bidirectional signaling transmembrane protein as disclosed herein. Such a vector may further comprise a nucleotide sequence encoding the interaction partner. Such a vector may also further comprise a nucleotide sequence encoding the exogenous antigen-recognition receptor. In this context, a preferred exogenous antigen-recognition receptor is a gamma-delta TCR. Alternatively, such a vector may comprise a nucleotide sequence encoding the chimeric bidirectional signaling transmembrane protein and the interaction partner and does not comprise a nucleotide sequence encoding an exogenous antigen-recognition receptor.

Such a vector may be a viral vector. Suitable vectors are disclosed later herein.

Disclosed herein, in some embodiments, are methods of making engineered cells. The methods can comprise expressing a MIDIS protein of the disclosure in at least one cell in a population of cells, and culturing the population of cells in a condition suitable for expansion of the population of engineered cells.

Expressing a MIDIS protein of the disclosure in engineered cells can increase, for example, fitness, proliferation, survival, and/or effector function of the engineered cells.

In some embodiments, the engineered cells can be cultured for extended periods without stimulation or with reduced stimulation compared to conventional methods of expanding engineered cells, and the MIDIS protein can support expansion and fitness of the population of cells. For example, the MIDIS protein can provide signaling necessary to support survival and proliferation of the engineered cells, reducing or eliminating the need for exogenous stimulating agents.

In some embodiments, methods of making engineered cells can comprise stimulation, such as by contact with an anti-CD3 antibody or antigen-binding fragment thereof, or an anti-CD2 antibody immobilized on a surface, or by contact with a protein kinase C activator (e.g., bryostatin) sometimes in conjunction with a calcium ionophore. For co-stimulation of an accessory molecule on the surface of the T cells, a ligand that binds the accessory molecule can be used. In some cases a population of T cells can be CD3-CD28 co-stimulated, for example, contacted with an anti-CD3 antibody and an anti-CD28 antibody, under conditions that can stimulate proliferation of the T cells.

Conditions appropriate for T cell culture can include an appropriate media (e.g., Minimal Essential Media or RPMI Media 1640, TexMACS (Miltenyi) or, X-vivo 5, (Lonza)) that may contain factors necessary for proliferation and viability, including serum. In some cases, serum-free medium is used. In an aspect, cells can be maintained under conditions necessary to support growth; for example, an appropriate temperature (e.g., 37° C.) and atmosphere (e.g., air plus 5% CO2).

T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005, which are incorporated by reference for such disclosure.

Cells can be obtained from any suitable source for the generation of engineered cells. Cells can be primary cells. Cells can be recombinant cells. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. Cells can be derived from a healthy donor, from a patient diagnosed with cancer, or from a patient diagnosed with an infection. Cells can also be obtained from a cell therapy bank. Cells can also be obtained from whole food, apheresis, or a tumor sample of a subject. A cell can be a tumor infiltrating lymphocytes (TIL). In some cases an apheresis can be a leukapheresis.

A desirable cell population can also be selected prior to modification. A selection can include at least one of: magnetic separation, flow cytometric selection, antibiotic selection. The one or more cells can be any blood cells, such as peripheral blood mononuclear cell (PBMC), lymphocytes, monocytes or macrophages. The one or more cells can be any immune cells such as a lymphocyte, a T cell, an alpha-beta T cell, a gamma-delta T cell, a Jurkat cell, CD4+ T cell, CD8+ T cell, a T effector cell, a lymphocyte, a B cell, an NK cell, an NKT cell, a myeloid cell, a monocyte, a macrophage, or a neutrophil, preferably an alpha-beta T cell or a gamma-delta T cell, more preferably an alpha beta T cell.

Methods of making engineered cells can comprise the use of a vector to introduce a polynucleotide described herein, such as, for example, a nucleic acid sequence that encodes a MIDIS protein of the disclosure, and/or an exogenous antigen-recognition receptor. A vector can be any genetic element, e.g., a plasmid, chromosome, virus, transposon, behaving either as an autonomous unit of polynucleotide replication within a cell. (i.e. capable of replication under its own control) or being rendered capable of replication by insertion into a cell chromosome, having attached to it another polynucleotide segment, so as to bring about the replication and/or expression of the attached segment. Suitable vectors include, but are not limited to, plasmids, transposons, bacteriophages and cosmids. Vectors can contain polynucleotide sequences which are necessary to effect ligation or insertion of the vector into a desired host cell and to affect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors can be capable of directly expressing nucleic acid sequence products encoded therein without ligation or integration of the vector into host cell DNA sequences. A vector can comprise a selectable marker gene. In some embodiments, the vector is an “episomal expression vector” or “episome,” which is able to replicate in a host cell, and persists as an extrachromosomal segment of DNA within the host cell in the presence of appropriate selective pressure.

A polynucleotide vector useful for the methods and compositions described herein can be a good manufacturing practices (GMP) compatible vector. For example, a GMP vector can be purer than a non-GMP vector. In some cases, purity can be measured by bioburden. For example, bioburden can be the presence or absence of aerobes, anaerobes, sporeformers, fungi, or combinations thereof in a vector composition. In some cases, a pure vector can be endotoxin low or endotoxin free. Purity can also be measured by double-stranded primer-walking sequencing. Plasmid identity can be a source of determining purity of a vector. A GMP vector of the invention can be from 10% to 99% more pure than a non-GMP vector. A GMP vector can be from 10%, 15%, 20%, 25%, 30%, 35%, 40%, 45%, 50%, 55%, 60%, 65%, 70%, 75%, 80%, 85%, 90%, 95%, 96%, 97%, 98%, or 99% more pure than a non-GMP vector as measured by the presence of bioburden, endotoxin, sequencing, or combinations thereof.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems.

A CRISPR system can be utilized to facilitate insertion of a polynucleotide sequence encoding a membrane protein or a component thereof into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA.

A transposon based system can be utilized for insertion of a polynucleic acid encoding a protein of the disclosure or a component thereof into a genome.

In some cases, cells are genetically engineered to comprise a protein of the disclosure in vivo. In some cases, cells are genetically engineered to comprise a protein of the disclosure in vitro or ex vivo.

Methods to introduce gene editing components into a cell include, but are not limited to, electroporation, sonoporation, use of a gene gun, lipofection, calcium phosphate transfection, use of dendrimers, microinjection, and use of viral vectors. Viral vector delivery systems can include DNA and RNA viruses, which have either episomal or integrated genomes after delivery to the cell. Examples of viral vectors include, but are not limited to, retroviral vectors, lentiviral vectors, adenovirus vectors, poxvirus vectors; herpesvirus vectors and adeno-associated virus (AAV) vectors, helper-dependent adenovirus vectors, hybrid adenovirus vectors, Epstein-Bar virus vectors, herpes simplex virus vectors, hemagglutinating virus of Japan (HVJ) vectors, and Moloney murine leukemia virus vectors.

### VIII. Pharmaceutical Compositions for Use in Methods of Treatment

The disclosure provides pharmaceutical compositions, compositions for use in methods of treatment, and methods of treatment that utilize MIDIS proteins disclosed herein. In some embodiments, disclosed herein are compositions comprising a MIDIS protein disclosed herein, a polynucleotide encoding the same, or an engineered cell expressing the same, for administration in a subject.

In some embodiments, engineered cells that express a MIDIS protein of the disclosure are formulated into a pharmaceutical composition that can be administered to a subject in need thereof. In some embodiments, pharmaceutical compositions include a vector for introduction of a MIDIS protein of the disclosure into cells in vitro, ex vivo, or in vivo. In some embodiments, viral vectors containing MIDIS gene are administered to a subject with cancer.

In an embodiment, a chimeric bidirectional signaling transmembrane protein as disclosed herein, a polynucleotide encoding it, a vector comprising said polynucleotide, a cell comprising, preferably expressing said chimeric protein or a population of cells comprising said cell are for use for treating a disease or a condition wherein the at least two, optionally inducible, intracellular signals contribute to an improvement of a biological parameter and/or function of a cell expressing the chimeric protein and/or an improvement of a biological parameter and/or function induced by such a cell, said biological parameter contributing to the treatment of the disease or condition.

In an embodiment, a method of treatment of a disease or condition is provided comprising administering a chimeric bidirectional signaling transmembrane protein as disclosed herein, a polynucleotide encoding it, a vector comprising said polynucleotide, a cell comprising, preferably expressing said chimeric protein or a population of cells comprising said cell wherein the at least two, optionally inducible, intracellular signals contribute to an improvement of a biological parameter and/or function of a cell expressing the chimeric protein and/or an improvement of a biological parameter and/or function induced by such a cell, and said biological parameter contributes to the treatment of the disease or condition.

In an embodiment, the use of a chimeric bidirectional signaling transmembrane protein as disclosed herein, a polynucleotide encoding it, a vector comprising said polynucleotide, a cell comprising, preferably expressing said chimeric protein or a population of cells comprising said cell is provided for the manufacture of a medicament for treating a disease or a condition wherein the at least two, optionally inducible, intracellular signals contribute to an improvement of a biological parameter and/or function of a cell expressing the chimeric protein and/or an improvement of a biological parameter and/or function induced by such a cell and said biological parameter contributes to the treatment of the disease or condition.

In an embodiment, a chimeric bidirectional signaling transmembrane protein as disclosed herein, a polynucleotide encoding it, a vector comprising said polynucleotide, a cell comprising, preferably expressing said chimeric protein or a population of cells comprising said cell are for use wherein
the biological parameter is selected from proliferation, survival, cytotoxicity, antitumor activity, persistence and/or tumor cell killing,
the cell is an immune cell and/or
the disease is cancer.

In an embodiment, a chimeric bidirectional signaling transmembrane protein as disclosed herein, a polynucleotide encoding it, a vector comprising said polynucleotide, a cell comprising, preferably expressing said chimeric protein or a population of cells comprising said cell are for use wherein
the biological parameter is selected from proliferation, survival, cytotoxicity, antitumor activity, persistence and/or tumor cell killing,
the cell is an alpha-beta T cell expressing said chimeric protein and its interaction partner and/or
the disease is cancer.

In an embodiment, a chimeric bidirectional signaling transmembrane protein as disclosed herein, a polynucleotide encoding it, a vector comprising said polynucleotide, a cell comprising, preferably expressing said chimeric protein or a population of cells comprising said cell are for use wherein
the biological parameter is selected from proliferation, survival, cytotoxicity, antitumor activity, persistence and/or tumor cell killing,
the cell is an alpha-beta T cell expressing a gamma-delta TCR, expressing said chimeric protein and its interaction partner and
the disease is cancer.

Each of the features has already been earlier defined herein.

A subject in need thereof can have a disorder, for example, a cancer. In some cases, the cancer is a metastatic cancer. In other cases, the cancer is a relapsed or refractory cancer. In some cases, a cancer is a solid tumor or a hematologic malignancy. In some instances, the cancer is a solid tumor. In other instances, the cancer is a hematologic malignancy. In some embodiments, the disorder is an infectious disease.

Cells administered to a subject in need thereof can be autologous to the subject. Cells administered to a subject in need thereof can be allogeneic to the subject, for example, fully HLA-matched, HLA matched at 1, 2, 3, 4, 5, 6, 7, or 8 HLA alleles, or at least 1, at least 2, at least 3, at least 4, at least 5, at least 6, at least 7, or at least 8 HLA alleles. Cells administered to a subject in need thereof can be haploidentical to the subject. Cells administered to a subject in need thereof can be from a donor that is related to the subject. Cells administered to a subject in need thereof can be from a donor that is not related to the subject.

In certain embodiments, cryopreserved cells (e.g., engineered cells) are thawed and washed as described herein and allowed to rest for one hour at room temperature prior to activation using the methods of the present disclosure. In an aspect, a composition comprising an engineered cell can include a dosage form of a cell, e.g., a unit dosage form.

In some instances, pharmaceutical compositions are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In certain embodiments, compositions can also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some embodiments, compositions can also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein can be administered by any suitable administration route, including but not limited to, parenteral (e.g., intravenous, intratumoral, subcutaneous, intramuscular, intracerebral, intracerebroventricular, intra-articular, intraperitoneal, or intracranial), intranasal, buccal, sublingual, oral, or rectal administration routes. In some instances, the pharmaceutical composition is formulated for parenteral (e.g., intravenous, intratumoral, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous dispersions, liquids, gels, syrups, elixirs, slurries, suspensions and the like, for administration to a subject to be treated. In some embodiments, the pharmaceutical compositions are formulated into solutions (for example, for IV administration). In some cases, the pharmaceutical composition is formulated as an infusion. In some cases, the pharmaceutical composition is formulated as an injection.

Parenteral administration can be, for example, by bolus injection or by gradual perfusion over time. Administration can also be by surgical deposition of a bolus or pellet of cells, or positioning of a medical device.

In some embodiments, pharmaceutical compositions described herein are formulated into solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

A therapeutically effective amount of a composition of the disclosure can be administered to a subject. A “therapeutically effective amount” can refer to an amount effective, at dosages and for periods of time necessary, to achieve a desired therapeutic result. The therapeutically effective amount can vary according to factors such as the disease state, age, sex, and weight of the individual, and the ability of the inventive nucleic acid sequences to elicit a desired response in the individual.

### IX. Additional Polynucleotides

A polynucleotide may encode a heterodimeric receptor as described earlier herein. Such a polynucleotide may be multicistronic. The present inventors have surprisingly found that the expression of a heterodimeric receptor by a cell described earlier herein may be significantly enhanced when the cell comprises a polynucleotide comprising at least one nucleic acid encoding a polypeptide other than the monomers of the receptor inserted between the nucleic acids encoding the receptor monomers, and when the nucleic acids are operably linked to the same promoter sequence.

Improved expression of a heterodimeric receptor may be assessed by any standard technique available to the skilled person discussed earlier herein, such as flow cytometry, FACS, and the like. Further non-limiting examples are provided in the experimental section.

Improved expression of a heterodimeric receptor may result in an improvement of a biological parameter and/or function of a cell expressing the receptor, which can be or can comprise, for example, cellular proliferation, cellular survival, magnitude of immune effector function, duration of immune effector function, cytotoxic effects on a target cell (e.g., a cancer cell), production of inflammatory mediators, an anti-cancer immune response, cellular differentiation, cellular dedifferentiation, and the like, as discussed earlier herein. The improvement may be at least 5%, at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 fold compared to a cell which expresses the receptor without at least one nucleic acid encoding a polypeptide other than the monomers of the receptor having been inserted in the polynucleotide encoding said receptor between the nucleic acids encoding the receptor monomers. Assays to measure these biological parameters and/or functions are described earlier herein and further non-limiting examples are provided in the experimental section.

Accordingly, the invention further provides a polynucleotide encoding each of the monomers of a heterodimeric receptor, wherein said polynucleotide comprises at least one nucleic acid encoding a polypeptide other than said monomers inserted between the nucleic acids encoding each of said monomers, and wherein said nucleic acids are operably linked to the same promoter sequence. It is understood that in cases wherein more than one nucleic acids are inserted between the nucleic acids encoding each of the receptor monomers, the inserted nucleic acids may encode for the same or different polypeptides.

In some embodiments, at least two nucleic acids, at least three nucleic acids, or at least four nucleic acids, encoding a polypeptide other than the monomers of the heterodimeric receptor are inserted between the nucleic acids encoding each of the monomers.

In some embodiments, the polynucleotide encoding the heterodimeric receptor is tricistronic. This refers to a single nucleic acid encoding a polypeptide other than the monomers of the heterodimeric receptor being inserted between the nucleic acids encoding each of the monomers.

In some embodiments, the polynucleotide encoding the heterodimeric receptor is tetracistronic. This refers to two nucleic acids encoding a polypeptide other than the monomers of the heterodimeric receptor being inserted between the nucleic acids encoding each of the monomers. The polypeptides may be the same or different.

Suitable promoter sequences that may be operably linked to the nucleic acids are discussed earlier herein. In some embodiments, the promoter sequence is selected from the group of EF1α, MSCV, EF1 alpha-HTLV-1 hybrid promoter, Moloney murine leukemia virus (MoMuLV or MMLV), Gibbon Ape Leukemia virus (GALV), murine mammary tumor virus (MuMTV or MMTV), Rous sarcoma virus (RSV), MHC class II, clotting Factor IX, insulin promoter, PDX1 promoter, CD11, CD4, CD2, gp47 promoter, PGK, Beta-globin, UbC, and MND, preferably from MSCV, MMLV, EF1α, and MND. In some embodiments, the promoter sequence is a derivative sequence (i.e. variant sequence) of a promoter sequence described herein. In some embodiments, a promoter sequence comprises a sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity with any one of SEQ ID NOs: 202-205.

In some embodiments, a polynucleotide encoding a heterodimeric receptor described herein comprises a nucleotide sequence inserted between each of the nucleic acids which facilitates their co-expression. As used herein “facilitates their co-expression” is to be understood that these sequences function so as to ensure that the distinct polypeptides (i.e., the heterodimeric receptor monomers and the at least one additional polypeptide as described herein) are translated from the singe mRNA transcribed from the polynucleotide. Such a nucleotide sequence may, for example, be selected from, but is not limited to, an internal ribosome entry site (IRES) sequence or a sequence encoding a 2A-self cleaving peptide. In some embodiments, the nucleotide sequence inserted between each of the nucleic acids is a sequence encoding a 2A self-cleaving peptide or is an IRES sequence. An IRES sequence functions by allowing the assembly of a new translation initiation complex after the ribosome dissociates from the mRNA following the synthesis of the first polypeptide. Suitable IRES sequences will be known to the skilled person and examples are further available in public databases such as IRESite: The database of experimentally verified IRES structures, described in Mokrejs et al., Nucleic Acids Res. 2006; 34(Database issue): D125-D130, which is incorporated herein by reference in its entirety. In preferred embodiments, the nucleotide sequence inserted between each of the nucleic acids is a sequence encoding a 2A self-cleaving peptide. 2A self-cleaving peptides (abbreviated herein as “2A peptides”) may be advantageous for expression of multicistronic polynucleotides described herein due to their small size and self-cleavage ability, which allows for facilitation of polypeptide co-expression. 2A peptides are typically composed of 16-22 amino acids and originate from viral RNA. 2A peptide-mediated polypeptide cleavage is typically triggered by ribosomal skipping of the peptide bond between the proline (P) and glycine (G) in the C-terminal of a 2A peptide, resulting in the polypeptide located upstream of the 2A peptide to have extra amino acids on its C-terminal end while the peptide located downstream the 2A peptide has an extra proline on its N-terminal end. Examples of nucleic acid sequences encoding 2A peptides may be found in Xu Y., et al (2019), and Pincha M., et al, (2011) (supra). Non-limiting examples of suitable 2A peptides are F2A (2A peptide derived from the foot-and-mouth disease virus), E2A (2A peptide derived from the equine rhinitis virus), P2A (2A peptide derived from the porcine teschovirus-1), or T2A (2A peptide derived from the Thosea asigna virus). In some embodiments, the 2A self-cleaving peptide is a F2A peptide. In some embodiments, the 2A self-cleaving peptide is an E2A peptide. In some embodiments, the 2A self-cleaving peptide is a P2A peptide. In some embodiments, the 2A self-cleaving peptide is a T2A peptide. The skilled person understands that a polynucleotide described herein may also comprise nucleotide sequences encoding different 2A self-cleaving peptides. As a non-limiting example, in a tricistronic construct, a P2A peptide-encoding sequence may be inserted between the nucleic acid encoding the first and the second polypeptide, and a T2A peptide-encoding sequence may be inserted between the nucleic acid encoding the second and third polypeptide. Accordingly, polynucleotides comprising nucleotide sequences encoding multiple different 2A self-cleaving peptides are also provided. A preferred polynucleotide comprises a P2A peptide-encoding sequence and a T2A peptide-encoding sequence. A further preferred polynucleotide comprises a nucleotide sequence encoding a 2A self-cleaving peptide having at least at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity with SEQ ID NO: 207 or 209, or comprises a nucleotide sequence encoding a 2A self-cleaving peptide represented by an amino acid sequence having an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% with SEQ ID NO: 206 or 208.

In some embodiments, the heterodimeric receptor encoded by a polynucleotide described herein is an exogenous antigen-recognition receptor. In some embodiments, the exogenous antigen-recognition receptor is selected from a B-cell receptor heavy and light chain heterodimer, a Toll-like receptor 1 and 2 heterodimer, a phagocytic receptor Mac-1, a CD94 NKG2C or NKG2E receptor, a T-cell receptor (TCR), an alpha beta (αβ) T-cell receptor, a gamma delta (γδ) T-cell receptor, and functional fragments thereof. In some embodiments, the heterodimeric receptor is a B-cell receptor heavy and light chain heterodimer or a functional fragment thereof. In some embodiments, the heterodimeric receptor is a Toll-like receptor 1 and 2 heterodimer, or a functional fragment thereof. In some embodiments, the heterodimeric receptor is a phagocytic receptor Mac-1 or a functional fragment thereof. In some embodiments, the heterodimeric receptor is a CD94 NKG2C or NKG2E receptor, or a functional fragment thereof.

In preferred embodiments, the exogenous antigen-recognition receptor is a T-cell receptor or a functional fragment thereof. Among T-cell receptors, αβT-cell receptors, γδT-cell receptors or functional fragments thereof are preferred, with γδT-cell receptors or functional fragments thereof being most preferred. Suitable T-cell receptors, αβT-cell receptors, and γδT-cell receptors are described earlier herein.

The nucleic acids encoding each of the monomers of the heterodimeric receptor may preferably be in a specific order with respect to their positions in the polynucleotide. The term “specific order” as used herein refers to the order in which the encoded polypeptides are translated from the multicistronic mRNA by the ribosome. In other words, it refers to the positions of the nucleic acids encoding the polypeptides in the multicistronic polynucleotide. Thus, a nucleic acid that is located at the “first position” is understood to be the first nucleic acid to be expressed, and a nucleic acid that is located at the “last position” is understood to be the last nucleic acid to be expressed. As a non-limiting example, in a polynucleotide encoding an αβT-cell receptor as described herein, the order of the nucleic acids may be: α chain-encoding nucleic acid (first position), followed by at least one nucleic acid encoding a polypeptide other than an α or β chain of an αβT-cell receptor, followed by a β chain-encoding nucleic acid (last position). Alternatively, a preferred order of the nucleic acids may be: β chain-encoding nucleic acid (first position), followed by at least one nucleic acid encoding a polypeptide other than an α or β chain of an αβT-cell receptor, followed by an α chain-encoding nucleic acid (last position). As another non-limiting example, in a polynucleotide encoding a γδT-cell receptor as described herein, a preferred order of the nucleic acids may be: γ chain-encoding nucleic acid (first position), followed by at least one nucleic acid encoding a polypeptide other than a γ or δ chain of a γδT-cell receptor, followed by a δ chain-encoding nucleic acid (last position). Alternatively, the order of nucleic acids may be: δ chain-encoding nucleic acid (first position), followed by at least one nucleic acid encoding a polypeptide other than a γ or δ chain of an γδT-cell receptor, followed by a γ chain-encoding nucleic acid (last position). Preferably, the polynucleotides described above are tricistronic or tetracistronic.

In some embodiments a polynucleotide encoding a γδT-cell receptor is tricistronic, and comprises a nucleic acid encoding a γ-chain at the first position and a nucleic acid encoding a δ-chain at the third position.

In some embodiments a polynucleotide encoding a γδT-cell receptor is tricistronic, and comprises a nucleic acid encoding a δ-chain at the first position and a nucleic acid encoding a γ-chain at the third position.

In some embodiments a polynucleotide encoding a γδT-cell receptor is tetracistronic, and comprises a nucleic acid encoding a γ-chain at the first position and a nucleic acid encoding a δ-chain at the fourth position.

In some embodiments a polynucleotide encoding a γδT-cell receptor is tetracistronic, and comprises a nucleic acid encoding a δ-chain at the first position and a nucleic acid encoding a γ-chain at the fourth position.

Accordingly, in some embodiments, a polynucleotide described herein comprises A, B, C, or D, wherein:
(A) is a nucleic acid represented by (i)-(ii)-(iii), wherein:
(i) is a nucleic acid encoding an α chain of an αβT-cell receptor or a functional fragment thereof,
(ii) is at least one nucleic acid encoding a polypeptide other than an α or β chain of an αβT-cell receptor or a functional fragment thereof, and;
(iii) is a nucleic acid encoding a β chain of an αβT-cell receptor or a functional fragment thereof, wherein (ii) is inserted between (i) and (iii)
(B) is a nucleic acid represented by (iv)-(v)-(vi), wherein:
(iv) is a nucleic acid encoding a β chain of an αβT-cell receptor or a functional fragment thereof,
(v) is at least one nucleic acid encoding a polypeptide other than an α or β chain of an αβT-cell receptor or a functional fragment thereof, and;
(vi) is a nucleic acid encoding an α chain of an αβT-cell receptor or a functional fragment thereof, wherein (v) is inserted between (iv) and (vi)
(C) is a nucleic acid represented by (vii)-(viii)-(ix), wherein:
(vii) is a nucleic acid encoding a γ chain of a γδT-cell receptor or a functional fragment thereof,
(viii) is at least one nucleic acid encoding a polypeptide other than a γ or δ chain of a γδT-cell receptor or a functional fragment thereof, and;
(ix) is a nucleic acid encoding a δ chain of a γδT-cell receptor or a functional fragment thereof, wherein (viii) is inserted between (vi) and (ix)
(D) is a nucleic acid represented by (x)-(xi)-(xii), wherein:
(x) is a nucleic acid encoding a δ chain of a γδT-cell receptor or a functional fragment thereof,
(xi) is at least one nucleic acid encoding a polypeptide other than a γ or δ chain of a γδT-cell receptor or a functional fragment thereof, and;
(xii) is a nucleic acid encoding a γ chain of a γδT-cell receptor or a functional fragment thereof, wherein (xi) is inserted between (x) and (xii)
(i), (iv), (vii), and (x) represent nucleic acids located at the first position of the respective polynucleotides comprising them. (iii), (vi), (ix), and (xii) represent nucleic acids located at the last position of the respective polynucleotides comprising them.

Among A, B, C, or D, the polynucleotides characterized by B, C and D are preferred, with C and D being more preferred, and with C being most preferred. Preferably, the polynucleotides further comprise a 2A peptide-encoding nucleotide sequence inserted between each of the nucleic acids.

Non-limiting examples of such polynucleotides have been constructed and experimentally verified herein (e.g. Examples 14-17).

Preferably, A, B, C, and/or D are such that:
(i) and (vi) are nucleic acids comprising a nucleotide sequence encoding a polypeptide having at least 60%, 70%, 80%, 90%, 95%, or 100% identity or similarity with an amino acid sequence selected from SEQ ID NOs: 199, 210, 214, 216, 218, and 220, preferably selected from SEQ ID NOs: 210, 216, and 220, and/or;
(iii) and (iv) are nucleic acids comprising a nucleotide sequence encoding a polypeptide having at least 60%, 70%, 80%, 90%, 95%, or 100% identity or similarity with an amino acid sequence selected from SEQ ID NOs: 198, 211, 215, 217, 219, and 221, preferably selected from SEQ ID NOs: 211, 217, and 221, and/or;
(vii) and (xii) are nucleic acids comprising a nucleotide sequence encoding a polypeptide having at least 60%, 70%, 80%, 90%, 95%, or 100% identity or similarity with an amino acid sequence selected from SEQ ID NOs: 85, 86, 87, 89, 91, 93, 94, 95, 96, 101, 104, 106, 108, 110, 112, 113, 115, 117, 119, 121, 123, 125, 127, 129, 130, and 132, preferably selected from SEQ ID NOs: 85, 86, 87, 94, 95, 96, 101, 113, 115, 117, 119, 127, and 130, and/or;
(ix) and (x) are nucleic acids comprising a nucleotide sequence encoding a polypeptide having at least 60%, 70%, 80%, 90%, 95%, or 100% identity or similarity with an amino acid sequence selected from SEQ ID NOs: 82, 83, 84, 88, 90, 92, 97, 98, 99, 100, 102, 103, 105, 107, 109, 111, 114, 116, 118, 120, 122, 124, 126, 128, 131, and 133, preferably selected from SEQ ID NOs: 82, 83, 84, 97, 98, 99, 100, 102, 114,116, 118, 126, and 131.

A preferred nucleic acid encoding a polypeptide other than the monomers of a heterodimeric receptor, that may be inserted between the nucleic acids encoding each of said monomers in a polynucleotide encoding said receptor, is a chimeric bidirectional signaling transmembrane protein (MIDIS) described earlier herein.

Accordingly, in some embodiments, a preferred polynucleotide encoding a heterodimeric receptor described herein comprises a nucleic acid inserted between the nucleic acids encoding each of the receptor monomers which encodes a chimeric bidirectional signaling transmembrane protein able to transduce at least two intracellular signals, said protein comprising:
an extracellular ligand domain, able to interact with the extracellular domain of its interaction partner
a transmembrane domain, and
a heterologous intracellular signaling domain transducing a first signal after binding of the extracellular ligand domain to its interaction partner,
   wherein the second intracellular signal is transduced via the intracellular domain of the interaction partner. Preferred MIDIS proteins that may be comprised in the polynucleotide are described earlier herein.

In some embodiments the MIDIS protein is such that,
the extracellular ligand domain comprises an amino acid sequence from 41BBL, OX40L, CD86, or RANK, or CD70, as described earlier herein, and
the heterologous intracellular signaling domain comprises an amino acid sequence from OX40, 41BB, NKp80, or IL18RAP, or IL2RB, as described earlier herein.

In some embodiments, the polynucleotide is a tricistronic polynucleotide encoding a γδTCR and a chimeric bidirectional signaling transmembrane protein. The first nucleic acid encodes the gamma chain of the TCR, followed by the nucleic acid encoding the chimeric bidirectional signaling transmembrane protein, subsequently followed by the nucleic acid encoding the delta chain of the TCR.

In some embodiments, the polynucleotide is a tricistronic polynucleotide encoding a γδTCR and a chimeric bidirectional signaling transmembrane protein. The first nucleic acid encodes the delta chain of the TCR, followed by the nucleic acid encoding the chimeric bidirectional signaling transmembrane protein, subsequently followed by the nucleic acid encoding the gamma chain of the TCR. Preferred gamma and delta chains of TCRs and preferred chimeric bidirectional signaling transmembrane proteins are described earlier herein.

In some embodiments, the polynucleotide is a tetracistronic polynucleotide encoding a γδTCR and a chimeric bidirectional signaling transmembrane protein. The first nucleic acid encodes the gamma chain of the TCR, followed by two nucleic acids, wherein at least one nucleic acid encodes a chimeric bidirectional signaling transmembrane protein, subsequently followed by the nucleic acid encoding the delta chain of the TCR.

In some embodiments, the polynucleotide is a tetracistronic polynucleotide encoding a γδTCR and a chimeric bidirectional signaling transmembrane protein. The first nucleic acid encodes the delta chain of the TCR, followed by two nucleic acids, wherein at least one nucleic acid encodes a chimeric bidirectional signaling transmembrane protein, subsequently followed by the nucleic acid encoding the gamma chain of the TCR.

Preferred gamma and delta chains of TCRs and preferred chimeric bidirectional signaling transmembrane proteins are described earlier herein.

In some embodiments, the polynucleotide is a tricistronic polynucleotide encoding an αβTCR and a chimeric bidirectional signaling transmembrane protein. The first nucleic acid encodes the alpha chain of the TCR, followed by the nucleic acid encoding the chimeric bidirectional signaling transmembrane protein, subsequently followed by the nucleic acid encoding the beta chain of the TCR.

In some embodiments, the polynucleotide is a tricistronic polynucleotide encoding an αβTCR and a chimeric bidirectional signaling transmembrane protein. The first nucleic acid encodes the beta chain of the TCR, followed by the nucleic acid encoding the chimeric bidirectional signaling transmembrane protein, subsequently followed by the nucleic acid encoding the alpha chain of the TCR. Preferred alpha and beta chains of TCRs and preferred chimeric bidirectional signaling transmembrane proteins are described earlier herein.

In some embodiments, the polynucleotide is a tetracistronic polynucleotide encoding an αβTCR and a chimeric bidirectional signaling transmembrane protein. The first nucleic acid encodes the alpha chain of the TCR, followed by two nucleic acids, wherein at least one nucleic acid encodes a chimeric bidirectional signaling transmembrane protein, subsequently followed by the nucleic acid encoding the beta chain of the TCR.

In some embodiments, the polynucleotide is a tetracistronic polynucleotide encoding an αβTCR and a chimeric bidirectional signaling transmembrane protein. The first nucleic acid encodes the beta chain of the TCR, followed two nucleic acids, wherein at least one nucleic acid encodes the chimeric bidirectional signaling transmembrane protein, subsequently followed by the nucleic acid encoding the alpha chain of the TCR.

Preferred alpha and beta chains of TCRs and preferred chimeric bidirectional signaling transmembrane proteins are described earlier herein.

In some embodiments, the polynucleotide further comprises a nucleic acid encoding the interaction partner of a chimeric bidirectional signaling protein as described earlier herein.

In some embodiments, the polynucleotide is comprised in a vector described earlier herein. A preferred vector is a viral vector, with a lentiviral vector being most preferred.

A cell described earlier herein comprising a polynucleotide and/or a vector preferably expresses said polynucleotide and/or vector. A preferred cell is a T cell. Among T cells, αβT cells are preferred.

The polynucleotides encoding a heterodimeric receptor as described herein may be used in the pharmaceutical compositions, methods, treatments, and therapeutic uses described earlier herein.

In particular, the disclosure provides polynucleotides, vectors, polypeptides encoded by the polynucleotides and/or vectors, cells (or populations of cells) comprising the polynucleotides and/or vectors, preferably additionally expressing the polypeptides, and pharmaceutical compositions comprising said polynucleotides, vectors, polypeptides, and/or cells, described earlier herein, for use as a medicament, or a method of treatment comprising administering said polynucleotides, vectors, polypeptides, cells and/or compositions to a subject in need thereof. A subject in need thereof is defined earlier herein. Such a subject can have a disorder, for example, a cancer. In some cases, the cancer is a metastatic cancer. In other cases, the cancer is a relapsed or refractory cancer. In some cases, a cancer is a solid tumor or a hematologic malignancy. In some instances, the cancer is a solid tumor. In other instances, the cancer is a hematologic malignancy. In some embodiments, the disorder is an infectious disease.

Suitable additional components of pharmaceutical compositions, and modes of administration are discussed earlier herein.

In an embodiment, a polynucleotide, a vector, and/or a heterodimeric receptor encoded by the polynucleotide and/or vector, described earlier herein, are for use in the treatment and/or prevention of a disease, wherein
a biological parameter and/or function of a cell expressing the heterodimeric receptor selected from proliferation, survival, cytotoxicity, antitumor activity, persistence and/or tumor cell killing is improved,
the cell is an immune cell and/or
the disease is cancer.

In an embodiment, a polynucleotide, a vector, and/or a heterodimeric receptor encoded by the polynucleotide and/or vector, described earlier herein, are for use in the treatment and/or prevention of a disease, wherein
a biological parameter and/or function of a cell expressing the heterodimeric receptor selected from proliferation, survival, cytotoxicity, antitumor activity, persistence and/or tumor cell killing is improved,
the cell is an αβT-cell and/or
the disease is cancer.

In an embodiment, a polynucleotide, a vector, and/or a heterodimeric receptor encoded by the polynucleotide and/or vector, described earlier herein, are for use in the treatment and/or prevention of a disease, wherein
a biological parameter and/or function of a cell expressing the heterodimeric receptor selected from proliferation, survival, cytotoxicity, antitumor activity, persistence and/or tumor cell killing is improved,
the cell is an αβT-cell
the heterodimeric receptor is a γδT-cell receptor, and/or
the disease is cancer.

Methods of engineering cells so as to achieve expression of a heterodimeric receptor by introduction of a polynucleotide and/or vector described herein are discussed earlier herein.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a product (chimeric protein, polynucleotide, vector, cell, population) as described herein may comprise additional component(s) than the ones specifically identified, said additional component(s) not altering the unique characteristic of the invention. In addition, the verb “to consist” may be replaced by “to consist essentially of” meaning that a product as described herein may comprise component(s) than the ones specifically identified, said additional component(s) not altering the unique characteristic of the invention.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value (of 10) more or less 0.1% of the value.

As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated.

One skilled in the art will recognize many methods and materials similar or equivalent to those described herein, which could be used in the practice of the present invention. Indeed, the present invention is in no way limited to the methods and materials described.

### X. Examples

The following examples are included for illustrative purposes only and are not intended to limit the scope of the invention.

In cases wherein commercially available reagents and equipment were utilized, protocols used were according to manufacturer instructions unless otherwise indicated.

### Example 1: Design and Expression of MIDIS Proteins in Alpha-Beta T Cells Engineered to Express a Defined Gamma-Delta TCR (TEG)

MIDIS proteins of the disclosure were designed incorporating the extracellular ligand domains and heterologous intracellular signaling domains as shown in Tables 8-14, 15-17. Lentiviral vectors were generated to allow for genomic integration of DNA sequences that encode MIDIS proteins of the disclosure. The lentiviral vectors contained a tricistronic sequence encoding a gamma TCR chain and a delta TCR chain of the disclosure, with the MIDIS protein-encoding sequence between the gamma chain and the delta chain, and 2A self-cleaving peptide sequences connecting the three protein sequences (Xu Y., et al (2019), Cancer Immunology, Immunotherapy, 68: 1979-1993 and Pincha M., et al, (2011), Gene Therapy, 18: 750-764).

Cryopreserved αβTCR T cells were thawed, seeded in 48 well plates at density of 1.6×10\hat{ }6 cells per well in TexMACS (Miltenyi, DE) medium with Penicillin/Streptomycin (0.5%), human serum (2.4%), IL-7 (1700 IU/ml), and IL-15 (150 IU/ml) (TEG medium). The T cells were activated with CD3/CD28 TransAct for 24 h, then transduced with the selected lentiviral vector at a prior set multiplicity of infection (MOI; 0.3-25) by diluting the lentivirus in TexMACS medium. Lentiviral MOI was determined by FACS based titration on J76 Jurkat cells similar to Pirona et al. 2020, Biology Methods and Protocols 5:1. In brief, J76 Jurkat cells were seeded at 0.5E6/ml viable cells in a 96 well plate. Lentiviral supernatant was concentrated and serial diluted in cold medium with 10% FBS, starting at 100× dilution. After serial dilution lentivirus containing supernatant was diluted 1:1 with J76 Jurkat cells and mixture was incubate for 3 days at 37 degrees Celsius. Titer was determined by analyzing percentage of CD3⁺γδTCR⁺ viable cells. On the second day medium was refreshed and T cells transferred to 12 well plates. On day 5 medium was refreshed once more, and T cells were transferred to a T25 cell culture flask with a final volume of 10 ml. On day 7, T cells were transferred to a T75 cell culture flask with a final volume of 25 ml. After 48 h, medium was refreshed one more time, to a final volume of 25 ml. T cells were harvested 12 days after thawing them.

Cells present after the 12 day protocol were characterized by flow cytometry. Cells were washed with FACS buffer (PBS with 2% fetal bovine serum), and stained with selected fluorescently-conjugated antibodies in FACS buffer for 30 min at 4 degrees Celsius. After staining, cells were washed twice with FACS buffer, and fixed by 4% paraformaldehyde for 10 min at 4 degrees Celsius. Following fixation, cells were washed once more with FACS buffer, before resuspension in 100-200 μl of FACS buffer, followed by flow cytometric analysis (BD LSR Fortessa).

Tables 8-14, 15-17 provide details of the fold-expansion and proportion of gamma-delta TCR+ cells after the 12 day protocol. Each table is from a separate production run and/or uses cells from a different donor. EC: extracellular. Hetero IC: heterologous intracellular. These results suggest that certain MIDIS proteins of the disclosure can enhance expansion of engineered cells.

TABLE 8

| | | Hetero | | | | total | |
|---|---|---|---|---|---|---|---|
| | | IC | | | day 12 | γδTCR | |
| | EC ligand | signaling | | MOI | fold | cells | CD4:CD8 |
| TEG# | domain | domain | notes | transduced | expansion | (%) | ratio |
| O1 | none | none | Enhanced | 7 | 46.6 | 16.7% | 0.22 |
| | | | GFP, | | | | |
| | | | tricistronic | | | | |
| | | | control | | | | |
| | | | protein | | | | |
| | | | without a | | | | |
| | | | cellular | | | | |
| | | | function | | | | |
| | | | SEQ ID | | | | |
| | | | NO: 81 | | | | |
| O2 | 41BBLmincyto | none | WT 41BBL | 7 | 46.0 | 51.8% | 0.31 |
| | | | of which | | | | |
| | | | intracellular | | | | |
| | | | domain is | | | | |
| | | | swapped | | | | |
| | | | for small | | | | |
| | | | linker | | | | |
| | | | MSKSTGS; | | | | |
| | | | SEQ ID | | | | |
| | | | NO: 55 | | | | |
| O3 | 41BBLmincyto | NKp80 | 41BBL | 7 | 44.3 | 43.7% | 0.33 |
| | | | ligand | | | | |
| | | | domain | | | | |
| | | | and TM | | | | |
| | | | linked to an | | | | |
| | | | NKp80 | | | | |
| | | | intracellular | | | | |
| | | | signaling | | | | |
| | | | domain, | | | | |
| | | | lacking the | | | | |
| | | | intracellular | | | | |
| | | | sequence | | | | |
| | | | of 41BBL; | | | | |
| | | | SEQ ID | | | | |
| | | | NO: 48 | | | | |
| O4 | 41BBL | NKp80 | 41BBL WT | 7 | 46.1 | 38.8% | 0.42 |
| | | | with 41BBL | | | | |
| | | | TM without | | | | |
| | | | first 2 | | | | |
| | | | amino | | | | |
| | | | acids with | | | | |
| | | | NKp80 | | | | |
| | | | intracellular | | | | |
| | | | signaling | | | | |
| | | | domain; | | | | |
| | | | SEQ ID | | | | |
| | | | NO: 47 | | | | |
| O5 | 41BBL | OX40 | WT 41BBL | 7 | 47.4 | 71.3% | 0.35 |
| | | | with 41BBL | | | | |
| | | | TM and | | | | |
| | | | OX40 | | | | |
| | | | intracellular | | | | |
| | | | signaling | | | | |
| | | | domain; | | | | |
| | | | SEQ ID | | | | |
| | | | NO: 45 | | | | |
| O6 | 41BBL | OX40rev | 41BBL WT | 7 | 60.8 | 29.6% | 0.36 |
| | | | including | | | | |
| | | | TM and IC | | | | |
| | | | domain | | | | |
| | | | without first | | | | |
| | | | 2 amino | | | | |
| | | | acids with | | | | |
| | | | inverted | | | | |
| | | | OX40 | | | | |
| | | | intracellular | | | | |
| | | | signaling | | | | |
| | | | domain; | | | | |
| | | | SEQ ID | | | | |
| | | | NO: 57 | | | | |

TABLE 9

| | | Hetero | | | | total | |
|---|---|---|---|---|---|---|---|
| | | IC | | | day 12 | γδTCR | |
| | EC ligand | signaling | | MOI | fold | cells | CD4:CD8 |
| TEG# | domain | domain | notes | transduced | expansion | (%) | ratio |
| O7 | none | none | Enhanced | 7 | 60.0 | 30.5% | 0.74 |
| | | | GFP, | | | | |
| | | | tricistronic | | | | |
| | | | control | | | | |
| | | | protein | | | | |
| | | | without a | | | | |
| | | | cellular | | | | |
| | | | function; | | | | |
| | | | SEQ ID | | | | |
| | | | NO: 81 | | | | |
| O8 | 41BBLmincyto | none | WT 41BBL | 7 | 46.6 | 77.4% | 1.25 |
| | | | of which | | | | |
| | | | intracellular | | | | |
| | | | domain is | | | | |
| | | | swapped | | | | |
| | | | for small | | | | |
| | | | linker | | | | |
| | | | MSKSTGS | | | | |
| | | | SEQ ID | | | | |
| | | | NO: 55 | | | | |
| O9 | 41BBLmincyto | NKp80 | 41BBL | 7 | 43.6 | 70.6% | 1.2 |
| | | | ligand | | | | |
| | | | domain | | | | |
| | | | and TM | | | | |
| | | | linked to an | | | | |
| | | | NKp80 | | | | |
| | | | intracellular | | | | |
| | | | signaling | | | | |
| | | | domain, | | | | |
| | | | lacking the | | | | |
| | | | intracellular | | | | |
| | | | sequence | | | | |
| | | | of 41BBL; | | | | |
| | | | SEQ ID | | | | |
| | | | NO: 48 | | | | |
| O10 | 41BBL | NKp80 | 41BBL WT | 7 | 44.9 | 56.1% | 3.99 |
| | | | with 41BBL | | | | |
| | | | TM without | | | | |
| | | | first 2 | | | | |
| | | | amino | | | | |
| | | | acids with | | | | |
| | | | NKp80 | | | | |
| | | | intracellular | | | | |
| | | | signaling | | | | |
| | | | domain; | | | | |
| | | | SEQ ID | | | | |
| | | | NO: 47 | | | | |
| O11 | 41BBL | OX40 | WT 41BBL | 7 | 43.6 | 88.2% | 0.70 |
| | | | with 41BBL | | | | |
| | | | TM and | | | | |
| | | | OX40 | | | | |
| | | | intracellular | | | | |
| | | | signaling | | | | |
| | | | domain; | | | | |
| | | | SEQ ID | | | | |
| | | | NO: 45 | | | | |
| O12 | 41BBL | OX40rev | 41BBL WT | 7 | 50.6 | 48.9% | 0.79 |
| | | | including | | | | |
| | | | TM and IC | | | | |
| | | | domain | | | | |
| | | | without first | | | | |
| | | | 2 amino | | | | |
| | | | acids with | | | | |
| | | | inverted | | | | |
| | | | OX40 | | | | |
| | | | intracellular | | | | |
| | | | signaling | | | | |
| | | | domain; | | | | |
| | | | SEQ ID | | | | |
| | | | NO: 57 | | | | |

TABLE 10

| | | | | | | total |
|---|---|---|---|---|---|---|
| | | Hetero IC | | | day 12 | γδTCR |
| | EC ligand | signaling | | MOI | fold | cells |
| TEG# | domain | domain | notes | transduced | expansion | (%) |
| O13 | 41BBL | OX40tm- | 41BBL ligand | 5 | 23.6 | 42.7% |
| | | cyto | binding comain | | | |
| | | | core in a type I | | | |
| | | | orientation with | | | |
| | | | IgK leader | | | |
| | | | sequence and | | | |
| | | | inverted | | | |
| | | | extracellular N- | | | |
| | | | terminal part | | | |
| | | | with a OX40 TM | | | |
| | | | and intracellular | | | |
| | | | signaling | | | |
| | | | domain; SEQ ID | | | |
| | | | NO: 58 | | | |
| O14 | none | none | γδTCR control | 5 | 22.2 | 37.6% |
| O15 | 41BBL | OX40 | WT 41BBL with | | 33.9 | 64.4% |
| | | | 41BBL TM and | | | |
| | | | OX40 | | | |
| | | | intracellular | | | |
| | | | signaling | | | |
| | | | domain; SEQ ID | | | |
| | | | NO: 45 | | | |
| O16 | none | none | untransduced | n/a | 24.8 | 0.1% |
| O17 | none | none | γδTCR lentigen | 25 | 25.6 | 64.0% |
| | | | control | | | |

TABLE 11

| | | | | | | total |
|---|---|---|---|---|---|---|
| | | Hetero IC | | | day 12 | γδTCR |
| | EC ligand | signaling | | MOI | fold | cells |
| TEG# | domain | domain | notes | transduced | expansion | (%) |
| N13 | CD86 WT | none | CD86 control; | 3 | 40.8 | 52.0% |
| | | | SEQ ID NO: | | | |
| | | | 68 | | | |
| N14 | CD86 | OX40 | CD86 WT | 3 | 38.6 | 48.3% |
| | | | including TM | | | |
| | | | and IC domain | | | |
| | | | linked to OX40 | | | |
| | | | intracellular | | | |
| | | | signaling | | | |
| | | | domain; SEQ | | | |
| | | | ID NO: 52 | | | |
| N15 | CD86delP276 | none | CD86 with a | 3 | 41.3 | 61.8% |
| | | | deletion of the | | | |
| | | | IC part up to | | | |
| | | | the point which | | | |
| | | | is shown to be | | | |
| | | | involved in | | | |
| | | | correct cellular | | | |
| | | | localization of | | | |
| | | | CD86 (deletion | | | |
| | | | of amino acid | | | |
| | | | 277-329 of | | | |
| | | | CD86); SEQ | | | |
| | | | ID NO: 69 | | | |
| N16 | CD86delP276 | OX40 | CD86 ligand | 3 | 37.5 | 69.9% |
| | | | binding | | | |
| | | | domain and | | | |
| | | | TM domain, | | | |
| | | | with a deletion | | | |
| | | | of amino acids | | | |
| | | | 277-329 of | | | |
| | | | CD86, and an | | | |
| | | | OX40 | | | |
| | | | intracellular | | | |
| | | | signaling | | | |
| | | | domain; SEQ | | | |
| | | | ID NO: 53 | | | |
| N17 | 41BBL | OX40 | WT 41BBL | 3 | 41.9 | 73.6% |
| | | | with 41BBL | | | |
| | | | TM and OX40 | | | |
| | | | intracellular | | | |
| | | | signaling | | | |
| | | | domain; SEQ | | | |
| | | | ID NO: 45 | | | |
| N18 | none | none | γδTCR | 25 | 49.2 | 46.9% |
| | | | bicistronic | | | |
| | | | control; only | | | |
| | | | TCR | | | |
| N19 | none | none | untransduced | n/a | 47.3 | 0.4% |

TABLE 12

| | | Hetero | | | | total | |
|---|---|---|---|---|---|---|---|
| | | IC | | | day 12 | γδTCR | |
| | EC ligand | signaling | | MOI | fold | cells | CD4:CD8 |
| TEG# | domain | domain | notes | transduced | expansion | (%) | ratio |
| N5 | OX40L WT | none | OX40L | 5 | 41.4 | 57.6% | 1.75 |
| | | | control; | | | | |
| | | | SEQ ID | | | | |
| | | | NO: 66 | | | | |
| N6 | OX40Lmincyto | 41BB | OX40L WT | 5 | 35.9 | 42.7% | 1.46 |
| | | | EC and TM | | | | |
| | | | linked to | | | | |
| | | | 41BB IC | | | | |
| | | | signaling | | | | |
| | | | domain by | | | | |
| | | | a GGS | | | | |
| | | | linker; SEQ | | | | |
| | | | ID NO: 67 | | | | |
| N7 | OX40Lmincyto | 41BBrev | OX40L WT | 5 | 50.4 | 39.1% | 2.17 |
| | | | EC and TM | | | | |
| | | | linked to | | | | |
| | | | the | | | | |
| | | | inverted IC | | | | |
| | | | singaling | | | | |
| | | | domain by | | | | |
| | | | a short | | | | |
| | | | linker; SEQ | | | | |
| | | | ID NO: 65 | | | | |
| N8 | OX40L | 41BBrev | OX40L | 5 | 38.4 | 51.5% | 1.47 |
| | | | ligand | | | | |
| | | | binding | | | | |
| | | | and TM | | | | |
| | | | domain | | | | |
| | | | with 41BB | | | | |
| | | | intracellular | | | | |
| | | | signaling | | | | |
| | | | domain | | | | |
| | | | that is | | | | |
| | | | inverted; | | | | |
| | | | SEQ ID | | | | |
| | | | NO: 51 | | | | |
| N9 | OX40L | 41BB | OX40L WT | 5 | 39.3 | 46.6% | 1.3 |
| | | | including | | | | |
| | | | TM and IC | | | | |
| | | | domain | | | | |
| | | | with 41BB | | | | |
| | | | intracellular | | | | |
| | | | signaling | | | | |
| | | | domain | | | | |
| | | | separated | | | | |
| | | | by GGSA | | | | |
| | | | linker; SEQ | | | | |
| | | | ID NO: 50 | | | | |
| N10 | 41BBL | OX40 | WT 41BBL | 5 | 42.3 | 75.1% | |
| | | | with 41BBL | | | | |
| | | | TM and | | | | |
| | | | OX40 | | | | |
| | | | intracellular | | | | |
| | | | signaling | | | | |
| | | | domain; | | | | |
| | | | SEQ ID | | | | |
| | | | NO: 45 | | | | |
| N11 | CD8 | non | tricistronic | | 53.2 | 64.0% | |
| | | | control | | | | |
| N12 | none | none | lengigen | 25 | 42.3 | 52.4% | 1.74 |
| | | | γδTCR | | | | |
| | | | control | | | | |

TABLE 13

| | | | | | | total |
|---|---|---|---|---|---|---|
| | | Hetero IC | | | day 12 | γδTCR |
| | EC ligand | signaling | | MOI | fold | cells |
| TEG# | domain | domain | notes | transduced | expansion | (%) |
| O18 | RANK WT | none | RANK | 5 | 48.8 | 6.2% |
| | | | control; SEQ | | | |
| | | | ID NO: 74 | | | |
| O19 | RANKmincyto | none | RANK WT | 5 | 47.6 | 55.0% |
| | | | without the IC | | | |
| | | | domain; SEQ | | | |
| | | | ID NO: 75 | | | |
| O20 | RANKmincyto | IL18RAP | EC domain of | 5 | 46.4 | 23.6% |
| | | | RANK linked | | | |
| | | | to the TM and | | | |
| | | | IC domain of | | | |
| | | | IL18RAP; | | | |
| | | | SEQ ID NO: | | | |
| | | | 78 | | | |
| O21 | RANK | 41BB | EC domain of | 5 | 46.2 | 34.5% |
| | | | RANK linked | | | |
| | | | to TM and IC | | | |
| | | | domain of | | | |
| | | | 41BB; SEQ | | | |
| | | | ID NO: 77 | | | |
| O22 | RANK | OX40 | EC domain of | 5 | 41.0 | 38.6% |
| | | | RANK linked | | | |
| | | | to TM and IC | | | |
| | | | of OX40; | | | |
| | | | SEQ ID NO: | | | |
| | | | 76 | | | |
| O23 | none | none | eGFP control | 5 | 43.8 | 24.9% |
| | | | SEQ ID NO: | | | |
| | | | 81 | | | |
| O24 | none | none | γδTCR control | 5 | 42.7 | 88.6% |

TABLE 14

| | | Hetero IC | | | total | |
|---|---|---|---|---|---|---|
| | EC ligand | signaling | | day 12 fold | γδTCR | CD4:CD8 |
| TEG# | domain | domain | notes | expansion | cells (%) | ratio |
| N1 | none | none | γδTCR control | 32.6 | 74.0% | 2.62 |
| N2 | 41BBL | OX40 | WT 41BBL | 40.3 | 81.3% | 1.9 |
| | | | with 41BBL | | | |
| | | | TM and OX40 | | | |
| | | | intracellular | | | |
| | | | signaling | | | |
| | | | domain; SEQ | | | |
| | | | ID NO: 45 | | | |
| N3 | 41BBL | none | WT 41BBL | 55.1 | 74.9% | 1.46 |
| | | | control; SEQ | | | |
| | | | ID NO: 54 | | | |
| N4 | none | none | untransduced | 42.0 | 0.4% | |
| | | | control | | | |

TABLE 15

| | | Hetero IC | | | total | |
|---|---|---|---|---|---|---|
| | EC ligand | signaling | | day 12 fold | γδTCR | CD4:CD8 |
| TEG# | domain | domain | notes | expansion | cells (%) | ratio |
| O25 | none | none | γδTCR control | 32.6 | 74.0% | 2.62 |
| O26 | 41BBL | OX40 | WT 41BBL | 40.3 | 81.3% | 1.9 |
| | | | with 41BBL | | | |
| | | | TM and OX40 | | | |
| | | | intracellular | | | |
| | | | signaling | | | |
| | | | domain; SEQ | | | |
| | | | ID NO: 45 | | | |
| O27 | 41BBL | none | WT 41BBL | 55.1 | 74.9% | 1.46 |
| | | | control; SEQ | | | |
| | | | ID NO: 54 | | | |
| O28 | none | none | untransduced | 42.0 | 0.4% | |
| | | | control | | | |

TABLE 16

| | | Hetero IC | | | total | |
|---|---|---|---|---|---|---|
| | EC ligand | signaling | | day 12 fold | γδTCR | CD4:CD8 |
| TEG# | domain | domain | notes | expansion | cells (%) | ratio |
| O29 | CD86 | IL18RAP- | CD86 WT | 24.4 | 40.9% | 54.3% |
| | | ICD | including TM | | | |
| | | | and IC | | | |
| | | | domain linked | | | |
| | | | to IL18RAP | | | |
| | | | intracellular | | | |
| | | | signaling | | | |
| | | | domain; SEQ | | | |
| | | | ID NO: 72 | | | |
| O30 | CD86mincyto | IL18RAP- | CD86 WT EC | 26.1 | 40.6% | 54.8% |
| | | ICD | and TM linked | | | |
| | | | to IC domain | | | |
| | | | of IL18RAP; | | | |
| | | | SEQ ID NO: | | | |
| | | | 71 | | | |
| O31 | CD8-Q8 (Q8) | none | γδTCR control | 35.7 | 36.8% | 58.1% |
| O32 | none | none | untransduced | 31.94 | 28.2% | 67.0% |
| | | | control | | | |

TABLE 17

| | | Hetero IC | | | total | |
|---|---|---|---|---|---|---|
| | EC ligand | signaling | | day 12 fold | γδTCR | CD4:CD8 |
| TEG# | domain | domain | notes | expansion | cells (%) | ratio |
| P11 | CD70 | none | WT CD70; | 42.75 | 50.9% | 0.13 |
| | | | SEQ ID NO: | | | |
| | | | 180 | | | |
| P12 | CD70mincyto | none | CD70 EC and | 41.63 | 54.0% | 0.12 |
| | | | TM; SEQ ID | | | |
| | | | NO: 181 | | | |
| P13 | CD70mincyto | OX40 | CD70 EC and | 43.88 | 51.1% | 0.12 |
| | | | TM linked to | | | |
| | | | IC signaling | | | |
| | | | domain of | | | |
| | | | OX40 | | | |
| | | | separated by | | | |
| | | | a TSGS | | | |
| | | | linker; SEQ ID | | | |
| | | | NO: 182 | | | |
| P14 | CD70 | 41BBL- | CD70 EC | 45.00 | 42.8% | 0.13 |
| | | OX40 | linked to | | | |
| | | | 41BBL TM | | | |
| | | | and IC with IC | | | |
| | | | signaling | | | |
| | | | domain of | | | |
| | | | OX40 | | | |
| | | | separated by | | | |
| | | | a TSGS | | | |
| | | | linker; SEQ ID | | | |
| | | | NO: 183 | | | |
| P15 | none | none | γδTCR control | 41.63 | 55.5% | 0.13 |
| P16 | none | none | untransduced | 45.00 | 0.6% | 0.13 |
| | | | control | | | |

### Example 2: Co-Culture Assays of Target Cells and Effector T Cells Expressing MIDIS Proteins and a Defined Gamma-Delta TCR

Target tumor cells, including HT-29, MZ1851 RC, RPMI-8226, MM1-S, OPM-2 cells, were seeded at defined concentrations in 96 well plates. Adherent target cells were seeded a day before effector cells were added, and suspension target cells were seeded on the same day that effector cells were added.

The variable domains of the defined gamma-delta TCR expressed was either the one represented by SEQ ID NO: 90 and 91 (clone 5) or 111 and 112 (E57).

Fresh (day 12 of production) or cryopreserved TEGs were used as effector cells. The number of effector cells added to all co-cultures were corrected to match the TEGs with the lowest transduction efficiency in the experiment. Untransduced cells which went through the same production process, but without adding lentiviral vectors, were added so that all co-cultures within one experiment contained the same number of TEGs and the same number of total T cells.

Co-cultures were setup at a effector to target ratio (E:T) of 1:1 or 0.3:1, and with or without pamidronate treatment (10 μm) on the day of effector cell addition. Untransduced, effector only, target only, or full lysis controls were included.

After 3 or 7 days, supernatant was collected from the co-cultures, nonadherent cells were resuspended gently, and cell suspension was transferred to round bottom 96 well plate. When cytotoxicity was determined by luminescence, 100 ul of assay medium together with D-luciferin was added to the residual co-culture plate containing any remaining target cells, and incubated for 12 minutes before measuring luminescence by Glomax (Promega). For Xcelligence based cytotoxicity, data was captured in real-time along the co-culture incubation, and after transferring the cell suspension plates were discarded.

Cell suspension was medium exchanged to 60 ul per well, 50 ul of this was transferred to a new plate of pre-seeded targets for a subsequent round of cytotoxicity evaluation, and the process was repeated as indicated for individual figures.

Residual cell suspension mix was characterized by FACS to measure proliferation, cell fitness, and other parameters. Proliferation was measured by determining the number of TEGs and total T cells, or via cell trace violet dilution when TEGs were stained at start of the serial cytotoxicity assay. Fitness of the TEGs was determined based on staining for various markers (e.g., 4-1BB, OX40, PD-1, TIM-3, LAG-3). Cells were washed with FACS buffer (PBS with 2% fetal bovine serum), and stained with selected fluorescently-conjugated antibodies in FACS buffer for 30 min at 4 degrees Celsius (e.g., appropriate combinations of antibodies specific for CD4, CD8α, CD3, αβTCR, γδTCR, 4-1BB, OX40, PD-1, TIM-3, LAG-3, 4-1BBL, OX40L, CD86, Fab2, CD107a and CD69, see table 14). After staining, cells were washed twice with FACS buffer, and fixed by 4% paraformaldehyde for 10 min at 4 degrees Celsius. Following fixation, cells were washed once more with FACS buffer, before resuspension in 100-200 μl of FACS buffer, followed by flow cytometric analysis.

In certain experiments, more co-culture plates were initially used, and at the end of a round of cytotoxicity evaluation, separate plates were used for cytotoxicity evaluation, effector T cell characterization, and passage of effector cells to a subsequent round of the experiment.

### Example 3: MIDIS Proteins Enhance Engineered Immune Cell Responses to Target Cells

This example evaluates the effects of MIDIS proteins of the disclosure on the abilities of engineered immune cells to respond to target cells.

Alpha-beta T cells were transduced with a defined gamma-delta TCR (SEQ ID NO: 90 and 91) to generate TEGs as outlined in example 1, with or without additional MIDIS proteins or other control proteins. The defined gamma-delta TCR used in this example was a Vγ9Vδ2 TCR of the disclosure. The MIDIS proteins and control proteins were:
(i) 41BBL—wild type 41BBL, in this example SEQ ID NO: 54 was used;
(ii) 41BBLmincyto—a protein containing an extracellular 41BBL ligand domain, a transmembrane domain from 41BBL, and lacking an intracellular signaling domain, in this example SEQ ID NO: 55 was used;
(iii) 41BBLmincyto_NKp80—a MIDIS containing an extracellular 41BBL ligand domain, a transmembrane domain from 41BBL, and an NKp80 heterologous intracellular signaling domain, and lacking the intracellular sequence of 41BBL, in this example SEQ ID NO: 48 was used;
(iv) 41BBL_NKp80—a MIDIS containing an extracellular 41BBL ligand domain, a transmembrane domain from 41BBL, and an NKp80 heterologous intracellular signaling domain, in this example SEQ ID NO: 47 was used;
(v) 41BBL_OX40—a MIDIS containing an extracellular 41BBL ligand domain, a transmembrane domain from 41BBL, and an OX40 heterologous intracellular signaling domain, in this example SEQ ID NO: 45 was used;
(vi) 41BBL13W_OX40rev—a MIDIS containing an extracellular 41BBL ligand domain, a transmembrane domain from 41BBL, a partial intracellular domain from 41BBL with deletion of the first 12 amino acids containing a putative casein kinase I motif, and an OX40 heterologous intracellular signaling domain that is inverted (i.e. expressed as a retro-protein), in this example SEQ ID NO: 46 was used;
and (vii) an eGFP control, in this example SEQ ID NO: 81.

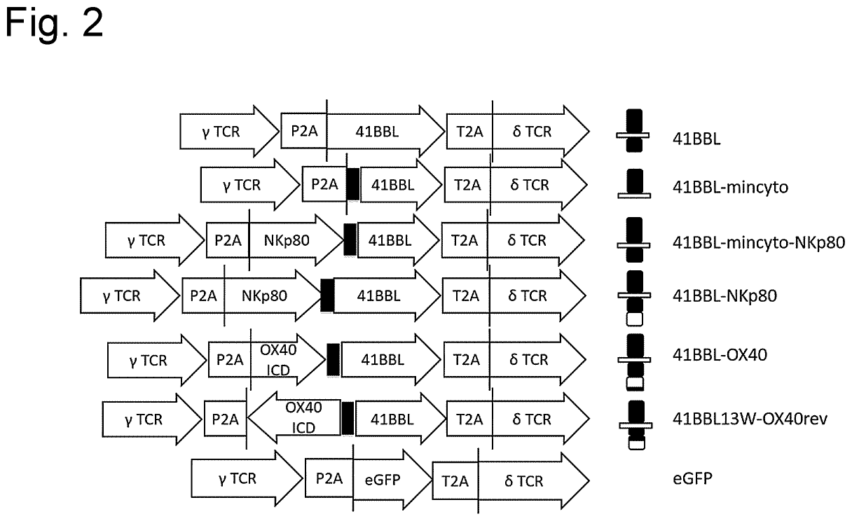

FIG. 2 shows a schematic of the constructs used to introduce the gamma-delta TCR and the other proteins. P2A and T2A represent self-cleaving peptides (SEQ ID NO: 206, SEQ ID NO: 208). The black bars N-terminal of 41BBL or eGFP represents a linker sequence. The diagrams to the right illustrate the domains present extracellularly (top) and intracellularly (bottom), with the horizontal bar representing the transmembrane domain.

The TEGs were co-incubated with target tumor cells recognized by the defined gamma-delta TCR as described in example 2, with transfer to fresh target cells every three days and measurement of residual target cell viability by luciferase assay. The experiment included conditions with or without pamidronate treatment (10 μm), and was continued until less than 90% cytotoxicity was observed.

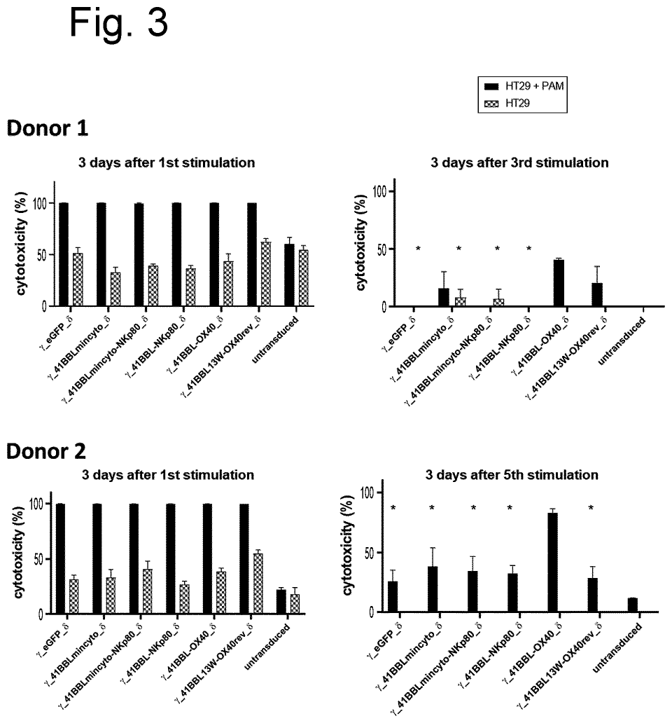

The cytotoxicity of TEGs from two donors co-incubated with HT-29 ectopically expressing luciferase-tdTomato at effector to target (E:T) ratio of 1:1 is shown in FIG. 3. Serial stimulation of TEGs was continued for 3 stimulations (donor 1) or 5 stimulations (donor 2). The results show that MIDIS proteins of the disclosure can enhance effector function of engineered immune cells. For example, the MIDIS protein containing an extracellular 41BBL ligand domain and an intracellular OX40 heterologous intracellular signaling domain retained strong cytotoxic ability after persistent exposure to target cells, significantly better than control cells or cells expressing the extracellular 41BBL ligand domain but lacking the OX40 intracellular domain.

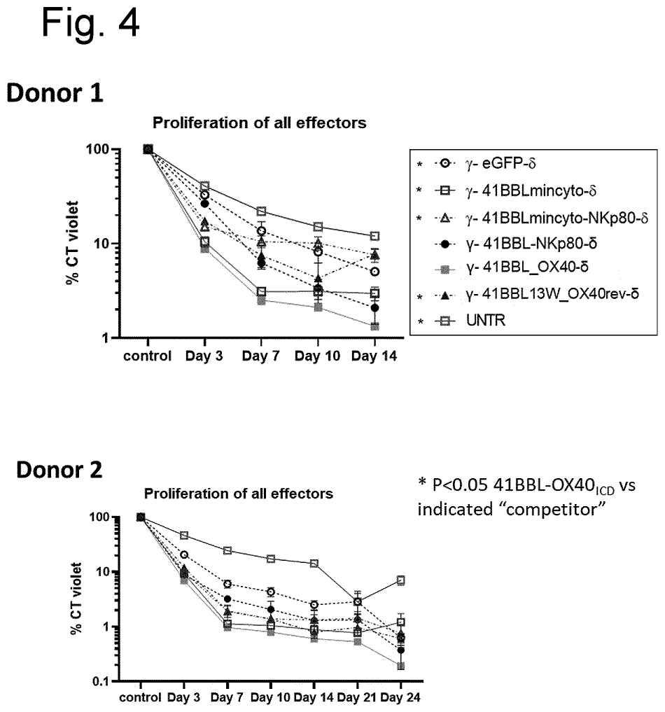

To measure proliferation, the effector cells from donor 1 and donor 2 were stained with cell trace violet (CTV), and dilution of the dye was used as a marker for proliferation. ⅙ of the total effector cells before re-challenge were evaluated by FACS at each transfer to new target HT-29 cells. As shown in FIG. 4, MIDIS proteins of the disclosure can enhance proliferation of engineered immune cells upon activation, including engineered T cell proliferation in response to recognition of target cells.

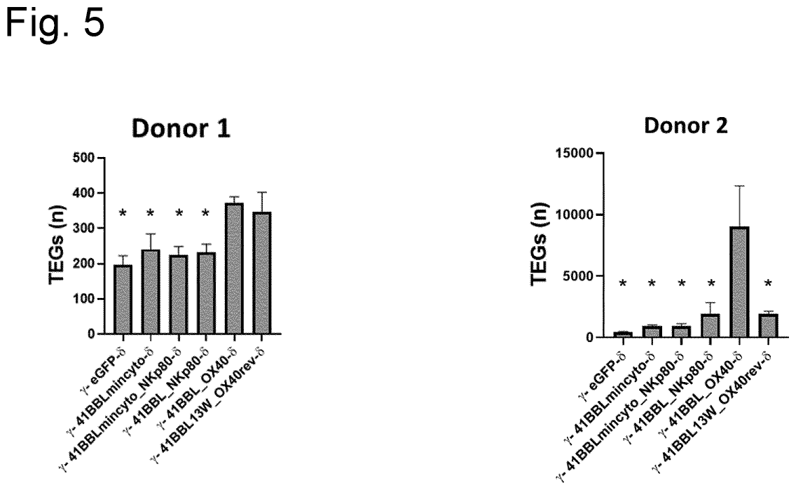

FIG. 5 shows the number of cells expressing the transduced gamma delta TCR after 3 stimulations (rounds of co-culture stimulation) with HT-29 cells (donor 1) or 5 stimulations (donor 2). These data demonstrate that MIDIS proteins of the disclosure can enhance proliferation of engineered immune cells upon activation, including engineered T cell proliferation in response to recognition of target cells. For example, particularly high numbers of TEGs are observed for cells that express the MIDIS protein containing an extracellular 41BBL ligand domain and an OX40 heterologous intracellular signaling domain.

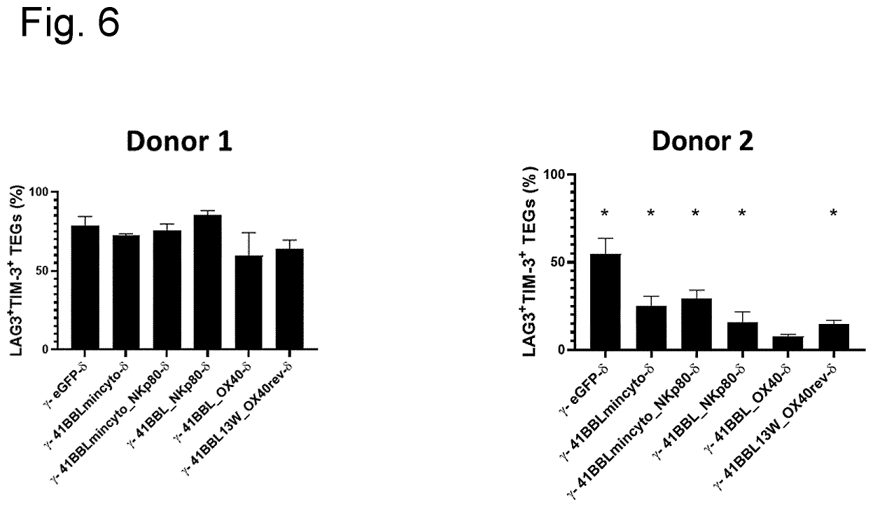

The proportion of TEGs that co-express the exhaustion markers LAG-3 and TIM-3 was measured after 3 stimulations with HT-29 cells (donor 1) or 5 stimulations (donor 2). LAG-3 and TIM-3 can be markers of T cell exhaustion, and it has been observed that many or most cells positive for or LAG-3 or TIM-3 are also PD-1 positive, especially the LAG-3⁺TIM-3⁺ cells. As shown in FIG. 6, MIDIS proteins of the disclosure can reduce exhaustion of engineered T cells after persistent exposure to target cells, as compared to cells that do not express MIDIS proteins. For example, particularly low levels of LAG-3⁺TIM-3⁺ cells were observed in TEGs expressing the MIDIS protein containing an extracellular 41BBL ligand domain and an OX40 heterologous intracellular signaling domain.

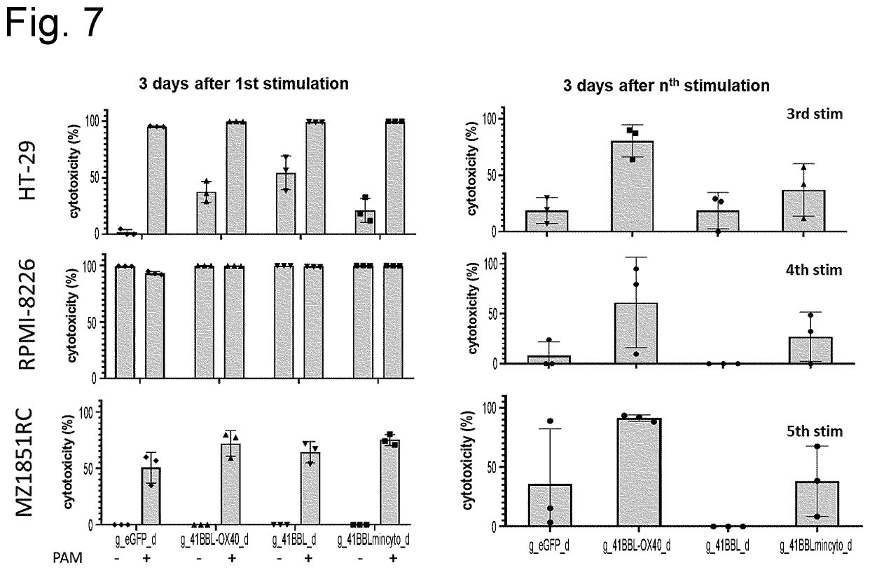

Cytotoxicity of TEGs from a third representative donor was also evaluated following co-culture with HT-29, RPMI-8226, and MZ1851 RC target cells. Serial stimulation of TEGs was continued for 3 stimulations (HT-29), 4 stimulations (RPMI-8226) or 5 stimulations (MZ1851 RC). Used MIDIS constructs are SEQ NO: 81, 45, 54, 55. The left panels of FIG. 7 show cytotoxicity data from the first co-culture stimulation, while the right panels show cytotoxicity data from the final co-culture stimulation, with PAM treatment (10 μm). The results further show that MIDIS proteins of the disclosure can enhance effector function of engineered immune cells. For example, engineered cells expressing the MIDIS protein containing an extracellular 41BBL ligand domain and an OX40 heterologous intracellular signaling domain retained strong cytotoxic ability after persistent exposure to target cells, substantially longer than control cells or cells expressing the extracellular 41BBL ligand domain but lacking the OX40 intracellular domain.

Cytotoxicity of TEGs from a fourth representative donor was also evaluated following co-culture with HT-29. Serial stimulation of TEGs was continued for 2 stimulations (HT-29). Used MIDIS constructs were SEQ NO: 46 and 54. FIG. 18 shows cytotoxicity of second stimulation with Pamidronate treatment (10 μm) (Luminescence of residual viable HT-29 cells is shown). The results further show that MIDIS proteins of the disclosure can enhance effector function of engineered immune cells. For example, engineered cells expressing the MIDIS protein containing an extracellular 41BBL ligand domain and an OX40 heterologous intracellular signaling domain retained strong cytotoxic ability after persistent exposure to target cells, substantially longer than control cells or cells expressing the extracellular 41BBL ligand domain but lacking the OX40 intracellular domain.

### Example 4: In Vitro and In Vivo Benefits of a 41BBL-OX40 MIDIS Protein for Engineered Alpha-Beta T Cells Transduced with a Defined Gamma-Delta TCR

The ability of the MIDIS comprising an extracellular 41BBL ligand domain and an OX40 heterologous intracellular signaling domain was evaluated in the context of alpha beta T cells that express a different defined gamma delta TCR compared to the previous examples.

In this example, alpha-beta T cells were transduced with a defined Vγ4Vδ5 TCR SEQ NO: 111-112 of the disclosure (γδTCR) to generate TEGs as in example 1, with or without additional the 41BBL-OX40 MIDIS protein (in this example SEQ ID NO: 45 was used) or 41BBL—wild type 41BBL, in this example SEQ ID NO: 54 was used.

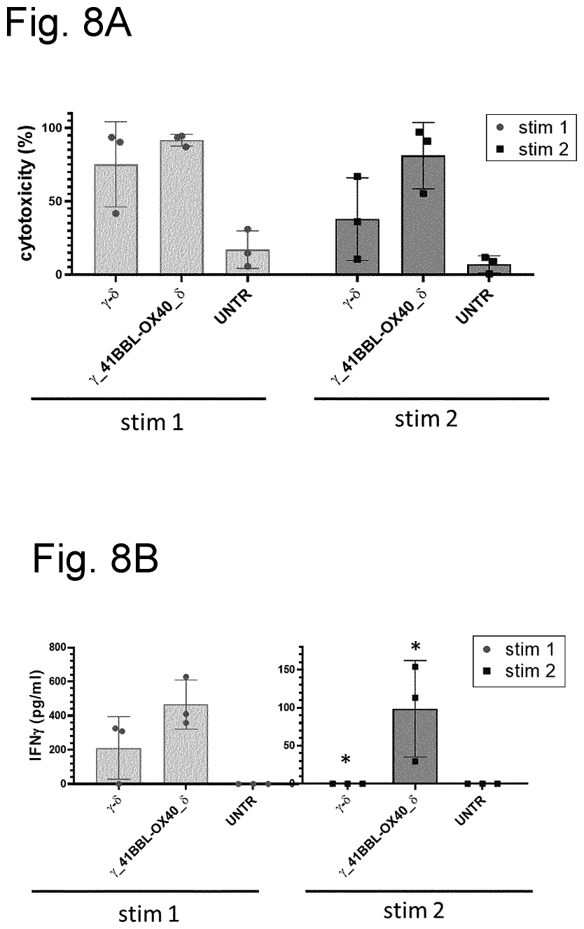

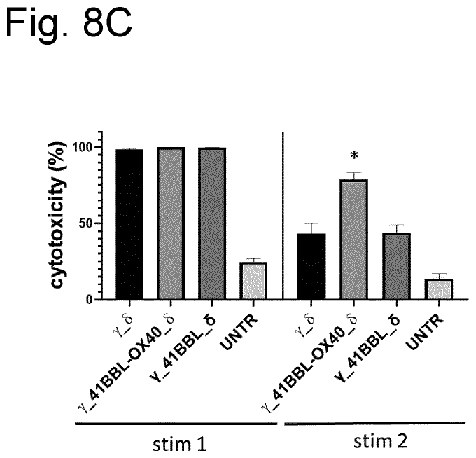

The TEGs were co-incubated with target HT-29 tumor cells ectopically expressing luciferase-tdTomato at E:T ratio 1:1 (the HT-29 cells are recognized by the defined gamma-delta TCR). TEGs were transferred to plates with fresh target cells after 3 (FIG. 8A,B) or 7 (FIG. 8C) days. Residual target cell viability was measured by luciferase assay, and IFNγ production by ELISA. As shown in FIG. 8A, the 41BBL-OX40 MIDIS protein enhanced killing of tumor cells by the engineered cells after one stimulation, and as shown in FIG. 8B, significantly more IFNγ was produced by the TEG cells that co-expressed the 41BBL-OX40 MIDIS protein. As shown in FIG. 8C, the 41BBL-OX40 MIDIS protein significantly enhanced killing of tumor cells by engineered cells compared to 41BBL or without extra protein after one stimulation.

These data further demonstrate that MIDIS proteins of the disclosure can improve the function of engineered immune cells.

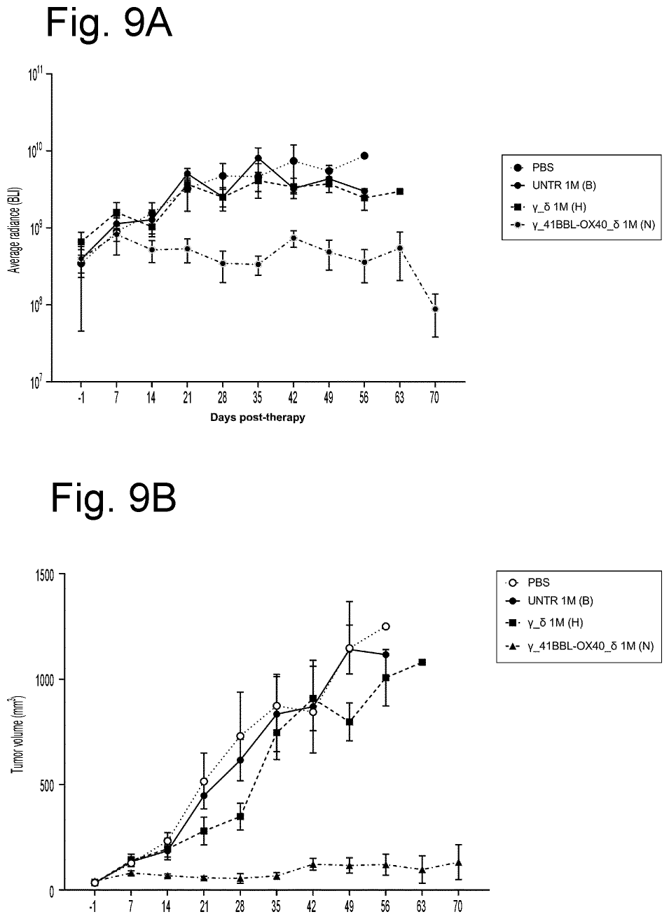

To test whether the 41BBL-OX40 MIDIS protein could enhance an anti-cancer response in vivo, a xenograft model was used. 0.5×10\hat{ }6 HT-29 luciferase-tdTomato cells were injected into the flank of NSG mice on day −14 (n=6 per group). On day 0, TEGs expressing a Vγ4Vδ5 TCR of the disclosure (γδTCR) SEQ NO: 111-112, with or without the 41BBL-OX40 MIDIS protein SEQ NO: 45 were systemically administrated at the indicated dose (e.g., 1M=1 million cells). Bioluminescence (BLI) and tumor volume were measured weekly. TEGs that expressed the MIDIS protein restricted tumor growth to a significant greater extent and significant improved survival than TEGs lacking the MIDIS protein, as shown in FIG. 9A-B and FIG. 10, which show average radiance (FIG. 9A) and tumor volume (FIG. 9B) over time, and overall survival FIG. 10.

These data demonstrate that expression of a MIDIS protein of the disclosure can improve the anti-tumor efficacy of engineered immune effector cells.

### Example 5: OX40L-41BB and CD86-OX40 MIDIS Proteins Enhance Engineered Immune Cell Killing of Target Cells

This example evaluates the effects of MIDIS proteins of the disclosure on the abilities of engineered immune cells to kill target cells.

Alpha-beta T cells were transduced with a defined gamma-delta TCR (SEQ ID NO: 90 and 91) to generate TEGs as in example 1, with or without additional MIDIS proteins or other control proteins. In this example the defined gamma-delta TCR was a Vγ9Vδ2 TCR of the disclosure.

In a first experiment, the MIDIS proteins and control proteins were:
(i) OX40L—wild type OX40L, in this example SEQ ID NO: 66 was used;
(ii) OX40Lmincyto-41BB—a protein containing an extracellular OX40L ligand domain, an OX40L transmembrane domain, lacking the intracellular signaling domain of OX40L, and containing a 41BB intracellular domain, in this example SEQ ID NO: 67 was used;
(iii) OX40Lmincyto-41BBrev—a protein containing an extracellular OX40L ligand domain, an OX40L transmembrane domain, lacking the intracellular signaling domain of OX40L, and containing a 41BB intracellular domain that is inverted (i.e. expressed as a retro-protein), in this example SEQ ID NO: 65 was used;
(iv) OX40L-41BBrev—a protein containing an extracellular OX40L ligand domain, an OX40L transmembrane domain, and a 41BB heterologous intracellular signaling domain that is inverted (i.e. expressed as a retro-protein), in this example SEQ ID NO: 51 was used;
(v) OX40L-41BB—a protein containing an extracellular OX40L ligand domain, an OX40L transmembrane domain, and a 41BB heterologous intracellular signaling domain, in this example SEQ ID NO: 50 was used; and
(vi) Q8—a control protein containing a CD8-Q8 tag; SEQ ID NO: 80.

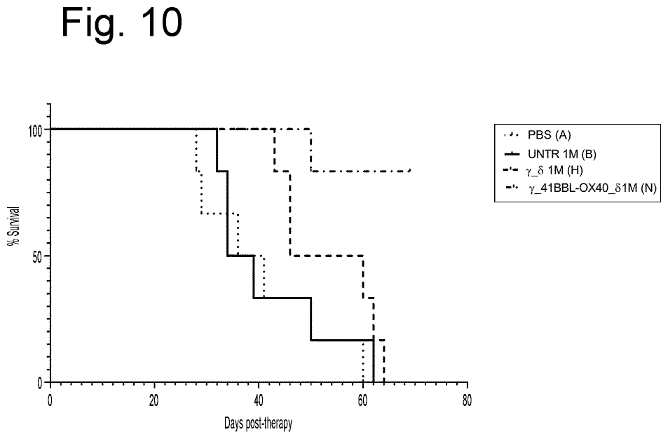

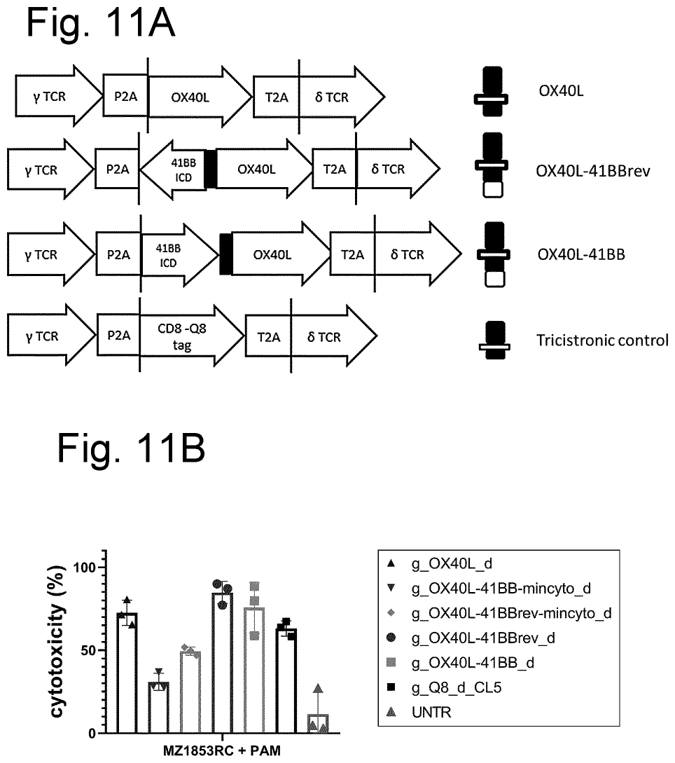

FIG. 11A shows a schematic of select constructs used to introduce the gamma-delta TCR and the other proteins. P2A and T2A represent self-cleaving peptides (SEQ ID NO: 206, SEQ ID NO: 208). The diagrams to the right illustrate the domains present extracellularly (top) and intracellularly (bottom).

The TEGs were co-incubated with target MZ1851 RC tumor cells recognized by the defined gamma-delta TCR as described in example 2, deviation is that experiment was stopped after first stimulation, and measurement of residual target cell viability by luciferase assay after the first stimulation.

The cytotoxicity of TEGs co-incubated with MZ1851 RCtarget cells ectopically expressing luciferase-tdTomato at effector to target (E:T) ratio of 1:1 is shown in FIG. 11B. The results show that MIDIS proteins of the disclosure can enhance effector function of engineered immune cells. For example, the TEGs expressing the MIDIS protein containing an extracellular OX40L ligand domain, and a 41BB heterologous intracellular signaling domain exhibited higher cytotoxicity than the TEG that expressed wild type OX40L.

In a second experiment also utilizing a Vγ9Vδ2 TCR of the disclosure, the MIDIS proteins and control proteins were:
(i) γδTCR—a Vγ9Vδ2 TCR of the disclosure, without any co-expressed protein;
(ii) CD86—wild type CD86, in this example SEQ ID NO: 68 was used;
(iii) CD86-OX40—a protein containing an extracellular CD86 ligand domain, a transmembrane domain from CD86, and an OX40 heterologous intracellular signaling domain, in this example SEQ ID NO: 52 was used;
(iv) CD86delP276—CD86 with a deletion of the cytoplasmic part up to the point which is shown to be involved in correct cellular localization of CD86 (deletion of amino acid 277-329 of CD86), in this example SEQ ID NO: 69 was used;
(v) CD86delP276-OX40—a protein containing an extracellular CD86 ligand domain, a transmembrane domain from CD86, with a deletion of amino acids 277-329 of CD86, and containing an OX40 heterologous intracellular signaling domain, in this example SEQ ID NO: 53 was used; and
(vi) lentigen γδTCR,—a Vγ9Vδ2 TCR of the disclosure from an alternate lentivirus preparation.

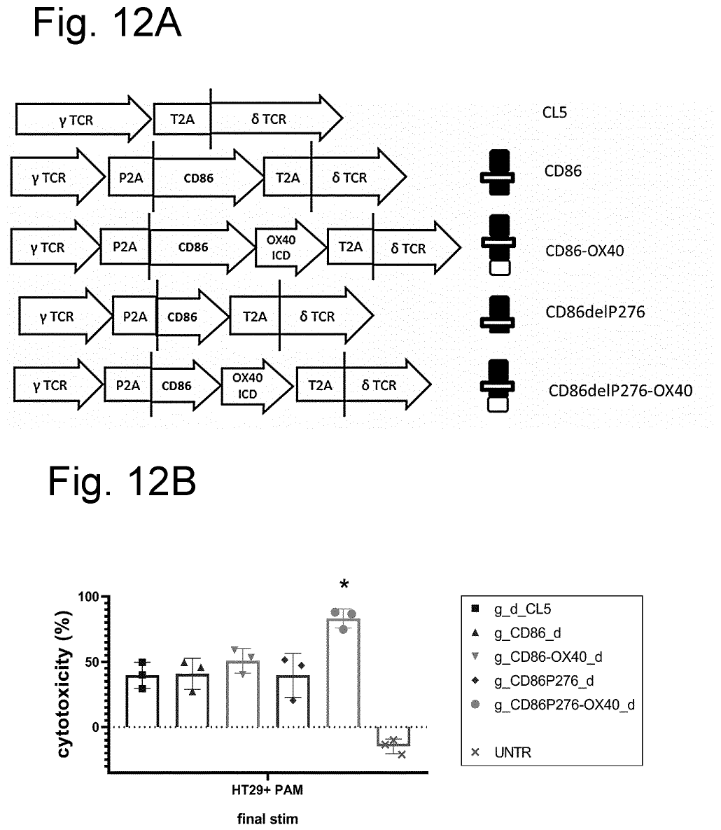

FIG. 12A shows a schematic of select constructs used to introduce the gamma-delta TCR and the other proteins. P2A and T2A represent self-cleaving peptides (SEQ ID NO: 206, SEQ ID NO: 208). The diagrams to the right illustrate the domains present extracellularly (top) and intracellularly (bottom). The horizontal bars represent he membrane/transmembrane domain.

The TEGs were co-incubated with target HT-29 tumor cells recognized by the defined gamma-delta TCR as described in example 2, with transfer to fresh target cells and addition of pamidronate (10 μm) every seven days, and measurement of residual target cell viability by luciferase assay after the second stimulation.

The cytotoxic effects of TEGs co-incubated with HT-29 target cells ectopically expressing luciferase-tdTomato at effector to target (E:T) ratio of 1:1 is shown in FIG. 12B. The results show that MIDIS proteins of the disclosure can enhance effector function of engineered immune cells. For example, the TEGs expressing MIDIS proteins containing an extracellular CD86 ligand domain, a transmembrane and truncated intracellular signaling domain from CD86 (delP276), and an OX40 heterologous intracellular signaling domain, exhibited higher cytotoxicity than the TEG that expressed wild type CD86 or truncated CD86 (delP276).

### Example 6: Surface Expression of MIDIS Proteins is Dependent on the Combination of Fusion Partners

This example evaluates cell surface expression of several MIDIS proteins of the disclosure.

Alpha-beta T cells were transduced with a defined gamma-delta TCR (SEQ ID NO: 90 and SEQ ID NO: 91) to generate TEGs, with or without additional MIDIS proteins or other control proteins, as in example 1. The MIDIS proteins and control proteins were:
(i) an eGFP control SEQ ID NO: 81;
(ii) 41BBLmincyto—a protein containing an extracellular 41BBL ligand domain, a transmembrane domain from 41BBL, and lacking an intracellular signaling domain, in this example SEQ ID NO: 55 was used;
(iii) 41BBLmincyto_NKp80—a MIDIS containing an extracellular 41BBL ligand domain, a transmembrane domain from 41BBL, and an NKp80 heterologous intracellular signaling domain, and lacking the intracellular sequence of 41BBL, in this example SEQ ID NO: 48 was used;
(iv) 41BBL_NKp80—a MIDIS containing an extracellular 41BBL ligand domain, a transmembrane domain from 41BBL, and an NKp80 heterologous intracellular signaling domain, in this example SEQ ID NO: 47 was used;
(v) 41BBL_OX40—a MIDIS containing an extracellular 41BBL ligand domain, a transmembrane domain from 41BBL, and an OX40 heterologous intracellular signaling domain, in this example SEQ ID NO: 45 was used; and
(vi) 41BBL13W_OX40rev—a MIDIS containing an extracellular 41BBL ligand domain, a transmembrane domain from 41BBL, a partial intracellular domain from 41BBL with deletion of the first 12 amino acids containing a putative casein kinase I motif, and an OX40 heterologous intracellular signaling domain that is inverted (i.e. expressed as a retro-protein), in this example SEQ ID NO: 60 was used.

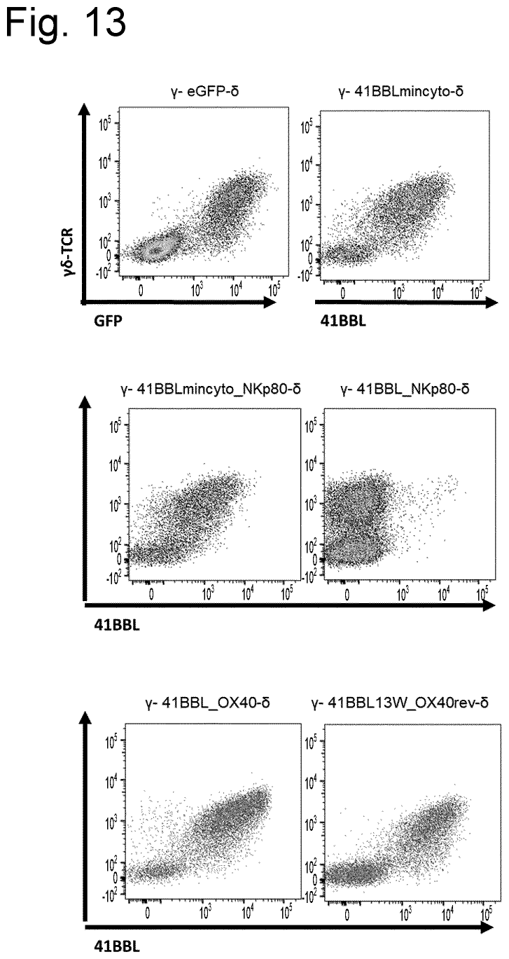

Cells present after the 12 day protocol were characterized by flow cytometry as in example 1, with staining to identify cells expressing gamma-delta TCR (Y-axis) and GFP or 41BBL (X-axis, as indicated for each panel). As shown in FIG. 13, populations expressing the gamma delta TCR were identified for each construct, confirming transduction efficiency. Detection of surface expression of 41BBL surprisingly varied by construct. For example, large differences in 41BBL detection were observed between 41BBL_NKp80 versus 41BBL_OX40, despite both containing a complete 41BBL sequence. Additionally, 41BBL13W_OX40rev exhibited surface expression, and was shown to be functional in other experiments (FIG. 18). This is surprising as many retro-proteins cannot fold properly, and/or do not retain functionality of the native orientation parent protein.

An additional experiment was conducted in which alpha-beta T cells were transduced with the defined gamma-delta TCR and MIDIS constructs with a 41BBL extracellular ligand domain and an OX40 heterologous intracellular signaling domain. In different vectors, the 41BBL was oriented at either the C terminus of the protein, with the transmembrane domain of 41BBL included (in this example SEQ ID NO: 45 was used), or at the N-terminus of the protein, with the OX40 transmembrane domain included (in this example SEQ ID NO: 58 was used).

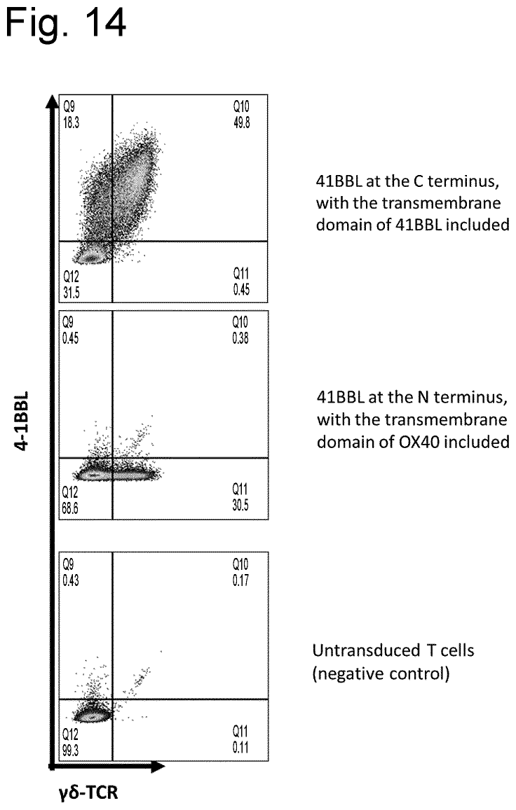

As shown in FIG. 14, 41BBL could not be detected on the cell surface when it was positioned at the N-terminus of the protein, with the OX40 transmembrane domain included.

These results show that cell surface expression of MIDIS proteins is dependent on the combination of fusion partners in the construct, and in some cases, unexpectedly high expression can be achieved. Without wishing to be bound by theory, the ability of a MIDIS protein to be expressed on the cell surface may be affected by the type I/type II orientation of the parent proteins, the choice of transmembrane domain, the combination of specific fusion partners, the native versus retro-orientation of either domain, or a combination thereof.

### Example 7: Evaluation of the Effects of MIDIS Proteins on Additional Types of Engineered Cells

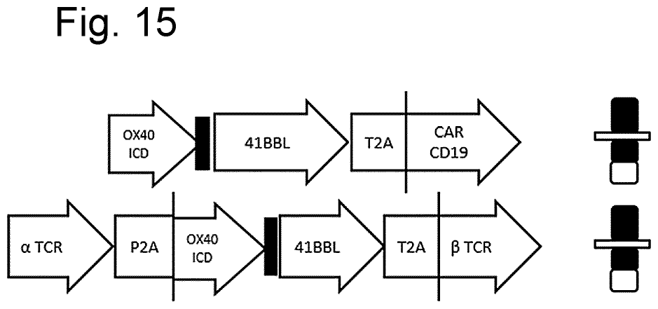

Vectors are designed to introduce MIDIS proteins of the disclosure into additional types of cells, alone or in combination with other proteins. For example, MIDIS proteins are introduced into immune cells, e.g., immune cells that express an exogenous antigen-recognition receptor, such as an alpha-beta TCR or a chimeric antigen receptor (CAR). Vectors are designed to introduce the MIDIS protein alone or in combination with the exogenous antigen-recognition receptor. Non-limiting examples of construct are provided in FIG. 15.

The MIDIS protein is introduced into the population of cells, for example, as described in example 1. The effects of the MIDIS protein is evaluated in various types of immune cells, such as TIL, NK cells, gamma delta T cells, alpha beta T cells, B cells, or other types of immune cells.

The engineered cells that express the MIDIS protein are evaluated to determine the impact of the MIDIS protein on cell function and biological outcomes, for example, using assays described in examples 3-6, and/or other known assays.

### Example 8: Use of MIDIS Proteins in Making Engineered Cells for Adoptive Cell Therapy

One or more MIDIS protein(s) of the disclosure is expressed in a population of cells to be used for adoptive cell therapy. A nucleic acid encoding the MIDIS protein is introduced into the population of cells, for example, introduced ex vivo/in vitro as described in Example 1. The population of engineered cells is expanded in culture. In some cases, the MIDIS protein increases proliferation of the engineered cells, allowing for preparation of a larger number of engineered cells. In some cases, the MIDIS protein selectively increases proliferation of a desired subset of cells, such as cells that recognize and respond to an antigen of interest. The population of engineered cells can optionally be stored in a cell bank. The population of engineered cells can be administered to a subject in need thereof as part of an adoptive cell therapy, such as a cellular immunotherapy to treat cancer. The population of engineered cells can be autologous or allogeneic to the subject. In some cases, efficacy of the adoptive cell therapy is enhanced based on beneficial effects of the MIDIS protein as disclosed herein.

This example evaluates enrichment of TEGs by production and after co-culture.

Alpha-beta T cells were transduced with a defined gamma-delta TCR (SEQ ID NO: 90 and SEQ ID NO: 91) to generate TEGs, with or without additional MIDIS proteins or other control proteins, as in example 1. The MIDIS proteins and control proteins were:
(i) an eGFP control SEQ ID NO: 81;
(ii) 41BBLmincyto—a protein containing an extracellular 41BBL ligand domain, a transmembrane domain from 41BBL, and lacking an intracellular signaling domain, in this example SEQ ID NO: 55 was used;
(iii) 41BBL_OX40—a MIDIS containing an extracellular 41BBL ligand domain, a transmembrane domain from 41BBL, and an OX40 heterologous intracellular signaling domain, in this example SEQ ID NO: 45 was used.

The TEGs were co-incubated with target tumor cells recognized by the defined gamma-delta TCR as described in example 2 for 1 time with RPMI-8226 E:T ratio 1:1 as stimulation and enrichment was assessed after 7 days by FACS staining to identify cells expressing gamma-delta TCR (Y-axis) and alpha beta TCR (X-axis).

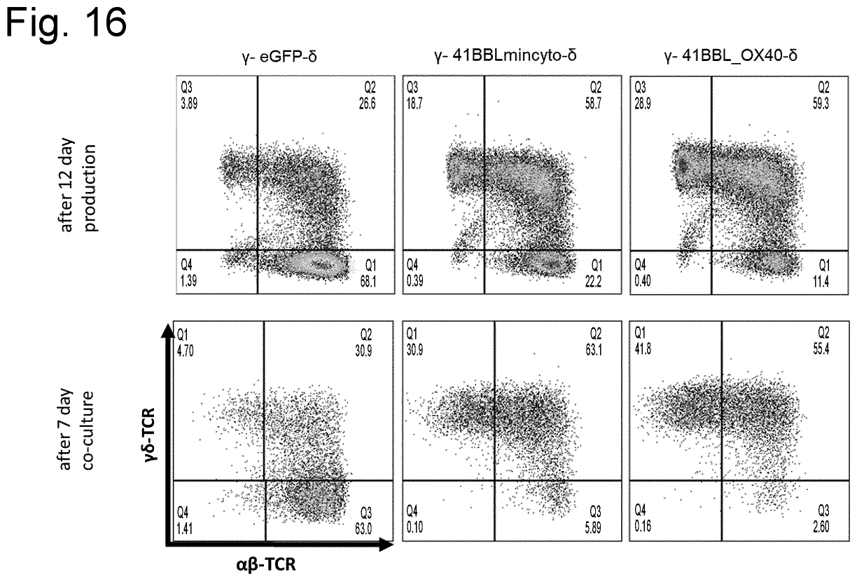

Already after standard culture as described in Example 1 an enrichment in γδ+ TEGs and γδ+αβ−TEGs was observed (FIG. 16) and both populations were further enriched after stimulating them. These results show that MIDIS proteins enhance outgrowth of a selective population highly expressing the exogenous receptor of interest.

### Example 9: 41BBL-OX40 MIDIS Enhances CAR Engineered Immune Cell Responses to Target Cells

This example evaluates the effects of MIDIS proteins of the disclosure on the abilities of CAR engineered immune cells to kill target cells.

Alpha-beta T cells were transduced with a defined CD19.BB.Z CAR (SEQ ID NO: 184) and eGFP to generate CAR-T as demonstrated in Example 1 with as deviation that 2 vectors were used in this case. The MIDIS or control proteins were expressed by a separate vector and alpha beta T cells were transduced with both CD19.BB.Z and the MIDIS containing vector at equal MOI when a MIDIS or control protein was introduced.

In an experiment, the MIDIS protein and control protein were:
(i) 41BBL—wild type 41BBL, in this example SEQ ID NO: 54 was used;
(ii) 41BBL-OX40—a MIDIS containing an extracellular 41BBL ligand domain, a transmembrane domain from 41BBL, and an OX40 heterologous intracellular signaling domain, in this example SEQ ID NO: 45 was used.

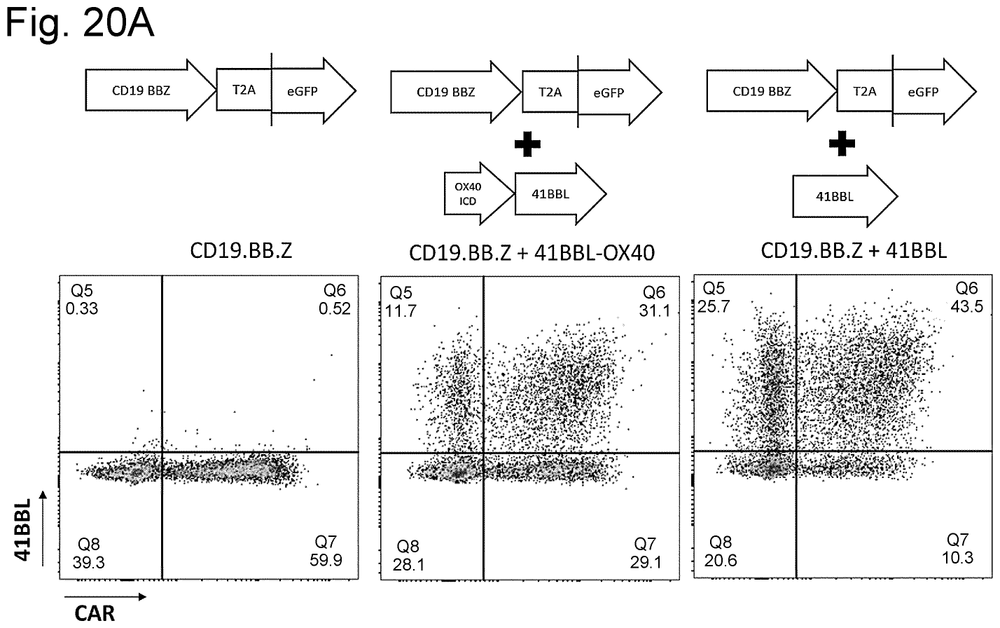

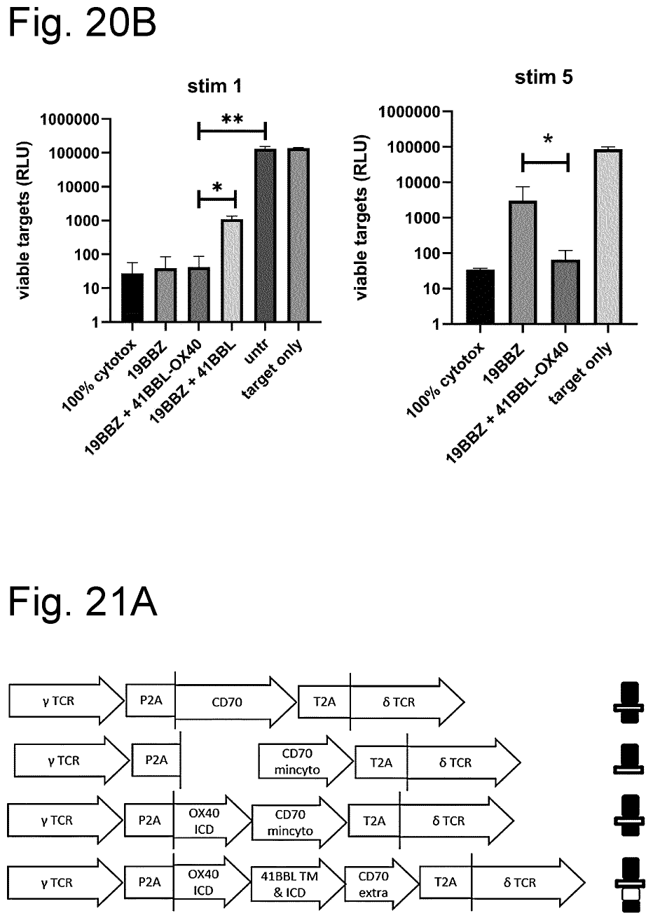

FIG. 20A upper part shows a schematic of select constructs used to introduce the CAR and the other proteins. P2A and T2A represent self-cleaving peptides (SEQ ID NO: 206, SEQ ID NO: 208). Presence of both vectors in transduced CAR T cells was evaluated by flow cytometry. Expression of the CD19.BB.Z and 41BBL is shown in FIG. 20A lower part.

The transduced alpha-beta cells were co-incubated with target NALM6 tumor cells recognized by the CD19 BBZ CAR as described in Example 2, measurement of residual target cell viability by luciferase assay after each stimulation. At stimulation 1 41BBL-OX40 and without additional vector have improved cytotoxicity compared to 41BBL co-transduced alpha-beta cells.

The cytotoxicity of CAR T cells co-incubated with NALM6 target cells ectopically expressing luciferase-tdTomato at effector to target (E:T) ratio of 1:1 after the first stimulation and the fifth stimulation is shown in FIG. 20B (Luminescence of residual HT-29 is demonstrated in relative luminescence units (RLU)). The results show that MIDIS proteins of the disclosure can enhance effector function of engineered immune cells. For example, the CAR T cells expressing the MIDIS protein containing an extracellular 41BBL ligand domain, and a OX40 heterologous intracellular signaling domain exhibited higher cytotoxicity than the CAR T cells that expressed wild type 41BBL.

### Example 10. Expression of Additional MIDIS

This example evaluates the expression MIDIS proteins of the disclosure in engineered immune cells at the cell surface.

Jurkat 76 were transduced with a defined gamma-delta TCR (SEQ ID NO: 90 and SEQ ID NO: 91) with or without additional MIDIS proteins or other control proteins.

In an experiment, the MIDIS protein and control protein were:
(i) CD70—wild type CD70, in this example SEQ ID NO: 180 was used;
(ii) CD70mincyto—wild type CD70 without its cytoplasmic signaling domain, in this example SEQ ID NO: 181 was used;
(iii) CD70mincyto-OX40—a MIDIS containing an extracellular and transmembrane domain from CD70, and an OX40 heterologous intracellular signaling domain, in this example SEQ ID NO: 182 was used.
(iv) CD70-41BBL-OX40—a MIDIS containing an extracellular CD70 domain, a transmembrane domain and cytoplasmic domain from 41BBL, and an OX40 heterologous intracellular signaling domain, in this example SEQ ID NO: 183 was used.

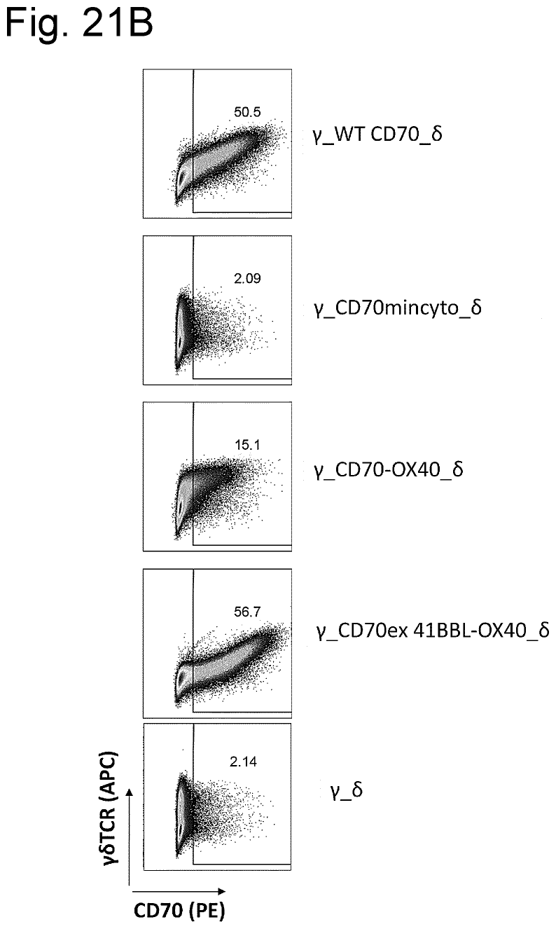

FIG. 21A shows a schematic of select constructs used to introduce the defined gamma-delta TCR and the other proteins. P2A and T2A represent self-cleaving peptides (SEQ ID NO: 206, SEQ ID NO: 208). Expression of the CD70 is shown in FIG. 21B in the different constructs from top to bottom, including control containing no CD70 encoding nucleic acid.

In an additional experiment a defined gamma-delta TCR of the disclosure (SEQ ID NO: 90 and 91) was used to generate TEGs as in Example 1, with or without additional MIDIS proteins or other control proteins. The MIDIS proteins were:
(i) 41BBL-OX40-IL2RB—a MIDIS containing an extracellular 41BBL ligand domain, a transmembrane domain from 41BBL, and an OX40 and IL2RB heterologous intracellular signaling domain, in this example SEQ ID NO: 179 was used;
(ii) 41BBL-OX40—a MIDIS containing an extracellular 41BBL ligand domain, a transmembrane domain from 41BBL, and an OX40 heterologous intracellular signaling domain, in this example SEQ ID NO: 45 was used.

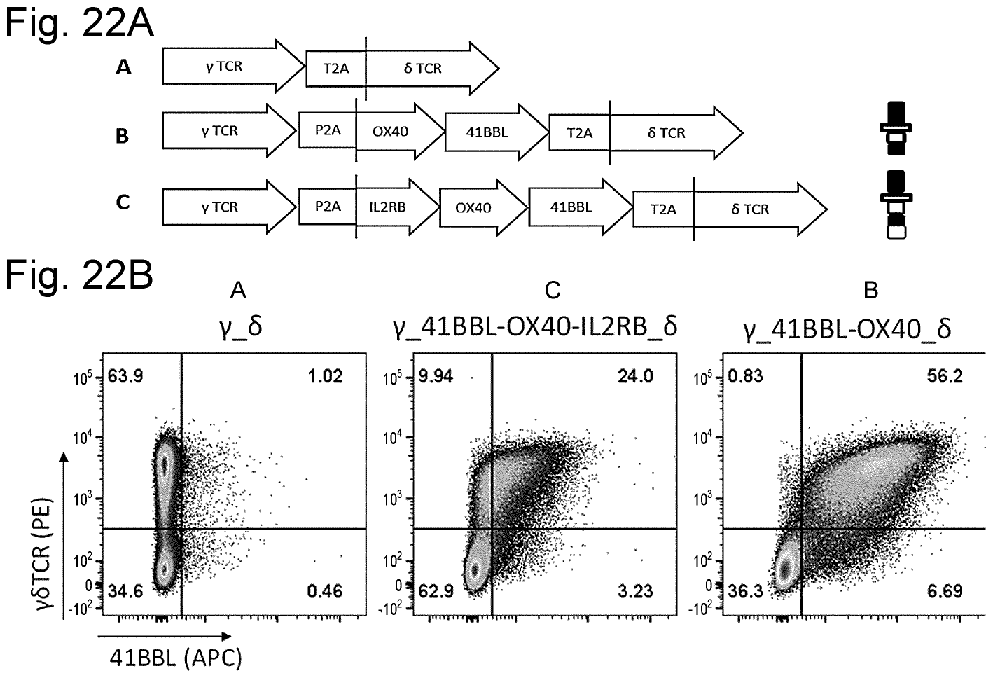

FIG. 22A shows a schematic of select constructs of the defined gamma delta without or with MIDIS proteins. P2A and T2A represent self-cleaving peptides (SEQ ID NO: 206, SEQ ID NO: 208). Expression of the MIDIS as measured by flow cytometry is shown in FIG. 22B.

The results show that MIDIS proteins of the disclosure can contain multiple heterologous intracellular signaling domains and will be expressed at the cell surface of engineered immune cells.

### Example 11: MIDIS Proteins Enhance Expansion of Engineered Immune Cells

This example evaluates the effects of MIDIS proteins of the disclosure on the abilities of engineered non-alpha-beta T cells to expand.

PBMCs were isolated from a fresh buffy coat by density gradient separation using Ficoll. The γδT cells were directly isolated from PBMCs using anti-TCR γ/δ MicroBead kit (Miltenyi) using the autoMACS (Miltenyi). After isolation (day 0), the γδT cells were activated with CD3/CD28 TransAct (Miltenyi), or with 1 μg/mL coated antibody anti-γδTCR clone B1 (Biolegend) and anti-CD28 (ThermoFisher), and cultured in TexMACS (Miltenyi) medium with Penicillin/Streptomycin (0.5%), human serum (2.4%), IL-2 (50 IU/ml), and IL-15 (250 IU/ml) (T cell medium). After 5 days of activation, cells were counted and seeded at 0.35-0.4E6 cells per well, activated with the same procedure as day 0, and transduced with the selected lentiviral vectors at a prior set multiplicity of infection (MOI; 10) by diluting the lentivirus in T cell medium. Lentiviral titer was determined as described in Example 1. Gamma delta T cells were transduced with MIDIS proteins or other control proteins.

In this experiment, the MIDIS proteins and control proteins were:
(i) 41BBL—wild type 41BBL, in this example SEQ ID NO: 54 was used;
(ii) 41BBL-OX40—a MIDIS containing an extracellular 41BBL ligand domain, a transmembrane domain from 41BBL, and an OX40 heterologous intracellular signaling domain, in this example SEQ ID NO: 45 was used.
(iii) eGFP—a fluorescent control protein, in this example SEQ ID NO: 81 was used;

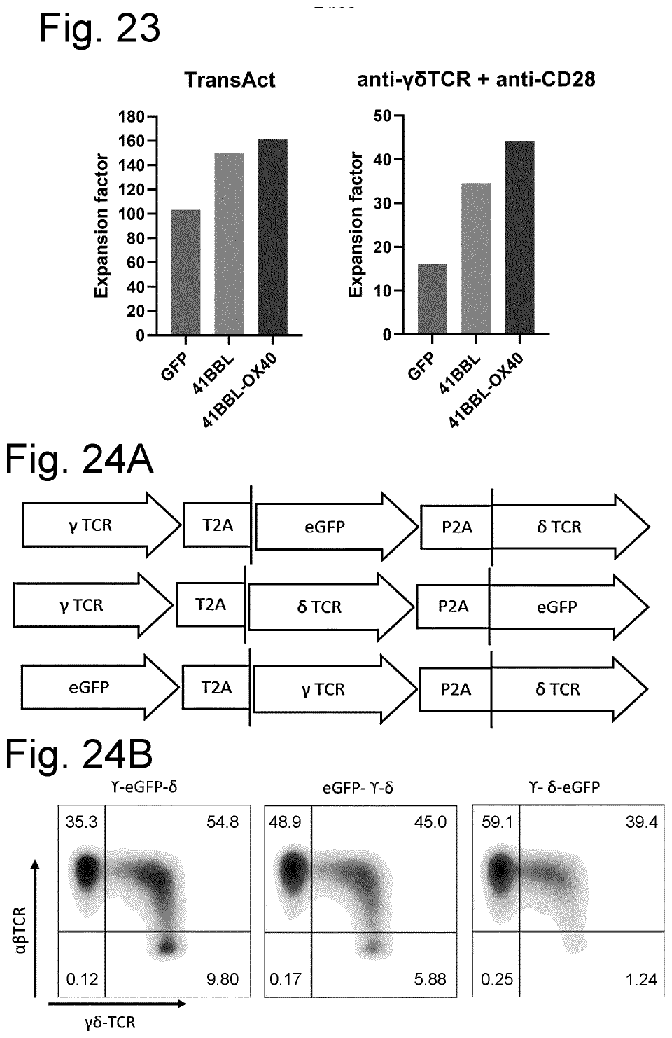

On day 8, 13, 16 and 20 the medium was refreshed with T cell medium and on day 22 the γδT cells were harvested. Cells present at the harvesting day were counted using NucleoCounter NC-200 automated cell counter (Chemometec), and expansion shown in FIG. 23 was calculated based on final yield divided by seeding number at day 0.

The results show that MIDIS proteins of the disclosure can be expressed in non-alpha-beta T cells and enhance their expansion.

### Example 12: Design and Expression of Multicistronic Constructs in T Cells Engineered to Express a Defined Gamma-Delta or Alpha-Beta TCR

Multicistronic constructs of the disclosure were designed to test the effect of inserting at least one nucleic acid between the nucleic acids encoding the receptor monomers of a heterodimeric receptor on the expression of the receptor. Lentiviral vectors were generated to allow for genomic integration of DNA sequences that encode the multiple proteins in different order included of the disclosure. The lentiviral vectors contained a tricistronic or tetracistronic sequence encoding a gamma TCR chain and a delta TCR chain or an alpha and a beta TCR chain of the disclosure located varying positions with the ORF, one or two additional nucleotide sequences encoding eGFP, MIDIS proteins of the disclosure, and/or CD8-Q8, and 2A self-cleaving peptide sequences connecting the three or four protein encoding sequences (described in Xu Y., et al (2019) and Pincha M., et al, (2011), referenced above).

Cryopreserved T cells (αβ T cells or J76 Jurkat cells) were thawed, seeded in 48 well plates at density of 1.6×10\hat{ }6 cells per well in TexMACS (Miltenyi) medium with Penicillin/Streptomycin (0.5%), human serum (2.4%), IL-7 (1700 IU/ml), and IL-15 (150 IU/ml) (TEG medium). The T cells were activated with CD3/CD28 TransAct for 24 h, then transduced with the selected lentiviral vector at a prior set multiplicity of infection (MOI; 0.3-25) by diluting the lentivirus in TexMACS medium. Lentiviral MOI was determined by FACS based titration on J76 Jurkat cells similar to Pirona et al. 2020 (referenced above). In brief, J76 Jurkat cells were seeded at 0.5E6/ml viable cells in a 96 well plate. Lentiviral supernatant was concentrated and serial diluted in cold medium with 10% FBS, starting at 100× dilution. After serial dilution lentivirus containing supernatant was diluted 1:1 with J76 Jurkat cells and the mixture was incubated for 3 days at 37 degrees Celsius. Titer was determined by analyzing percentage of CD3⁺ viable cells.

On the second day medium was refreshed and T cells (αβ T cells or J76 Jurkat cells) transduced with a vector containing a γδTCR or an αβTCR encoding sequence were transferred to T25 flasks with a final volume of 5 ml. In the case of αβTCRs, T cells may alternatively be transduced with a vector containing an αβTCR and nucleofected with protein-RNA complexes of SpCAS9 and single guide RNA (CAS9:sgRNA) using a nucleofector 2b, program T-023 and Nucleofector kit T (Lonza) according to general guidelines of Synthego; sgRNAs that may be used are ACAAAACUGUGCUAGACAUG (SEQ ID NO: 191), GAGAAUCAAAAUCGGUGAAU (SEQ ID NO: 192), CUCUCAGCUGGUACACGGCA (SEQ ID NO: 193) for TRAC and ACCCGAGGUCGCUGUGUUUG (SEQ ID NO: 194), AGGUCGCUGUGUUUGAGCCA (SEQ ID NO: 195), CGACCACGUGGAGCUGAGCU (SEQ ID NO: 196), GAUACUGCCUGAGCAGCCGC (SEQ ID NO: 197) for TRBC1 and TRBC2. After nucleofection cells may be transferred to T25 flasks as mentioned above.

On day 6 medium was refreshed once more, and T cells were transferred to a T75 cell culture flask with a final volume of 10 ml. T cells were harvested 8 days after thawing them.

Cells present after the 8-day protocol were characterized by flow cytometry. Cells were washed with FACS buffer (PBS with 2% fetal bovine serum), and stained with selected fluorescently-conjugated antibodies in FACS buffer for 30 min at 4 degrees Celsius. After staining, cells were washed twice with FACS buffer, and fixed by 4% paraformaldehyde for 10 min at 4 degrees Celsius. Following fixation, cells were washed once more with FACS buffer, before resuspension in 100-200 μl of FACS buffer, followed by flow cytometric analysis.

### Example 13: Co-Culture Assays of Targets Cells and Effector T Cells Expressing a Defined Gamma-Delta TCR of Alpha-Beta TCR in a Multicistronic Construct

Target tumor cells, including HT-29, RKO, U266 cells, were seeded at defined concentrations in 96 well plates. Adherent target cells were seeded a day before effector cells were added, and suspension target cells were seeded on the same day that effector cells were added.

The variable domains of the defined gamma-delta TCR expressed in the multicistronic constructs was either the one represented by SEQ ID NO: 90 and 91 (cI5) or 111 and 112 (E57).

Cryopreserved TEGs were used as effector cells. The number of effector cells added to all co-cultures were corrected to match the TEGs with the lowest transduction efficiency in the experiment. Untransduced cells which went through the same production process, but without adding lentiviral vectors comprising the multicistronic constructs, were added so that all co-cultures within one experiment contained the same number of TEGs and the same number of total T cells.

Co-cultures were setup at a effector to target ratio (E:T) of 9:1, 3:1, 1:1, 0.3:1 or 0.11:1, and with or without pamidronate treatment (10 μm) on the day of effector cell addition. Untransduced, effector only, target only, or full lysis controls were included.

After 3 or 4 days, supernatant was collected from the co-cultures for IFNγ ELISA (R&D systems). Non-adherent cells were resuspended gently, and the cell suspension was transferred to round bottom 96 well plate. When cytotoxicity was determined by luminescence, 100 μl of assay medium together with D-luciferin was added to the residual co-culture plate containing any remaining target cells, and incubated for 12 minutes before measuring luminescence by Glomax (Promega).

### Example 14: Multicistronic Constructs where Gamma-Chain-Encoding Nucleic Acid is Positioned at First Position and Delta-Chain-Encoding Nucleic Acid is Positioned at Last Position Enhance the Expression of the Defined Gamma-Delta TCR

This example evaluates the effects of the position of a gamma-chain-encoding nucleic acid and delta-chain-encoding nucleic acid in a multicistronic construct described herein on the abilities of engineered immune cells to express a defined gamma-delta TCR.

Alpha-beta T cells were transduced with nucleic acids encoding the following gamma-delta TCR chain combinations: a defined gamma (SEQ ID NO: 90) and delta (SEQ ID NO:91) chain or gamma (SEQ ID NO: 111) and delta (SEQ ID NO: 112) chain, to generate TEGs as outlined in Example 12, and with a nucleic acid encoding eGFP (SEQ ID NO: 81) inserted between the nucleic acids encoding the gamma and delta chains in a tricistronic construct. Control tricistronic constructs in which the nucleic acid encoding eGFP was placed at the first or last position of the construct were also designed.

FIG. 24A shows a schematic of the constructs used to introduce expression of the gamma-delta TCR and the other proteins. P2A and T2A represent self-cleaving peptides (SEQ ID NO: 206, SEQ ID NO: 208).

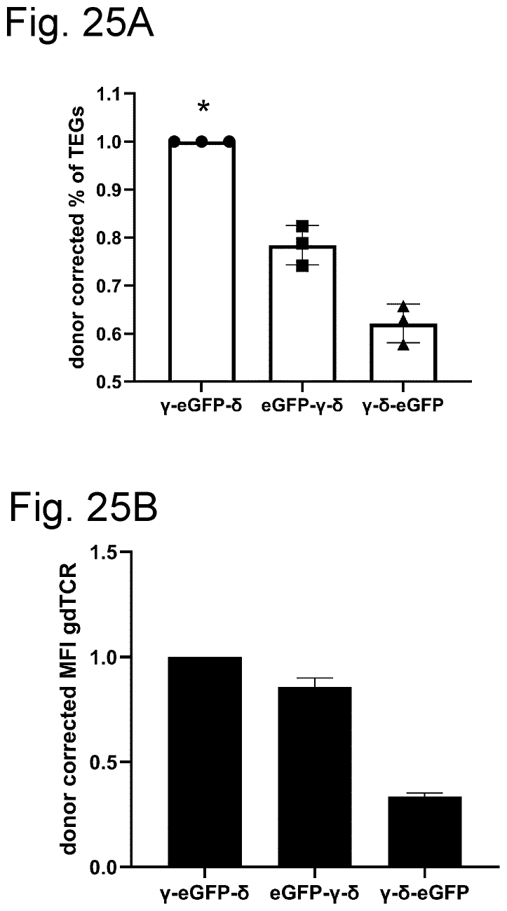

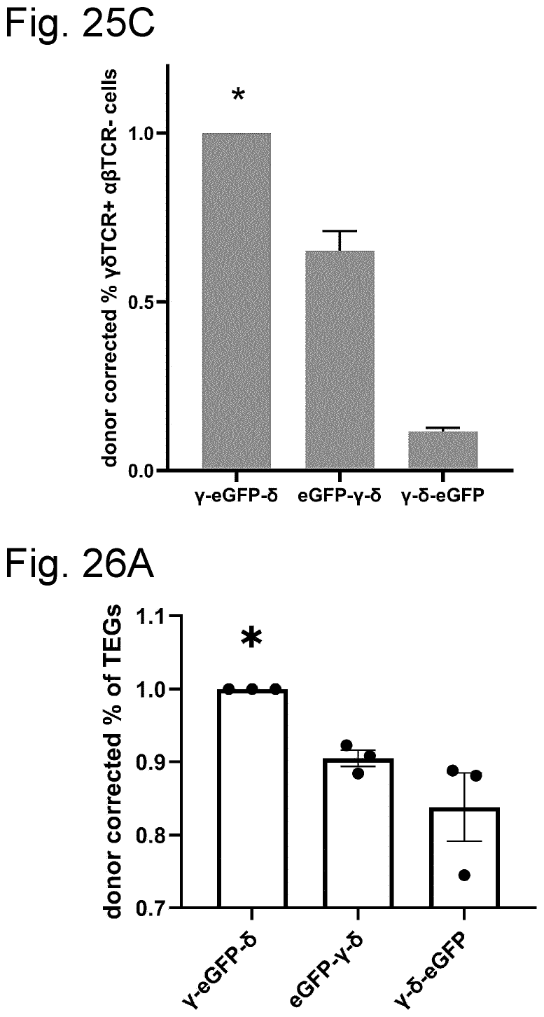

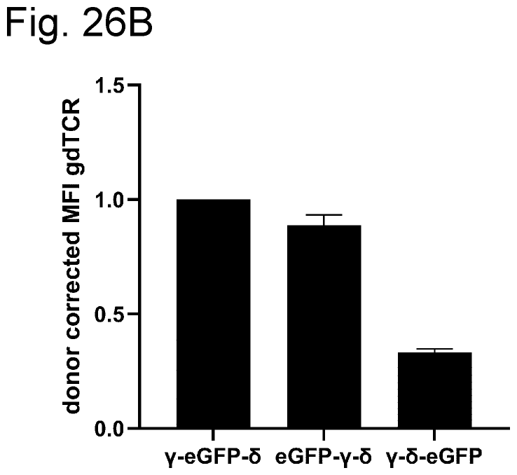

TEGs were stained as described in Example 12 for three donors and % of TEGs (γδ TCR+ T cells), γδ TCR MFI of γδ TCR+ cells or γδ TCR+αβ TCR− % of live single cells were obtained as a measure of γδ TCR expression. An example (defined gamma delta TCR cl5, SEQ ID NO: 90 and 91) of the flow plots is shown in FIG. 24B. The combined donor corrected measurements are shown in FIG. 25A-25C (TEGs expressing SEQ ID NO: 90-91) and FIG. 26A-26B (SEQ ID NO: 111-112). For donor correction, the value obtained for γ-eGFP-δ per donor was set to 1 and relative change of the other constructs was calculated accordingly (relative comparison between the constructs per donor). Tricistronic constructs with gamma at first position and delta at last position showed highest expression of the define gamma-delta TCR compared to the other combinations.

In another experiment, alpha-beta T cells were transduced with nucleic acids encoding a defined gamma chain (SEQ ID NO: 90) and delta chain (SEQ ID NO:91) to generate TEGs as outlined in Example 12, and with a nucleic acid encoding 41BBL-OX40 (SEQ ID NO: 45) or CD8-Q8 (SEQ ID NO: 80) inserted between the nucleic acids encoding the gamma and delta chains in a tricistronic construct. Control tricistronic constructs in which the nucleic acids encoding 41BBL-OX40 or Q8 were placed at the last position of the construct were also designed.

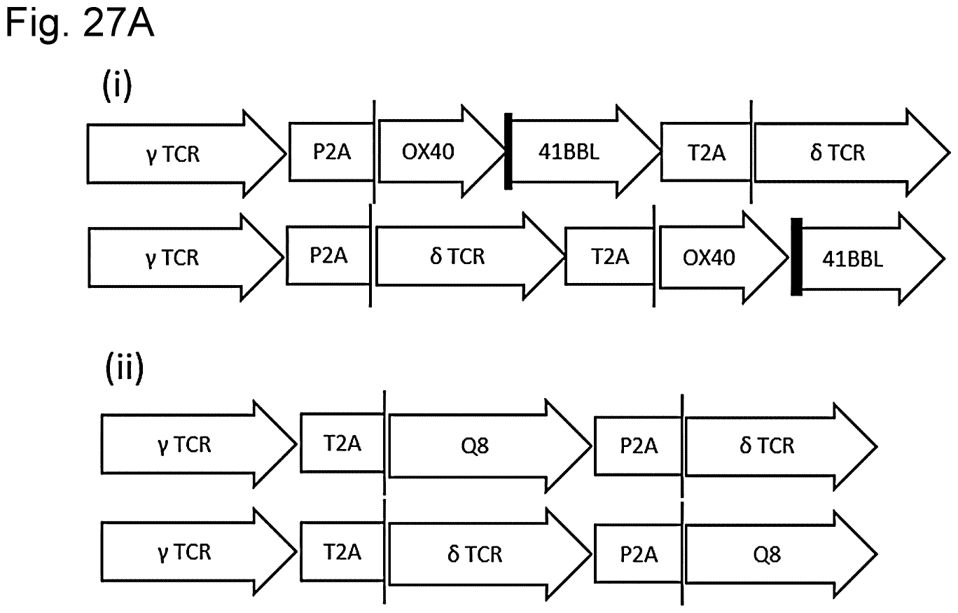

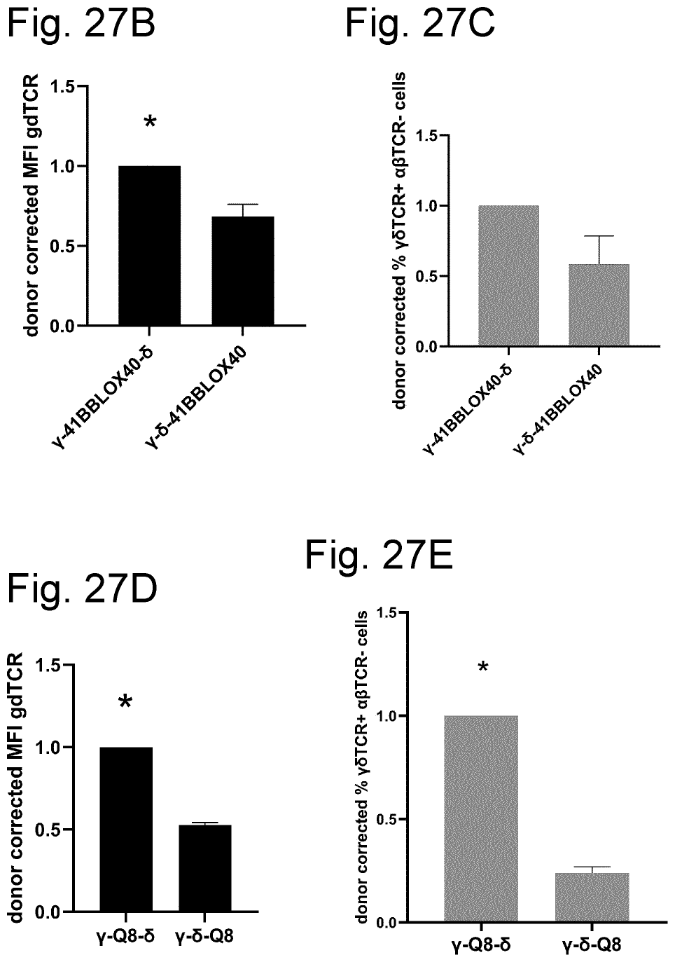

FIG. 27A shows a schematic of the constructs used to introduce expression of the gamma-delta TCR and the other proteins. P2A and T2A represent self-cleaving peptides (SEQ ID NO: 206, SEQ ID NO: 208). The black bars at the N-terminal end of 41BBL represents a linker sequence (SEQ ID NO:28).

TEGs were stained as described in Example 12 for three donors and γδ TCR MFI of γδ TCR+ or γδ TCR+αβ TCR− % of live single T cells were obtained as a measure of expression. The combined donor corrected measurement are shown in FIG. 27B-C (41BBL-OX40) and FIG. 27D-E (CD8-Q8). Donor correction was performed similarly to described above. Tricistronic constructs with the gamma-chain-encoding nucleic acid at first position and delta-chain-encoding nucleic acid at last position showed highest expression of the defined gamma-delta TCR compared to the other combinations.

In another experiment, alpha-beta T cells were transduced with nucleic acids encoding a defined gamma chain (SEQ ID NO: 90) and delta chain (SEQ ID NO: 91) to generate TEGs as outlined in Example 12, and with nucleic acids encoding 41BBL-OX40 (SEQ ID NO: 45) and eGFP (SEQ ID NO: 81) inserted between the nucleic acids encoding the gamma and delta chains in a tetracistronic construct. Control tetracistronic constructs in which the nucleic acids encoding 41BBL-OX40 and eGFP were placed at the first two or last two positions of the constructs were also designed.

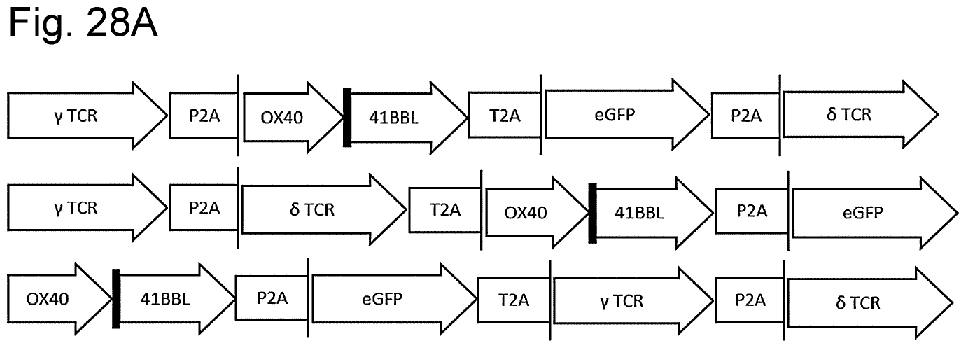

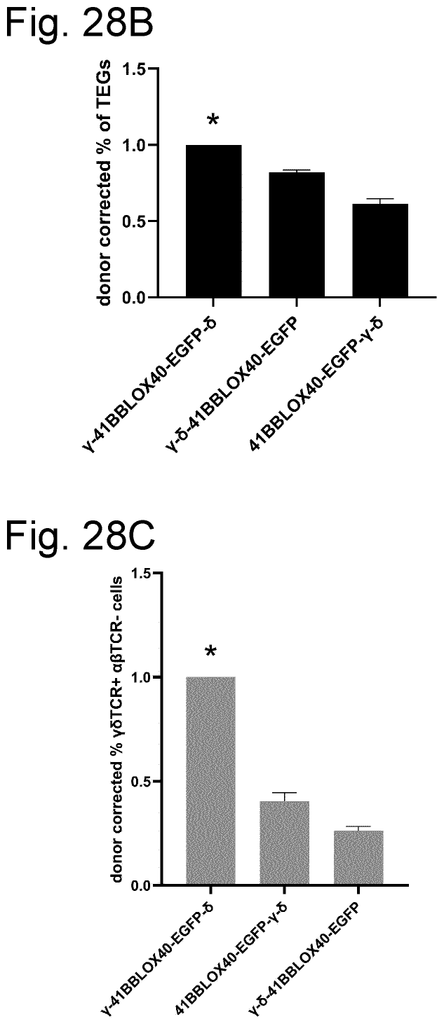

FIG. 28A shows a schematic of the constructs used to introduce expression of the gamma-delta TCR and the other proteins. P2A and T2A represent self-cleaving peptides (SEQ ID NO: 206, SEQ ID NO: 208). TEGs were stained as described in Example 12 for three donors and % of TEGs or γδ TCR+αβ TCR− % of live single T cells were obtained as a measure of expression. Donor correction was performed similarly to described above. The combined donor corrected measurements are shown in FIG. 28B-C. Tetracistronic constructs with the gamma-chain-encoding nucleic acid at first position and delta-chain-encoding nucleic acid at last position showed highest expression of the defined gamma-delta TCR compared to the other combinations. These data further demonstrate that inserting one or more nucleic acids encoding a polypeptide other than the receptor monomers, with a nucleic acid encoding one part of the heterodimer at first position and the other at last position in a multicistronic construct, can improve the expression of the heterodimer in engineered immune cells.

### Example 15: Multicistronic Constructs where Gamma-Chain- or Delta-Chain-Encoding Nucleic Acid is Positioned at First Position and Delta-Chain- or -Gamma-Chain-Encoding Nucleic Acid is Positioned at Last Position Leads to Highest Cell Response to Target Cells

This example evaluates the effect of the position of the nucleic acids encoding the heterodimer monomers of a receptor of the disclosure in a multicistronic construct on the abilities of engineered immune cells to respond to target cells. Alpha-beta T cells were transduced with nucleic acids encoding the following gamma-delta TCR chain combinations: a defined gamma (SEQ ID NO: 90) and delta (SEQ ID NO: 91) chain or gamma (SEQ ID NO: 111) and delta (SEQ ID NO: 112) chain, and with a nucleic acid encoding eGFP (SEQ ID NO: 81) inserted between the nucleic acids encoding the gamma and delta chains in a tricistronic construct, as outlined in Example 12. Control tricistronic constructs in which the nucleic acid encoding eGFP was placed at the first or last position of the construct were also designed.

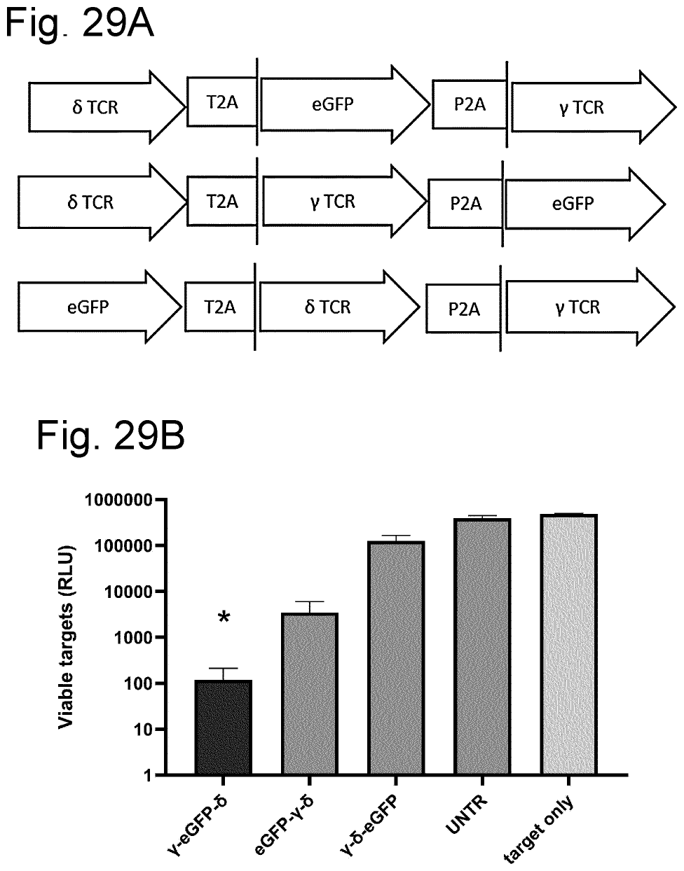

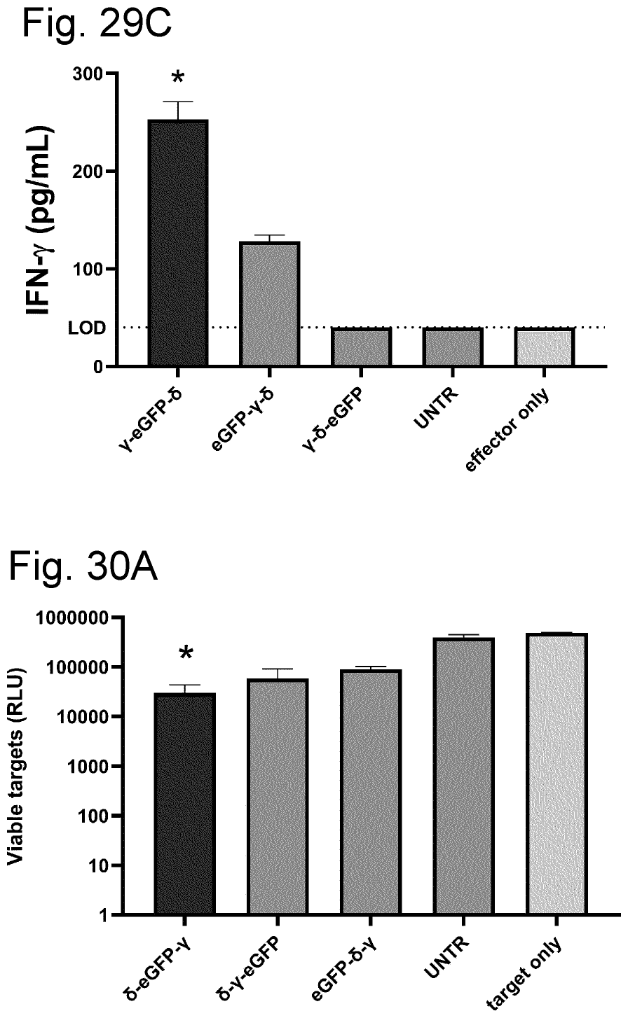

FIG. 24A and FIG. 29A shows a schematic of the constructs used to introduce expression of the gamma-delta TCRs and the other proteins. P2A and T2A represent self-cleaving peptides (SEQ ID NO: 206, SEQ ID NO: 208).

The TEGs were co-incubated with target tumor cells recognized by the defined gamma-delta TCRs as described in Example 13, including collection of supernatant and measurement of residual target cell viability by luciferase assay. The experiment included conditions with or without 10 pM pamidronate treatment.

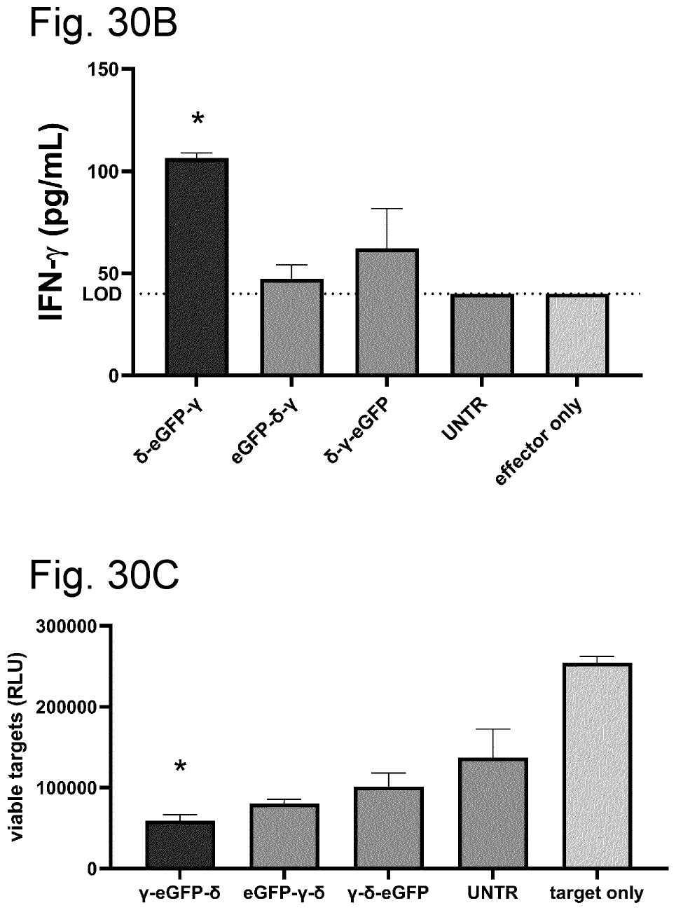

The cytotoxicity (by relative luminescence units of the viable targets) and IFNγ release of TEGs expressing a nucleic acid encoding defined gamma chain (SEQ ID NO: 90) at first position and a delta chain at last position (SEQ ID NO: 91) of the tricistronic construct or a nucleic acid encoding a delta chain at first position and a gamma chain at last position of a representative donor co-incubated with HT-29 ectopically expressing luciferase-tdTomato at effector to target (E:T) ratio of 1:1 is shown in FIG. 29B-C (gamma -eGFP- delta order) and in FIG. 30A-B (delta -eGFP- gamma order) respectively. Co-cultures with TEGs expressing a gamma chain or delta chain from the first or last position of the construct had significant less viable target cells and significant higher level of IFNγ release.

The cytotoxicity of TEGs, expressing a defined gamma chain (SEQ ID NO: 111) from the first position and delta chain from the last position (SEQ ID NO: 112) of a tricistronic construct, of a representative donor co-incubated with RKO ectopically expressing luciferase-tdTomato at effector to target (E:T) ratio of 0.11:1 is shown in FIG. 30C, with the results being in agreement with results obtained for SEQ ID NO: 90 and SEQ ID NO:91.

In another experiment, alpha-beta T cells were transduced with a nucleic acid encoding a defined gamma (SEQ ID NO: 90) chain and delta (SEQ ID NO:91) chain to generate TEGs as outlined in Example 12, with additional nucleic acids encoding proteins inserted between the nucleic acids encoding the gamma and delta chains in tricistronic constructs. Controls were also designed as outlined in Example 14. The additional proteins in this example were 41BBL-OX40 (SEQ ID NO: 45) and CD8-Q8 (SEQ ID NO: 80). The TEGs were co-incubated with target tumor cells recognized by the defined gamma-delta TCRs as described in Example 13, including collection of supernatant and measurement of residual target cell viability by luciferase assay.

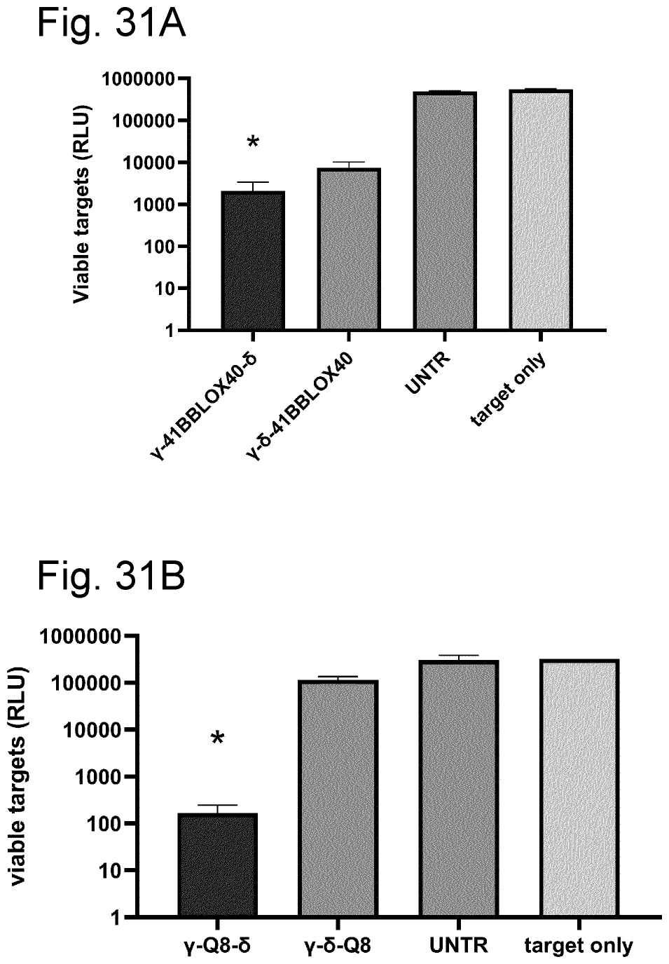

The cytotoxicity (by relative luminescence units of the viable targets) of TEGs expressing a defined gamma delta of a representative donor co-incubated with HT-29 ectopically expressing luciferase-tdTomato at effector to target (E:T) ratio of 0.3:1 is shown in FIG. 31-B. TEGs expressing constructs with a gamma-chain encoding nucleic acid positioned first and a delta-encoding nucleic acid positioned last in the construct, wherein the 41BBL-OX40- or CD8-Q8-encoding nucleic acids were positioned between the gamma and delta chain encoding nucleic acids, showed significantly higher cytotoxicity.

### Example 16: Multicistronic Constructs where Gamma-Chain-Encoding Nucleic Acid is Positioned at First Position and Delta-Chain Encoding Nucleic Acid is Positioned at Last Position Lead to Higher Cell Response to Target Cells Compared to Delta-Chain-Encoding Nucleic Acid at First Position and Gamma-Chain-Encoding Nucleic Acid at Last Position

This example evaluates the effects of the position (order) of the gamma-chain- and delta-chain encoding nucleic acids in the multicistronic construct of the disclosure on the ability of cells expressing them to respond to target cells. Alpha-beta T cells were transduced with nucleic acids encoding the following gamma-delta TCR chain combinations: a defined gamma (SEQ ID NO: 90) and delta (SEQ ID NO: 91) chain or gamma (SEQ ID NO: 111) and delta (SEQ ID NO: 112) chain, and with a nucleic acid encoding eGFP (SEQ ID NO: 81) or 41BBL-OX40 (SEQ ID NO: 45) inserted between the nucleic acids encoding the gamma and delta chains in a tricistronic construct, as outlined in Example 12.

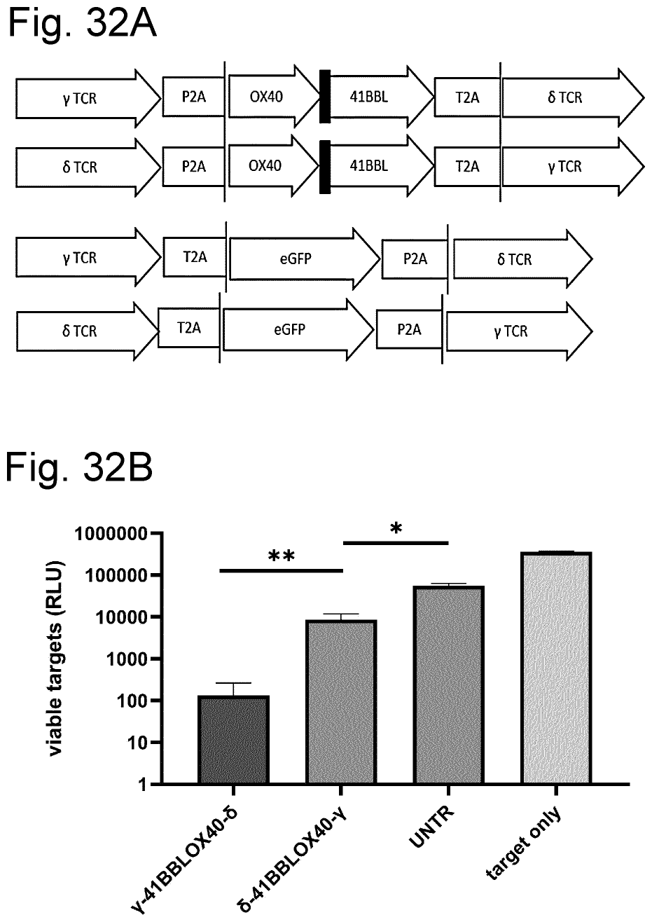

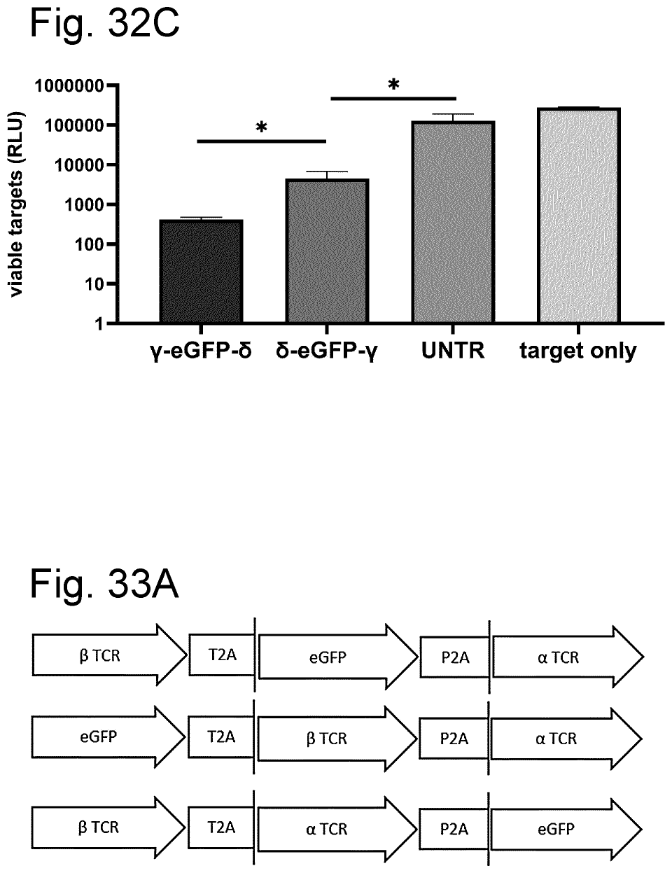

FIG. 32A shows a schematic of the constructs used to introduce the gamma-delta TCR and the other proteins. P2A and T2A represent self-cleaving peptides (SEQ ID NO: 206, SEQ ID NO: 208).

The TEGs were co-incubated with target tumor cells recognized by the defined gamma-delta TCR as described in example 13, measurement of residual target cell viability by luciferase assay. The experiment included conditions with or without pamidronate treatment (10 μm).

The cytotoxicity (by relative luminescence units of the viable targets) of TEGs expressing a defined gamma delta (SEQ ID NO: 90-91) of a representative donor co-incubated with HT-29 ectopically expressing luciferase-tdTomato at effector to target (E:T) ratio of 3:1 is shown in FIG. 32B. Cytotoxicity of TEGs with a defined gamma delta (SEQ ID NO: 111-112) of a representative donor co-incubated with RKO ectopically expressing luciferase-tdTomato at effector to target (E:T) ratio of 3:1 is shown in FIG. 32C. Although TEGs significantly kill the target cells irrespective of when the gamma-chain-encoding nucleic acid and the delta-chain-encoding nucleic acid is positioned as the first or last nucleic acid in the tricistronic constructs, compared to untransduced T cells (UNTR), TEGs expressing constructs with the gamma-chain-encoding nucleic acid positioned first and the delta-chain-encoding nucleic acid positioned last showed significant higher cytotoxicity compared to TEGs expressing construct with the delta-chain-encoding nucleic acid positioned first and the gamma-chain encoding nucleic acid positioned last.

### Example 17: Multicistronic Constructs where Beta-Chain-Encoding Nucleic Acid is Positioned at First Position and Alpha-Chain Encoding Nucleic Acid is Positioned at Last Position Leads to Higher Expression of an Alpha-Beta TCR

This example evaluates the effect of the position (order) of an alpha-chain-encoding nucleic acid and a beta-chain encoding nucleic acid in a multicistronic construct of the disclosure on the abilities of engineered immune cells to respond to target cells.

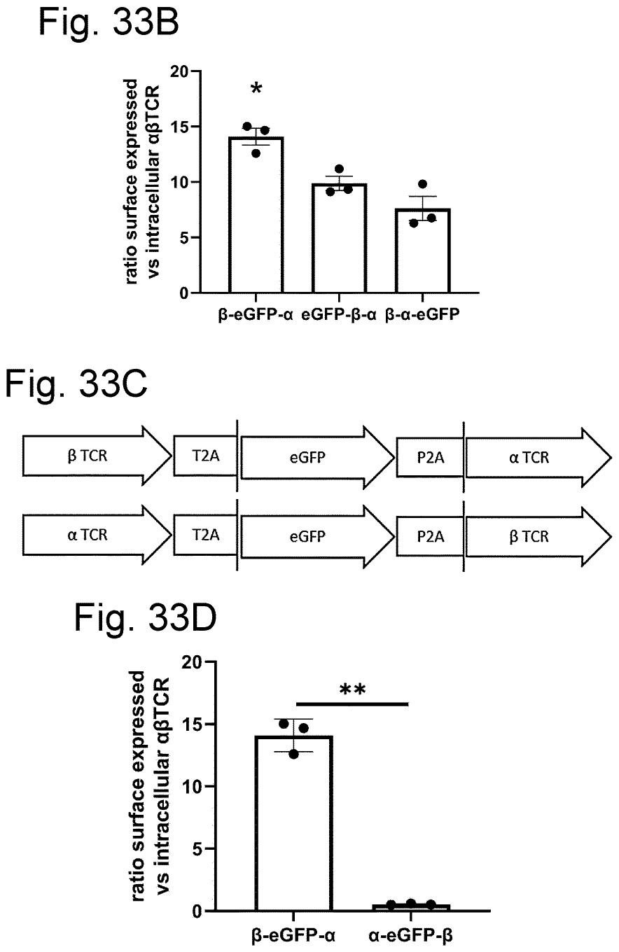

Jurkat 76 T cells were transduced with a tricistronic construct comprising a nucleic acid encoding a defined alpha (SEQ ID NO: 199) and beta (SEQ ID NO: 198) chain, and a nucleic acid encoding eGFP (SEQ ID NO: 81) inserted between the nucleic acids encoding the alpha and beta chains, to generate exogenous alpha beta TCR expressing engineered T cells. Control tricistronic constructs in which the nucleic acid encoding eGFP was placed at the first or last position of the construct were also designed. Transduction was performed as outlined in example 12 with fixed MOI, with the difference that Jurkat T cells were not subjected to CRISPR editing. 3 days after transduction the expression of the alpha beta TCR and transduction efficiency was assessed by flow cytometry as described in Example 12. Surface expression of alpha beta TCR was assessed with anti-CD3ε and total expression of alpha beta TCR was assessed by staining cells after fixation with anti-alpha beta TCR antibody (Table 18). Ratio between cell surface expression and intracellular expression demonstrates the efficiency of alpha beta TCR trafficking and surface expression. FIG. 33A shows a schematic of the constructs used to introduce expression of the defined alpha beta TCR and the other proteins. P2A and T2A represent self-cleaving peptides (SEQ ID NO: 206, SEQ ID NO: 208).

Ratio of alpha beta TCR surface expression divided by intracellular alpha beta TCR expression is shown in FIG. 33B was higher in the Jurkat T cells expressing a tricistronic construct with the beta-chain-encoding nucleic acid at first position and the alpha-chain-encoding nucleic acid at last position compared to the controls.

In another experiment, the order of beta-chain-encoding nucleic acid at first position and alpha-chain encoding nucleic acid at last position was compared to the order of alpha-chain encoding nucleic acid at first position and beta-chain-encoding nucleic acid at last position in tricistronic constructs. The constructs further comprised a nucleic acid encoding eGFP (SEQ ID NO: 81) inserted between the nucleic acids encoding the alpha and beta chains. Jurkat 76 T cells were transduced with constructs comprising nucleic acids encoding a defined alpha (SEQ ID NO: 199) and beta (SEQ ID NO: 198) chain to generate exogenous alpha beta TCR expressing engineered T cells. FIG. 33C shows a schematic of the constructs used in this experiment. P2A and T2A represent self-cleaving peptides. Staining of transduced cells was performed as described in the first experiment of this example. Ratio of alpha beta TCR surface expression divided by intracellular alpha beta TCR expression is shown in FIG. 33D.

TABLE 18
Antibodies used

| antigen | Fluorochrome | Clone | anti- | article # | Vendor |
|---|---|---|---|---|---|
| CD4 | AF700 | RPA-T4 | human | 56-0049-42 | eBioscience/Thermo |
| CD8a | BV510 | RPA-T8 | human | 301048 | Biolegend |
| CD3 | BV421 | UCHT-1 | human | 300434 | Biolegend |
| αβTCR | APC | IP26 | human | 17-9986-42 | eBioscience/Thermo |
| γδTCR | FITC | IMMU510 | human | B49175 | Beckman Coulter |
| γδTCR | APC | REA591 | human | 130-113-508 | Miltenyi |
| γδTCR | PE | IMMU510 | human | B49176 | Beckman Coulter |
| 41BB | PE-Dazzle594 | 4B4-1 | human | 309826 | Biolegend |
| OX40 | PerCP-Cy5,5 | ACT35 | human | 563659 | BD Bioscience |
| PD1 | PE | EH12.2H7 | human | 329906 | Biolegend |
| PD1 | BV421 | EH12.2H7 | human | 329920 | Biolegend |
| TIM3 | BV785 | F38-2E2 | human | 311608 | BioLegend |
| LAG3 | BV711 | 11C3C65 | human | 345031 | BioLegend |
| 41BBL | APC | 5F4 | human | 311506 | Biolegend |
| 41BBL | PE | 5F4 | human | 311503 | Biolegend |
| OX40L | PE | ik-1 | human | 563766 | BD Bioscience |
| CD86 | APC | IT2.2 | human | 305412 | biolegend |
| CD86 | FITC | BU63 | human | 374203 | biolegend |
| CD86 | AF488 | IT2.2 | human | 305413 | biolegend |
| Fab2 | AF647 | polyclonal | human | 115-605-072 | Jackson Immunoresearch |
| | | | | | Europe Ltd\Sanbio |
| CD107a | BV711 | H4A3 | human | 328640 | Biolegend |
| CD69 | AF700 | FN50 | human | 310922 | Biolegend |

TABLE 19
Additional sequences

| SEQ | | | |
|---|---|---|---|
| ID NO: | Name | Description | Sequence |
| 134 | TRDC | constant domain | SQPHTKPSVFVMKNGTNVACLVKEFYPKDI |
| | | of TCR delta, | RINLVSSKKITEFDPAIVISPSGKYNAVKLGK |
| | | UniProtKB | YEDSNSVTCSVQHDNKTVHSTDFEVKTDS |
| | | database- | TDHVKPKETENTKQPSKSCHKPKAIVHTEK |
| | | B7Z8K6 | VNMMSLTVLGLRMLFAKTVAVNFLLTAKLF |
| | | | FL |
| 135 | TRGC1 | constant domain | DKQLDADVSPKPTIFLPSIAETKLQKAGTYL |
| | | 1 of TCR | CLLEKFFPDVIKIHWQEKKSNTILGSQEGNT |
| | | gamma, | MKTNDTYMKFSWLTVPEKSLDKEHRCIVR |
| | | UniProtKB | HENNKNGVDQEIIFPPIKTDVITMDPKDNCS |
| | | database- | KDANDTLLLQLTNTSAYYMYLLLLLKSVVYF |
| | | POCF51 | AIITCCLLRRTAFCONGEKS |
| 136 | TRGC2 | constant domain | DKQLDADVSPKPTIFLPSIAETKLQKAGTYL |
| | | 2 of TCR | CLLEKFFPDIIKIHWQEKKSNTILGSQEGNT |
| | | gamma, | MKTNDTYMKFSWLTVPEESLDKEHRCIVR |
| | | UniProtKB | HENNKNGIDQEIIFPPIKTDVTTVDPKYNYS |
| | | database- | KDANDVITMDPKDNWSKDANDTLLLQLTNT |
| | | P03986 | SAYYTYLLLLLKSVVYFAIITCCLLRRTAFCC |
| | | | NGEKS |
| 137 | ICOSL- | Extracellular | MRLGSPGLLFLLFSSLRADTQEKEVRAMV |
| | 41BB | and tm part of | GSDVELSCACPEGSRFDLNDVYVYWQTSE |
| | | ICOSL and the | SKTVVTYHIPQNSSLENVDSRYRNRALMSP |
| | | cytoplasmic part | AGMLRGDFSLRLFNVTPQDEQKFHCLVLS |
| | | of 41BB | QSLGFQEVLSVEVTLHVAANFSVPVVSAPH |
| | | | SPSQDELTFTCTSINGYPRPNVYWINKTDN |
| | | | SLLDQALQNDTVFLNMRGLYDVVSVLRIAR |
| | | | TPSVNIGCCIENVLLQQNLTVGSQTGNDIG |
| | | | ERDKITENPVSTGEKNAATWSILAVLCLLVV |
| | | | VAVAIGWVCKRGRKKLLYIFKQPFMRPVQT |
| | | | TQEEDGCSCRFPEEEEGGCEL |
| 138 | ICOSL | Extracellular | MRLGSPGLLFLLFSSLRADTQEKEVRAMV |
| | extra + | and | GSDVELSCACPEGSRFDLNDVYVYWQTSE |
| | tm | transmembrane | SKTVVTYHIPQNSSLENVDSRYRNRALMSP |
| | | part of ICOSL | AGMLRGDFSLRLFNVTPQDEQKFHCLVLS |
| | | | QSLGFQEVLSVEVTLHVAANFSVPVVSAPH |
| | | | SPSQDELTFTCTSINGYPRPNVYWINKTDN |
| | | | SLLDQALQNDTVFLNMRGLYDVVSVLRIAR |
| | | | TPSVNIGCCIENVLLQQNLTVGSQTGNDIG |
| | | | ERDKITENPVSTGEKNAATWSILAVLCLLVV |
| | | | VAVAIGWVC |
| 139 | ICOSL | Extracellular | MRLGSPGLLFLLFSSLRADTQEKEVRAMV |
| | extra | part of ICOSL | GSDVELSCACPEGSRFDLNDVYVYWQTSE |
| | | | SKTVVTYHIPQNSSLENVDSRYRNRALMSP |
| | | | AGMLRGDFSLRLFNVTPQDEQKFHCLVLS |
| | | | QSLGFQEVLSVEVTLHVAANFSVPVVSAPH |
| | | | SPSQDELTFTCTSINGYPRPNVYWINKTDN |
| | | | SLLDQALQNDTVFLNMRGLYDVVSVLRIAR |
| | | | TPSVNIGCCIENVLLQQNLTVGSQTGNDIG |
| | | | ERDKITENPVSTGEKNAAT |

TABLE 20
Description of nucleotide sequences encoding
aminoacid sequences of Table 5

| | |
|---|---|
| 140 | Sequence encoding SEQ ID NO: 45 (41BBL-OX40) |
| 141 | Sequence encoding SEQ ID NO: 46 (41BBL13W-OX40rev) |
| 142 | Sequence encoding SEQ ID NO: 47 (41BBL-NKp80) |
| 143 | Sequence encoding SEQ ID NO: 48 (41BBLmincyto-NKp80) |
| 144 | Sequence encoding SEQ ID NO: 50 (OX40L-41BB) |
| 145 | Sequence encoding SEQ ID NO: 51 (OX40L-41BBrev) |
| 146 | Sequence encoding SEQ ID NO: 52 (CD86-OX40) |
| 147 | Sequence encoding SEQ ID NO: 53 (CD86delP276-OX40) |
| 148 | Sequence encoding SEQ ID NO: 54 (41BBL) |
| 149 | Sequence encoding SEQ ID NO: 55 (41BBLmincyto) |
| 150 | Sequence encoding SEQ ID NO: 57 (41BBL-OX40rev) |
| 151 | Sequence encoding SEQ ID NO: 58 (41BBL-rev extra-OX40tm- |
| | cyto) |
| 152 | Sequence encoding SEQ ID NO: 59 (41BBLmincyto-OX40) |
| 153 | Sequence encoding SEQ ID NO: 60 (41BBL13W-OX40rev) |
| 154 | Sequence encoding SEQ ID NO: 61 (41BBL-mincyto-OX40rev) |
| 155 | Sequence encoding SEQ ID NO: 62 (41BBL-OX40ENrev) |
| 156 | Sequence encoding SEQ ID NO: 63 (41BBL-OX40EN) |
| 157 | Sequence encoding SEQ ID NO: 64 (41BBLmincyto-13alink- |
| | OX40) |
| 158 | Sequence encoding SEQ ID NO: 65 (OX40Lmincyto-41BBrev) |
| 159 | Sequence encoding SEQ ID NO: 66 (OX40L WT) |
| 160 | Sequence encoding SEQ ID NO: 67 (OX40Lmincyto-41BB) |
| 161 | Sequence encoding SEQ ID NO: 68 (CD86 WT) |
| 162 | Sequence encoding SEQ ID NO: 69 (CD86delP276) |
| 163 | Sequence encoding SEQ ID NO: 70 (CD86mincyto) |
| 164 | Sequence encoding SEQ ID NO: 71 (CD86mincyto-IL18RAP) |
| 165 | Sequence encoding SEQ ID NO: 72 (CD86-IL18RAP) |
| 166 | Sequence encoding SEQ ID NO: 73 (CD86IgV-41BBL-OX40) |
| 167 | Sequence encoding SEQ ID NO: 74 (RANK WT) |
| 168 | Sequence encoding SEQ ID NO: 75 (RANKmincyto) |
| 169 | Sequence encoding SEQ ID NO: 76 (RANK-OX40) |
| 170 | Sequence encoding SEQ ID NO: 77 (RANK-41BB) |
| 171 | Sequence encoding SEQ ID NO: 78 (RANK-IL18RAP) |
| 172 | Sequence encoding SEQ ID NO: 80 (Q8) |
| 173 | Sequence encoding SEQ ID NO: 81 (eGFP) |
| 185 | Sequence encoding SEQ ID NO: 178 (41BBL-13W-OX40) |
| 186 | Sequence encoding SEQ ID NO: 179 (41BBL-OX40-IL2RB) |
| 187 | Sequence encoding SEQ ID NO: 180 (CD70) |
| 188 | Sequence encoding SEQ ID NO: 181 (CD70-mincyto) |
| 189 | Sequence encoding SEQ ID NO: 182 (CD70-mincyto-OX40) |
| 190 | Sequence encoding SEQ ID NO: 183 (CD70EC-41BBL-OX40) |

TABLE 21
Additional sequences

| SEQ | | | |
|---|---|---|---|
| ID | | | |
| NO: | Name | Description | Sequence |
| 184 | CD19.BB.Z | anti CD19 | MALPVTALLLPLALLLHAARPDIQMTQTTSS |
| | | scFv linked to | LSASLGDRVTISCRASQDISKYLNWYQQKP |
| | | CD8 stalk and | DGTVKLLIYHTSRLHSCVPSRFSGSGSGTD |
| | | transmembran | YSLTISNLEQEDIATYFCQQGNTLPYTFGG |
| | | e with 41BB | GTKLEITGGGGSGGGGSGGGGSEVKLQE |
| | | and CD3z | SGPGLVAPSQSLSVTCTVSGVSLPDYGVS |
| | | intracellular | WIRQPPRKCLEWLGVIWGSETTYYNSALK |
| | | signaling | SRLTIIKDNSKSQVFLKMNSLQTDDTAIYYC |
| | | domains | AKHYYYGGSYAMDYWGQGTSVTVSSTTT |
| | | | PAPRPPTPAPTIASQPLSLRPEACRPAAGG |
| | | | AVHTRGLDFACDIYIWAPLAGTCGVLLLSLV |
| | | | ITLYCKRGRKKLLYIFKQPFMRPVQTTQEE |
| | | | DGCSCRFPEEEEGGCELRVKFSRSADAPA |
| | | | YKQGQNQLYNELNLGRREEYDVLDKRRGR |
| | | | DPEMGGKPRRKNPQEGLYNELQKDKMAE |
| | | | AYSEIGMKGERRRGKGHDGLYQGLSTATK |
| | | | DTYDALHMQALPPR |
| 191 | sgRNA | | ACAAAACUGUGCUAGACAUG |
| 192 | sgRNA | | GAGAAUCAAAAUCGGUGAAU |
| 193 | sgRNA | | CUCUCAGCUGGUACACGGCA |
| 194 | sgRNA | | ACCCGAGGUCGCUGUGUUUG |
| 195 | sgRNA | | AGGUCGCUGUGUUUGAGCCA |
| 196 | sgRNA | | CGACCACGUGGAGCUGAGCU |
| 197 | sgRNA | | GAUACUGCCUGAGCAGCCGC |
| 198 | NY-ESO-1 | | MSIGLLCCAALSLLWAGPVNAGVTQTPKFQ |
| | TRB with | | VLKTGQSMTLQCAQDMNHEYMSWYRQDP |
| | furin site | | GMGLRLIHYSVGAGITDQGEVPNGYNVSR |
| | | | STTEDFPLRLLSAAPSQTSVYFCASSYVGN |
| | | | TGELFFGEGSRLTVLEDLKNVFPPEVAVFE |
| | | | PSEAEISHTQKATLVCLATGFYPDHVELSW |
| | | | WVNGKEVHSGVSTDPQPLKEQPALNDSRY |
| | | | CLSSRLRVSATFWQNPRNHFRCQVQFYGL |
| | | | SENDEWTQDRAKPVTQIVSAEAWGRADC |
| | | | GFTSESYQQGVLSATILYEILLGKATLYAVL |
| | | | VSALVLMAMVKRKDSRGRAKRS |
| 199 | NY-ESO-1 | | METLLGLLILWLQLQWVSSKQEVTQIPAAL |
| | TRA | | SVPEGENLVLNCSFTDSAIYNLQWFRQDP |
| | | | GKGLTSLLLIQSSQREQTSGRLNASLDKSS |
| | | | GRSTLYIAASQPGDSATYLCAVRPLYGGSY |
| | | | IPTFGRGTSLIVHPYIQNPDPAVYQLRDSKS |
| | | | SDKSVCLFTDFDSQTNVSQSKDSDVYITDK |
| | | | TVLDMRSMDFKSNSAVAWSNKSDFACANA |
| | | | FNNSIIPEDTFFPSPESSCDVKLVEKSFETD |
| | | | TNLNFQNLSVIGFRILLLKVAGFNLLMTLRL |
| | | | WSS |
| 200 | NY-ESO-1 | | ATGTCTATCGGCCTGCTGTGTTGTGCCG |
| | TRB DNA | | CTCTGTCTCTGCTTTGGGCCGGACCTGTT |
| | | | AATGCCGGCGTGACCCAGACACCTAAGT |
| | | | TCCAGGTGCTGAAAACCGGCCAGAGCAT |
| | | | GACCCTGCAGTGCGCCCAGGATATGAAC |
| | | | CACGAGTACATGAGCTGGTACAGACAGG |
| | | | ACCCTGGCATGGGCCTGAGACTGATCCA |
| | | | CTATTCTGTCGGAGCCGGCATCACCGAC |
| | | | CAGGGCGAAGTTCCTAATGGCTACAACG |
| | | | TGTCCAGAAGCACCACCGAGGACTTCCC |
| | | | ACTGAGACTGCTGTCTGCCGCTCCTAGC |
| | | | CAGACCAGCGTGTACTTTTGTGCCAGCA |
| | | | GCTACGTGGGCAACACCGGCGAGCTGTT |
| | | | TTTTGGCGAGGGCAGCAGACTGACCGTG |
| | | | CTGGAAGATCTGAAGAACGTGTTCCCAC |
| | | | CAGAGGTGGCCGTGTTCGAGCCTTCTGA |
| | | | GGCCGAGATCAGCCACACACAGAAAGCC |
| | | | ACACTCGTGTGTCTGGCCACCGGCTTCT |
| | | | ATCCCGATCACGTGGAACTGTCTTGGTG |
| | | | GGTCAACGGCAAAGAGGTGCACAGCGG |
| | | | CGTCAGCACAGATCCCCAGCCTCTGAAA |
| | | | GAACAGCCCGCTCTGAACGACAGCCGGT |
| | | | ACTGTCTGTCCTCCAGACTGAGAGTGTC |
| | | | CGCCACCTTCTGGCAGAACCCCAGAAAC |
| | | | CACTTCAGATGCCAGGTGCAGTTCTACG |
| | | | GCCTGAGCGAGAACGATGAGTGGACCCA |
| | | | GGATAGAGCCAAGCCTGTGACACAGATC |
| | | | GTGTCTGCCGAAGCCTGGGGCAGAGCC |
| | | | GATTGTGGCTTTACCAGCGAGAGCTACC |
| | | | AGCAGGGCGTTCTGTCTGCCACCATCCT |
| | | | GTACGAGATCCTGCTGGGCAAAGCCACT |
| | | | CTGTACGCCGTGCTGGTGTCTGCCCTGG |
| | | | TGCTGATGGCCATGGTCAAGCGGAAGGA |
| | | | CAGTAGAGGCAGAGCCAAGAGAAGC |
| 201 | NY-ESO-1 | | ATGGAAACACTGCTGGGCCTGCTGATCC |
| | TRA DNA | | TGTGGCTGCAACTGCAATGGGTGTCCTC |
| | | | CAAGCAAGAAGTGACTCAGATCCCAGCC |
| | | | GCTCTGAGTGTGCCAGAGGGCGAAAACC |
| | | | TGGTCCTGAACTGCAGCTTCACCGACAG |
| | | | CGCCATCTACAACCTGCAGTGGTTCAGA |
| | | | CAAGATCCCGGCAAGGGACTGACCAGCC |
| | | | TGCTGCTGATTCAGAGCAGCCAGAGAGA |
| | | | GCAGACCTCCGGCAGACTGAATGCCAGC |
| | | | CTGGATAAGAGCAGCGGCCGGTCTACAC |
| | | | TGTATATCGCCGCATCTCAGCCTGGCGA |
| | | | TAGCGCCACATATCTGTGTGCCGTGCGA |
| | | | CCTCTGTACGGCGGCAGCTACATCCCTA |
| | | | CATTTGGCAGAGGCACCAGCCTGATCGT |
| | | | GCACCCCTACATTCAGAACCCCGATCCT |
| | | | GCCGTGTATCAGCTGAGAGACAGCAAGT |
| | | | CCAGCGACAAGAGCGTGTGCCTGTTCAC |
| | | | CGACTTCGACTCCCAGACCAATGTGTCC |
| | | | CAGAGCAAGGACAGCGACGTGTACATCA |
| | | | CCGATAAGACAGTGCTGGACATGCGGAG |
| | | | CATGGACTTCAAGAGCAACAGCGCCGTG |
| | | | GCCTGGTCCAACAAGAGCGATTTCGCCT |
| | | | GCGCCAACGCCTTCAACAACAGCATTATC |
| | | | CCCGAGGACACATTCTTCCCAAGTCCTG |
| | | | AGAGCAGCTGCGACGTGAAGCTGGTGGA |
| | | | AAAGAGCTTCGAGACAGACACCAACCTG |
| | | | AACTTCCAGAACCTGAGCGTGATCGGCT |
| | | | TCCGGATTCTGCTGCTCAAGGTGGCCGG |
| | | | CTTCAACCTGCTGATGACCCTGAGACTCT |
| | | | GGTCCAGC |
| 202 | MSCV | | tgaaagaccccacctgtaggtttggcaagctagcttaagtaa |
| | promoter | | cgccattttgcaaggcatggaaaatacataactgagaatag |
| | | | agaagttcagatcaaggttaggaacagagagacagcaga |
| | | | atatgggccaaacaggatatctgtggtaagcagttcctgccc |
| | | | cggctcagggccaagaacagatggtccccagatgcggtcc |
| | | | cgccctcagcagtttctagagaaccatcagatgtttccaggg |
| | | | tgccccaaggacctgaaaatgaccctgtgccttatttgaact |
| | | | aaccaatcagttcgcttctcgcttctgttcgcgcgtttctgctcc |
| | | | ccgagctcaataaaagagcccacaacccctcact |
| 203 | MMLV | | aatgaaagaccccacctgtaggtttggcaagctagcttaagt |
| | promoter | | aacgccattttgcaaggcatggaaaaatacataactgaga |
| | | | atagaaaagttcagatcaaggtcaggaacagatggaaca |
| | | | gctgaatatgggccaaacaggatatctgtggtaagcagttcc |
| | | | tgccccggctcagggccaagaacagatggaacagctgaa |
| | | | tatgggccaaacaggatatctgtggtaagcagttcctgcccc |
| | | | ggctcagggccaagaacagatggtccccagatgcggtcc |
| | | | agccctcagcagtttctagagaaccatcagatgtttccaggg |
| | | | tgccccaaggacctgaaatgaccctgtgccttatttgaacta |
| | | | accaatcagttcgcttctcgcttctgttcgcgcgcttatgctccc |
| | | | cgagctcaataaaagagcccacaacccctcactcggggc |
| | | | gccagtcctccgattgactgagtcgcccgggtacccgtgtat |
| | | | ccaataaaccctcttgcagttgcatccgacttgtggtctcgctg |
| | | | ttccttgggagggtctcctctgagtgattgactacccgtcagc |
| | | | gggggtctttcatt |
| 204 | EF1α | | gctccggtgcccgtcagtgggcagagcgcacatcgcccac |
| | promoter | | agtccccgagaagttggggggaggggtcggcaattgaacc |
| | | | ggtgcctagagaaggtggcgcggggtaaactgggaaagt |
| | | | gatgtcgtgtactggctccgcctttttcccgagggtgggggag |
| | | | aaccgtatataagtgcagtagtcgccgtgaacgttctttttcgc |
| | | | aacgggtttgccgccagaacacaggtaagtgccgtgtgtgg |
| | | | ttcccgcgggcctggcctctttacgggttatggcccttgcgtgc |
| | | | cttgaattacttccacgcccctggctgcagtacgtgattcttgat |
| | | | cccgagcttcgggttggaagtgggtgggagagttcgaggcc |
| | | | ttgcgcttaaggagccccttcgcctcgtgcttgagttgaggcct |
| | | | ggcttgggcgctggggccgccgcgtgcgaatctggtggca |
| | | | ccttcgcgcctgtctcgctgctttcgataagtctctagccattta |
| | | | aaatttttgatgacctgctgcgacgctttttttctggcaagatagt |
| | | | cttgtaaatgcgggccaagatctgcacactggtatttcggttttt |
| | | | ggggccgcgggcggcgacggggcccgtgcgtcccagcg |
| | | | cacatgttcggcgaggcggggcctgcgagcgcggccacc |
| | | | gagaatcggacgggggtagtctcaagctggccggcctgct |
| | | | ctggtgcctggcctcgcgccgccgtgtatcgccccgccctgg |
| | | | gcggcaaggctggcccggtcggcaccagttgcgtgagcg |
| | | | gaaagatggccgcttcccggccctgctgcagggagctcaa |
| | | | aatggaggacgcggcgctcgggagagcgggcgggtgag |
| | | | tcacccacacaaaggaaaagggcctttccgtcctcagccgt |
| | | | cgcttcatgtgactccacggagtaccgggcgccgtccaggc |
| | | | acctcgattagttctcgagcttttggagtacgtcgtctttaggttg |
| | | | gggggaggggttttatgcgatggagtttccccacactgagtg |
| | | | ggtggagactgaagttaggccagcttggcacttgatgtaatt |
| | | | ctccttggaatttgccctttttgagtttggatcttggttcattctcaa |
| | | | gcctcagacagtggttcaaagtttttttcttccatttcaggtgtcg |
| | | | tga |
| 205 | MND | | tttatttagtctccagaaaaaggggggaatgaaagacccca |
| | promoter | | cctgtaggtttggcaagctaggatcaaggttaggaacagag |
| | | | agacagcagaatatgggccaaacaggatatctgtggtaag |
| | | | cagttcctgccccggctcagggccaagaacagttggaaca |
| | | | gcagaatatgggccaaacaggatatctgtggtaagcagttc |
| | | | ctgccccggctcagggccaagaacagatggtccccagatg |
| | | | cggtcccgccctcagcagtttctagagaaccatcagatgtttc |
| | | | cagggtgccccaaggacctgaaatgaccctgtgccttatttg |
| | | | aactaaccaatcagttcgcttctcgcttctgttcgcgcgcttctg |
| | | | ctccccgagctcaataaaagagccca |
| 206 | P2A | | ATNFSLLKQAGDVEENPGP |
| 207 | P2A DNA | | gccaccaatttcagcctgctgaaacaggctggcgacgtgg |
| | | | aagagaaccctgggccc |
| 208 | T2A | | EGRGSLLTCGDVEENPGP |
| 209 | T2A DNA | | gaaggccgcggcagcctgctgacctgcggagacgtggaa |
| | | | gaaaaccctggcccg |
| 210 | NY-ESO-1 | | CAVRPLYGGSYIPTF |
| | CDR3 TCR | | |
| | alpha | | |
| 211 | NY-ESO-1 | | CASSYVGNTGELFF |
| | CDR3 TCR | | |
| | beta | | |
| 212 | EGFR.BB.Z | anti EGFR | QVQLKQSGPGLVQPSQSLSITCTVSGFSLT |
| | | scFv linked to | NYGVHWVRQSPGKGLEWLGVIW |
| | | CD8 stalk and | SGGNTDYNTPFTSRLSINKDNSKSQVFFKM |
| | | transmembrane | NSLQSNDTAIYYCARALTYYDYEF |
| | | with 41BB | AYWGQGTLVTVSAGGGGSGGGGGGGG |
| | | and CD3z | SGGGGSDILLTQSPVILSVSPGER |
| | | intracellular | VSFSCRASQSIGTNIHWYQQRTNGSPRLLI |
| | | signaling | KYASESISGIPSRFSGSGSGTDFTL |
| | | domains | SINSVESEDIADYYCQQNNNWPTTFGAGTK |
| | | | LELKTTTPAPRPPTPAPTIASQPLSLRPEAC |
| | | | RPAAGGAVHTRGLDFACDIYIWAPLAGT |
| | | | CGVLLLSLVITLYCKRGRKKLLYIFKQPFMR |
| | | | PVQTTQEEDGCSCRFPEEEEGGCEL |
| | | | RVKFSRSADAPAYQQGQNQLYNELNLGRR |
| | | | EEYDVLDKRRGRDPEMGGKPRR |
| | | | KNPQEGLYNELQKDKMAEAYSEIGMKGER |
| | | | RRGKGHDGLYQGLSTATKDTYDA |
| | | | LHMQALPPR |
| 213 | CD19.28.z | anti CD19 | DIQMTQTTSSLSASLGDRVTISCRASQDISK |
| | | scFv linked to | YLNWYQQKPDGTVKLLIYHTSRLHSGVPSR |
| | | CD28 stalk | FSGSGSGTDYSLTISNLEQEDIATYFCQQG |
| | | and | NTLPYTFGGGTKLEITGSTSGSGKPGSGEG |
| | | transmembrane | STKGEVKLQESGPGLVAPSQSLSVTCTVS |
| | | with CD28 | GVSLPDYGVSWIRQPPRKGLEWLGVIWGS |
| | | and CD3z | ETTYYNSALKSRLTIIKDNSKSQVFLKMNSL |
| | | intracellular | QTDDTAIYYCAKHYYYGGSYAMDYWGQG |
| | | signaling | TSVTVSSAAAGTTTTPAPRPPTPAPTIASQP |
| | | domains | LSLRPEACRPAAGGAVHTRGLDFACDRRP |
| | | | PSKPFWVLVVVGGVLACYSLLVTVAFIIFWV |
| | | | RSKRSRLLHSDYMNMTPRRPGPTRKHYQP |
| | | | YAPPRDFAAYRSASLRVKFSRSADAPAYQ |
| | | | QGQNQLYNELNLGRREEYDVLDKRRGRD |
| | | | PEMGGKPQRRKNPQEGLYNELQKDKMAE |
| | | | AYSEIGMKGERRRGKGHDGLYQGLSTATK |
| | | | DTYDALHMQALPPR |
| 214 | MUC1 TRA | Anti MUC-1 | METLLGLLILWLQLQWVSSKQEVTQIPAALSVP |
| | | TCR alpha | EGENLVLNCSFTDSAIYNLQWFRQDPGKGLTS |
| | | | LLLIQ |
| | | | SSQREQTSGRLNASLDKSSGRSTLYIAASQPG |
| | | | DSATYLCAVTSSYGKLTFGQGTILTVHPNIQNP |
| | | | DPAVY |
| | | | QLRDSKSSDKSVCLFTDFDSQTSVSQSKDSD |
| | | | VYITDKTVLDMRSMDFKSNSAVAWSNKSDFA |
| | | | CANAFNNS |
| | | | IIPEDTFFPSPESSCDVKLVEKSFETDTNLNFQ |
| | | | NLSVIGFRILLLKVAGFNLLMTLRLWSS |
| 215 | MUC1 TRB | Anti MUC-1 | MATRLLCCVVLCLLGEELIDARVTQTPRHKVTE |
| | | TCR beta | MGQEVTMRCQPILGHNTVFWYRQTMMQGLE |
| | | | LLAYFRN |
| | | | RAPLDDSGMPKDRFSAEMPDATLATLKIQPSE |
| | | | PRDSAVYFCASGLGEGRGYTFGSGTRLTVVE |
| | | | DLNKVFP |
| | | | PEVAVFEPSEAEISHTQKATLVCLATGFFPDHV |
| | | | ELSWWVNGKEVHSGVSTDPQPLKEQPALNDS |
| | | | RYCLSS |
| | | | RLRVSATFWQNPRNHFRCQVQFYGLSENDE |
| | | | WTQDRAKPVTQIVSAEAWGRADCGFTSVSYQ |
| | | | QGVLSATIL |
| | | | YEILLGKATLYAVLVSALVLMAMVKRKDF |
| 216 | MUC1 TRA | Anti MUC-1 | CAVTSSYGKLTF |
| | CDR3 | TCR alpha | |
| | | CDR3 | |
| 217 | MUC1 TRB | Anti MUC-1 | CASGLGEGRGYTF |
| | CDR3 | TCR beta | |
| | | CDR3 | |
| 218 | MAGE-A4 | Anti MAGE- | METLLGLLILWLQLQWVSSKQEVTQIPAALSVP |
| | TRA | A4 TCR alpha | EGENLVLNCSFTDSAIYNLQWFRQDPG |
| | | | KGLTSLLLIQSSQREQTSGRLNASLDKSSGRS |
| | | | TLYIAASQPGDSATYLCAVGGYSTLTFG |
| | | | KGTVLLVSPDNIQNPDPAVYQLRDSKSSDKSV |
| | | | CLFTDFDSQTNVSQSKDSDVYITDKTVL |
| | | | DMRSMDFKSNSAVAWSNKSDFACANAFNNSII |
| | | | PEDTFFPSPESSCDVKLVEKSFETDTNL |
| | | | NFQNLSVIGFRILLLKVAGFNLLMTLRLWSS |
| 219 | MAGE-A4 | Anti MAGE- | MSISLLCCAAFPLLWAGPVNAGVTQTPKFRILK |
| | TRB | A4 TCR beta | IGQSMTLQCAQDMNHNYMYWYRQDPGM |
| | | | GLKLIYYSVGAGITDKGEVPNGYNVSRSTTED |
| | | | FPLRLELAAPSQTSVYFCASSYSRWSPL |
| | | | HFGNGTRLTVTEDLNKVFPPEVAVFEPSEAEIS |
| | | | HTQKATLVCLATGFFPDHVELSWWVNG |
| | | | KEVHSGVSTDPQPLKEQPALNDSRYCLSSRL |
| | | | RVSATFWQNPRNHFRCQVQFYGLSENDEW |
| | | | TQDRAKPVTQIVSAEAWGRADCGFTSVSYQQ |
| | | | GVLSATILYEILLGKATLYAVLVSALVLM |
| | | | AMVKRKDF |
| 220 | MAGE-A4 | Anti MAGE- | CAVGGYSTLTF |
| | TRA CDR3 | A4 TCR alpha | |
| | | CDR3 | |
| 221 | MAGE-A4 | Anti MAGE- | CASSYSRWSPLHF |
| | TRB CDR3 | A4 TCR beta | |
| | | CDR3 | |
| 222 | NKG2D.z | | MRVKFSRSADAPAYQQGQNQLYNELNLGRRE |
| | | | EYDVLDKRRGRDPEMGGKPQRRKNPQEGLY |
| | | | NELQKDKMAEAYSEIGMKGERRRGKGHDGLY |
| | | | QGLSTATKDTYDALHMQALPPREFGWIRGRR |
| | | | SRHSWEMSEFHNYNLDLKKSDFSTRWQKQR |
| | | | CPVVKSKCRENASPFFFCCFIAVAMGIRFIIMV |
| | | | AIWSAVFLNSLFNQEVQIPLTESYCGPCPKNWI |
| | | | CYKNNCYQFFDESKNWYESQASCMSQNASLL |
| | | | KVYSKEDQDLLKLVKSYHWMGLVHIPTNGSW |
| | | | QWEDGSILSPNLLTIIEMQKGDCALYASSFKGY |
| | | | IENCSTPNTYICMQRTV |

### XI. Embodiments

Embodiment 1. A multi-directional signal transducer (MIDIS) protein comprising an extracellular ligand domain, a transmembrane domain, and a heterologous intracellular signaling domain, wherein binding of the extracellular ligand domain to an interaction partner induces multi-directional signaling that comprises a first signaling pathway mediated by the heterologous intracellular signaling domain of the MIDIS protein and a second signaling pathway mediated by an intracellular domain of the interaction partner, wherein the first signaling pathway and the second signaling pathway jointly induce a target biological outcome. This first and second signaling pathways may occur in the same cell.

Embodiment 2. The MIDIS protein of embodiment 1, wherein the target biological outcome is cellular proliferation, cellular survival, cellular differentiation, cellular dedifferentiation, or cellular transdifferentiation, or a combination thereof.

Embodiment 3. The MIDIS protein of embodiment 1, wherein the target biological outcome is immune effector function, an anti-cancer immune response, or a combination thereof.

Embodiment 4. The MIDIS protein of any one of embodiments 1-3, wherein the multi-directional signaling comprises inside-out signaling and outside-in signaling.

Embodiment 5. The MIDIS protein of any one of embodiments 1-4, wherein the extracellular ligand domain comprises an amino acid sequence from an immune co-receptor ligand.

Embodiment 6. The MIDIS protein of any one of embodiments 1-5, wherein the extracellular ligand domain comprises an amino acid sequence from a type I or type II transmembrane protein.

Embodiment 7. The MIDIS protein of any one of embodiments 1-6, wherein the extracellular ligand domain comprises an amino acid sequence from an immune co-stimulatory ligand.

Embodiment 8. The MIDIS protein of any one of embodiments 1-4, wherein the extracellular ligand domain comprises an antigen-binding fragment derived from an antibody.

Embodiment 9. The MIDIS protein of any one of embodiments 1-4, wherein the extracellular ligand domain comprises a short chain variable fragment (scFv), single domain antibody, Fab, Fab′, F(ab′)2, Fv, minibody, diabody, triabody, tetrabody, affibody, ankyrin repeat, darpin, nanobody, avimer, adnectin, anticalin, Fynomer, Kunitz domain, knottin, or β-hairpin mimetic.

Embodiment 10. The MIDIS protein of any one of embodiments 1-4, wherein the extracellular ligand domain comprises an amino acid sequence from a tumor necrosis factor superfamily member, a cytokine, a C-type lectin, an immunoglobulin superfamily member, or an antibody or antigen-binding fragment thereof.

Embodiment 11. The MIDIS protein of any one of embodiments 1-7, wherein extracellular ligand domain comprises an amino acid sequence from 41BBL, OX40L, CD86, RANK, or CD70.

Embodiment 12. The MIDIS protein of any one of embodiments 1-7, wherein the extracellular ligand domain comprises an amino acid sequence from 41BBL.

Embodiment 13. The MIDIS protein of any one of embodiments 1-7 and 10-12, wherein the extracellular ligand domain comprises an amino acid sequence with at least 90%, 95%, 96%, 97%, 98%, 99%, or 100% sequence identity to any one of SEQ ID NOs: 01-06, or 174.

Embodiment 14. The MIDIS protein of any one of embodiments 1-13, wherein the heterologous intracellular signaling domain comprises an amino acid sequence from a tumor necrosis factor receptor superfamily member, a cytokine receptor, or a C-type lectin receptor.

Embodiment 15. The MIDIS protein of any one of embodiments 1-14, wherein the heterologous intracellular signaling domain comprises an amino acid sequence from a type I or type II transmembrane protein.

Embodiment 16. The MIDIS protein of any one of embodiments 1-15, wherein the heterologous intracellular signaling domain comprises an amino acid sequence from a co-stimulatory immune receptor.

Embodiment 17. The MIDIS protein of any one of embodiments 1-16, wherein the heterologous intracellular signaling domain comprises an amino acid sequence from 41BB, NKp80, IL18RAP, CD70, or IL2RB.

Embodiment 18. The MIDIS protein of any one of embodiments 1-17, wherein the heterologous intracellular signaling domain comprises an amino acid sequence from OX40.

Embodiment 19. The MIDIS protein of any one of embodiments 1-18, wherein the heterologous intracellular signaling domain comprises an amino acid sequence with at least 90%, 95%, 96%, 97%, 98%, 99%, or 100% sequence identity to any one of SEQ ID NOs: 07-19, 175, or 176.

Embodiment 20. The MIDIS protein of any one of embodiments 1-19, wherein the interaction partner is 41BB, OX40, CD28, or RANKL.

Embodiment 21. The MIDIS protein of any one of embodiments 1-20, further comprising one or more additional extracellular ligand domains.

Embodiment 22. The MIDIS protein of any one of embodiments 1-21, further comprising one or more additional intracellular signaling domains.

Embodiment 23. The MIDIS protein of any one of embodiments 1-22, wherein N-terminal to C-terminal orientation of the extracellular ligand domain is reversed compared to a wild type amino acid sequence.

Embodiment 24. The MIDIS protein of any one of embodiments 1-23, wherein N-terminal to C-terminal orientation of the heterologous intracellular signaling domain is reversed compared to a wild type amino acid sequence.

Embodiment 25. The MIDIS protein of any one of embodiments 1-24, wherein the MIDIS protein signals as a monomer, a dimer, a trimer, a tetramer, a pentamer, a hexamer, or a multimer upon binding of the extracellular ligand domain to the interaction partner.

Embodiment 26. The MIDIS protein of any one of embodiments 1-25, wherein the interaction partner is expressed by an immune cell.

Embodiment 27. The MIDIS protein of any one of embodiments 1-25, wherein the interaction partner is expressed by a T cell.

Embodiment 28. The MIDIS protein of any one of embodiments 1-27, wherein the interaction partner is an immune co-receptor.

Embodiment 29. The MIDIS protein of any one of embodiments 1-28, wherein the transmembrane domain and the extracellular ligand domain are from the same protein.

Embodiment 30. The MIDIS protein of any one of embodiments 1-29, wherein the MIDIS protein comprises, consists essentially of, or consists of an amino acid sequence with at least 90%, 95%, 97%, 98%, 99%, or 99.5%, or 100% sequence identity to SEQ ID NO: 45-53, 57-65,67, 71-73,76-78, 178-179, or 182-183.

Embodiment 31. The MIDIS protein of any one of embodiments 1-30, wherein the MIDIS protein does not contain an ITAM, an intracellular domain from a TCR signaling complex, or an intracellular domain from a CD3 chain.

Embodiment 32. The MIDIS protein of any one of embodiments 1-30, wherein the MIDIS protein contains a hemITAM but does not contain an ITAM.

Embodiment 33. The MIDIS protein of any one of embodiments 1-31, wherein the MIDIS protein is not phosphorylated upon binding to the interaction partner.

Embodiment 34. The MIDIS protein of any one of embodiments 1-32, wherein the MIDIS protein is phosphorylated upon binding to the interaction partner.

Embodiment 35. The MIDIS protein of any one of embodiments 1-34, wherein the multi-directional signaling comprises at least two, at least three, at least four, or at least five signaling pathways mediated by the heterologous intracellular signaling domain of the MIDIS protein.

Embodiment 36. The MIDIS protein of any one of embodiments 1-35, wherein the multi-directional signaling comprises at least two, at least three, at least four, or at least five signaling pathways mediated by the intracellular domain of the interaction partner.

Embodiment 37. The MIDIS protein of any one of embodiments 1-34, wherein the multi-directional signaling is bi-directional signaling involving one signaling pathway mediated by the heterologous intracellular signaling domain of the MIDIS protein and one signaling pathway mediated by the intracellular domain of the interaction partner.

Embodiment 38. The MIDIS protein of any one of embodiments 1-37 for use in a population of cells engineered to express the MIDIS protein, wherein the multi-directional signaling induces the target biological outcome.

Embodiment 39. A method of making a population of engineered cells, comprising: (a) expressing the MIDIS protein of any one of embodiments 1-37 in at least one cell in the population of engineered cells, and (b) culturing the population of engineered cells in a condition suitable for expansion of the population of engineered cells.

Embodiment 40. A method of enriching a population of cells for cells that respond to an antigen, comprising: (a) expressing the MIDIS protein of any one of embodiments 1-37 in at least one cell in the population of cells; and (b) culturing the population of cells with the antigen or cells that present the antigen.

Embodiment 41. An engineered cell that expresses the MIDIS protein of any one of embodiments 1-37.

Embodiment 42. The engineered cell of embodiment 41 for use in treating a subject in need thereof.

Embodiment 43. A method of treating a subject in need thereof, comprising administering to the subject the engineered cell of embodiment 41.

Embodiment 44. A polynucleotide encoding the MIDIS protein of any one of embodiments 1-37.

Embodiment 45. The polynucleotide of embodiment 4244, wherein the polynucleotide encodes an exogenous antigen-recognition receptor.

Embodiment 46. The polynucleotide of embodiment 45, wherein the exogenous antigen-recognition receptor is a chimeric antigen receptor, a T cell receptor, an alpha-beta T cell receptor, or a gamma-delta T cell receptor.

Embodiment 47. The polynucleotide of any one of embodiments 45-46, wherein the MIDIS protein and the exogenous antigen recognition receptor are expressed in one transcript.

Embodiment 48. The polynucleotide of any one of embodiments 45-46, wherein the MIDIS protein and the exogenous antigen recognition receptor are expressed in one transcript that encodes a self-cleaving peptide.

Embodiment 49. A vector comprising the polynucleotide of any one of embodiments 44-48.

Embodiment 50. The vector of embodiment 49 for use in treating a subject in need thereof.

Embodiment 51. A method of treatment, comprising administering to a subject in need thereof the vector of embodiment 50.

Embodiment 52. An engineered cell that expresses a multi-directional signal transducer (MIDIS) protein comprising an extracellular ligand domain, a transmembrane domain, and a heterologous intracellular signaling domain, wherein binding of the extracellular ligand domain to an interaction partner expressed by a cell induces multi-directional signaling that comprises a first signaling pathway mediated by the heterologous intracellular signaling domain of the MIDIS protein and a second signaling pathway mediated by an intracellular domain of the interaction partner, wherein the first signaling pathway modulates a target biological function of the engineered cell and the second signaling pathway modulates a target biological function of the cell that expresses the interaction partner.

Embodiment 53. The engineered cell of embodiment 52, wherein the target biological function of the engineered cell is induced.

Embodiment 54. The engineered cell of embodiment 52, wherein the target biological function of the engineered cell is reduced.

Embodiment 55. The engineered cell of any one of embodiments 52-54, wherein the target biological function of the engineered cell comprises survival, proliferation, immune effector function, a cytotoxic response, an anti-cancer response, cellular differentiation, cellular dedifferentiation, or cellular trans-differentiation.

Embodiment 56. The engineered cell of any one of embodiments 52-55, wherein upon binding of the extracellular ligand domain to the interaction partner, the target biological function of the engineered cell is modulated for at least 10% longer than a corresponding cell that does not express the MIDIS protein.

Embodiment 57. The engineered cell of any one of embodiments 52-56, wherein upon binding of the extracellular ligand domain to the interaction partner, the target biological function of the engineered cell is increased at least 10% or decreased at least 10% compared to a corresponding cell that does not express the MIDIS protein.

Embodiment 58. An engineered cell that expresses a multi-directional signal transducer (MIDIS) protein comprising an extracellular ligand domain, a transmembrane domain, and a heterologous intracellular signaling domain, wherein binding of the extracellular ligand domain to an interaction partner expressed by a cell induces multi-directional signaling that comprises a first signaling pathway mediated by the heterologous intracellular signaling domain of the MIDIS protein and a second signaling pathway mediated by an intracellular domain of the interaction partner, wherein the multi-directional signaling modulates a target biological function of the cell that expresses the interaction partner and does not induce a cytotoxic response against the cell that expresses the interaction partner.

Embodiment 59. The engineered cell of any one of embodiments 52-58, wherein the target biological function of the cell that expresses the interaction partner is induced.

Embodiment 60. The engineered cell of any one of embodiments 52-58, wherein the target biological function of the cell that expresses the interaction partner is reduced.

Embodiment 61. The engineered cell of any one of embodiments 52-60, wherein the target biological function of the cell that expresses the interaction partner comprises survival, proliferation, immune effector function, an anti-cancer response, cellular differentiation, cellular dedifferentiation, or cellular trans-differentiation.

Embodiment 62. The engineered cell of any one of embodiments 52-61, wherein the target biological function of the cell that expresses the interaction partner comprises a cytotoxic response against a cancer cell.

Embodiment 63. The engineered cell of any one of embodiments 52-62, wherein the cell that expresses the interaction partner is an immune cell, a T cell, an alpha-beta T cell, a gamma-delta T cell, CD4+ T cell, CD8+ T cell, a T effector cell, a lymphocyte, a B cell, an NK cell, an NKT cell, a myeloid cell, a monocyte, a macrophage, a neutrophil, a fibroblast, a keratinocyte, a mesenchymal stem cell, an endothelial cell, or a stromal cell.

Embodiment 64. The engineered cell of any one of embodiments 52-63, wherein the engineered cell is an immune cell, a T cell, an alpha-beta T cell, a gamma-delta T cell, a Jurkat cell, CD4+ T cell, CD8+ T cell, a T effector cell, a lymphocyte, a B cell, an NK cell, an NKT cell, a myeloid cell, a monocyte, a macrophage, or a neutrophil.

Embodiment 65. The engineered cell of any one of embodiments 52-64, wherein the engineered cell and the cell that expresses the interaction partner are of the same cell type.

Embodiment 66. The engineered cell of any one of embodiments 52-64, wherein the engineered cell and the cell that expresses the interaction partner are different cell types.

Embodiment 67. The engineered cell of any one of embodiments 52-66, wherein the engineered cell or the cell that expresses the interaction partner is a mammalian cell.

Embodiment 68. The engineered cell of any one of embodiments 52-67, wherein the engineered cell or the cell that expresses the interaction partner is a human cell.

Embodiment 69. The engineered cell of any one of embodiments 52-68, wherein the engineered cell expresses an exogenous antigen-recognition receptor.

Embodiment 70. The engineered cell of embodiment 69, wherein the exogenous antigen-recognition receptor is a transgenic TCR, an alpha-beta TCR, a gamma-delta TCR, or a chimeric antigen receptor.

Embodiment 71. The engineered cell of embodiment 69, wherein the exogenous antigen-recognition receptor is a gamma-delta TCR.

Embodiment 72. The engineered cell of embodiment 71, wherein the gamma-delta TCR comprises: (a) a γ-chain selected from the group consisting of γ2, γ3, γ4, γ5, γ8, γ9, and γ11; (b) a δ-chain selected from the group consisting of δ1, δ2, δ3, and δ5; or (c) any combination of (a) and (b).

Embodiment 73. The engineered cell of embodiment 72, wherein the γ-chain is the γ9 and the δ-chain is the 62.

Embodiment 74. The engineered cell of any one of embodiments 71-73, wherein the gamma-delta TCR comprises a γ-chain that comprises a CDR sequence with at least about 65%, 70%, 75%, 80%, 85%, 90%, 95%, 97%, 99%, or 100% identity to any one of SEQ ID NOs: 85, 86, 87, 94, 95, 96, 101, 113, 115, 117, 119, 127, 130, and wherein the gamma-delta TCR comprises a 6-chain that comprises a CDR sequence with at least about 65%, 70%, 75%, 80%, 85%, 90%, 95%, 97%, 99%, or 100% identity to one of SEQ ID NOs: 82, 83, 84, 97, 98, 99, 100, 102, 114, 116, 118, 126, 131.

Embodiment 75. The engineered cell of any one of embodiments 82-74, wherein the engineered cell further comprises a deletion or disruption of one or more genes.

Embodiment 76. The engineered cell of any one of embodiments 52-75, wherein the engineered cell further comprises a deletion or disruption of a TRAC, TCRB, or immune checkpoint gene.

Embodiment 77. The engineered cell of any one of embodiments 52-76, wherein the engineered cell expresses the interaction partner.

Embodiment 78. The engineered cell of any one of embodiments 52-77, wherein the multi-directional signaling comprises at least two, at least three, at least four, or at least five signaling pathways mediated by the heterologous intracellular signaling domain of the MIDIS protein.

Embodiment 79. The engineered cell of any one of embodiments 52-78, wherein the multi-directional signaling comprises at least two, at least three, at least four, or at least five signaling pathways mediated by the intracellular domain of the interaction partner.

Embodiment 80. The engineered cell of any one of embodiments 52-77, wherein the multi-directional signaling is bi-directional signaling.

Embodiment 81. The engineered cell of any one of embodiments 52-80 for use in treating a subject in need thereof.

Embodiment 82. A method of treatment, comprising administering to a subject in need thereof the engineered cell of any one of embodiments 82-80.

Embodiment 83. A method of making a population of engineered cells, comprising culturing a population of engineered cells comprising a plurality of the engineered cell of any one of embodiments 52-77 in a suitable condition.

Embodiment 84. A population of engineered cells comprising at least one cell that expresses a multi-directional signal transducer (MIDIS) protein and at least one cell that expresses an interaction partner, wherein the MIDIS protein comprises: an extracellular ligand domain, wherein the extracellular ligand domain is capable of binding to the interaction partner; a transmembrane domain; and a heterologous intracellular signaling domain; wherein binding of the extracellular ligand domain to the interaction partner induces multi-directional signaling that comprises a first signaling pathway mediated by the heterologous intracellular signaling domain of the MIDIS protein and a second signaling pathway mediated by an intracellular domain of the interaction partner.

Embodiment 85. The population of engineered cells of embodiment 84, wherein at least one cell in the population of engineered cells expresses an exogenous antigen-recognition receptor.

Embodiment 86. The population of engineered cells of embodiment 85, wherein the exogenous antigen-recognition receptor is a chimeric antigen receptor, a T cell receptor, an alpha-beta T cell receptor, or a gamma-delta T cell receptor.

Embodiment 87. The population of engineered cells of any one of embodiments 85-86, wherein upon exposure of the population of engineered cells to cells that express or present an antigen that binds to the exogenous antigen-recognition receptor, proliferation of the population of engineered cells is increased by at least 10% compared to a corresponding population of engineered cells that do not express the MIDIS protein.

Embodiment 88. The population of engineered cells of any one of embodiments 85-87, wherein upon exposure of the population of engineered cells to cells that express or present an antigen that binds to the exogenous antigen-recognition receptor, killing of the cells that express or present the antigen is increased by at least 10% compared to killing when the cells that express or present the antigen are exposed to a corresponding population of engineered cells that do not express the MIDIS protein.

Embodiment 89. The population of engineered cells of any one of embodiments 85-88, wherein upon exposure of the population of engineered cells to cells that express or present an antigen that binds to the exogenous antigen-recognition receptor, an ability of the engineered cells to kill at least 50% of the cells that express or present the antigen persists at least 3 days longer than a corresponding population of engineered cells that do not express the MIDIS protein.

Embodiment 90. The population of engineered cells of any one of embodiments 85-89, wherein upon exposure of the population of engineered cells to cells that express or present an antigen that binds to the exogenous antigen-recognition receptor for at least 5 days, expression of an exhaustion marker by the population of engineered cells is at least 10% lower than a corresponding population of engineered cells that do not express the MIDIS protein.

Embodiment 91. The population of engineered cells of any one of embodiments 85-90, wherein upon exposure of the population of engineered cells to cells that express or present an antigen that binds to the exogenous antigen-recognition receptor, production of an immune effector molecule by the population of engineered cells is at least 10% higher than a corresponding population of engineered cells that do not express the MIDIS protein.

Embodiment 92. The population of engineered cells of any one of embodiments 84-91, wherein at least one cell in the population of engineered cells expresses the MIDIS protein and the interaction partner.

Embodiment 93. A method of treating a subject in need thereof, comprising administering to the subject the population of engineered cells of any one of embodiments 84-92.

Embodiment 94. The population of engineered cells of any one of embodiments 84-93, wherein the multi-directional signaling comprises at least two, at least three, at least four, or at least five signaling pathways mediated by the heterologous intracellular signaling domain of the MIDIS protein.

Embodiment 95. The population of engineered cells of any one of embodiments 84-94, wherein the multi-directional signaling comprises at least two, at least three, at least four, or at least five signaling pathways mediated by the intracellular domain of the interaction partner.

Embodiment 96. The population of engineered cells of any one of embodiments 84-93, wherein the multi-directional signaling is bi-directional signaling.

Embodiment 97. A method of making a population of engineered cells, comprising expressing a multi-directional signal transducer (MIDIS) protein in at least one cell in the population of engineered cells, and culturing the population of engineered cells in a condition suitable for expansion of the population of engineered cells; wherein the MIDIS protein comprises: an extracellular ligand domain, wherein the extracellular ligand domain is capable of binding to an interaction partner expressed by at least one cell in the population of engineered cells; a transmembrane domain; and a heterologous intracellular signaling domain; wherein binding of the extracellular ligand domain to the interaction partner induces multi-directional signaling that comprises a first signaling pathway mediated by the heterologous intracellular signaling domain of the MIDIS protein and a second signaling pathway mediated by an intracellular domain of the interaction partner.

Embodiment 98. The method of any one of embodiments 43, 51, 76, and 87, wherein the subject has cancer.

Embodiment 99. The method of any one of embodiments 43, 82, 93, and 98, wherein the engineered cell or the population of engineered cells is autologous to the subject.

Embodiment 100. The method of any one of embodiments 43, 82, 93, and 98, the engineered cell or the population of engineered cells is allogeneic, HLA matched, HLA-mismatched, or haploidentical to the subject.

Embodiment 101. A chimeric bidirectional signaling transmembrane protein able to transduce at least two intracellular signals, said protein comprising:
an extracellular ligand domain, able to interact with the extracellular domain of its interaction partner
a transmembrane domain, and
a heterologous intracellular signaling domain transducing a first signal after binding of the extracellular ligand domain to its interaction partner,
   wherein the second intracellular signal is transduced via the intracellular domain of the interaction partner and
   wherein the chimeric protein is not a protein comprising or consisting of the extracellular ligand domain and the transmembrane domain of the ICOSL and the heterologous intracellular signaling domain of 41BB.

Embodiment 102. The chimeric protein of embodiment 101, wherein the at least two intracellular signals are generated in one single cell.

Embodiment 103. The chimeric protein of embodiment 101 or 102, wherein the interaction partner comprises:
an extracellular domain able to interact with the extracellular ligand domain of the chimeric protein,
a transmembrane domain, and
an intracellular domain transducing a second signal after binding of the extracellular domain of the interaction partner to the extracellular ligand domain of the chimeric protein.

Embodiment 104. The chimeric protein of any one of embodiments 101-103, wherein the at least two intracellular signals contribute to an improvement of a biological parameter and/or function of a cell expressing the chimeric protein and/or an improvement of a biological parameter and/or function induced by such a cell.

Embodiment 105. The chimeric protein of any one of embodiments 101-104, wherein
a. The extracellular ligand domain is from or derived from a type I transmembrane protein and the heterologous intracellular signaling domain is from or derived from a type 11 transmembrane protein or
b. The extracellular ligand domain is from or derived from a type 11 transmembrane protein and the heterologous intracellular signaling domain is from or derived from a type I transmembrane protein.

Embodiment 106. The chimeric protein of any one of embodiments 102-105, wherein the cell is an immune cell, preferably a T or NK cell.

Embodiment 107. The chimeric protein of any one of embodiments 104-106, wherein the biological parameter and/or function is selected from proliferation, cellular survival, cytotoxicity, antitumor activity, persistence and/or tumor cell killing,

Embodiment 108. The chimeric protein of any one of embodiments 101-107, wherein:
the extracellular ligand domain comprises an amino acid sequence from a tumor necrosis factor superfamily member, a cytokine, a C-type lectin, an immunoglobulin superfamily member, or an antibody or antigen-binding fragment thereof; and
the heterologous intracellular signaling domain comprises an amino acid sequence from a tumor necrosis factor receptor superfamily member, a cytokine receptor, or a C-type lectin receptor.

Embodiment 109. The chimeric protein of embodiment 108, wherein:
the extracellular ligand domain comprises an amino acid sequence from 41BBL, OX40L, CD86, or RANK, and
the heterologous intracellular signaling domain comprises an amino acid sequence from OX40, 41BB, NKp80, or IL18RAP.

Embodiment 110. The chimeric protein of embodiment 109, wherein:
(a) the extracellular ligand domain comprises an amino acid sequence from 41BBL and the heterologous intracellular signaling domain comprises an amino acid sequence from OX40, preferably wherein the extracellular ligand domain is from or is derived from a type II transmembrane protein 41BBL and the heterologous intracellular signaling domain is from or is derived from a type I transmembrane protein OX40,
(b) the extracellular ligand domain comprises an amino acid sequence from CD86 and the heterologous intracellular signaling domain comprises an amino acid sequence from OX40,
(c) the extracellular ligand domain comprises an amino acid sequence from 41BBL and the heterologous intracellular signaling domain comprises an amino acid sequence from NKp80,
(d) the extracellular ligand domain comprises an amino acid sequence from RANK and the heterologous intracellular signaling domain comprises an amino acid sequence from IL18RAP,
(e) the extracellular ligand domain comprises an amino acid sequence from RANK and the heterologous intracellular signaling domain comprises an amino acid sequence from OX40,
(f) the extracellular ligand domain comprises an amino acid sequence from RANK and the heterologous intracellular signaling domain comprises an amino acid sequence from 41BB,
(g) the extracellular ligand domain comprises an amino acid sequence from OX40L and the heterologous intracellular signaling domain comprises an amino acid sequence from 41BB, or
(h) the extracellular ligand domain comprises an amino acid sequence from CD86 and the heterologous intracellular signaling domain comprises an amino acid sequence from IL18RAP.

Embodiment 111. The chimeric protein of any one of embodiments 101-110, wherein the chimeric protein does not contain an ITAM or an intracellular domain from a TCR signaling complex.

Embodiment 112. A polynucleotide encoding the chimeric protein as defined in any one of embodiments 101-111.

Embodiment 113. A vector comprising the polynucleotide as defined in embodiment 112, wherein preferably the vector is a viral vector.

Embodiment 114. A cell comprising the polynucleotide as defined in embodiment 112 or the vector as defined in embodiment 113, preferably wherein said cell expresses said chimeric protein, more preferably wherein said cell also expresses the interaction partner.

Embodiment 115. A population of cells, wherein the population of cells comprises at least one cell as defined in embodiment 114.

Embodiment 116. The cell of embodiment 114 or the population of cells of embodiment 115, wherein the cells are immune cells, preferably T cells or NK cells.

Embodiment 117. The population of cells of embodiment 115 or 116, wherein the population of cells further comprises at least one cell that expresses an exogenous antigen-recognition receptor.

Embodiment 118. The population of cells of embodiment 117, wherein at least one cell that expresses the chimeric protein as defined in any one of embodiments 101-111 also expresses an exogenous antigen-recognition receptor.

Embodiment 119. The population of cells of embodiments 117 or 118, wherein the exogenous antigen-recognition receptor is a chimeric antigen receptor, a T cell receptor, an alpha-beta T cell receptor, or a gamma-delta T cell receptor.

Embodiment 120. The population of cells of any one of embodiments 115-119, wherein the T cells are alpha-beta T cells that express a gamma-delta T cell receptor.

Embodiment 121. The population of cells of any one of embodiments 115-120, wherein upon exposure of the cells that express the chimeric protein as defined in any one of embodiments 101-111 to cells that express or present an antigen that binds to the exogenous antigen-recognition receptor, proliferation, cellular survival, cytotoxicity, antitumor activity, persistence and/or tumor cell killing of the population of said cells is increased by at least 10% compared to a corresponding population of cells that do not express the chimeric protein.

Embodiment 122. The chimeric protein of any one of embodiments 101-111, the polynucleotide of embodiment 112, the vector of embodiment 113, the cell according of embodiment 114 or the population of cells of any one of embodiments 115-121, wherein the chimeric protein, the polynucleotide, the vector, the cell or the population of cells is for use for treating a disease or a condition wherein the at least two intracellular signals contribute to an improvement of a biological parameter and/or function of a cell expressing the chimeric protein and/or an improvement of a biological parameter and/or function induced by such a cell, said biological parameter contributing to the treatment of the disease or condition.

Embodiment 123. The chimeric protein of embodiment 122, the polynucleotide of embodiment 122, the vector of embodiment 122, the cell of embodiment 122 or the population of cells of embodiment 122, wherein:
the biological parameter selected from proliferation, cellular survival, cytotoxicity, antitumor activity, persistence and/or tumor cell killing,
the cell is an immune cell and/or
the disease is cancer.

Embodiment 124. A chimeric bidirectional signaling transmembrane protein able to transduce at least two inducible intracellular signals, said protein comprising:
an extracellular ligand domain, able to interact with the extracellular domain of its interaction partner
a transmembrane domain, and
a heterologous intracellular signaling domain transducing a first signal after binding of the extracellular ligand domain to its interaction partner,
   wherein the second intracellular signal is transduced via the intracellular domain of the interaction partner.

Embodiment 125. The chimeric protein of embodiment 124, wherein the interaction partner comprises:
an extracellular domain able to interact with the extracellular ligand domain of the chimeric protein,
a transmembrane domain, and
an intracellular domain transducing a second signal after binding of the extracellular domain of the interaction partner to the extracellular ligand domain of the chimeric protein.

Embodiment 126. The chimeric protein of embodiment 124 or 125, wherein the at least two inducible intracellular signals contribute to an improvement of a biological parameter and/or function of a cell expressing the chimeric protein and/or an improvement of a biological parameter and/or function induced by such a cell.

Embodiment 127. The chimeric protein of any one of embodiments 124-126, wherein
a. The extracellular ligand domain is from or derived from a type I transmembrane protein and the heterologous intracellular signaling domain is from or derived from a type 11 transmembrane protein or
b. The extracellular ligand domain is from or derived from a type 11 transmembrane protein and the heterologous intracellular signaling domain is from or derived from a type I transmembrane protein.

Embodiment 128. The chimeric protein of any one of embodiments 126-127, wherein the cell is an immune cell, preferably a T or NK cell.

Embodiment 129. The chimeric protein of any one of embodiments 126-128, wherein the biological parameter and/or function is selected from proliferation, cellular survival, cytotoxicity, antitumor activity, persistence and/or tumor cell killing,

Embodiment 130. The chimeric protein of any one of embodiments 124-129, wherein:
the extracellular ligand domain comprises an amino acid sequence from a tumor necrosis factor superfamily member, a cytokine, a C-type lectin, an immunoglobulin superfamily member, or an antibody or antigen-binding fragment thereof; and
the heterologous intracellular signaling domain comprises an amino acid sequence from a tumor necrosis factor receptor superfamily member, a cytokine receptor, or a C-type lectin receptor.

Embodiment 131. The chimeric protein of embodiment 130, wherein:
the extracellular ligand domain comprises an amino acid sequence from 41BBL, OX40L, CD86, or RANK, and
the heterologous intracellular signaling domain comprises an amino acid sequence from OX40, 41BB, NKp80, or IL18RAP.

Embodiment 132. The chimeric protein of embodiment 131, wherein:
(a) the extracellular ligand domain comprises an amino acid sequence from 41BBL and the heterologous intracellular signaling domain comprises an amino acid sequence from OX40, preferably wherein the extracellular ligand domain is from or is derived from a type II transmembrane protein 41BBL and the heterologous intracellular signaling domain is from or is derived from a type I transmembrane protein OX40,
(b) the extracellular ligand domain comprises an amino acid sequence from CD86 and the heterologous intracellular signaling domain comprises an amino acid sequence from OX40,
(c) the extracellular ligand domain comprises an amino acid sequence from 41BBL and the heterologous intracellular signaling domain comprises an amino acid sequence from NKp80,
(d) the extracellular ligand domain comprises an amino acid sequence from RANK and the heterologous intracellular signaling domain comprises an amino acid sequence from IL18RAP,
(e) the extracellular ligand domain comprises an amino acid sequence from RANK and the heterologous intracellular signaling domain comprises an amino acid sequence from OX40,
(f) the extracellular ligand domain comprises an amino acid sequence from RANK and the heterologous intracellular signaling domain comprises an amino acid sequence from 41BB,
(g) the extracellular ligand domain comprises an amino acid sequence from OX40L and the heterologous intracellular signaling domain comprises an amino acid sequence from 41BB, or
(h) the extracellular ligand domain comprises an amino acid sequence from CD86 and the heterologous intracellular signaling domain comprises an amino acid sequence from IL18RAP.

Embodiment 133. The chimeric protein of any one of embodiments 124-132, wherein the chimeric protein does not contain an ITAM or an intracellular domain from a TCR signaling complex.

Embodiment 134. A polynucleotide encoding the chimeric protein as defined in any one of embodiments 124-133.

Embodiment 135. A vector comprising the polynucleotide as defined in embodiment 134, wherein preferably the vector is a viral vector.

Embodiment 136. A cell comprising the polynucleotide as defined embodiment 134 or the vector as defined in embodiment 135, preferably wherein said cell expresses said chimeric protein, more preferably wherein said cell also expresses the interaction partner.

Embodiment 137. A population of cells, wherein the population of cells comprises at least one cell as defined embodiment 136.

Embodiment 138. The cell of embodiment 136 or the population of cells of embodiment 137, wherein the cells are immune cells, preferably T cells or NK cells.

Embodiment 139. The population of cells of embodiment 137 or 138, wherein the population of cells further comprises at least one cell that expresses an exogenous antigen-recognition receptor.

Embodiment 140. The population of cells of embodiment 139, wherein at least one cell that expresses the chimeric protein as defined in any one of embodiments 124-133 also expresses an exogenous antigen-recognition receptor.

Embodiment 141. The population of cells of embodiment 139 or 140, wherein the exogenous antigen-recognition receptor is a chimeric antigen receptor, a T cell receptor, an alpha-beta T cell receptor, or a gamma-delta T cell receptor.

Embodiment 142. The population of cells of any one of embodiments 137-141, wherein the T cells are alpha-beta T cells that express a gamma-delta T cell receptor.

Embodiment 143. The population of cells of any one of embodiments 137-142, wherein upon exposure of the cells that express the chimeric protein as defined in any one of embodiments 124-133 to cells that express or present an antigen that binds to the exogenous antigen-recognition receptor, proliferation, cellular survival, cytotoxicity, antitumor activity, persistence and/or tumor cell killing of the population of said cells is increased by at least 10% compared to a corresponding population of cells that do not express the chimeric protein.

Embodiment 144. The chimeric protein of any one of embodiments 124-133, the polynucleotide of embodiment 134, the vector of embodiment 135, the cell according of embodiment 136 or the population of cells of any one of embodiments 137-143, wherein the chimeric protein, the polynucleotide, the vector, the cell or the population of cells is for use for treating a disease or a condition wherein the at least two inducible intracellular signals contribute to an improvement of a biological parameter and/or function of a cell expressing the chimeric protein and/or an improvement of a biological parameter and/or function induced by such a cell, said biological parameter contributing to the treatment of the disease or condition.

Embodiment 145. The chimeric protein of embodiment 144, the polynucleotide of embodiment 144, the vector of embodiment 144, the cell of embodiment 144 or the population of cells of embodiment 144, wherein:
the biological parameter selected from proliferation, cellular survival, cytotoxicity, antitumor activity, persistence and/or tumor cell killing,
the cell is an immune cell and/or
the disease is cancer.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A polynucleotide encoding each of the monomers of a heterodimeric receptor, wherein said polynucleotide comprises at least one nucleic acid encoding a polypeptide other than said monomers inserted between the nucleic acids encoding each of said monomers, and wherein said nucleic acids are operably linked to the same promoter sequence.

2. A polynucleotide according to claim 1, wherein said promoter sequence is selected from the group of EF1α, MSCV, EF1 alpha-HTLV-1 hybrid promoter, Moloney murine leukemia virus, Gibbon Ape Leukemia virus, murine mammary tumor virus, Rous sarcoma virus, MHC class II, clotting Factor IX, insulin promoter, PDX1 promoter, CD11, CD4, CD2, gp47 promoter, PGK, Beta-globin, UbC, and MND, preferably from MSCV, MMLV, EF1α, and MND.

3. A polynucleotide according to claim 1, wherein said polynucleotide comprises a nucleotide sequence inserted between each of the nucleic acids which facilitates their co-expression.

4. A polynucleotide according to claim 3, wherein said nucleotide sequence is a sequence encoding a 2A self-cleaving peptide or is an IRES sequence.

5. (canceled)

6. A polynucleotide according to claim 1, wherein said polynucleotide is tricistronic or tetracistronic.

7. A polynucleotide according to claim 1, wherein said heterodimeric receptor is an exogenous antigen-recognition receptor.

8. A polynucleotide according to claim 7, wherein said exogenous antigen-recognition receptor is selected from a B-cell receptor heavy and light chain heterodimer, a Toll-like receptor 1 and 2 heterodimer, a phagocytic receptor Mac-1, a CD94 NKG2C or NKG2E receptor, a T-cell receptor, an αβT-cell receptor, a γδT-cell receptor, and functional fragments thereof.

9. A polynucleotide according to claim 8, wherein the exogenous antigen-recognition receptor is an αβT-cell receptor, a γδT-cell receptor, or a functional fragment thereof.

10. A polynucleotide according to claim 9, comprising A, B, C, or D, wherein:
(A) is a nucleic acid represented by (i)-(ii)-(iii), wherein:
(i) is a nucleic acid encoding an α chain of an αβT-cell receptor or a functional fragment thereof,
(ii) is at least one nucleic acid encoding a polypeptide other than an α or β chain of an αβT-cell receptor or a functional fragment thereof, and;
(iii) is a nucleic acid encoding a β chain of an αβT-cell receptor or a functional fragment thereof,
wherein (ii) is inserted between (i) and (iii)
(B) is a nucleic acid represented by (iv)-(v)-(vi), wherein:
(iv) is a nucleic acid encoding a β chain of an αβT-cell receptor or a functional fragment thereof,
(v) is at least one nucleic acid encoding a polypeptide other than an α or β chain of an αβT-cell receptor or a functional fragment thereof, and;
(vi) is a nucleic acid encoding an α chain of an αβT-cell receptor or a functional fragment thereof,
wherein (v) is inserted between (iv) and (vi) (C) is a nucleic acid represented by (vii)-(viii)-(ix), wherein:
(vii) is a nucleic acid encoding a γ chain of a γδT-cell receptor or a functional fragment thereof,
(viii) is at least one nucleic acid encoding a polypeptide other than a γ or δ chain of a γδT-cell receptor or a functional fragment thereof, and;
(ix) is a nucleic acid encoding a δ chain of a γδT-cell receptor or a functional fragment thereof,
wherein (viii) is inserted between (vi) and (ix) (D) is a nucleic acid represented by (x)-(xi)-(xii), wherein:
(x) is a nucleic acid encoding a δ chain of a γδT-cell receptor or a functional fragment thereof,
(xi) is at least one nucleic acid encoding a polypeptide other than a γ or δ chain of a γδT-cell receptor or a functional fragment thereof, and;
(xii) is a nucleic acid encoding a γ chain of a γδT-cell receptor or a functional fragment thereof,
wherein (xi) is inserted between (x) and (xii)

11. A polynucleotide according to claim 10, wherein:
(i) and (vi) are nucleic acids comprising a nucleotide sequence encoding a polypeptide having at least 60%, 70%, 80%, 90%, 95%, or 100% identity or similarity with an amino acid sequence selected from SEQ ID NOs: 199, 210, 214, 216, 218, and 220, preferably selected from SEQ ID NOs: 210, 216, and 220, and/or;
(iii) and (iv) are nucleic acids comprising a nucleotide sequence encoding a polypeptide having at least 60%, 70%, 80%, 90%, 95%, or 100% identity or similarity with an amino acid sequence selected from SEQ ID NOs: 198, 211, 215, 217, 219, and 221, preferably selected from SEQ ID NOs: 211, 217, and 221, and/or;
(vii) and (xii) are nucleic acids comprising a nucleotide sequence encoding a polypeptide having at least 60%, 70%, 80%, 90%, 95%, or 100% identity or similarity with an amino acid sequence selected from SEQ ID NOs: 85, 86, 87, 89, 91, 93, 94, 95, 96, 101, 104, 106, 108, 110, 112, 113, 115, 117, 119, 121, 123, 125, 127, 129, 130, and 132, preferably selected from SEQ ID NOs: 85, 86, 87, 94, 95, 96, 101, 113, 115, 117, 119, 127, and 130, and/or;
(ix) and (x) are nucleic acids comprising a nucleotide sequence encoding a polypeptide having at least 60%, 70%, 80%, 90%, 95%, or 100% identity or similarity with an amino acid sequence selected from SEQ ID NOs: 82, 83, 84, 88, 90, 92, 97, 98, 99, 100, 102, 103, 105, 107, 109, 111, 114, 116, 118, 120, 122, 124, 126, 128, 131, and 133, preferably selected from SEQ ID NOs: 82, 83, 84, 97, 98, 99, 100, 102, 114, 116, 118, 126, and 131.

12. A polynucleotide according to claim 1, wherein said polynucleotide comprises a nucleic acid inserted between the nucleic acids encoding each of the receptor monomers which encodes a chimeric bidirectional signaling transmembrane protein able to transduce at least two intracellular signals, said protein comprising:
an extracellular ligand domain, able to interact with the extracellular domain of its interaction partner
a transmembrane domain, and
a heterologous intracellular signaling domain transducing a first signal after binding of the extracellular ligand domain to its interaction partner,
wherein the second intracellular signal is transduced via the intracellular domain of the interaction partner.

13. (canceled)

14. A polynucleotide according to claim 12, wherein the at least two intracellular signals are inducible.

15. (canceled)

16. A polynucleotide according to claim 12, wherein the interaction partner comprises:
an extracellular domain able to interact with the extracellular ligand domain of the chimeric protein,
a transmembrane domain, and
an intracellular domain transducing a second signal after binding of the extracellular domain of the interaction partner to the extracellular ligand domain of the chimeric protein.

17-20. (canceled)

21. A polynucleotide according to claim 12, wherein:
the extracellular ligand domain comprises an amino acid sequence from a tumor necrosis factor superfamily member, a cytokine, a C-type lectin, an immunoglobulin superfamily member, or an antibody or antigen-binding fragment thereof; and
the heterologous intracellular signaling domain comprises an amino acid sequence from a tumor necrosis factor receptor superfamily member, a cytokine receptor, or a C-type lectin receptor.

22. A polynucleotide according to claim 21, wherein:
the extracellular ligand domain comprises an amino acid sequence from 41BBL, OX40L, CD86, RANK, or CD70, and
the heterologous intracellular signaling domain comprises an amino acid sequence from OX40, 41BB, NKp80, IL18RAP, or IL2RB.

23. A polynucleotide according to claim 22, wherein:
(a) the extracellular ligand domain comprises an amino acid sequence from 41BBL and the heterologous intracellular signaling domain comprises an amino acid sequence from OX40, preferably wherein the extracellular ligand domain is from or is derived from a type II transmembrane protein 41BBL and the heterologous intracellular signaling domain is from or is derived from a type I transmembrane protein OX40,
(b) the extracellular ligand domain comprises an amino acid sequence from CD86 and the heterologous intracellular signaling domain comprises an amino acid sequence from OX40, preferably wherein the extracellular ligand domain is from or is derived from a type I transmembrane protein CD86 and the heterologous intracellular signaling domain is from or is derived from a type I transmembrane protein OX40,
(c) the extracellular ligand domain comprises an amino acid sequence from 41BBL and the heterologous intracellular signaling domain comprises an amino acid sequence from NKp80, preferably wherein the extracellular ligand domain is from or is derived from a type II transmembrane protein 41BBL and the heterologous intracellular signaling domain is from or is derived from a type II transmembrane protein NpK80,
(d) the extracellular ligand domain comprises an amino acid sequence from RANK and the heterologous intracellular signaling domain comprises an amino acid sequence from IL18RAP, preferably wherein the extracellular ligand domain is from or is derived from a type I transmembrane protein RANK and the heterologous intracellular signaling domain is from or is derived from a type I transmembrane protein IL18RAP,
(e) the extracellular ligand domain comprises an amino acid sequence from RANK and the heterologous intracellular signaling domain comprises an amino acid sequence from OX40, preferably wherein the extracellular ligand domain is from or is derived from a type I transmembrane protein RANK and the heterologous intracellular signaling domain is from or is derived from a type I transmembrane protein OX40,
(f) the extracellular ligand domain comprises an amino acid sequence from RANK and the heterologous intracellular signaling domain comprises an amino acid sequence from 41BB, preferably wherein the extracellular ligand domain is from or is derived from a type I transmembrane protein RANK and the heterologous intracellular signaling domain is from or is derived from a type I transmembrane protein 41BB,
(g) the extracellular ligand domain comprises an amino acid sequence from OX40L and the heterologous intracellular signaling domain comprises an amino acid sequence from 41BB, preferably wherein the extracellular ligand domain is from or is derived from a type II transmembrane protein OX40L and the heterologous intracellular signaling domain is from or is derived from a type I transmembrane protein 41BB,
(h) the extracellular ligand domain comprises an amino acid sequence from CD86 and the heterologous intracellular signaling domain comprises an amino acid sequence from IL18RAP, preferably wherein the extracellular ligand domain is from or is derived from a type I transmembrane protein CD86 and the heterologous intracellular signaling domain is from or is derived from a type I transmembrane protein IL18RAP,
(i) the extracellular ligand domain comprises an amino acid sequence from CD70 and the heterologous intracellular signaling domain comprises an amino acid sequence from OX40, preferably wherein the extracellular ligand domain is from or is derived from a type II transmembrane protein CD70 and the heterologous intracellular signaling domain is from or is derived from a type I transmembrane protein OX40, or
(j) the extracellular ligand domain comprises an amino acid sequence from 41BBL and the heterologous intracellular signaling domain comprises an amino acid sequence from OX40 and an amino acid sequence from IL2RB, preferably wherein the extracellular ligand domain is from or is derived from a type II transmembrane protein 41BBL and the heterologous intracellular signaling domain is from or is derived from a type I transmembrane protein OX40 and from a type I transmembrane protein IL2RB.

24. A polynucleotide according to claim 23, wherein:
k) the chimeric protein identified under a) is represented by an amino acid sequence having at least 80% identity or similarity with SEQ ID NO: 45, 46, 57, 58, 59, 60, 61, 62, 63, 64, 65, 178, or 179,
l) the chimeric protein identified under b) is represented by an amino acid sequence having at least 80% identity or similarity with SEQ ID NO:52, 53, or 73,
m) the chimeric protein identified under c) is represented by an amino acid sequence having at least 80% identity or similarity with SEQ ID NO:47, or 48,
n) the chimeric protein identified under d) is represented by an amino acid sequence having at least 80% identity or similarity with SEQ ID NO: 78,
o) the chimeric protein identified under e) is represented by an amino acid sequence having at least 80% identity or similarity with SEQ ID NO: 76,
p) the chimeric protein identified under f) is represented by an amino acid sequence having at least 80% identity or similarity with SEQ ID NO: 77,
q) the chimeric protein identified under g) is represented by an amino acid sequence having at least 80% identity or similarity with SEQ ID NO: 49, 50, or 51,
r) the chimeric protein identified under h) is represented by an amino acid sequence having at least 80% identity or similarity with SEQ ID NO: 71, or 72,
s) the chimeric protein identified under i) is represented by an amino acid sequence having at least 80% identity or similarity with SEQ ID NO: 182, or 183, or
t) the chimeric protein identified under j) is represented by an amino acid sequence having at least 80% identity or similarity with SEQ ID NO: 179.

25. (canceled)

26. A polynucleotide encoding the chimeric bidirectional signaling transmembrane protein as defined in claim 12.

27-28. (canceled)

29. A polypeptide encoded by a polynucleotide as defined in claim 12.

30. A cell comprising a polynucleotide as defined in claim 12.

31-39. (canceled)

40. A method of treating a disease or a condition comprising administering a cell according to claim 30 to a subject in need thereof, wherein the at least two intracellular signals contribute to an improvement of a biological parameter and/or function of a cell expressing the chimeric protein and/or an improvement of a biological parameter and/or function induced by such a cell, said biological parameter contributing to the treatment of the disease or condition.


SEQUENCE LISTING (222 sequences)

SEQ ID NO: 1 - protein, 205 aa, Artificial sequence
  41BBL-EC
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 133 aa, Artificial sequence
  OX40L
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 3 - protein, 247 aa, Artificial sequence
  CD86
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIP

SEQ ID NO: 4 - protein, 212 aa, Artificial sequence
  RANK
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLP

SEQ ID NO: 5 - protein, 220 aa, Artificial sequence
  Reverse 41BBL
MLLLVTSLLLCELPHPAFLLIPDQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSERLDLLGAPDDPSLEPGERLRPSAASGPSARA

SEQ ID NO: 6 - protein, 347 aa, Artificial sequence
  41BBL  CD86 IgV domain
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSESLNGGGGSGGGGSGGGGSGGGGSGGGGSTSAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLAN

SEQ ID NO: 7 - protein, 36 aa, Artificial sequence
  OX40-ICD
RDQRLPPDAHKPPGGGSFRTPIQEEQADAHSTLAKI

SEQ ID NO: 8 - protein, 36 aa, Artificial sequence
  OX40-ICD-REV
IKALTSHADAQEEQIPTRFSGGGPPKHADPPLRQDR

SEQ ID NO: 9 - protein, 63 aa, Artificial sequence
  OX40-ICD-TM
VAAILGLGLVLGLLGPLAILLALYLLRRDQRLPPDAHKPPGGGSFRTPIQEEQADAHSTLAKI

SEQ ID NO: 10 - protein, 42 aa, Artificial sequence
  41BB-ICD
KRGRKKLLYIFKQPFMRPVQTTQEEDGCSCRFPEEEEGGCEL

SEQ ID NO: 11 - protein, 45 aa, Artificial sequence
  41BB-ICD-REV
MLGKRGRKKLLYIFKQPFMRPVQTTQEEDGCSCRFPEEEEGGCEL

SEQ ID NO: 12 - protein, 36 aa, Artificial sequence
  NKp80-ICD
MQDEDGYMTLNVQSKKRSSAQTSQLTFKDYSVTLHW

SEQ ID NO: 13 - protein, 224 aa, Artificial sequence
  IL18RAP-ICD
AASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 14 - protein, 69 aa, Artificial sequence
  41BB-ICD-TM
IISFFLALTSTALLFLLFFLTLRFSVVKRGRKKLLYIFKQPFMRPVQTTQEEDGCSCRFPEEEEGGCEL

SEQ ID NO: 15 - protein, 42 aa, Artificial sequence
  OX40-ICD-ALYLLR
IKALTSHADAQEEQIPTRFSGGGPPKHADPPLRQDRRLLYLA

SEQ ID NO: 16 - protein, 42 aa, Artificial sequence
  OX40-ICD-ALYLLR-REV
ALYLLRRDQRLPPDAHKPPGGGSFRTPIQEEQADAHSTLAKI

SEQ ID NO: 17 - protein, 6 aa, Artificial sequence
  TRAF domain OX40
PIQEEQ

SEQ ID NO: 18 - protein, 5 aa, Artificial sequence
  YMTLN motif
YMTLN

SEQ ID NO: 19 - protein, 18 aa, Artificial sequence
  TRAF domain 41BB
QTTQEEDGCSCRFPEEEE

SEQ ID NO: 20 - protein, 21 aa, Artificial sequence
  OX40-TM
VAAILGLGLVLGLLGPLAILL

SEQ ID NO: 21 - protein, 27 aa, Artificial sequence
  OX40L-TM
LLLVASVIQGLGLLLCFTYICLHFSAL

SEQ ID NO: 22 - protein, 27 aa, Artificial sequence
  41BB-TM
IISFFLALTSTALLFLLFFLTLRFSVV

SEQ ID NO: 23 - protein, 21 aa, Artificial sequence
  41BBL-TM
WALVAGLLLLLLLAAACAVFL

SEQ ID NO: 24 - protein, 21 aa, Artificial sequence
  NKp80-TM
ILLGISGTVNGILTLTLISLI

SEQ ID NO: 25 - protein, 21 aa, Artificial sequence
  CD86-TM
WITAVLPTVIICVMVFCLILW

SEQ ID NO: 26 - protein, 19 aa, Artificial sequence
  IL18RAP-TM
GVVLLYILLGTIGTLVAVL

SEQ ID NO: 27 - protein, 21 aa, Artificial sequence
  RANK-TM
GLIILLLFASVALVAAIIFGV

SEQ ID NO: 28 - protein, 4 aa, Artificial sequence
  Flexible linker
TSGS

SEQ ID NO: 29 - protein, 5 aa, Artificial sequence
  Flexible linker
GGGGS

SEQ ID NO: 30 - protein, 4 aa, Artificial sequence
  Flexible linker
GGGS

SEQ ID NO: 31 - protein, 2 aa, Artificial sequence
  Flexible linker
GG

SEQ ID NO: 32 - protein, 18 aa, Artificial sequence
  Flexible linker
KESGSVSSEQLAQFRSLD

SEQ ID NO: 33 - protein, 14 aa, Artificial sequence
  Flexible linker
EGKSSGSGSESKST

SEQ ID NO: 34 - protein, 12 aa, Artificial sequence
  Flexible linker
GSAGSAAGSGEF

SEQ ID NO: 35 - protein, 5 aa, Artificial sequence
  Rigid linker
EAAAK

SEQ ID NO: 36 - protein, 5 aa, Artificial sequence
  Rigid linker
EAAAR

SEQ ID NO: 37 - protein, 5 aa, Artificial sequence
  Rigid linker
PAPAP

SEQ ID NO: 38 - protein, 12 aa, Artificial sequence
  Rigid linker
AEAAAKEAAAKA

SEQ ID NO: 39 - protein, 51 aa, Artificial sequence
  Rigid linker
ILTHDSSIRYLQEIYNSNNQKIVNLKEKVAQLEAQCQEPCKDTVQIHDITG

SEQ ID NO: 40 - protein, 3 aa, Artificial sequence
  Flexible linker
GGS

SEQ ID NO: 41 - protein, 30 aa, Artificial sequence
  Flexible linker
SLNGGGGSGGGGSGGGGSGGGGSGGGGSTS

SEQ ID NO: 42 - protein, 20 aa, Artificial sequence
  Flexible linker
SGGSGGGGSGGGSGGGGSLQ

SEQ ID NO: 43 - protein, 26 aa, Artificial sequence
  Flexible linker
SGGGSGGGGSGGGGSGGGGSGGGSLQ

SEQ ID NO: 44 - protein, 15 aa, Artificial sequence
  Flexible linker
GGGGSGGGGSGGGGS

SEQ ID NO: 45 - protein, 295 aa, Artificial sequence
  41BBL-OX40
MLGRDQRLPPDAHKPPGGGSFRTPIQEEQADAHSTLAKITSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 46 - protein, 269 aa, Artificial sequence
  41BBL13W-OX40rev
MLGIKALTSHADAQEEQIPTRFSGGGPPKHADPPLRQDRTSGSWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 47 - protein, 288 aa, Artificial sequence
  41BBL-NKp80
MQDEDGYMTLNVQSKKRSSAQTSQLTFKDYSVTLHWYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 48 - protein, 264 aa, Artificial sequence
  41BBLmincyto-NKp80
MQDEDGYMTLNVQSKKRSSAQTSQLTFKDYSVTLHWYKWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 49 - protein, 231 aa, Artificial sequence
  OX40L-41BB
MLGKRGRKKLLYIFKQPFMRPVQTTQEEDGCSCRFPEEEEGGCELTSGSERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 50 - protein, 231 aa, Artificial sequence
  OX40L-41BB
MLGKRGRKKLLYIFKQPFMRPVQTTQEEDGCSCRFPEEEEGGCELGGSAERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 51 - protein, 232 aa, Artificial sequence
  OX40L-41BBrev
MLGLECGGEEEEPFRCSCGDEEQTTQVPRMFPQKFIYLLKKRGRKLGGSAERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 52 - protein, 368 aa, Artificial sequence
  CD86-OX40
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCFGSGRDQRLPPDAHKPPGGGSFRTPIQEEQADAHSTLAKI

SEQ ID NO: 53 - protein, 313 aa, Artificial sequence
  CD86delP276-OX40
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPGRDQRLPPDAHKPPGGGSFRTPIQEEQADAHSTLAKI

SEQ ID NO: 54 - protein, 254 aa, Homo sapiens
  41BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 55 - protein, 233 aa, Artificial sequence
  41BBL-mincyto
MSKSTGSWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 56 - protein, 228 aa, Artificial sequence
  41BBL13W
MEWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 57 - protein, 295 aa, Artificial sequence
  41BBL-OX40rev
MLGIKALTSHADAQEEQIPTRFSGGGPPKHADPPLRQDRTSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 58 - protein, 283 aa, Artificial sequence
  41BBL-rev extra-OX40tm-cyto
MLLLVTSLLLCELPHPAFLLIPDQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSERLDLLGAPDDPSLEPGERLRPSAASGPSARAVAAILGLGLVLGLLGPLAILLALYLLRRDQRLPPDAHKPPGGGSFRTPIQEEQADAHSTLAKI

SEQ ID NO: 59 - protein, 269 aa, Artificial sequence
  41BBLmincyto-OX40
MLGRDQRLPPDAHKPPGGGSFRTPIQEEQADAHSTLAKITSGSWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 60 - protein, 281 aa, Artificial sequence
  41BBL13W-OX40rev
MEIKALTSHADAQEEQIPTRFSGGGPPKHADPPLRQDRRLLWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 61 - protein, 269 aa, Artificial sequence; identical to SEQ ID NO: 46
  41BBL-mincyto-OX40rev

SEQ ID NO: 62 - protein, 301 aa, Artificial sequence
  41BBL-OX40ENrev
MLGIKALTSHADAQEEQIPTRFSGGGPPKHADPPLRQDRRLLYLATSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 63 - protein, 301 aa, Artificial sequence
  41BBL-OX40EN
MLGALYLLRRDQRLPPDAHKPPGGGSFRTPIQEEQADAHSTLAKITSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 64 - protein, 291 aa, Artificial sequence
  41BBLmincyto-13alink-OX40
MLGRDQRLPPDAHKPPGGGSFRTPIQEEQADAHSTLAKISLNGGGGSGGGGSGGGGSGGGGSGGPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 65 - protein, 209 aa, Artificial sequence
  OX40Lmincyto-41BBrev
MLGLECGGEEEEPFRCSCGDEEQTTQVPRMFPQKFIYLLKKRGRKLGGSLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 66 - protein, 183 aa, Homo sapiens
  OX40L WT
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 67 - protein, 208 aa, Artificial sequence
  OX40Lmincyto-41BB
MLGKRGRKKLLYIFKQPFMRPVQTTQEEDGCSCRFPEEEEGGCELGGSLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 68 - protein, 329 aa, Homo sapiens
  CD86 WT
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 69 - protein, 276 aa, Artificial sequence
  CD86delP276
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRP

SEQ ID NO: 70 - protein, 268 aa, Artificial sequence
  CD86mincyto
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILW

SEQ ID NO: 71 - protein, 490 aa, Artificial sequence
  CD86mincyto-IL18RAP
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWSALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 72 - protein, 551 aa, Artificial sequence
  CD86-IL18RAP
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCFSALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 73 - protein, 437 aa, Artificial sequence
  CD86IgV-41BBL-OX40
MLGRDQRLPPDAHKPPGGGSFRTPIQEEQADAHSTLAKITSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSESLNGGGGSGGGGSGGGGSGGGGSGGGGSTSAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLAN

SEQ ID NO: 74 - protein, 613 aa, Homo sapiens
  RANK WT
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 75 - protein, 230 aa, Artificial sequence
  RANKmincyto
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPGLIILLLFASVALVAAIIFGV

SEQ ID NO: 76 - protein, 272 aa, Artificial sequence
  RANK-OX40
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPVAAILGLGLVLGLLGPLAILLALYLLRRDQRLPPDAHKPPGGGSFRTPIQEEQADAHSTLAKI

SEQ ID NO: 77 - protein, 278 aa, Artificial sequence
  RANK-41BB
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPIISFFLALTSTALLFLLFFLTLRFSVVKRGRKKLLYIFKQPFMRPVQTTQEEDGCSCRFPEEEEGGCEL

SEQ ID NO: 78 - protein, 452 aa, Artificial sequence
  RANK-IL18RAP
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSGSRKPPNEPHVYLPGVVLLYILLGTIGTLVAVLAASALLYRHWIEIVLLYRTYQSKDQTLGDKKDFDAFVSYAKWSSFPSEATSSLSEEHLALSLFPDVLENKYGYSLCLLERDVAPGGVYAEDIVSIIKRSRRGIFILSPNYVNGPSIFELQAAVNLALDDQTLKLILIKFCYFQEPESLPHLVKKALRVLPTVTWRGLKSVPPNSRFWAKMRYHMPVKNSQGFTWNQLRITSRIFQWKGLSRTETTGRSSQPKEW

SEQ ID NO: 79 - protein, 165 aa, Artificial sequence
  OX40WT mincyto
MERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 80 - protein, 138 aa, Artificial sequence
  CD8-Q8
MGLVRRGARAGPRIPRGWTALCLLSLLPSGFMAELPTQGTFSNVSTNVSPAKPTTTPAPRPPTPAPTIASQPLSLRPEACRPAAGGAVHTRGLDFACDIYIWAPLAGTCGVLLLSLVITLYCNHRNRRRVCKCPRPVV

SEQ ID NO: 81 - protein, 239 aa, Artificial sequence
  eGFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 82 - protein, 19 aa, Artificial sequence
  CDR3 VD1 Fe11
CALGDSYGGGPLYTDKLIF

SEQ ID NO: 83 - protein, 14 aa, Artificial sequence
  CDR3 VD2 cl3
CACDLLGYTDKLIF

SEQ ID NO: 84 - protein, 17 aa, Artificial sequence
  CDR3 VD2 cl5
CACDALKRTDTDKLIFG

SEQ ID NO: 85 - protein, 15 aa, Artificial sequence
  CDR3 VG5 Fe11
CATWDRPEIYYKKLF

SEQ ID NO: 86 - protein, 15 aa, Artificial sequence
  CDR3 VG9 cl3
CALWEEELGKKIKVF

SEQ ID NO: 87 - protein, 16 aa, Artificial sequence
  CDR3 VG9 cl5
CALWEIQELGKKIKVF

SEQ ID NO: 88 - protein, 135 aa, Homo sapiens
  TRD cl3
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDLLGYTDKLIFGKGTRVTVEPR

SEQ ID NO: 89 - protein, 140 aa, Homo sapiens
  TRG cl3
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEEELGKKIKVFGPGTKLIIT

SEQ ID NO: 90 - protein, 137 aa, Homo sapiens
  TRD cl5
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPR

SEQ ID NO: 91 - protein, 141 aa, Homo sapiens
  TRG cl5
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIIT

SEQ ID NO: 92 - protein, 140 aa, Homo sapiens
  TRD Fe11
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGDSYGGGPLYTDKLIFGKGTRVTVEPR

SEQ ID NO: 93 - protein, 136 aa, Homo sapiens
  TRG Fe11
MGWALLVLLAFLSPASQKSSNLEGGTKSVTRPTRSSAEITCDLTVINAFYIHWYLHQEGKAPQRLLYYDVSNSKDVLESGLSPGKYYTHTPRRWSWILILRNLIENDSGVYYCATWDRPEIYYKKLFGSGTTLVVT

SEQ ID NO: 94 - protein, 14 aa, Artificial sequence
  CDR3 VG4 E113
CATWDGPPYYKKLF

SEQ ID NO: 95 - protein, 11 aa, Artificial sequence
  CDR3 VG2 F4
CATWDGQKKLF

SEQ ID NO: 96 - protein, 11 aa, Artificial sequence
  CDR3 VG8 Zi11
CATWDNYKKLF

SEQ ID NO: 97 - protein, 18 aa, Artificial sequence
  CDR3 VD5 D37
CAASSPIRGYTGSDKLIF

SEQ ID NO: 98 - protein, 18 aa, Artificial sequence
  CDR3 VD5 E113
CAASSPIRGYTGSDKLIF

SEQ ID NO: 99 - protein, 17 aa, Artificial sequence
  CDR3 VD1 F4
CALGELRYWGIVDKLIF

SEQ ID NO: 100 - protein, 25 aa, Artificial sequence
  CDR3 VD1 Zi11
CALGELRGQISFLYLLGDTTDKLIF

SEQ ID NO: 101 - protein, 13 aa, Artificial sequence
  CDR3 VG4 E57
CATWDGFYYKKLF

SEQ ID NO: 102 - protein, 18 aa, Artificial sequence
  CDR3 VD5 E57
CAASSPIRGYTGSDKLIF

SEQ ID NO: 103 - protein, 144 aa, Homo sapiens
  TRD E113
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPR

SEQ ID NO: 104 - protein, 135 aa, Homo sapiens
  TRG E113
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGPPYYKKLFGSGTTLVVT

SEQ ID NO: 105 - protein, 138 aa, Homo sapiens
  TRD F4
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGELRYWGIVDKLIFGKGTRVTVEPR

SEQ ID NO: 106 - protein, 132 aa, Homo sapiens
  TRG F4
MEWALAVLLAFLSPASQKSSNLEGRTKSVIRQTGSSAEITCDLAEGSNGYIHWYLHQEGKAPQRLQYYDSYNSKVVLESGVSPGKYYTYASTRNNLRLILRNLIENDSGVYYCATWDGQKKLFGSGTTLVVT

SEQ ID NO: 107 - protein, 146 aa, Homo sapiens
  TRD Zi11
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGELRGQISFLYLLGDTTDKLIFGKGTRVTVEPR

SEQ ID NO: 108 - protein, 132 aa, Homo sapiens
  TRG Zi11
MVLALALLLAFLPPASQKSSNLEGRTKSVTRPTGSSAVITCDLPVENAVYTHWYLHQEGKAPQRLLYYDSYNSRVVLESGISREKYHTYASTGKSLKFILENLIERDSGVYYCATWDNYKKLFGSGTTLVVT

SEQ ID NO: 109 - protein, 144 aa, Homo sapiens; identical to SEQ ID NO: 103
  TRD D37

SEQ ID NO: 110 - protein, 132 aa, Homo sapiens
  TRG D37
MVLALALLLAFLPPASQKSSNLEGRTKSVTRPTGSSAVITCDLPVENAVYTHWYLHQEGKAPQRLLYYDSYNSRVVLESGISREKYHTYASTGKSLKFILENLIERDSGVYYCATWDNYMKLFGSGTTLVVT

SEQ ID NO: 111 - protein, 144 aa, Homo sapiens; identical to SEQ ID NO: 103
  TRD E57

SEQ ID NO: 112 - protein, 134 aa, Homo sapiens
  TRG E57
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVT

SEQ ID NO: 113 - protein, 11 aa, Artificial sequence
  CDR3 VG8 D37
CATWDNYMKLF

SEQ ID NO: 114 - protein, 23 aa, Artificial sequence
  CDR3 VD3 F2
CASSYTLKLGDTPGRVRDWKLIF

SEQ ID NO: 115 - protein, 14 aa, Artificial sequence
  CDR3 VG4 F2
CATWDGPPYYKKLF

SEQ ID NO: 116 - protein, 26 aa, Artificial sequence
  CDR3 VD1 Ze11
CALGDYLGDKYPSYDLLGDTTDKLIF

SEQ ID NO: 117 - protein, 11 aa, Artificial sequence
  CDR3 VG8 Ze11
CATWDNYKKLF

SEQ ID NO: 118 - protein, 18 aa, Artificial sequence
  CDR3 VD5 B23
CAASSPIRGYTGSDKLIF

SEQ ID NO: 119 - protein, 11 aa, Artificial sequence
  CDR3 VG8 B23
CATWDNYKKLF

SEQ ID NO: 120 - protein, 144 aa, Homo sapiens
  TRD F2
MILTVGFSFLFFYRGTLCDKVTQSSPDQTVASGSEVVLLCTYDTVYSNPDLFWYRIRPDYSFQFVFYGDNSRSEGADFTQGRFSVKHILTQKAFHLVISPVRTEDSATYYCASSYTLKLGDTPGRVRDWKLIFGKGTRVTVEPR

SEQ ID NO: 121 - protein, 135 aa, Homo sapiens; identical to SEQ ID NO: 104
  TRG F2

SEQ ID NO: 122 - protein, 147 aa, Homo sapiens
  TRD Ze11
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGDYLGDKYPSYDLLGDTTDKLIFGKGTRVTVEPR

SEQ ID NO: 123 - protein, 132 aa, Homo sapiens; identical to SEQ ID NO: 108
  TRG Ze11

SEQ ID NO: 124 - protein, 144 aa, Homo sapiens; identical to SEQ ID NO: 103
  TRD B23

SEQ ID NO: 125 - protein, 132 aa, Homo sapiens; identical to SEQ ID NO: 108
  TRG B23

SEQ ID NO: 126 - protein, 20 aa, Artificial sequence
  CDR3 VD1 B9
CALGNGNHIGYWRYTDKLIF

SEQ ID NO: 127 - protein, 13 aa, Artificial sequence
  CDR3 VG5  B9
CATWDRLYYKKLF

SEQ ID NO: 128 - protein, 141 aa, Homo sapiens
  TRD B9
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGNGNHIGYWRYTDKLIFGKGTRVTVEPR

SEQ ID NO: 129 - protein, 134 aa, Homo sapiens
  TRG B9
MVWALLVLLAFLSPASQKSSNLEGGTKSVTRPTRSSAEITCDLTVINAFYIHWYLHQEGKAPQRLLYYDVSNSKDVLESGLSPGKYYTHTPRRWSWILILRNLIENDSGVYYCATWDRLYYKKLFGSGTTLVVT

SEQ ID NO: 130 - protein, 10 aa, Artificial sequence
  CDR3 VG8 An2
CATWDSSKLF

SEQ ID NO: 131 - protein, 17 aa, Artificial sequence
  CDR3 VD3 An2
CAFTGLGDTSHADKLIF

SEQ ID NO: 132 - protein, 131 aa, Homo sapiens
  TRG An2
MLLALALLLAFLPPASQKSSNLEGRTKSVTRPTGSSAVITCDLPVENAVYTHWYLHQEGKAPQRLLYYDSYNSRVVLESGISREKYHTYASTGKSLKFILENLIERDSGVYYCATWDSSKLFGSGTTLVVT

SEQ ID NO: 133 - protein, 138 aa, Homo sapiens
  TRD An2
MILTVGFSFLFFYRGTLCDKVTQSSPDQTVASGSEVVLLCTYDTVYSNPDLFWYRIRPDYSFQFVFYGDNSRSEGADFTQGRFSVKHILTQKAFHLVISPVRTEDSATYYCAFTGLGDTSHADKLIFGKGTRVTVEPR

SEQ ID NO: 134 - protein, 153 aa, Homo sapiens
  TRDC
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 135 - protein, 173 aa, Homo sapiens
  TRGC1
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 136 - protein, 189 aa, Homo sapiens
  TRGC2
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 137 - protein, 319 aa, Artificial sequence
  ICOSL -41BB
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCKRGRKKLLYIFKQPFMRPVQTTQEEDGCSCRFPEEEEGGCEL

SEQ ID NO: 138 - protein, 277 aa, Artificial sequence
  ICOSL extra + tm
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVC

SEQ ID NO: 139 - protein, 256 aa, Artificial sequence
  ICOSL extra
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAAT

SEQ ID NO: 140 - DNA, 885 bp, Artificial sequence
  Sequence encoding SEQ ID NO 45 (41BBL-OX40)
atgctggggcgcgaccagaggctgccacctgacgcacacaagccacctggtgggggctcatttcgaactcccatccaggaagagcaggccgacgcacacagtacccttgccaaaatcactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 141 - DNA, 843 bp, Artificial sequence
  Sequence encoding SEQ ID NO 46 (41BBL13W- OX40rev)
atggaaatcaaggctctaaccagccacgcggacgctcaggaagaacaaattcccacccgctttagtggtggggggccacctaaacatgctgatcctccattgcgacaggataggaggctgctgtggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 142 - DNA, 864 bp, Artificial sequence
  Sequence encoding SEQ ID NO 47 (41BBL-NKp80)
atgcaggacgaggacgggtatatgactctcaacgttcagagcaagaagaggtcttccgctcagacgagtcagctgacctttaaagactatagcgttacactccattggtatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 143 - DNA, 792 bp, Artificial sequence
  Sequence encoding SEQ ID NO 48 (41BBLmincyto- NKp80)
atgcaggacgaggacgggtatatgactctcaacgttcagagcaagaagaggtcttccgctcagacgagtcagctgacctttaaagactatagcgttacactccattggtacaaatgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 144 - DNA, 693 bp, Artificial sequence
  Sequence encoding SEQ ID NO 50 (OX40L-41BB)
atgctggggaagcggggcagaaagaaactgctgtacatctttaagcagcccttcatgcggcccgtgcagaccacccaggaagaggacggctgctcctgcagattccccgaggaagaagaaggcggctgcgagctgggaggcagcgctgagcgtgttcagccactggaggaaaatgtcggcaacgccgccaggcctcgattcgagcgtaacaaactgctccttgttgctagtgtgattcaggggctgggactgttgttgtgcttcacatacatttgtctccacttttctgcattgcaggtcagccataggtatccccgcattcagtctatcaaagtgcaattcactgagtacaagaaggagaaaggctttattttaactagtcaaaaggaagacgagataatgaaggtgcagaacaacagcgttattatcaattgcgacgggttctatttaatctctctgaaagggtacttctcacaggaggttaacattagtttacattatcaaaaggacgaggaacccttgttccagctgaaaaaagtgcggtctgttaattcccttatggtggcgagtcttacatacaaggacaaggtgtatctcaacgtgacaacggacaatacctctctggacgattttcacgttaacggcggggagcttatacttatccatcaaaacccaggagaattttgcgtcctt

SEQ ID NO: 145 - DNA, 696 bp, Artificial sequence
  Sequence encoding SEQ ID NO 51 (OX40L-41BBrev)
atgctggggctggagtgcggcggcgaggaggaggagcccttcaggtgcagctgcggcgacgaggagcagaccacccaggtgcccaggatgttcccccagaagttcatctacctgctgaagaagaggggcaggaagctgggaggcagcgctgagcgtgttcagccactggaggaaaatgtcggcaacgccgccaggcctcgattcgagcgtaacaaactgctccttgttgctagtgtgattcaggggctgggactgttgttgtgcttcacatacatttgtctccacttttctgcattgcaggtcagccataggtatccccgcattcagtctatcaaagtgcaattcactgagtacaagaaggagaaaggctttattttaactagtcaaaaggaagacgagataatgaaggtgcagaacaacagcgttattatcaattgcgacgggttctatttaatctctctgaaagggtacttctcacaggaggttaacattagtttacattatcaaaaggacgaggaacccttgttccagctgaaaaaagtgcggtctgttaattcccttatggtggcgagtcttacatacaaggacaaggtgtatctcaacgtgacaacggacaatacctctctggacgattttcacgttaacggcggggagcttatacttatccatcaaaacccaggagaattttgcgtcctt

SEQ ID NO: 146 - DNA, 1104 bp, Artificial sequence
  Sequence encoding SEQ ID NO 52 (CD86-OX40)
atggaccctcagtgtaccatgggcctgagcaacatcctgttcgtgatggcctttctgctgagcggagccgctcctctgaagatccaggcctacttcaacgagacagccgacctgccttgccagttcgccaacagccagaatcagagcctgagcgagctggtggtgttctggcaggaccaagagaacctggtgctgaacgaggtgtacctgggcaaagaaaagttcgacagcgtgcacagcaagtacatgggcagaaccagcttcgactccgacagctggacactgagactgcacaacctgcagatcaaggacaagggcctgtaccagtgcatcatccaccacaagaaacccaccggcatgatcagaatccaccagatgaacagcgagctgagcgtgctggccaacttcagccagcctgagatcgtgcccatcagcaacatcaccgagaacgtgtacatcaacctgacctgcagcagcatccacggctaccccgagcctaagaaaatgtccgtgctgctgcggaccaagaacagcaccatcgagtacgacggcgtgatgcagaaaagccaggacaacgtgaccgagctgtacgacgtgtccatcagcctgtccgtgtcattccccgacgtgaccagcaatatgaccatcttctgcatcctggaaaccgacaagacccggctgctgtctagccccttcagcatagagctggaagatccccagcctcctcctgatcacatcccttggattaccgccgtgctgcccaccgtgatcatctgcgtgatggtgttttgcctgatcctgtggaagtggaagaagaagaaacggcctcggaacagctacaagtgcggcaccaacaccatggaacgggaagagagcgagcagaccaagaagagagagaagattcacatccccgagcggagcgacgaagcccagagagtgtttaagagcagcaagaccagcagctgcgacaagagcgatacctgcttcggatcagggcgggatcagcgcctgcctccagatgcacataaaccccccggcggcgggtcttttagaacaccaatacaagaggaacaagctgacgcccacagtaccctcgcaaaaatt

SEQ ID NO: 147 - DNA, 939 bp, Artificial sequence
  Sequence encoding SEQ ID NO 53 (CD86delP276- OX40)
atggaccctcagtgtaccatgggcctgagcaacatcctgttcgtgatggcctttctgctgagcggagccgctcctctgaagatccaggcctacttcaacgagacagccgacctgccttgccagttcgccaacagccagaatcagagcctgagcgagctggtggtgttctggcaggaccaagagaacctggtgctgaacgaggtgtacctgggcaaagaaaagttcgacagcgtgcacagcaagtacatgggcagaaccagcttcgactccgacagctggacactgagactgcacaacctgcagatcaaggacaagggcctgtaccagtgcatcatccaccacaagaaacccaccggcatgatcagaatccaccagatgaacagcgagctgagcgtgctggccaacttcagccagcctgagatcgtgcccatcagcaacatcaccgagaacgtgtacatcaacctgacctgcagcagcatccacggctaccccgagcctaagaaaatgtccgtgctgctgcggaccaagaacagcaccatcgagtacgacggcgtgatgcagaaaagccaggacaacgtgaccgagctgtacgacgtgtccatcagcctgtccgtgtcattccccgacgtgaccagcaatatgaccatcttctgcatcctggaaaccgacaagacccggctgctgtctagccccttcagcatagagctggaagatccccagcctcctcctgatcacatcccttggattaccgccgtgctgcccaccgtgatcatctgcgtgatggtgttttgcctgatcctgtggaagtggaagaagaagaaacggcctgggcgggatcagcgcctgcctccagatgcacataaaccccccggcggcgggtcttttagaacaccaatacaagaggaacaagctgacgcccacagtaccctcgcaaaaatt

SEQ ID NO: 148 - DNA, 762 bp, Homo sapiens
  Sequence encoding SEQ ID NO 54 (41BBL)
atggaatatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 149 - DNA, 699 bp, Artificial sequence
  Sequence encoding SEQ ID NO 55 (41BBLmincyto)
atgtcaaagagcacgggcagctgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 150 - DNA, 885 bp, Artificial sequence
  Sequence encoding SEQ ID NO 57 (41BBL-OX40rev)
atgctggggatcaaggctctaaccagccacgcggacgctcaggaagaacaaattcccacccgctttagtggtggggggccacctaaacatgctgatcctccattgcgacaggataggactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 151 - DNA, 849 bp, Artificial sequence
  Sequence encoding SEQ ID NO 58 (41BBL-rev extra-OX40tm-cyto)
atgctgctgttggtcacctccttgttactctgcgagcttccacatcccgctttcctcttaattcccgaccagggcatgtttgcgcagctcgtcgcccagaatgtcctcctaatagatggcccactatcctggtacagtgatccaggcctggccggggtgtctcttacgggaggcctgagctataaggaagacaccaaagagttggtggtcgcgaaggccggagtgtattatgttttttttcaacttgagctgcgacgtgtggtcgccggagaaggctcgggatctgtttcactcgctcttcacctgcaaccactgcggtccgccgcgggggctgccgctctcgccctaaccgtggatctgcctcccgcctcttccgaagcacgcaattctgcttttggatttcagggtaggctactgcatctgtccgcaggccaacggttaggtgtgcatctgcatacagaagcccgggctcggcacgcctggcaactaacacagggagctaccgtgctgggtctgtttagagtgacacctgaaatccctgccgggttaccaagccctaggtccgagcgcttggacctgctgggagcaccagatgatcctagcttggagcccggggagagacttcgcccaagtgccgctagcggcccctctgctagggctgttgctgccatcctaggactgggtcttgttctcggcctgctgggcccgttggccattctgcttgccctgtacctgctaagaagagatcaaagactgcctcccgatgcccacaagcctccaggaggagggtcattcagaacccccatccaggaagaacaggcagacgctcactctacgctcgcgaagatc

SEQ ID NO: 152 - DNA, 807 bp, Artificial sequence
  Sequence encoding SEQ ID NO 59 (41BBLmincyto- OX40)
atgctggggcgcgaccagaggctgccacctgacgcacacaagccacctggtgggggctcatttcgaactcccatccaggaagagcaggccgacgcacacagtacccttgccaaaatcactagtggaagttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 153 - DNA, 843 bp, Artificial sequence; identical to SEQ ID NO: 141
  Sequence encoding SEQ ID NO 60 (41BBL13W- OX40rev)

SEQ ID NO: 154 - DNA, 807 bp, Artificial sequence
  Sequence encoding SEQ ID NO 61 (41BBL- mincyto-OX40rev)
atgctggggatcaaggctctaaccagccacgcggacgctcaggaagaacaaattcccacccgctttagtggtggggggccacctaaacatgctgatcctccattgcgacaggataggactagtggaagttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 155 - DNA, 903 bp, Artificial sequence
  Sequence encoding SEQ ID NO 62 (41BBL- OX40ENrev)
atgctggggatcaaggctctaaccagccacgcggacgctcaggaagaacaaattcccacccgctttagtggtggggggccacctaaacatgctgatcctccattgcgacaggataggaggctgctctacctggctactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 156 - DNA, 903 bp, Artificial sequence
  Sequence encoding SEQ ID NO 63 (41BBL- OX40EN)
atgctgggggccctgtacctgctcaggcgcgaccagaggctgccacctgacgcacacaagccacctggtgggggctcatttcgaactcccatccaggaagagcaggccgacgcacacagtacccttgccaaaatcactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 157 - DNA, 873 bp, Artificial sequence
  Sequence encoding SEQ ID NO 64 (41BBLmincyto- 13alink-OX40)
atgctggggcgcgaccagaggctgccacctgacgcacacaagccacctggtgggggctcatttcgaactcccatccaggaagagcaggccgacgcacacagtacccttgccaaaatcagtttaaacggcggaggtgggagcggaggtggcggctccgggggaggtggcagcggtgggggaggcagcggcgggccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 158 - DNA, 627 bp, Artificial sequence
  Sequence encoding SEQ ID NO 65 (OX40Lmincyto- 41BBrev)
atgctggggctggagtgcggcggcgaggaggaggagcccttcaggtgcagctgcggcgacgaggagcagaccacccaggtgcccaggatgttcccccagaagttcatctacctgctgaagaagaggggcaggaagctgggaggcagcctgctccttgttgctagtgtgattcaggggctgggactgttgttgtgcttcacatacatttgtctccacttttctgcattgcaggtcagccataggtatccccgcattcagtctatcaaagtgcaattcactgagtacaagaaggagaaaggctttattttaactagtcaaaaggaagacgagataatgaaggtgcagaacaacagcgttattatcaattgcgacgggttctatttaatctctctgaaagggtacttctcacaggaggttaacattagtttacattatcaaaaggacgaggaacccttgttccagctgaaaaaagtgcggtctgttaattcccttatggtggcgagtcttacatacaaggacaaggtgtatctcaacgtgacaacggacaatacctctctggacgattttcacgttaacggcggggagcttatacttatccatcaaaacccaggagaattttgcgtcctt

SEQ ID NO: 159 - DNA, 549 bp, Homo sapiens
  Sequence encoding SEQ ID NO 66 (OX40L WT)
atggaacgtgttcagccactggaggaaaatgtcggcaacgccgccaggcctcgattcgagcgtaacaaactgctccttgttgctagtgtgattcaggggctgggactgttgttgtgcttcacatacatttgtctccacttttctgcattgcaggtcagccataggtatccccgcattcagtctatcaaagtgcaattcactgagtacaagaaggagaaaggctttattttaactagtcaaaaggaagacgagataatgaaggtgcagaacaacagcgttattatcaattgcgacgggttctatttaatctctctgaaagggtacttctcacaggaggttaacattagtttacattatcaaaaggacgaggaacccttgttccagctgaaaaaagtgcggtctgttaattcccttatggtggcgagtcttacatacaaggacaaggtgtatctcaacgtgacaacggacaatacctctctggacgattttcacgttaacggcggggagcttatacttatccatcaaaacccaggagaattttgcgtcctt

SEQ ID NO: 160 - DNA, 624 bp, Artificial sequence
  Sequence encoding SEQ ID NO 67 (OX40Lmincyto- 41BB)
atgctggggaagcggggcagaaagaaactgctgtacatctttaagcagcccttcatgcggcccgtgcagaccacccaggaagaggacggctgctcctgcagattccccgaggaagaagaaggcggctgcgagctgggaggcagcctgctccttgttgctagtgtgattcaggggctgggactgttgttgtgcttcacatacatttgtctccacttttctgcattgcaggtcagccataggtatccccgcattcagtctatcaaagtgcaattcactgagtacaagaaggagaaaggctttattttaactagtcaaaaggaagacgagataatgaaggtgcagaacaacagcgttattatcaattgcgacgggttctatttaatctctctgaaagggtacttctcacaggaggttaacattagtttacattatcaaaaggacgaggaacccttgttccagctgaaaaaagtgcggtctgttaattcccttatggtggcgagtcttacatacaaggacaaggtgtatctcaacgtgacaacggacaatacctctctggacgattttcacgttaacggcggggagcttatacttatccatcaaaacccaggagaattttgcgtcctt

SEQ ID NO: 161 - DNA, 987 bp, Homo sapiens
  Sequence encoding SEQ ID NO 68 (CD86 WT)
atggaccctcagtgtaccatgggcctgagcaacatcctgttcgtgatggcctttctgctgagcggagccgctcctctgaagatccaggcctacttcaacgagacagccgacctgccttgccagttcgccaacagccagaatcagagcctgagcgagctggtggtgttctggcaggaccaagagaacctggtgctgaacgaggtgtacctgggcaaagaaaagttcgacagcgtgcacagcaagtacatgggcagaaccagcttcgactccgacagctggacactgagactgcacaacctgcagatcaaggacaagggcctgtaccagtgcatcatccaccacaagaaacccaccggcatgatcagaatccaccagatgaacagcgagctgagcgtgctggccaacttcagccagcctgagatcgtgcccatcagcaacatcaccgagaacgtgtacatcaacctgacctgcagcagcatccacggctaccccgagcctaagaaaatgtccgtgctgctgcggaccaagaacagcaccatcgagtacgacggcgtgatgcagaaaagccaggacaacgtgaccgagctgtacgacgtgtccatcagcctgtccgtgtcattccccgacgtgaccagcaatatgaccatcttctgcatcctggaaaccgacaagacccggctgctgtctagccccttcagcatagagctggaagatccccagcctcctcctgatcacatcccttggattaccgccgtgctgcccaccgtgatcatctgcgtgatggtgttttgcctgatcctgtggaagtggaagaagaagaaacggcctcggaacagctacaagtgcggcaccaacaccatggaacgggaagagagcgagcagaccaagaagagagagaagattcacatccccgagcggagcgacgaagcccagagagtgtttaagagcagcaagaccagcagctgcgacaagagcgatacctgcttc

SEQ ID NO: 162 - DNA, 828 bp, Artificial sequence
  Sequence encoding SEQ ID NO 69 (CD86delP276)
atggaccctcagtgtaccatgggcctgagcaacatcctgttcgtgatggcctttctgctgagcggagccgctcctctgaagatccaggcctacttcaacgagacagccgacctgccttgccagttcgccaacagccagaatcagagcctgagcgagctggtggtgttctggcaggaccaagagaacctggtgctgaacgaggtgtacctgggcaaagaaaagttcgacagcgtgcacagcaagtacatgggcagaaccagcttcgactccgacagctggacactgagactgcacaacctgcagatcaaggacaagggcctgtaccagtgcatcatccaccacaagaaacccaccggcatgatcagaatccaccagatgaacagcgagctgagcgtgctggccaacttcagccagcctgagatcgtgcccatcagcaacatcaccgagaacgtgtacatcaacctgacctgcagcagcatccacggctaccccgagcctaagaaaatgtccgtgctgctgcggaccaagaacagcaccatcgagtacgacggcgtgatgcagaaaagccaggacaacgtgaccgagctgtacgacgtgtccatcagcctgtccgtgtcattccccgacgtgaccagcaatatgaccatcttctgcatcctggaaaccgacaagacccggctgctgtctagccccttcagcatcgagctggaagatccccagcctcctcctgatcacatcccttggattaccgccgtgctgcccaccgtgatcatctgcgtgatggtgttttgcctgatcctgtggaagtggaagaagaagaaacggcct

SEQ ID NO: 163 - DNA, 804 bp, Artificial sequence
  Sequence encoding SEQ ID NO 70 (CD86mincyto)
atggaccctcagtgtaccatgggcctgagcaacatcctgttcgtgatggcctttctgctgagcggagccgctcctctgaagatccaggcctacttcaacgagacagccgacctgccttgccagttcgccaacagccagaatcagagcctgagcgagctggtggtgttctggcaggaccaagagaacctggtgctgaacgaggtgtacctgggcaaagaaaagttcgacagcgtgcacagcaagtacatgggcagaaccagcttcgactccgacagctggacactgagactgcacaacctgcagatcaaggacaagggcctgtaccagtgcatcatccaccacaagaaacccaccggcatgatcagaatccaccagatgaacagcgagctgagcgtgctggccaacttcagccagcctgagatcgtgcccatcagcaacatcaccgagaacgtgtacatcaacctgacctgcagcagcatccacggctaccccgagcctaagaaaatgtccgtgctgctgcggaccaagaacagcaccatcgagtacgacggcgtgatgcagaaaagccaggacaacgtgaccgagctgtacgacgtgtccatcagcctgtccgtgtcattccccgacgtgaccagcaatatgaccatcttctgcatcctggaaaccgacaagacccggctgctgtctagccccttcagcatagagctggaagatccccagcctcctcctgatcacatcccttggattaccgccgtgctgcccaccgtgatcatctgcgtgatggtgttttgcctgatcctgtgg

SEQ ID NO: 164 - DNA, 1470 bp, Artificial sequence
  Sequence encoding SEQ ID NO 71 (CD86mincyto- IL18RAP)
atggaccctcagtgtaccatgggcctgagcaacatcctgttcgtgatggcctttctgctgagcggagccgctcctctgaagatccaggcctacttcaacgagacagccgacctgccttgccagttcgccaacagccagaatcagagcctgagcgagctggtggtgttctggcaggaccaagagaacctggtgctgaacgaggtgtacctgggcaaagaaaagttcgacagcgtgcacagcaagtacatgggcagaaccagcttcgactccgacagctggacactgagactgcacaacctgcagatcaaggacaagggcctgtaccagtgcatcatccaccacaagaaacccaccggcatgatcagaatccaccagatgaacagcgagctgagcgtgctggccaacttcagccagcctgagatcgtgcccatcagcaacatcaccgagaacgtgtacatcaacctgacctgcagcagcatccacggctaccccgagcctaagaaaatgtccgtgctgctgcggaccaagaacagcaccatcgagtacgacggcgtgatgcagaaaagccaggacaacgtgaccgagctgtacgacgtgtccatcagcctgtccgtgtcattccccgacgtgaccagcaatatgaccatcttctgcatcctggaaaccgacaagacccggctgctgtctagccccttcagcatcgagctggaagatccccagcctcctcctgatcacatcccttggattaccgccgtgctgcccaccgtgatcatctgcgtgatggtgttttgcctgatcctgtggagcgccctgctgtaccggcactggatcgagatcgtgctgctgtacagaacctaccagagcaaggaccagacactgggcgacaagaaggacttcgacgccttcgtgtcctacgccaagtggtccagctttcctagcgaggccacaagcagcctgagcgaggaacatctggccctgagcctgtttcctgacgtgctggaaaacaaatacggctacagcctgtgcctgctcgagagagatgttgctcctggcggagtgtacgccgaggacatcgtgtccatcatcaagcggagcagacggggcatcttcattctgagccccaactacgtgaacggccccagcatctttgaactgcaagccgccgtgaacctggctctggatgatcagaccctgaagctgatcctgatcaagttctgctacttccaagagcctgagagcctgcctcacctggtcaagaaagccctgagagtgctgcctaccgtgacttggagaggcctgaagtccgtgcctcctaacagcagattctgggccaagatgagataccacatgcctgtgaagaacagccagggcttcacctggaaccagctgcggatcaccagccggatctttcagtggaagggcctgagcagaaccgagacaaccggcagaagcagccagcctaaagagtgg

SEQ ID NO: 165 - DNA, 1653 bp, Artificial sequence
  Sequence encoding SEQ ID NO 72 (CD86-IL18RAP)
atggaccctcagtgtaccatgggcctgagcaacatcctgttcgtgatggcctttctgctgagcggagccgctcctctgaagatccaggcctacttcaacgagacagccgacctgccttgccagttcgccaacagccagaatcagagcctgagcgagctggtggtgttctggcaggaccaagagaacctggtgctgaacgaggtgtacctgggcaaagaaaagttcgacagcgtgcacagcaagtacatgggcagaaccagcttcgactccgacagctggacactgagactgcacaacctgcagatcaaggacaagggcctgtaccagtgcatcatccaccacaagaaacccaccggcatgatcagaatccaccagatgaacagcgagctgagcgtgctggccaacttcagccagcctgagatcgtgcccatcagcaacatcaccgagaacgtgtacatcaacctgacctgcagcagcatccacggctaccccgagcctaagaaaatgtccgtgctgctgcggaccaagaacagcaccatcgagtacgacggcgtgatgcagaaaagccaggacaacgtgaccgagctgtacgacgtgtccatcagcctgtccgtgtcattccccgacgtgaccagcaatatgaccatcttctgcatcctggaaaccgacaagacccggctgctgtctagccccttcagcatcgagctggaagatccccagcctcctcctgatcacatcccttggattaccgccgtgctgcccaccgtgatcatctgcgtgatggtgttttgcctgatcctgtggaagtggaagaagaagaaacggcctcggaacagctacaagtgcggcaccaacaccatggaacgggaagagagcgagcagaccaagaagagagagaagattcacatccccgagcggagcgacgaagcccagagagtgtttaagagcagcaagaccagcagctgcgacaagagcgatacctgcttcagcgccctgctgtaccggcactggatcgagatcgtgctgctgtacagaacctaccagagcaaggaccagacactgggcgacaagaaggacttcgacgccttcgtgtcctacgccaagtggtccagctttcctagcgaggccacaagcagcctgagcgaggaacatctggccctgagcctgtttcctgacgtgctggaaaacaaatacggctacagcctgtgcctgctcgagagagatgttgctcctggcggagtgtacgccgaggacatcgtgtccatcatcaagcggagcagacggggcatcttcattctgagccccaactacgtgaacggccccagcatctttgaactgcaagccgccgtgaacctggctctggatgatcagaccctgaagctgatcctgatcaagttctgctacttccaagagcctgagagcctgcctcacctggtcaagaaagccctgagagtgctgcctaccgtgacttggagaggcctgaagtccgtgcctcctaacagcagattctgggccaagatgagataccacatgcctgtgaagaacagccagggcttcacctggaaccagctgcggatcaccagccggatctttcagtggaagggcctgagcagaaccgagacaaccggcagaagcagccagcctaaagagtgg

SEQ ID NO: 166 - DNA, 1311 bp, Artificial sequence
  Sequence encoding SEQ ID NO 73 (CD86IgV- 41BBL-OX40)
atgctggggcgcgaccagaggctgccacctgacgcacacaagccacctggtgggggctcatttcgaactcccatccaggaagagcaggccgacgcacacagtacccttgccaaaatcactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaaagtttaaacggcggaggtgggagcggaggtggcggctccgggggaggtggcagcggtgggggaggcagcggcgggggaggtagcactagtgcccctctgaagatccaggcttacttcaacgaaaccgcagatttgccctgccagtttgccaattctcagaatcagtctttatctgaactggtggtcttctggcaggaccaggagaatcttgtactgaatgaagtttatctcggcaaggaaaagtttgatagtgtacatagtaagtacatgggtagaacctccttcgattctgactcgtggactttaaggctgcacaacctgcagatcaaggataaagggctttaccagtgcattatccaccataagaagccaaccggcatgattcggattcatcagatgaactctgagctgtctgtactcgccaat

SEQ ID NO: 167 - DNA, 1839 bp, Homo sapiens
  Sequence encoding SEQ ID NO 74 (RANK WT)
atggcccctagagccagaagaagaaggcctctgtttgccctgctgctgctgtgcgctctgctggctagactgcaagtggccctgcagattgcccctccatgtaccagcgagaagcactacgagcacctgggcagatgctgcaacaagtgcgagcccggcaagtacatgagcagcaagtgtaccaccaccagcgacagcgtgtgtctgccttgtggccctgacgagtacctggacagctggaacgaagaggacaagtgcctgctgcacaaagtgtgcgataccggcaaagccctggtggcagtggtggccggcaatagcacaacccctagaagatgtgcctgcaccgccggctatcactggtcccaggattgcgagtgctgcagacggaataccgagtgtgctcctggactgggagcacagcatcctctgcagctgaacaaggacaccgtgtgcaagccttgtctggccggctacttcagcgacgcctttagcagcaccgacaagtgcagaccctggaccaactgcaccttcctgggcaagagagtggaacaccacggcaccgagaagtccgatgccgtgtgtagcggcagcagaaagcctcctaatgagccccacgtgtacctgcctggcctgatcatcctgctgctttttgctagcgtggccctggtcgccgccatcatctttggcgtgtgctaccggaagaaaggcaaggccctgaccgccaatctgtggcactggatcaatgaggcctgcggcagactgagcggcgacaaagaaagcagcggcgactcctgtgtgtctacccacaccgccaatttcggacagcagggcgcttgtgaaggcgtgctgcttctgaccctggaagagaaaacattccccgaggacatgtgctaccccgaccaaggcggagtgtgtcagggcacatgtgttggcggaggcccttatgctcagggcgaagatgccagaatgctgagcctggtgtccaagaccgagatcgaggaagatagcttccggcagatgcccaccgaggatgagtacatggatagacccagccagcctaccgaccagctgctgtttctgacagagcctggcagcaagagcacccctccattttccgagcctctggaagtgggcgagaacgacagcctgagccagtgctttacaggcacccagagcacagtgggcagcgagagctgtaattgcaccgagccactgtgccggaccgactggacacctatgagcagcgagaactacctgcagaaagaggtggactccggccactgtcctcattgggctgctagcccctctcctaactgggccgatgtgtgtaccggctgccgaaatccacctggcgaagattgcgaacctctcgtgggcagccctaaacgaggacctctgcctcagtgtgcctacggcatgggactgcctccagaggaagaggccagcagaaccgaagccagagatcagcctgaagatggcgccgatggcagactgcctagctctgctagagctggcgctggctctggatcttctcctggcggacaatctcctgccagcggcaatgtgaccggcaacagcaacagcaccttcatcagcagcggccaagtgatgaacttcaagggcgacatcatcgtggtgtacgtgtcccagacaagccaagaaggcgctgccgcagctgctgaacctatgggtagacccgtgcaagaggaaaccctggccagaagagattccttcgccggcaacggccctagattccctgatccttgtggcggccctgaaggcctgagagagcctgaaaaagccagccggcctgtgcaagaacaaggcggcgctaaagct

SEQ ID NO: 168 - DNA, 690 bp, Artificial sequence
  Sequence encoding SEQ ID NO 75 (RANKmincyto)
atggcccctagagccagaagaagaaggcctctgtttgccctgctgctgctgtgcgctctgctggctagactgcaagtggccctgcagattgcccctccatgtaccagcgagaagcactacgagcacctgggcagatgctgcaacaagtgcgagcccggcaagtacatgagcagcaagtgtaccaccaccagcgacagcgtgtgtctgccttgtggccctgacgagtacctggacagctggaacgaagaggacaagtgcctgctgcacaaagtgtgcgataccggcaaagccctggtggcagtggtggccggcaatagcacaacccctagaagatgtgcctgcaccgccggctatcactggtcccaggattgcgagtgctgcagacggaataccgagtgtgctcctggactgggagcacagcatcctctgcagctgaacaaggacaccgtgtgcaagccttgtctggccggctacttcagcgacgcctttagcagcaccgacaagtgcagaccctggaccaactgcaccttcctgggcaagagagtggaacaccacggcaccgagaagtccgatgccgtgtgtagcggcagcagaaagcctcctaatgagccccacgtgtacctgcctggcctgatcatcctgctgctttttgctagcgtggccctggtcgccgccatcatctttggcgtg

SEQ ID NO: 169 - DNA, 816 bp, Artificial sequence
  Sequence encoding SEQ ID NO 76 (RANK-OX40)
atggcccctcgtgctcgacgtaggcgtccattattcgctctcctgctcttgtgcgcgcttctcgctcggctgcaggtggccctccagattgcgcccccatgcacttccgagaagcactatgaacatcttggccgctgttgcaataaatgtgaacctggcaaatacatgtcctccaaatgtactactacgtcagacagcgtgtgcttgccctgtggacccgacgagtatctggattcatggaatgaggaggataaatgcctcttgcataaggtctgtgatactgggaaggcgctggtggctgtggtggctggaaacagtaccacaccgcgacgatgcgcttgtactgccggatatcactggtctcaggattgcgagtgctgtagacgtaatacagaatgtgcccccggccttggcgctcagcaccctctgcagttgaataaagacacagtctgtaaaccctgtctggcagggtatttctctgatgcatttagttctacagacaagtgtcgcccttggaccaactgtactttccttggtaagagggtggaacaccacggcacagaaaagtccgacgccgtctgttcaggctcccggaaaccgcccaacgaaccacatgtgtatctgcccgtggctgccattctgggactggggcttgtcttggggctgctcgggccattggcgatcctgctggccctgtacctgttgcggcgggatcagcgcctgcctccagatgcacataaaccccccggcggcgggtcttttagaacaccaatacaagaggaacaagctgacgcccacagtaccctcgcaaaaatt

SEQ ID NO: 170 - DNA, 834 bp, Artificial sequence
  Sequence encoding SEQ ID NO 77 (RANK-41BB)
atggcccctcgtgctcgacgtaggcgtccattattcgctctcctgctcttgtgcgcgcttctcgctcggctgcaggtggccctccagattgcgcccccatgcacttccgagaagcactatgaacatcttggccgctgttgcaataaatgtgaacctggcaaatacatgtcctccaaatgtactactacgtcagacagcgtgtgcttgccctgtggacccgacgagtatctggattcatggaatgaggaggataaatgcctcttgcataaggtctgtgatactgggaaggcgctggtggctgtggtggctggaaacagtaccacaccgcgacgatgcgcttgtactgccggatatcactggtctcaggattgcgagtgctgtagacgtaatacagaatgtgcccccggccttggcgctcagcaccctctgcagttgaataaagacacagtctgtaaaccctgtctggcagggtatttctctgatgcatttagttctacagacaagtgtcgcccttggaccaactgtactttccttggtaagagggtggaacaccacggcacagaaaagtccgacgccgtctgttcaggctcccggaaaccgcccaacgaaccacatgtgtatctgcccattatttcttttttcctggcactgacaagcacagcgctgttatttctcttattctttttgaccctccgcttcagtgtcgtgaagcgcgggcgcaaaaagttgctgtacatttttaagcagccttttatgagacccgtgcagaccacccaggaggaagatggctgcagctgcaggttccccgaggaggaagagggcggatgcgaactc

SEQ ID NO: 171 - DNA, 1356 bp, Artificial sequence
  Sequence encoding SEQ ID NO 78 (RANK-IL18RAP)
atggcccctagagccagaagaagaaggcctctgtttgccctgctgctgctgtgcgctctgctggctagactgcaagtggccctgcagattgcccctccatgtaccagcgagaagcactacgagcacctgggcagatgctgcaacaagtgcgagcccggcaagtacatgagcagcaagtgtaccaccaccagcgacagcgtgtgtctgccttgtggccctgacgagtacctggacagctggaacgaagaggacaagtgcctgctgcacaaagtgtgcgataccggcaaagccctggtggcagtggtggccggcaatagcacaacccctagaagatgtgcctgcaccgccggctatcactggtcccaggattgcgagtgctgcagacggaataccgagtgtgctcctggactgggagcacagcatcctctgcagctgaacaaggacaccgtgtgcaagccttgtctggccggctacttcagcgacgcctttagcagcaccgacaagtgcagaccctggaccaactgcaccttcctgggcaagagagtggaacaccacggcaccgagaagtccgatgccgtgtgtagcggcagcagaaagcctcctaatgagccccacgtgtacctgcctggtgtagtcctgctctacatcctgctgggcacaatcgggactctggtggctgtgctggctgccagcgccctgctgtaccggcactggatcgagatcgtgctgctgtacagaacctaccagagcaaggaccagacactgggcgacaagaaggacttcgacgccttcgtgtcctacgccaagtggtccagctttcctagcgaggccacaagcagcctgagcgaggaacatctggccctgagcctgtttcctgacgtgctggaaaacaaatacggctacagcctgtgcctgctcgagagagatgttgctcctggcggagtgtacgccgaggacatcgtgtccatcatcaagcggagcagacggggcatcttcattctgagccccaactacgtgaacggccccagcatctttgaactgcaagccgccgtgaacctggctctggatgatcagaccctgaagctgatcctgatcaagttctgctacttccaagagcctgagagcctgcctcacctggtcaagaaagccctgagagtgctgcctaccgtgacttggagaggcctgaagtccgtgcctcctaacagcagattctgggccaagatgagataccacatgcctgtgaagaacagccagggcttcacctggaaccagctgcggatcaccagccggatctttcagtggaagggcctgagcagaaccgagacaaccggcagaagcagccagcctaaagagtgg

SEQ ID NO: 172 - DNA, 414 bp, Artificial sequence
  Sequence encoding SEQ ID NO 80 (CD8-Q8)
atgggactcgttagaagaggcgccagagccggacctagaatccctagaggatggacagccctgtgcctgctgtctctgctgccttctggctttatggccgagctgcctacacagggcaccttcagcaatgtgtccaccaacgtgtccccagccaagcctacaacaacccctgctcctagacctcctacaccagctcctacaatcgccagccagccactgtctctgaggccagaagcttgtagacctgctgctggcggagccgtgcatacaagaggactggatttcgcctgcgacatctacatctgggcccctctggctggaacatgtggcgttctgctgctgagcctggtcatcaccctgtactgcaaccaccggaacaggcggagagtgtgcaagtgccctagacctgtggtt

SEQ ID NO: 173 - DNA, 717 bp, Artificial sequence
  Sequence encoding SEQ ID NO 81 (eGFP)
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 174 - protein, 155 aa, Artificial sequence
  CD70
QRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 175 - protein, 94 aa, Artificial sequence
  IL2RB ICD
VLHTPDQGQLEQLSLYADTNLPLLQTVKDRELVELPSIEPALGGPSFSSSPFPSSLWKQVDGGHESSLQSFFKSPDPTNCKLVKKLWPGTNRCN

SEQ ID NO: 176 - protein, 18 aa, Artificial sequence
  CD70 ICD
MLGPEEGSGCSVRRRPYG

SEQ ID NO: 177 - protein, 21 aa, Artificial sequence
  CD70 -TM
VLRAALVPLVAGLVICLVVCI

SEQ ID NO: 178 - protein, 283 aa, Artificial sequence
  41BBL-13W-OX40
MLGRDQRLPPDAHKPPGGGSFRTPIQEEQADAHSTLAKITSGSWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 179 - protein, 392 aa, Artificial sequence
  41BBL-OX40-IL2RB
MLGVLHTPDQGQLEQLSLYADTNLPLLQTVKDRELVELPSIEPALGGPSFSSSPFPSSLWKQVDGGHESSLQSFFKSPDPTNCKLVKKLWPGTNRCNGSGRDQRLPPDAHKPPGGGSFRTPIQEEQADAHSTLAKITSGSYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 180 - protein, 195 aa, Homo sapiens
  CD70
MLGPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 181 - protein, 180 aa, Artificial sequence
  CD70mincyto
MLGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 182 - protein, 221 aa, Artificial sequence
  CD70mincyto-OX40
MLGRDQRLPPDAHKPPGGGSFRTPIQEEQADAHSTLAKITSGSGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 183 - protein, 245 aa, Artificial sequence
  CD70-41BBL-OX40
MLGRDQRLPPDAHKPPGGGSFRTPIQEEQADAHSTLAKITSGSYASDASLDPEAPWPPAPRARACRVLPWALVAGLLLLLLLAAACAVFLQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 184 - protein, 486 aa, Artificial sequence
  CD19.BB.Z
MALPVTALLLPLALLLHAARPDIQMTQTTSSLSASLGDRVTISCRASQDISKYLNWYQQKPDGTVKLLIYHTSRLHSCVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKCLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 185 - DNA, 849 bp, Artificial sequence
  Sequence encoding SEQ ID NO 178 (41BBL-13W- OX40)
atgctggggcgcgaccagaggctgccacctgacgcacacaagccacctggtgggggctcatttcgaactcccatccaggaagagcaggccgacgcacacagtacccttgccaaaatcactagtggaagttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 186 - DNA, 1176 bp, Artificial sequence
  Sequence encoding SEQ ID NO 179 (41BBL-OX40- IL2RB)
atgctgggggtgctgcacacaccagatcagggacagctggaacagctgagcctgtacgccgacaccaatctgcctctgctgcagaccgtgaaggaccgcgaactggtggaactgcctagcatcgaacctgctctcggcggacctagctttagcagcagcccatttcctagcagcctgtggaaacaggtggacggcggccacgaatctagcctgcagagcttcttcaagagccccgatcctaccaactgcaagctggtcaagaagctgtggcccggcaccaacagatgcaacggcagcggccgcgaccagaggctgccacctgacgcacacaagccacctggtgggggctcatttcgaactcccatccaggaagagcaggccgacgcacacagtacccttgccaaaatcactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaa

SEQ ID NO: 187 - DNA, 585 bp, Artificial sequence
  Sequence encoding SEQ ID NO 180 (CD70)
atgctgggacctgaggagggcagcggctgcagcgtgaggaggaggccctacggctgcgtgctgagggccgccctggtgcccctggtggccggcctggtgatctgcctggtggtgtgcatccagaggttcgcccaggcccagcagcagctgcccctggagagcctgggctgggacgtggccgagctgcagctgaaccacaccggcccccagcaggaccctagactgtactggcaaggcggacctgctctgggcagatcttttctgcacggccccgagctggataagggccagctgagaatccaccgcgacggcatctacatggtgcacatccaagtgaccctggccatctgcagcagcaccacagcctctagacatcaccctaccacactggccgtgggcatctgttctccagccagcagatctatcagcctgctgcggctgagcttccaccagggatgtacaatcgccagccagagactgacccctctggctagaggcgataccctgtgcaccaatctgaccggcacactgctgcccagccggaacaccgatgagacattctttggcgtgcagtgggtccgacct

SEQ ID NO: 188 - DNA, 540 bp, Artificial sequence
  Sequence encoding SEQ ID NO 181 (CD70-mincyto)
atgctggggtgcgtgctgagggccgccctggtgcccctggtggccggcctggtgatctgcctggtggtgtgcatccagaggttcgcccaggcccagcagcagctgcccctggagagcctgggctgggacgtggccgagctgcagctgaaccacaccggcccccagcaggaccctagactgtactggcaaggcggacctgctctgggcagatcttttctgcacggccccgagctggataagggccagctgagaatccaccgcgacggcatctacatggtgcacatccaagtgaccctggccatctgcagcagcaccacagcctctagacatcaccctaccacactggccgtgggcatctgttctccagccagcagatctatcagcctgctgcggctgagcttccaccagggatgtacaatcgccagccagagactgacccctctggctagaggcgataccctgtgcaccaatctgaccggcacactgctgcccagccggaacaccgatgagacattctttggcgtgcagtgggtccgacct

SEQ ID NO: 189 - DNA, 663 bp, Artificial sequence
  Sequence encoding SEQ ID NO 182 (CD70-mincyto- OX40)
atgctggggcgcgaccagaggctgccacctgacgcacacaagccacctggtgggggctcatttcgaactcccatccaggaagagcaggccgacgcacacagtacccttgccaaaatcactagtggaagtggatgcgtgctgagggccgccctggtgcccctggtggccggcctggtgatctgcctggtggtgtgcatccagaggttcgcccaggcccagcagcagctgcccctggagagcctgggctgggacgtggccgagctgcagctgaaccacaccggcccccagcaggaccctagactgtactggcaaggcggacctgctctgggcagatcttttctgcacggccccgagctggataagggccagctgagaatccaccgcgacggcatctacatggtgcacatccaagtgaccctggccatctgcagcagcaccacagcctctagacatcaccctaccacactggccgtgggcatctgttctccagccagcagatctatcagcctgctgcggctgagcttccaccagggatgtacaatcgccagccagagactgacccctctggctagaggcgataccctgtgcaccaatctgaccggcacactgctgcccagccggaacaccgatgagacattctttggcgtgcagtgggtccgacct

SEQ ID NO: 190 - DNA, 735 bp, Artificial sequence
  Sequence encoding SEQ ID NO 183 (CD70EC-41BBL- OX40)
atgctggggcgcgaccagaggctgccacctgacgcacacaagccacctggtgggggctcatttcgaactcccatccaggaagagcaggccgacgcacacagtacccttgccaaaatcactagtggaagttatgccagtgacgcatctctcgatcccgaagccccttggccaccggcgccaagagctagagcttgcagagtgttgccttgggctttggtggccggactgctgctcctgctgctgctggctgctgcctgtgccgtgtttctccagaggttcgcccaggcccagcagcagctgcccctggagagcctgggctgggacgtggccgagctgcagctgaaccacaccggcccccagcaggaccctagactgtactggcaaggcggacctgctctgggcagatcttttctgcacggccccgagctggataagggccagctgagaatccaccgcgacggcatctacatggtgcacatccaagtgaccctggccatctgcagcagcaccacagcctctagacatcaccctaccacactggccgtgggcatctgttctccagccagcagatctatcagcctgctgcggctgagcttccaccagggatgtacaatcgccagccagagactgacccctctggctagaggcgataccctgtgcaccaatctgaccggcacactgctgcccagccggaacaccgatgagacattctttggcgtgcagtgggtccgacct

SEQ ID NO: 191 - RNA, 20 nt, Artificial sequence
  sgRNA
acaaaacugugcuagacaug

SEQ ID NO: 192 - RNA, 20 nt, Artificial sequence
  sgRNA
gagaaucaaaaucggugaau

SEQ ID NO: 193 - RNA, 20 nt, Artificial sequence
  sgRNA
cucucagcugguacacggca

SEQ ID NO: 194 - RNA, 20 nt, Artificial sequence
  sgRNA
acccgaggucgcuguguuug

SEQ ID NO: 195 - RNA, 20 nt, Artificial sequence
  sgRNA
aggucgcuguguuugagcca

SEQ ID NO: 196 - RNA, 20 nt, Artificial sequence
  sgRNA
cgaccacguggagcugagcu

SEQ ID NO: 197 - RNA, 20 nt, Artificial sequence
  sgRNA
gauacugccugagcagccgc

SEQ ID NO: 198 - protein, 316 aa, Homo sapiens
  NY-ESO-1 TRB with furin site
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRS

SEQ ID NO: 199 - protein, 274 aa, Homo sapiens [sequence omitted]

SEQ ID NO: 200 - DNA, 948 bp, Homo sapiens
  NY-ESO-1 TRB DNA
atgtctatcggcctgctgtgttgtgccgctctgtctctgctttgggccggacctgttaatgccggcgtgacccagacacctaagttccaggtgctgaaaaccggccagagcatgaccctgcagtgcgcccaggatatgaaccacgagtacatgagctggtacagacaggaccctggcatgggcctgagactgatccactattctgtcggagccggcatcaccgaccagggcgaagttcctaatggctacaacgtgtccagaagcaccaccgaggacttcccactgagactgctgtctgccgctcctagccagaccagcgtgtacttttgtgccagcagctacgtgggcaacaccggcgagctgttttttggcgagggcagcagactgaccgtgctggaagatctgaagaacgtgttcccaccagaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcacagatccccagcctctgaaagaacagcccgctctgaacgacagccggtactgtctgtcctccagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgttctgtctgccaccatcctgtacgagatcctgctgggcaaagccactctgtacgccgtgctggtgtctgccctggtgctgatggccatggtcaagcggaaggacagtagaggcagagccaagagaagc

SEQ ID NO: 201 - DNA, 822 bp, Homo sapiens
  NY-ESO-1 TRA DNA
atggaaacactgctgggcctgctgatcctgtggctgcaactgcaatgggtgtcctccaagcaagaagtgactcagatcccagccgctctgagtgtgccagagggcgaaaacctggtcctgaactgcagcttcaccgacagcgccatctacaacctgcagtggttcagacaagatcccggcaagggactgaccagcctgctgctgattcagagcagccagagagagcagacctccggcagactgaatgccagcctggataagagcagcggccggtctacactgtatatcgccgcatctcagcctggcgatagcgccacatatctgtgtgccgtgcgacctctgtacggcggcagctacatccctacatttggcagaggcaccagcctgatcgtgcacccctacattcagaaccccgatcctgccgtgtatcagctgagagacagcaagtccagcgacaagagcgtgtgcctgttcaccgacttcgactcccagaccaatgtgtcccagagcaaggacagcgacgtgtacatcaccgataagacagtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttccggattctgctgctcaaggtggccggcttcaacctgctgatgaccctgagactctggtccagc

SEQ ID NO: 202 - DNA, 367 bp, Artificial sequence
  MSCV promoter
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgtttctgctccccgagctcaataaaagagcccacaacccctcact

SEQ ID NO: 203 - DNA, 594 bp, Artificial sequence
  MMLV promoter
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 204 - DNA, 1182 bp, Artificial sequence
  EF1a  promoter
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 205 - DNA, 399 bp, Artificial sequence
  MND promoter
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 206 - protein, 19 aa, Artificial sequence
  P2A
ATNFSLLKQAGDVEENPGP

SEQ ID NO: 207 - DNA, 57 bp, Artificial sequence
  P2A DNA
gccaccaatttcagcctgctgaaacaggctggcgacgtggaagagaaccctgggccc

SEQ ID NO: 208 - protein, 18 aa, Artificial sequence
  T2A
EGRGSLLTCGDVEENPGP

SEQ ID NO: 209 - DNA, 54 bp, Artificial sequence
  T2A DNA
gaaggccgcggcagcctgctgacctgcggagacgtggaagaaaaccctggcccg

SEQ ID NO: 210 - protein, 15 aa, Artificial sequence
  NY-ESO-1 CDR3  TCR alpha
CAVRPLYGGSYIPTF

SEQ ID NO: 211 - protein, 14 aa, Artificial sequence
  NY-ESO-1 CDR3 TCR beta
CASSYVGNTGELFF

SEQ ID NO: 212 - protein, 469 aa, Artificial sequence
  EGFR.BB.Z
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGGGGSGGGGSGGGGSGGGGSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 213 - protein, 486 aa, Artificial sequence
  CD19.28.z
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAGTTTTPAPRPPTPAPTIASQPLSLRPEACRPAAGGAVHTRGLDFACDRRPPSKPFWVLVVVGGVLACYSLLVTVAFIIFWVRSKRSRLLHSDYMNMTPRRPGPTRKHYQPYAPPRDFAAYRSASLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 214 - protein, 271 aa, Homo sapiens [sequence omitted]

SEQ ID NO: 215 - protein, 309 aa, Homo sapiens [sequence omitted]

SEQ ID NO: 216 - protein, 12 aa, Artificial sequence
  MUC1 TRA CDR3
CAVTSSYGKLTF

SEQ ID NO: 217 - protein, 13 aa, Artificial sequence
  MUC1 TRB CDR3
CASGLGEGRGYTF

SEQ ID NO: 218 - protein, 271 aa, Homo sapiens
  MAGE-A4 TRA
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVGGYSTLTFGKGTVLLVSPDNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 219 - protein, 308 aa, Homo sapiens
  MAGE-A4 TRB
MSISLLCCAAFPLLWAGPVNAGVTQTPKFRILKIGQSMTLQCAQDMNHNYMYWYRQDPGMGLKLIYYSVGAGITDKGEVPNGYNVSRSTTEDFPLRLELAAPSQTSVYFCASSYSRWSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 220 - protein, 11 aa, Artificial sequence
  MAGE-A4 TRA CDR3
CAVGGYSTLTF

SEQ ID NO: 221 - protein, 13 aa, Artificial sequence
  MAGE-A4 TRB CDR3
CASSYSRWSPLHF

SEQ ID NO: 222 - protein, 331 aa, Artificial sequence
  NKG2D.z
MRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREFGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV